(19) United States
(12) Patent Application Publication
(10) Pub. No.: US 2024/0299651 A1
(43) Pub. Date: Sep. 12, 2024

(54) MEDICATION DELIVERY SYSTEM AND METHOD

(71) Applicant: Sadleir Laboratories Pty Ltd, Cottesloe (AU)

(72) Inventors: Paul Harold Martin SADLEIR, Cottesloe (AU); John Willoughby SADLEIR, Cottesloe (AU)

(73) Assignee: Sadleir Laboratories Pty Ltd, Cottesloe (AU)

(21) Appl. No.: 18/570,594

(22) PCT Filed: Jun. 15, 2022
(86) PCT No.: PCT/AU2022/050594

§ 371 Date: Dec. 14, 2023

(30) Foreign Application Priority Data
Jun. 15, 2021 (AU) 2021901792
Jun. 15, 2021 (AU) 2021901794

(51) Int. Cl. A61M 5/145; A61M 5/14; A61M 5/168

(52) U.S. Cl. CPC A61M 5/1456; A61M 5/1407; A61M 5/16804; A61M 2205/3334

(57) ABSTRACT

Medication Delivery System and Method Examples of the present application include an infusion device for controlling a medication delivery apparatus to deliver a pharmaceutical preparation to a patient, the infusion device comprising a processor and a memory storing instructions executable by the processor. The instructions include instructions to: determine, a number of infusion steps (h) that are to be performed within a time window, the time window comprising a first time window and a second time window, wherein a first number of infusion steps (h1) are to be performed within the first time window and a second number of infusion steps (h2) are to be performed within the second time window. To determine, for an infusion step of the first number of infusion steps (h1), a first infusion volume, using a cumulative delivery volume function. To determine, for an infusion step of the second number of infusion steps (h2), a second infusion volume, using a dose function. To control the medication delivery apparatus such that the first infusion volume of a fluid is expelled from the medication delivery apparatus during the infusion step of the first number of infusion steps (h1); and the second infusion volume of the fluid is expelled from the medication delivery apparatus during the infusion step of the second number of infusion steps (h2).

### TECHNICAL FIELD

The present disclosure relates to systems and methods for administering pharmaceutical preparations to patients.

The disclosure has been devised particularly, although not necessarily solely, in relation to administering pharmaceutical preparations to patients in particular test doses for, for example, detecting an adverse reaction during the administration of the pharmaceutical preparation, desensitising the patient to the pharmaceutical preparations or challenging a patient with the pharmaceutical preparation/s to determine if the pharmaceutical preparation/s are responsible for any adverse reaction in the patient.

### BACKGROUND ART

The following discussion of the background art is intended to facilitate an understanding of the present disclosure only. The discussion is not an acknowledgement or admission that any of the material referred to is or was part of the common general knowledge as at the priority date of the application.

Administering a pharmaceutical preparation/s (such as intravenous drugs) to patients has its risks. This is particularly true in patients that may have a drug hypersensitivity reaction to a particular intravenous drug during administration of the particular intravenous drug to these particular patients.

Unfortunately, drug hypersensitivity reactions to particular intravenous drugs are typically unpredictable; and in particular, it is unpredictable the specific dose of the drug that may induce a drug hypersensitivity reaction in a particular patient.

In order to reduce the risk of any patient suffering a life-threatening reaction to a drug, one method of administering a particular intravenous drug is to give the patient a specific dose (referred to as a test dose) that would cause a submaximal adverse response. Upon detection of any submaximal or minor adverse reaction in the particular patient, the process of administering the intravenous drug may be immediately aborted to impede that any more of the pharmaceutical preparation (drug) be administered to the patient and preventing a more serious adverse reaction from developing, or ultimate death of the patient.

However, the practice of administering a test dose is not routine nor recommended. This is particularly true because:
the test dose that will typically elicit a submaximal reaction is typically of the order of 0.01% or 0.1% of the total therapeutic dose to be given to the patient, the preparation of which is time-consuming and difficult; and
test doses that will elicit a detectable submaximal reaction vary between patients, and may be 0.01%, 1%, 10% or 100% of the therapeutic dose.

These two reasons among others make it difficult or even impossible for a clinician to choose the appropriate test dose with which to conduct a trial to confirm whether an adverse reaction will occur during administration of the total therapeutic dose. In particular: administering a test dose that is relatively small may not elicit or result in detection of an adverse reaction in the patient. In contrast, a relatively large dose (above a specific threshold particular to each patient) may cause an adverse reaction that may result in a life-threatening reaction in the patient. This reaction may lead to death of the patient. Thus, administering the test dose may lead to a life-threatening condition that the provision of the test dose had the intention to prevent.

The process for confirming that a particular drug is responsible for a particular adverse reaction in a particular patient by administering a test dose of the particular drug in one or more incremental steps is called Drug Challenge.

Another process where a relative low dose (a test dose) of a drug may be administered to a patient prior to administering the full dose is called Drug Desensitisation. Drug desensitization is the process of administering a test dose below the threshold that will produce an adverse reaction to a patient who is hypersensitive or allergic to a particular drug to induce a state of tolerance and allow administration of the therapeutic dose while avoiding any adverse reaction or inducing only minor non-life threatening reactions.

Typically, drug desensitisation comprises initially administering a dose (the test dose) that is lower than the actual dose that will elicit an adverse reaction in a patient. Subsequently, depending on whether the patient's reaction is favourable to the drug, larger doses are administered to the patient. Administration, typically, occurs at intervals of usually days or weeks; but, on occasions, it may take hours if express desensitisation is required in, for example, emergencies. The process of drug desensitisation is continued until it is certain that the actual dose can be safely administered to the patient without adverse reaction. In particular, for intravenous drugs, administration of the drug occurs as a constant infusion rate of the lower dose for a particular interval, and then the drug is administered as a constant infusion at a higher rate or higher concentration for an interval, and so on, until the therapeutic dose is tolerated.

Unfortunately, due the difficulties in determining what specific percentage of the total therapeutic dose to be administered to the patient is an appropriate test dose for that particular patient, the current practice is to administer intravenous drugs via constant infusion (either brief (‘push’) or over a fixed time period). This has its risks, as mentioned above. Administering the total therapeutic dose of a drug without confirming whether the patients is hypersensitive or allergic to that particular may result in administering a lethal drug dose to a particular patient, or cause a serious negative reaction.

Furthermore, currently any test dose that may be administered to a patient is necessarily done prior to, and separate from, infusion of the therapeutic dose that a particular patient requires. Preparation of separate test doses requires preparation of a multitude of pharmaceutical preparations for each test dose and also for the therapeutic dose. This process is cumbersome and therefore, typically, test doses are not provided to patients. Instead the therapeutic dose is provided to the patient without having tested the reaction of the patient to the drug. This increases the risk that particular patients (that have a drug hypersensitivity reaction to a particular drug) may suffer life threatening conditions while being administered this particular drug. This is particularly true because the current methods for administering the full therapeutic dose (a constant infusion or ‘push’) provide relatively large doses at the start of the infusion process compared to that typically required to cause a serious adverse reaction. This does not allow enough time for the clinician to detect that the patient being infused the pharmaceutical preparation is having a negative (i.e., adverse) reaction to the drug.

### SUMMARY OF DISCLOSURE

According to some examples there is provided a method for delivering an active ingredient into a patient, the method comprising the steps of preparing a pharmaceutical preparation having a particular volume, the pharmaceutical preparation comprising a solvent and therapeutic dose of the active ingredient and administering to a patient the pharmaceutical preparation, wherein the pharmaceutical preparation is administered to the patient in such a manner that at a first stage of administration of the pharmaceutical preparation at least one portion of the therapeutic dose is administered to the patient for detection of a negative reaction in the patient.

In some examples, the predetermined dose profile is such that the dose rate varies over the predetermined infusion time. In some examples, the cumulative dose delivered to the patient increases exponentially, or increases at a rate that increases over time, for at least a portion of the predetermined infusion time.

In some examples, the dose profile is such that there is a first time period between the cumulative dose reaching 0.01% and 0.1% and a second time period between the cumulative dose reaching 0.1% and 1% of the therapeutic dose; and the first period of time and the second period of time are selected from the group comprising: at least 6 minutes, at least 5 minutes, at least 4 minutes, at least 3 minutes, between 2 minutes and 10 minutes, and at least the latent period of adverse reaction.

The processor of the infusion device may control the medication delivery apparatus to deliver the pharmaceutical preparation according to the predetermined profile by controlling an actuator of the infusion device. For example the actuator may controlled to drive a plunger, or a pump, of the medication delivery apparatus such that the pharmaceutical preparation is delivered according to the predetermined dose profile. For instance, the processor may divide the predetermined infusion time into a number of infusion steps and determine a target flow rate or a target output volume for each infusion step such that the predetermined dose profile is realized when the actuator is controlled according to the target flow rate or target output volume for each infusion step. The target flow rates or target output volumes for the infusion steps for a predetermined dose profile may be determined by referring to a lookup table stored in the memory. The lookup table may be populated according to the techniques described herein, for instance calculating the target flow rate or target output volume for each infusion step based on modelling of the predetermined dose profile. In other examples, target flow rate or target output volume for each infusion step may be calculated by the processor in real time.

Some examples provide, an infusion device for controlling a medication delivery apparatus to deliver a pharmaceutical preparation to a patient, the infusion device comprising a processor and a memory storing instructions executable by the processor to: determine, a number of infusion steps (h) that are to be performed within a time window, the time window comprising a first time window and a second time window, wherein a first number of infusion steps (h_1) are to be performed within the first time window and a second number of infusion steps (h_2) are to be performed within the second time window; determine, for an infusion step of the first number of infusion steps (h_1), a first infusion volume, using a cumulative delivery volume function; determine, for an infusion step of the second number of infusion steps (h_2), a second infusion volume, using a dose function; control the medication delivery apparatus such that the first infusion volume of a fluid is expelled from the medication delivery apparatus during the infusion step of the first number of infusion steps (h_1); and the second infusion volume of the fluid is expelled from the medication delivery apparatus during the infusion step of the second number of infusion steps (h_2).

In some examples, a concentration of an active agent in the first infusion volume of the fluid that is to be expelled from the medication delivery apparatus is at least one order of magnitude lower than a concentration of the active agent in the second infusion volume of the fluid that is to be expelled from the medication delivery apparatus.

In some examples, a rate of a cumulative dose of an active agent of the pharmaceutical preparation that is expelled from the medication delivery apparatus increases over the time window.

In some examples, further comprise receiving a plurality of method inputs, wherein at least one of the method inputs is an input of the cumulative delivery volume function and at least one of the method inputs is an input of the dose function.

In some examples, the instructions further comprise: determining a first target flow rate of the infusion step of the first number of infusion steps, based at least in part on the first infusion volume; and determining a second target flow rate of the infusion step of the second number of infusion steps, based at least in part on the second infusion volume.

In some examples, the medication delivery apparatus is controlled such that the first infusion volume of the fluid is expelled from the medication delivery apparatus during the infusion step of the first number of infusion steps, at the first target flow rate.

In some examples, the medication delivery apparatus is controlled such that the second infusion volume is expelled from the medication delivery apparatus during the second infusion step, at the second target flow rate.

In some examples, the instructions further comprise determining a maximum dose time, the maximum dose time being indicative of a time at which a maximum infusion rate threshold is reached.

In some examples, the instructions further comprise determining a transitional time, the transitional time being indicative of a temporal point that divides the first time window and the second time window.

In some examples, the instructions further comprise: determining that the maximum dose time is within the first time window; and controlling the medication delivery apparatus such that a dose rate of the fluid expelled from the medication delivery apparatus after the maximum dose time is at or below the maximum infusion rate threshold.

In some examples, the cumulative delivery volume function is solved analytically to determine the first infusion volume.

In some examples, the instructions further comprise: the cumulative delivery volume function defines at least part of a dose profile for delivering a therapeutic dose of the pharmaceutical preparation to the patient, wherein the cumulative delivery volume function is such that the cumulative dose delivered to the patient increases exponentially, or increases at a rate that increases over time, over a time period between a first time at which 0.1% of the therapeutic dose has been delivered to the patient and a second time at which 10% of the therapeutic dose has been delivered to the patient.

In some examples, the cumulative delivery volume function defines at least part of a dose profile for delivering a therapeutic dose of the pharmaceutical preparation to the patient, wherein the cumulative delivery volume function is such that there is a first time period between the cumulative dose reaching 0.01% and 0.1% of therapeutic dose and a second time period between the cumulative dose reaching 0.1% and 1% of the therapeutic dose; wherein the first period of time and the second period of time are selected from the group comprising: at least 6 minutes, at least 5 minutes, at least 4 minutes, at least 3 minutes between 2 minutes and 10 minutes, and at least the latent period of adverse reaction.

In some examples, there is provided an infusion device for delivering a pharmaceutical preparation to a patient; the infusion device comprising a processor and a memory storing instructions executable by the processor to: receive: a concentration input (Cₚ) that is indicative of a concentration of a pharmaceutical preparation in an active agent chamber of a medication delivery apparatus; a volume input (Vₚ) that is indicative of a volume of the pharmaceutical preparation in the active agent chamber;
a dilution chamber volume input (V_{d}) that is indicative of a volume of a dilution chamber of the medication delivery apparatus; a time input (i) that is indicative of a time window over which the pharmaceutical preparation is to be delivered; determine: a number of infusion steps (h) that are to be performed within at least part of the time window; a first cumulative delivery volume (KV₁), wherein the first cumulative delivery volume (KV₁) is indicative of a cumulative volume of a fluid that is to be expelled from the medication delivery apparatus between an initial time and an initial infusion step time, the initial infusion step time corresponding to a start of a target infusion step of the number of infusion steps (h); a second cumulative delivery volume (KV₂), wherein the second cumulative delivery volume (KV₂) is indicative of a cumulative volume of the fluid that is to be expelled from the medication delivery apparatus between the initial time and a subsequent infusion step time, the subsequent infusion step time corresponding to an end of the target infusion step; and an infusion volume, based at least in part on the first cumulative delivery volume (KV₁) and the second cumulative delivery volume (KV₂), the infusion volume being indicative of a volume of the fluid that is to be expelled from the medication delivery apparatus during the target infusion step; and control the medication delivery apparatus such that the infusion volume of the fluid is expelled from the medication delivery apparatus during the target infusion step.

In some examples, a concentration of an active agent in the target infusion volume of the fluid that is to be expelled from the medication delivery apparatus is at least one order of magnitude higher than a concentration of the active agent in a previous infusion volume of the fluid that is to be expelled from the medication delivery apparatus before the target infusion volume of the fluid.

In some examples, the instructions further comprising determining a target flow rate, based at least in part on the infusion volume of the target infusion step; wherein the plunger is actuated such that the target infusion volume is expelled from the medication delivery apparatus during the target infusion step, at the target flow rate.

In some examples, the target flow rate of the target infusion step is equal to a previous target flow rate of a previous target infusion step that is performed earlier in the time window than the target infusion step.

In some examples, the target flow rate of the target infusion step is equal to a subsequent target flow rate of a subsequent target infusion step that is performed later in the time window than the target infusion step.

In some examples, the first cumulative delivery volume and the second cumulative delivery volume are determined by analytically solving a function which delivers a therapeutic dose of a pharmaceutical preparation to a patient in accordance with a predetermined dose profile.

In some examples, the dose profile is such that the cumulative dose delivered to the patient increases exponentially, or increases at a rate that increases over time, over a time period between a first time at which 0.1% of the therapeutic dose has been delivered to the patient and a second time at which 10% of the therapeutic dose has been delivered to the patient.

In some examples, the dose profile is such that there is a first time period between the cumulative dose reaching 0.01% and 0.1% of therapeutic dose and a second time period between the cumulative dose reaching 0.1% and 1% of the therapeutic dose; wherein the first period of time and the second period of time are selected from the group comprising: at least 6 minutes, at least 5 minutes, at least 4 minutes, at least 3 minutes between 2 minutes and 10 minutes, and at least the latent period of adverse reaction.

In some examples, there is provided an infusion device for use with a medication delivery apparatus comprising an active agent chamber for receiving a pharmaceutical preparation, a dilution chamber for receiving a diluent and a dilution chamber opening through which diluted pharmaceutical preparation can be expelled for intravenous delivery to a patient; the infusion device comprising: a processor and a memory storing instructions executable by the processor to cause the medication delivery apparatus to deliver the pharmaceutical preparation to the patient according to a dose profile, wherein the dose profile delivers a therapeutic dose of the pharmaceutical preparation to the patient over an infusion time, wherein the dose profile comprises a first stage and a second stage; and wherein during the first stage, a concentration of the pharmaceutical preparation in the dilution chamber increases and a dose rate at which the pharmaceutical preparation is delivered to the patient increases until a maximum dose rate for the pharmaceutical preparation is reached; wherein in the second stage, a concentration of the pharmaceutical preparation in the dilution chamber increases and a flow rate at which the diluted pharmaceutical preparation exits the dilution chamber decreases so that the maximum dose rate is not exceeded.

In some examples, the dose rate in the second stage is constant.

In some examples, the dose rate in the second stage is the maximum dose rate for the pharmaceutical preparation.

In some examples, the dose profile further comprises a third stage in which the concentration of the pharmaceutical preparation in the dilution chamber is constant.

In some examples, the dose rate in the third stage is constant and may, for example, be the maximum dose rate for the pharmaceutical preparation.

In some examples, the dose profile is such that for at least a part of the first stage of the dose profile, a cumulative dose of pharmaceutical preparation delivered to the patient increases exponentially over time.

In some examples, the first stage of the dose profile includes a first time period between the cumulative dose reaching 0.01% and 0.1% of the therapeutic dose and a second time period between the cumulative dose reaching 0.1% and 1% of the therapeutic dose; wherein the first period of time and the second period of time are selected from the group comprising: at least 6 minutes, at least 5 minutes, at least 4 minutes, at least 3 minutes between 2 minutes and 10 minutes, and at least the latent period of adverse reaction.

In some examples, the medication delivery device comprises a container, a first plunger and a second plunger in the container arranged so that the active agent chamber is defined by a space between the first plunger and the second plunger and the dilution chamber is defined by a space between the second plunger and a distal end of the container; and wherein the first stage and second stage of the dose profile correspond to a first time window in which the first plunger is moved towards the second plunger so as to expel pharmaceutical preparation from the active agent chamber to the dilution chamber for mixing with diluent and output of diluted pharmaceutical preparation through the dilution chamber opening.

In some examples, the third stage of the dose profile corresponds to a second time window in which the first plunger is in contact with the second plunger and the second plunger is moved towards the distal end of the container so as to reduce the volume of the dilution chamber and expel pharmaceutical preparation from the dilution chamber out through the dilution chamber opening.

In some examples there is provided, a medication delivery apparatus comprising an active agent chamber for receiving a pharmaceutical preparation, a dilution chamber for receiving a diluent and a dilution chamber opening which is to be attached to a conduit of predetermined volume through which diluted pharmaceutical preparation can be delivered to a patient by intravenous infusion; the infusion device comprising: a processor and a memory storing priming instructions executable by the processor to prime the medication delivery device and the conduit of predetermined volume by controlling the medication delivery device to expel pharmaceutical preparation from the active agent chamber into the dilution chamber to mix with the diluent and flow out through the dilution chamber opening into the tubing of known volume so as to fill the conduit of predetermined volume with diluted pharmaceutical preparation in such a manner that the diluted pharmaceutical in the conduit of predetermined volume has a concentration profile in accordance with accordance with a desired dose profile for a first part of the intravenous infusion.

In some examples, the concentration profile is such that a concentration of the diluted pharmaceutical preparation decreases along a length of the conduit of predetermined volume.

In some examples, the memory stores dose delivery instructions executable by the processor to cause the medication delivery apparatus to deliver the pharmaceutical preparation to the patient according to a predetermined dose profile which delivers a therapeutic dose of the pharmaceutical preparation to the patient over an infusion time in manner which facilitates safe detection of an adverse reaction of the patient to the pharmaceutical preparation, or desensitization the patient to the pharmaceutical preparation.

In some examples, the dose profile is such that for at least a part of the first stage of the dose profile, a cumulative dose of pharmaceutical preparation delivered to the patient increases exponentially over time.

In some examples, the first stage of the dose profile includes a first time period between the cumulative dose reaching 0.01% and 0.1% of the therapeutic dose and a second time period between the cumulative dose reaching 0.1% and 1% of the therapeutic dose; wherein the first period of time and the second period of time are selected from the group comprising: at least 6 minutes, at least 5 minutes, at least 4 minutes, at least 3 minutes between 2 minutes and 10 minutes, and at least the latent period of adverse reaction.

In some examples, an infusion rate used to prime the medication delivery apparatus and conduit of predetermined volume is higher than an initial infusion rate of the predetermined dose profile.

In some examples, an infusion device for use with a medication delivery apparatus comprising a syringe including an active agent chamber for receiving a pharmaceutical preparation, a dilution chamber for receiving a diluent and a dilution chamber opening through which diluted pharmaceutical preparation can be expelled for intravenous delivery to a patient; the infusion device comprising: a processor and a memory storing dose delivery instructions executable by the processor to cause the medication delivery apparatus to deliver the pharmaceutical preparation to the patient according to a dose profile which delivers a therapeutic dose of the pharmaceutical preparation to the patient over an infusion time in a manner that facilitates safe detection of an adverse reaction of the patient to the pharmaceutical preparation, or desensitization the patient to the pharmaceutical preparation; and wherein the dose delivery instructions comprise instructions to cause the infusion device to move a plunger of the syringe towards the dilution chamber opening in a plurality of infusion steps which implement the dose profile, wherein a maximum infusion rate is reached after 50% of the infusion time has passed and for infusion steps taking place after a first 3% of the infusion time and prior to the maximum dose rate being reached, each infusion step has a higher dose rate than the previous infusion step.

In some examples, a medication delivery system comprising an infusion device according to any of the above claims together with a medication delivery apparatus as described in any of the above claims, wherein the infusion device is a pump, peristaltic pump, vacuum pump or a syringe driver.

In some embodiments, there is provided a method for delivering a pharmaceutical preparation to a patient, the method comprising: determining, a number of infusion steps (h) that are to be performed within a time window, the time window comprising a first time window and a second time window, wherein a first number of infusion steps (h₁) are to be performed within the first time window and a second number of infusion steps (h₂) are to be performed within the second time window; determining, for an infusion step of the first number of infusion steps (h₁), a first infusion volume, using a cumulative delivery volume function; determining, for an infusion step of the second number of infusion steps (h₂), a second infusion volume, using a dose function; actuating a plunger of a medication delivery apparatus such that the first infusion volume of a fluid is expelled from a medication delivery apparatus during the infusion step of the first number of infusion steps (h₁); and actuating the plunger such that the second infusion volume of the fluid is expelled from the medication delivery apparatus during the infusion step of the second number of infusion steps (h₂).

In some embodiments, a concentration of an active agent in the first infusion volume of the fluid that is to be expelled from the medication delivery apparatus is at least one order of magnitude lower than a concentration of the active agent in the second infusion volume of the fluid that is to be expelled from the medication delivery apparatus.

In some embodiments, a rate of a cumulative dose of an active agent of the pharmaceutical preparation that is expelled from the medication delivery apparatus increases over the time window.

In some embodiments, the method further comprises receiving a plurality of method inputs, wherein at least one of the method inputs is an input of the cumulative delivery volume function and at least one of the method inputs is an input of the dose function.

In some embodiments, the method further comprises: determining a first target flow rate of the infusion step of the first number of infusion steps, based at least in part on the first infusion volume; and determining a second target flow rate of the infusion step of the second number of infusion steps, based at least in part on the second infusion volume.

In some embodiments, the plunger is actuated such that the first infusion volume of the fluid is expelled from the medication delivery apparatus during the infusion step of the first number of infusion steps, at the first target flow rate.

In some embodiments, the plunger is actuated such that the second infusion volume is expelled from the medication delivery apparatus during the second infusion step, at the second target flow rate.

In some embodiments, the method further comprises determining a maximum dose time, the maximum dose time being indicative of a time at which a maximum infusion rate threshold is reached.

In some embodiments, the method further comprises determining a transitional time, the transitional time being indicative of a temporal point that divides the first time window and the second time window.

In some embodiments, the method further comprises: determining that the maximum dose time is within the first time window; and actuating the plunger such that a dose rate of the fluid expelled from the medication delivery apparatus after the maximum dose time is at or below the maximum infusion rate threshold.

In some embodiments, there is provided a pharmaceutical preparation to a patient; the method comprising: receiving: a concentration input (Cₚ) that is indicative of a concentration of a pharmaceutical preparation in an active agent chamber of a medication delivery apparatus; a volume input (Vₚ) that is indicative of a volume of the pharmaceutical preparation in the active agent chamber; a dilution chamber volume input (V_{d}) that is indicative of a volume of a dilution chamber of the medication delivery apparatus; a time input (i) that is indicative of a time window over which the pharmaceutical preparation is to be delivered, the time window comprising a first time window and a second time window; determining a number of infusion steps (h) that are to be performed within the time window, wherein a first number of infusion steps (h₁) are to be performed within the first time window and a second number of infusion steps (h₂) are to be performed within the second time window; determining, for an infusion step of the first number of infusion steps (h₁): a first cumulative delivery volume (KV₁), wherein the first cumulative delivery volume (KV₁) is indicative of a cumulative volume of a fluid that is to be expelled from the medication delivery apparatus between an initial time and an initial infusion step time, the initial infusion step time corresponding to a start of the infusion step of the first number of infusion steps (h₁); a second cumulative delivery volume (KV₂), wherein the second cumulative delivery volume (KV₂) is indicative of a cumulative volume of the fluid that is to be expelled from the medication delivery apparatus between the initial time and a subsequent infusion step time, the subsequent infusion step time corresponding to an end of the infusion step of the first number of infusion steps (h₁); and a first infusion volume, based at least in part on the first cumulative delivery volume (KV₁) and the second cumulative delivery volume (KV₂), the first infusion volume being indicative of a volume of the fluid that is to be expelled from the medication delivery apparatus during the infusion step of the first number of infusion steps (h₁); and determining, for an infusion step of the second number of infusion steps (h₂): a first pharmaceutical dose (Dose_{c1}), wherein the first pharmaceutical dose (Dose_{c1}) is indicative of a cumulative pharmaceutical preparation dose that is to be output by the medication delivery apparatus between the initial time and an initial infusion dose time, the initial infusion dose time corresponding to a start of the infusion step of the second number of infusion steps (h₂); a second pharmaceutical dose (Dose_{c2}), wherein the second pharmaceutical dose (Dose_{c2}) is indicative of a cumulative pharmaceutical preparation dose that is to be output by the medication delivery apparatus between the initial time and a subsequent infusion dose time, the subsequent infusion dose time corresponding to an end of the infusion step of the second number of infusion steps (h₂); a dose target, based at least in part on the first pharmaceutical dose (Dose_{c1}) and the second pharmaceutical dose (Dose_{c2}), the dose target being indicative of a pharmaceutical preparation dose that is to be output by the medication delivery apparatus during the infusion step of the second number of infusion steps (h₂); a concentration estimate, the concentration estimate being indicative of a pharmaceutical preparation concentration of the fluid during the infusion step of the second number of infusion steps (h₂); a second infusion volume, based at least in part on the dose target and the concentration estimate; the second infusion volume being indicative of a volume of the fluid that is to be expelled from the medication delivery apparatus during the infusion step of the second number of infusion steps (h₂); actuating a plunger of the medication delivery apparatus such that the first infusion volume of the fluid is expelled from the medication delivery apparatus during the infusion step of the first number of infusion steps (h₁); and actuating the plunger such that the second infusion volume of the fluid is expelled from the medication delivery apparatus during the infusion step of the second number of infusion steps (h₂).

In some embodiments, the method further comprises: determining a first target flow rate, based at least in part on the first infusion volume; and determining a second target flow rate, based at least in part on the second infusion volume; wherein: the plunger is actuated such that the first infusion volume of the fluid is expelled from the medication delivery apparatus during the infusion step of the first number of infusion steps (h₁), at the first target flow rate; and the plunger is actuated such that the second infusion volume of the fluid is expelled from the medication delivery apparatus during the infusion step of the second number of infusion steps (h₂), at the second target flow rate.

In some embodiments, determining the number of infusion steps (h) comprises calculating:

g×i

where g is a number of infusion steps per minute that are to be performed and i is the time input.

In some embodiments, determining the number of infusion steps (h) comprises receiving the number of infusion steps (h) as an infusion step input.

In some embodiments, the first cumulative delivery volume (KV₁) is determined based at least in part on the initial infusion step time of the infusion step of the first number of infusion steps (h₁), the time input (i), the dilution chamber volume input (V_{d}), the volume input (Vₚ), and a principle branch (W₀) of a Lambert W function.

In some embodiments, determining the first cumulative delivery volume (KV₁) comprises calculating:

$${KV}_{1} = {V}_{d} + {V}_{d} {W}_{0} ( - {e}^{- \frac{( {( {2}^{\frac{30}{i}} )}^{\frac{{t}_{i}}{2}} - 1 ) {V}_{p}}{32767 \times {V}_{d}} - 1} ) + \frac{{V}_{p} ( {( {2}^{\frac{30}{i}} )}^{\frac{{t}_{i}}{2}} - 1 )}{32767}$$

where:
KV₁ is indicative of the first cumulative delivery volume;
V_{d} is the dilution chamber volume input;
W₀ is a principle branch of a Lambert W function;
i is the time input;
tᵢ is indicative of the initial infusion step time of the infusion step of the first number of infusion steps (h₁); and
Vₚ is the volume input.

In some embodiments, the second cumulative delivery volume (KV₂) is determined based at least in part on the subsequent infusion step time of the infusion step of the second number of infusion steps (h₂); the time input (i), dilution chamber volume input (V_{d}), the volume input (Vₚ), and a principle branch (W₀) of a Lambert W function.

In some embodiments, determining the second cumulative delivery volume (KV₂) comprises calculating:

$${KV}_{2} = {V}_{d} + {V}_{d} {W}_{0} ( - {e}^{- \frac{( {( {2}^{\frac{30}{i}} )}^{\frac{{t}_{s}}{2}} - 1 ) {V}_{p}}{32767 \times {V}_{d}} - 1} ) + \frac{{V}_{p} ( {( {2}^{\frac{30}{i}} )}^{\frac{{t}_{s}}{2}} - 1 )}{32767}$$

where:
KV₂ is indicative of the second cumulative delivery volume;
V_{d} is the dilution chamber volume input;
W₀ is a principle branch of a Lambert W function;
i is the time input;
tₛ is indicative of the subsequent infusion step time of the infusion step of the first number of infusion steps (h₁); and
Vₚ is the volume input.

In some embodiments, determining the first infusion volume comprises determining a difference between the second cumulative delivery volume (KV₂) and the first cumulative delivery volume (KV₁).

In some embodiments, the infusion step of the first number of infusion steps (h₁) is performed over a first infusion step duration.

In some embodiments, determining the first target flow rate comprises dividing the first infusion volume by the first infusion step duration.

In some embodiments, the first infusion step duration is different to an infusion step duration of another infusion step of the first number of infusion steps (h₁).

In some embodiments, the first infusion step duration is less than an infusion step duration of another infusion step of the first number of infusion steps (h₁); and the initial infusion step time of the infusion step of the first number of infusion steps (h₁) is closer to the initial time than an initial infusion step time of the another infusion step of the first number of infusion steps (h₁).

In some embodiments, the first pharmaceutical dose (Dose_{c1}) is determined based at least in part on the concentration input (Cₚ) and a value of a flow rate function at the initial infusion dose time.

In some embodiments, determining the first pharmaceutical dose (Dose_{c1}) comprises calculating:

$${Dose}_{c 1} = {C}_{p} \times {\int }_{0}^{{t}_{i 2}} {T}_{i 2} dt$$

where:
Dose_{c1} is indicative of the first pharmaceutical dose;
Cₚ is the concentration input;
tᵢ₂ is indicative of the initial infusion dose time;

$${T}_{i 2} = \frac{2 {V}_{p}}{{2}^{16} - 2} {e}^{\frac{{t}_{i 2}}{2} \times ln ( {2}^{\frac{30}{i}} )} - \frac{2 {V}_{p}}{{2}^{16} - 2} ;$$

i is the time input; and
Vₚ is the volume input.

In some embodiments, the second pharmaceutical dose (Dose_{c2}) is determined based at least in part on the concentration input (Cₚ) and a value of a flow rate function at the subsequent infusion dose time.

In some embodiments, determining the second pharmaceutical dose (Dose_{c2}) comprises calculating:

$${Dose}_{c 2} = {C}_{p} \times {\int }_{0}^{{t}_{s 2}} {T}_{s 2} dt$$

where:
Dose_{c2} is indicative of the second pharmaceutical dose;
Cₚ is the concentration input;
tₛ₂ is the subsequent infusion dose time;

$${T}_{s 2} = \frac{2 {V}_{p}}{{2}^{16} - 2} {e}^{\frac{{t}_{s 2}}{2} \times ln ( {2}^{\frac{30}{i}} )} - \frac{2 {V}_{p}}{{2}^{16} - 2} ;$$

i is the time input; and
Vₚ is the volume input.

In some embodiments, determining the dose target comprises determining a difference between the second pharmaceutical dose (Dose_{c2}) and the first pharmaceutical dose (Dose_{c1}).

In some embodiments, determining the concentration estimate comprises calculating:

$${C}_{d c} = {C}_{p} \times ( 1 - {e}^{- \frac{{V}_{p}}{{V}_{d}}} )$$

where:
C_{dc} is indicative of the concentration estimate;
Cₚ is the concentration input;
Vₚ is the volume input; and
V_{d} is the dilution chamber volume input.

In some embodiments, determining the second infusion volume comprises dividing the dose target by the concentration estimate.

In some embodiments, the infusion step of the second number of infusion steps (h₂) is performed over a second infusion step duration.

In some embodiments, the second infusion step duration is different to an infusion step duration of another infusion step of the second number of infusion steps (h₂).

In some embodiments, the second infusion step duration is less than an infusion step duration of another infusion step of the second number of infusion steps (h₂); and the initial infusion step time of the infusion step of the second number of infusion steps (h₂) is closer to the initial time than an initial infusion step time of the another infusion step of the second number of infusion steps (h₂).

In some embodiments, determining the second target flow rate comprises dividing the second infusion volume by the second infusion step duration.

In some embodiments, the method further comprises determining a transitional time, the transitional time being indicative of a temporal point that divides the first time window and the second time window.

In some embodiments, the method further comprises determining, for a transitional infusion step (hₜ): a transitional infusion volume that is indicative of a volume of the fluid that is to be expelled from the medication delivery apparatus during a first portion of the transitional infusion step (hₜ); a second transitional infusion volume that is indicative of a volume of the fluid that is to be expelled from the medication delivery apparatus during a second portion of the transitional infusion step (hₜ); a transitional step infusion volume, by summing the first transitional infusion volume and the second transitional infusion volume; and a transitional target flow rate, based at least in part on the transitional step infusion volume.

In some embodiments, the transitional infusion step (hₜ) is performed over a transitional infusion step duration that comprises the transitional time.

In some embodiments, the method further comprises actuating the plunger such that the transitional step infusion volume of the fluid is expelled from the medication delivery apparatus during the transitional infusion step (hₜ).

In some embodiments, the transitional infusion step (hₜ) is between the infusion step of the first number of infusion steps (h₁) and the infusion step of the second number of infusion steps (h₂).

In some embodiments, the method further comprises: determining a maximum dose time that is indicative of a time at which a maximum infusion rate threshold is reached; determining that the maximum dose time is in the first time window; determining a cumulative volume of fluid delivered between the initial time and the maximum dose time; actuating the plunger such a dose rate of the infusion is equal to or below the maximum infusion rate threshold over a remainder of the first time window and over the second time window.

In some embodiments, a rate of a cumulative dose of an active agent of the pharmaceutical preparation that is expelled from the medication delivery apparatus increases over the time window.

In some embodiments, there is provided a method for delivering a pharmaceutical preparation to a patient; the method comprising: receiving: a concentration input (Cₚ) that is indicative of a concentration of a pharmaceutical preparation in an active agent chamber of a medication delivery apparatus; a volume input (Vₚ) that is indicative of a volume of the pharmaceutical preparation in the active agent chamber; a dilution chamber volume input (V_{d}) that is indicative of a volume of a dilution chamber of the medication delivery apparatus; a time input (i) that is indicative of a time window over which the pharmaceutical preparation is to be delivered; determining: a number of infusion steps (h) that are to be performed within at least part of the time window; a first cumulative delivery volume (KV₁), wherein the first cumulative delivery volume (KV₁) is indicative of a cumulative volume of a fluid that is to be expelled from the medication delivery apparatus between an initial time and an initial infusion step time, the initial infusion step time corresponding to a start of a target infusion step of the number of infusion steps (h); a second cumulative delivery volume (KV₂), wherein the second cumulative delivery volume (KV₂) is indicative of a cumulative volume of the fluid that is to be expelled from the medication delivery apparatus between the initial time and a subsequent infusion step time, the subsequent infusion step time corresponding to an end of the target infusion step; and an infusion volume, based at least in part on the first cumulative delivery volume (KV₁) and the second cumulative delivery volume (KV₂), the infusion volume being indicative of a volume of the fluid that is to be expelled from the medication delivery apparatus during the target infusion step; actuating a plunger of the medication delivery apparatus such that the infusion volume of the fluid is expelled from the medication delivery apparatus during the target infusion step.

In some embodiments, a concentration of an active agent in the target infusion volume of the fluid that is to be expelled from the medication delivery apparatus is at least one order of magnitude higher than a concentration of the active agent in a previous infusion volume of the fluid that is to be expelled from the medication delivery apparatus before the target infusion volume of the fluid.

In some embodiments, the method further comprises determining a target flow rate, based at least in part on the infusion volume of the target infusion step; wherein the plunger is actuated such that the target infusion volume is expelled from the medication delivery apparatus during the target infusion step, at the target flow rate.

In some embodiments, the target flow rate of the target infusion step is equal to a previous target flow rate of a previous target infusion step that is performed earlier in the time window than the target infusion step.

In some embodiments, the target flow rate of the target infusion step is equal to a subsequent target flow rate of a subsequent target infusion step that is performed later in the time window than the target infusion step.

In some embodiments, determining the first cumulative delivery volume (KV₁) comprises calculating:

$$K {V}_{1} = \frac{1}{i} ( {V}_{d} i + {V}_{d} {iW}_{o} ( - {e}^{- ( \frac{{V}_{p} {t}_{i}}{{V}_{d} i} ) - 1} ) + {V}_{p} {t}_{i} )$$

where:
KV₁ is indicative of the first cumulative delivery volume;
V_{d} is the dilution chamber volume input;
W₀ is a principle branch of a Lambert W function;
i is the time input;
tᵢ is indicative of the initial infusion step time; and
Vₚ is the volume input.

In some embodiments, determining the second cumulative delivery volume (KV₂) comprises calculating:

$$K {V}_{2} = \frac{1}{i} ( {V}_{d} i + {V}_{d} {iW}_{o} ( - {e}^{- ( \frac{{V}_{p} {t}_{s}}{{V}_{d} i} ) - 1} ) + {V}_{p} {t}_{s} )$$

where:
KV₂ is indicative of the second cumulative delivery volume;
V_{d} is the dilution chamber volume input;
W₀ is a principle branch of a Lambert W function;
i is the time input;
tₛ is indicative of the subsequent infusion step time; and
Vₚ is the volume input.

In some embodiments, determining the first cumulative delivery volume (KV₁) comprises solving for KV₁ in:

$$( {V}_{d} {e}^{- \frac{K {V}_{1}}{{V}_{d}}} ) + K {V}_{1} - {V}_{d} = ( \frac{2 {V}_{p}}{{2}^{16} - 2} {e}^{\frac{{t}_{i}}{2} ln ( {2}^{30 / i} )} - \frac{2 {V}_{p}}{{2}^{16} - 2} ) \times β$$

where:
KV₁ is the first cumulative delivery volume;
V_{d} is the dilution chamber volume input;
i is the time input;
β is a volume parameter;
Vₚ is the volume input; and
tᵢ is indicative of the initial infusion step time.

In some embodiments, determining the second cumulative delivery volume (KV₂) comprises solving for KV₂ in:

$$( {V}_{d} {e}^{- \frac{K {V}_{2}}{{V}_{d}}} ) + K {V}_{2} - {V}_{d} = ( \frac{2 {V}_{p}}{{2}^{16} - 2} {e}^{\frac{{t}_{s}}{2} ln ( {2}^{30 / i} )} - \frac{2 {V}_{p}}{{2}^{16} - 2} ) \times β$$

where:
KV₂ is the second cumulative delivery volume;
V_{d} is the dilution chamber volume input;
i is the time input;
β is a volume parameter;
Vₚ is the volume input; and
tₛ is indicative of the subsequent infusion step time.

In some embodiments, the volume parameter (β) is equal to

$${V}_{p} - {V}_{d} ( 1 - {e}^{- \frac{{V}_{p}}{{V}_{d}}} ) .$$

In some embodiments, the method further comprises:
calculating an increased volume input Vₚ₂ by solving for Vₚ₂ in:

$${V}_{p 2} = {V}_{d} + {V}_{d} {W}_{0} ( - {e}^{- \frac{( ( {2}^{15} ) - 1 ) {V}_{0}}{32767 \times {V}_{d}} - 1} ) + \frac{{V}_{0} ( ( {2}^{15} ) - 1 )}{32767}$$

where:
V₀ is an intended delivery volume of the pharmaceutical preparation;
V_{d} is the dilution chamber volume input;
W₀ is a principle branch of a Lambert W function; and
Vₚ₂ is the increased volume input, the increased volume input being associated with the intended delivery volume of the pharmaceutical preparation; and
the method further comprises substituting the increased volume input Vₚ₂ for the volume input Vₚ.

In some embodiments, determining the infusion volume comprises determining a difference between the second cumulative delivery volume (KV₂) and the first cumulative delivery volume (KV₁).

In some embodiments, determining the target flow rate comprises dividing the infusion volume by an infusion step duration of the target infusion step.

In some embodiments, a rate of a cumulative dose of an active agent of the pharmaceutical preparation that is expelled from the medication delivery apparatus increases over the time window.

In some embodiments, there is provided a method for delivering a pharmaceutical preparation to a patient, the method comprising: determining, a number of infusion steps (h) that are to be performed within a time window, the time window comprising a first time window and a second time window, wherein a first number of infusion steps (h₁) are to be performed within the first time window and a second number of infusion steps (h₂) are to be performed within the second time window; determining, for an infusion step of the first number of infusion steps (h₁), a first infusion volume, using a first cumulative delivery volume function; determining, for an infusion step of the second number of infusion steps (h₂), a second infusion volume, using a second cumulative delivery function; actuating a plunger of a medication delivery apparatus such that the first infusion volume of a fluid is expelled from a medication delivery apparatus during the infusion step of the first number of infusion steps (h₁); and actuating the plunger such that the second infusion volume of the fluid is expelled from the medication delivery apparatus during the infusion step of the second number of infusion steps (h₂).

In some embodiments, a concentration of an active agent in the first infusion volume of the fluid that is to be expelled from the medication delivery apparatus is at least one order of magnitude lower than a concentration of the active agent in the second infusion volume of the fluid that is to be expelled from the medication delivery apparatus.

In some embodiments, a rate of a cumulative dose of an active agent of the pharmaceutical preparation that is expelled from the medication delivery apparatus increases over the time window.

In some embodiments, the method further comprises receiving a plurality of method inputs, wherein at least one of the method inputs is an input of the first cumulative delivery volume function and at least one of the method inputs is an input of the second cumulative delivery volume function.

In some embodiments, the method further comprises: determining a first target flow rate of the infusion step of the first number of infusion steps, based at least in part on the first infusion volume; and determining a second target flow rate of the infusion step of the second number of infusion steps, based at least in part on the second infusion volume.

In some embodiments, the plunger is actuated such that the first infusion volume of the fluid is expelled from the medication delivery apparatus during the infusion step of the first number of infusion steps, at the first target flow rate.

In some embodiments, the plunger is actuated such that the second infusion volume is expelled from the medication delivery apparatus during the second infusion step, at the second target flow rate.

In some embodiments, the method further comprises determining a maximum dose time, the maximum dose time being indicative of a dose temporal point at which a maximum infusion rate threshold is reached.

In some embodiments, the method further comprises determining a transitional time, the transitional time being indicative of a temporal point that divides the first time window and the second time window.

In some embodiments, the method further comprises: determining that the maximum dose time is within the first time window; and actuating the plunger such that a dose rate of the fluid expelled from the medication delivery apparatus after the maximum dose time is at or below the maximum infusion rate threshold.

In some embodiments, there is provided a method for delivering a pharmaceutical preparation to a patient; the method comprising: receiving: a concentration input (Cₚ) that is indicative of a concentration of a pharmaceutical preparation in an active agent chamber of a medication delivery apparatus; a volume input (Vₚ) that is indicative of a volume of the pharmaceutical preparation in the active agent chamber; a dilution chamber volume input (V_{d}) that is indicative of a volume of a dilution chamber of the medication delivery apparatus; a time input (i) that is indicative of a time window over which the pharmaceutical preparation is to be delivered, the time window comprising a first time window and a second time window; determining a number of infusion steps (h) that are to be performed within the time window, wherein a first number of infusion steps (h₁) are to be performed within the first time window and a second number of infusion steps (h₂) are to be performed within the second time window; determining, for an infusion step of the second number of infusion steps (h₂): a third cumulative delivery volume (KV₃), wherein the third cumulative delivery volume (KV₃) is indicative of a cumulative volume of a fluid that is to be expelled from a medication delivery apparatus between an initial time and a second initial infusion step time, the second initial infusion step time corresponding to a start of the infusion step of the second number of infusion steps (h₂); a fourth cumulative delivery volume (KV₄), wherein the fourth cumulative delivery volume (KV₄) is indicative of a cumulative volume of the fluid that is to be expelled from the medication delivery apparatus between the initial time and a second subsequent infusion step time, the second subsequent infusion step time corresponding to an end of the infusion step of the second number of infusion steps (h₂); and a second infusion volume, based at least in part on the third cumulative delivery volume (KV₃) and the fourth cumulative delivery volume (KV₄); the second infusion volume being indicative of a volume of the fluid that is to be expelled from the medication delivery apparatus during the infusion step of the second number of infusion steps (h₂); and actuating a plunger of the medication delivery apparatus such that the second infusion volume of the fluid is expelled from the medication delivery apparatus during the infusion step of the second number of infusion steps (h₂).

In some embodiments, the method further comprises: determining a second target flow rate, based at least in part on the second infusion volume; wherein the plunger is actuated such that the second infusion volume is expelled from the medication delivery apparatus during the second infusion step, at the second target flow rate.

In some embodiments, a rate of a cumulative dose of an active agent of the pharmaceutical preparation that is expelled from the medication delivery apparatus increases over the time window.

In some examples there is provided a non-transitory machine readable storage medium storing instructions executable by a processor to perform any of the above methods or storing any of the instructions of functions described above in relation to the infusion device and medication delivery apparatus. Any one or more features of any the examples, methods, devices and systems described herein may be combined unless explicitly stated otherwise or unless logic dictates otherwise.

### BRIEF DESCRIPTION OF THE DRAWINGS

Further features of the present disclosure are more fully described in the following description of several non-limiting embodiments thereof. This description is included solely for the purposes of exemplifying the present disclosure. It should not be understood as a restriction on the broad summary, disclosure or description of the disclosure as set out above. The description will be made with reference to the accompanying drawings in which:

FIG. 1a is a perspective view of a particular arrangement of a medication delivery apparatus for delivery of a pharmaceutical preparation in accordance with a first embodiment of the disclosure;

FIG. 1b is a block diagram of a particular arrangement of a medication delivery apparatus for delivery of a pharmaceutical preparation, according to some embodiments;

FIG. 2 is a perspective view of a particular arrangement of an apparatus for delivery of a pharmaceutical preparation (a medication delivery apparatus), according to some embodiments;

FIGS. 3 to 11e have been deleted. So the next figure after FIG. 2 is FIG. 12a

FIG. 12a depicts a flowchart illustrating a method of delivering a therapeutic dose of a drug, according to some embodiments, which may be referred to as a Tansy method;

FIG. 12b depicts a flowchart illustrating the Tansy method including the process of programming the infusion pump, according to some embodiments;

FIG. 13a depicts a flowchart illustrating a method of delivering the therapeutic dose of a drug, according to some embodiments, which may be referred to as a Sadleir method;

FIG. 13b depicts a flowchart illustrating the Sadleir method, comprising a Sadleir function configured to enable calculation of the infusion rates and volumes delivered at various points in time during the Sadleir method, according to some embodiments;

FIG. 13c depicts a flowchart illustrating a method of approximating the infusion rates and volumes calculated in FIG. 13b, using an infusion pump, according to some embodiments;

FIG. 13d illustrates utilisation of the flowchart of FIG. 13b, for an example where the Sadleir method was used for each interval n in the first 0.04 minutes of a 30 minute infusion of 50 mL of pharmaceutical preparation, each interval n in the first 0.04 minutes of the infusion. Illustrated are the value of: the target dose to be delivered (the modified Tansy function dose), the flow rates (infusion rate) as dictated by the Sadleir function, the concentration in dilution chamber and the % dose delivered in each interval, n;

FIGS. 14a (logarithmic y axis scale) and 14b (linear y axis scale) illustrate rates of drug administration, comparing a constant infusion method and the Tansy method for an infusion duration of 30 minutes;

FIGS. 15a (logarithmic y axis scale) and 15b (linear y axis scale) illustrate the difference in cumulative dose administered at each stage of a 30 minute infusion by the infusion method in accordance with the first embodiment of the disclosure (referred to as the Tansy Method) versus a constant infusion method;

FIG. 16 tabulates the infusion times and cumulative percentage of total dose delivered to the patient for a 30 minute infusion of 50 mL of pharmaceutical preparation with the constant infusion method, Tansy method and Sadleir method (using the same initial pharmaceutical preparation concentration and a 10 ml dilution chamber, t=1200/min);

FIGS. 17a (calculating the Sadleir function using 60 integration intervals per minute, i.e. τ=60) and 17c (calculating the Sadleir function using 1200 integration intervals per minute, i.e. τ=1200) illustrate variations of flow rates for different instances of the second embodiment of the disclosure that differ with respect to each as a consequence of selection of different initiating interval rates (30 minute infusion duration, 10 ml dilution chamber, 50 ml pharmaceutical preparation volume);

FIGS. 17b and 17d illustrate the differences in minimum flow rates for the Sadleir function as a consequence of the different initiating interval rates in FIGS. 17a and 17c, respectively;

FIG. 17e shows a graph plotting the value of minimum flow rate for each of the instances (shown in FIG. 17d) of the Sadleir method that differ with respect to each other in the initiating interval rate;

FIG. 18 illustrates the volume of administered drug during the first minute of Sadleir method according to different degrees of precision of calculation (number of integration intervals per minute, or tau), including or not including the volume of the initiating interval;

FIGS. 19a (linear y axis scale) and 19b (logarithmic y axis scale) illustrate the rates of infusion of the pharmaceutical preparation fluid from the dilution chamber into the patient when using the Tansy method for 50 ml infusions over various example durations of infusion (20 minutes, 25 minutes, 30 minutes, 45 minutes, 60 minutes, 120 minutes and 180 minutes);

FIGS. 20a to 21c have been deleted so the next figure after FIG. 19b is FIG. 22a.

FIG. 22a is a table of the calculated values of instantaneous rate, cumulative volume delivered and cumulative dose delivered for the Tansy method and Sadleir method at 45-second intervals over a 30-minute infusion of 50 mL pharmaceutical preparation; in this particular example the Sadleir function values were calculated using an integration interval of duration 50 milliseconds (τ=1200/min) and a dilution chamber of 10 mL volume;

FIGS. 22b and 22c illustrate the difference in pharmaceutical preparation fluid injection or infusion rates (ml/min) when using the first (Tansy) or second (Sadleir with 10 ml dilution chamber) embodiments of the disclosure for a 50 ml infusion over 30 minutes wherein FIG. 22b illustrates the first 15 minutes of the 30 minute infusion);

FIGS. 22d and 22e illustrate the difference in cumulative volume infused from the pharmaceutical preparation fluid syringe or container over the course of a 30 minute infusion when using the first (Tansy) or second (Sadleir with 10 ml dilution chamber) embodiments of the disclosure for a 50 ml infusion, wherein FIG. 22d illustrate the first 15 minutes of a 30 minute infusion);

FIGS. 23a to 29 have been deleted.

FIG. 30 shows a side view of a medication delivery apparatus, according to some embodiments;

FIG. 31 illustrates the process of filling the medication delivery apparatus, according to some embodiments;

FIG. 32 shows a side perspective view of the medication delivery apparatus shown in FIG. 30 filled with active agent and diluent, according to some embodiments;

FIG. 33 is a perspective view of the medication delivery apparatus shown in FIG. 32 during mounting on an infusion driver in the form of a syringe driver, according to some embodiments;

FIG. 34a illustrates a process for mixing the active agent and the diluent within the dilution chamber, according to some embodiments;

FIG. 34a(i) illustrates a block diagram of the medication delivery system of FIGS. 30 to 34a, according to some embodiments;

FIG. 34b illustrates a method of operation of the medication delivery apparatus, according to some embodiments;

FIG. 34c is a block diagram for calculating a method of delivering a therapeutic dose of a drug, according to some embodiments, which may be referred to as a Diocles infusion protocol or a Diocles method. The Diocles method is used during operation of the medication delivery apparatus depicted in FIGS. 30 to 41 while being mounted on an infusion device in the form of a syringe driver;

FIG. 34d is a flowchart illustrating a method of approximating the infusion rates and volumes calculated in FIG. 34c, using an infusion pump, according to some embodiments;

FIGS. 35 to 41 have been deleted.

FIG. 42 shows a side view of the medication delivery apparatus filled with active agent and diluent, according to some embodiments;

FIG. 43 shows a side view of the medication delivery apparatus, filled with active agent and diluent, being fed with the active agent remotely from the syringe driver, according to some embodiments;

FIG. 43b illustrates the method of operation of the medication delivery apparatus depicted in FIG. 43a, according to some embodiments;

FIG. 43c is a block diagram illustrating a method of delivering a therapeutic dose of a drug, according to some embodiments. The method may be for calculating a Sadleir infusion protocol used during operation of dilution chamber depicted in FIG. 43a;

FIG. 43d is a flowchart illustrating a method of approximating the infusion rates and volumes calculated in FIG. 43c, using an infusion pump, according to some embodiments;

FIGS. 44 to 48 have been deleted.

FIGS. 49a to 49h illustrate results of an example infusion performed according to the Diocles method;

FIGS. 50 to 54 have been deleted.

FIG. 55 is a process flow diagram of a method 5500 for delivering a pharmaceutical preparation to a patient, according to some embodiments;

FIG. 56 is a process flow diagram of a method 5600 for delivering a pharmaceutical preparation to a patient, according to some embodiments;

FIG. 57 is a process flow diagram of a method 5700 for delivering a pharmaceutical preparation to a patient, according to some embodiments;

FIG. 58 is a illustrates a chart of an infusion rate over an infusion, a chart of a cumulative volume delivered over the infusion, a chart of a concentration of an active agent in a dilution chamber of a medication delivery apparatus over the infusion, and a drug administration rate for an example infusion of Vancomycin, according to some embodiments;

FIG. 59 illustrates a chart of an infusion rate over an infusion, a chart of a cumulative volume delivered over the infusion, a chart of a concentration of an active agent in a dilution chamber of a medication delivery apparatus over the infusion, and a drug administration rate for an example infusion, according to some embodiments; and

FIG. 60 illustrates a chart of an infusion rate over an infusion, a chart of a cumulative volume delivered over the infusion, a chart of a concentration of an active agent in a dilution chamber of a medication delivery apparatus over the infusion, and a drug administration rate for an example infusion, according to some embodiments.

It should be noted that the figures are schematic only and the location and disposition of the components can vary according to the particular arrangements of the embodiments of the present disclosure as well as of the particular applications of the present disclosure.

### DESCRIPTION OF EMBODIMENTS

The methods and system in accordance with the present embodiments of the disclosure allow administration in a single infusion process of a therapeutic dose of a particular drug in conjunction with test doses. These methods and systems are particularly useful because they do not require given patients a multitude of test doses prior the infusion of the therapeutic dose. Instead, the test doses are given during infusion of the full therapeutic dose due to the test doses being part of the therapeutic dose. Provision of test doses without the use of the embodiments of the present embodiments of the disclosure requires (1) preparation of a multitude of pharmaceutical preparations (including the test doses) having different concentrations and (2) infusing the multitude of pharmaceutical preparations for each test dose to the patient for each of the pharmaceutical preparations. This process of infusing a multitude of pharmaceutical preparations containing test doses (prior infusion of the therapeutic dose) can be a cumbersome and time consuming task and can be unsuitable in situations where infusion of the therapeutic dose must be done immediately to, for example, preserve the life of a patient.

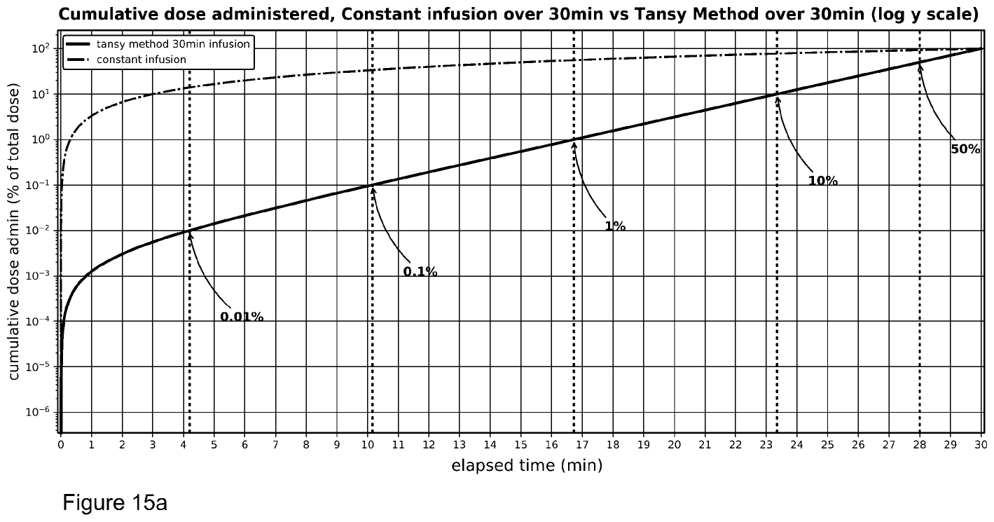

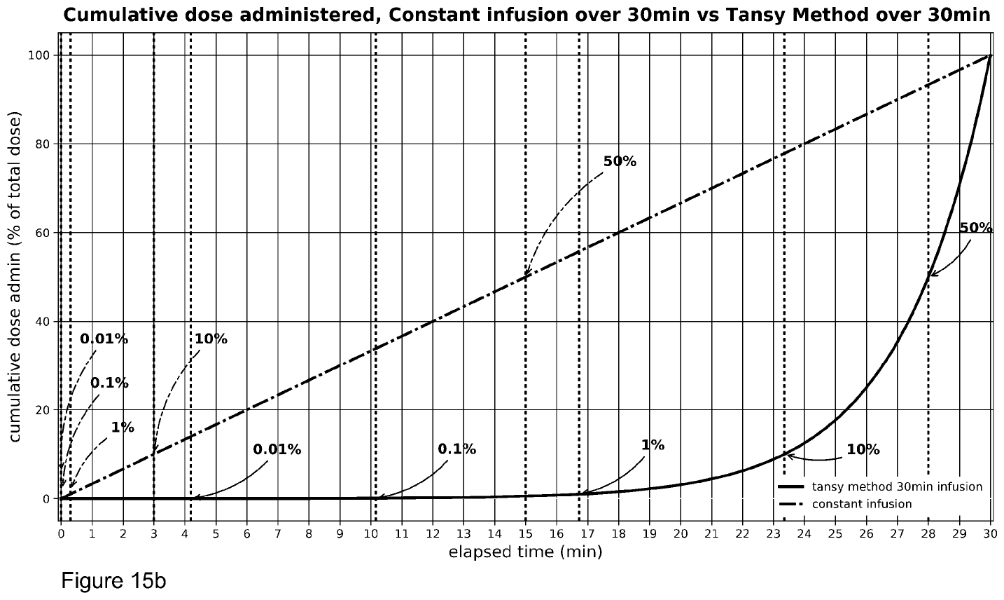

These methods and systems in accordance with the present disclosure are particularly useful because they increase the likelihood that an adverse reaction will be recognized before a specific dose (a particular amount of drug), that will induce a more severe negative reaction in the patient, has been administered (see FIGS. 15a and 15b). Thus, these methods and systems are adapted to safely provide the therapeutic dose to a patient when one or more specific doses, that will cause a submaximal reaction in the patient, are not known.

The present embodiments of the disclosure provide methods and system for the provision of test doses of a drug to a particular patient who may suffer a hypersensitivity reaction (hypersensitivity, or allergy or other adverse reaction), preferably with a short latency.

It will be understood that the term “active agent” as used in the description, may correspond to, or also be referred to as an “active ingredient” or a “drug”. That is, throughout this disclosure, the terms “active ingredient”, “active agent” and “drug” have been used to describe the active agent that is to be administered to a patient. In some embodiments, a pharmaceutical preparation can be delivered to a patient. The pharmaceutical preparation may comprise the active agent. The pharmaceutical preparation may also comprise one or more other constituents. For example, the pharmaceutical preparation may comprise a solvent. That is, in some embodiments, the pharmaceutical preparation may comprise the active agent and a solvent. The pharmaceutical preparation may comprise the active agent at a particular concentration. This may be referred to as an active agent concentration. The pharmaceutical preparation may be a solution. It will be understood that in some embodiments, the term “drug” as used in the description may correspond to the active agent of the “pharmaceutical preparation”.

The methods and system in accordance with the first embodiment of the disclosure uses a particular function (Tansy function) for delivering (infusing) sequentially to a patient a wide range of test doses of a pharmaceutical preparation, with the dose(s) increasing during the duration of the infusion. This has an objective of overcoming the problem of the sensitivity to a particular drug in a patient when the threshold for this sensitivity is not known prior to the administration of the particular drug. In some embodiments, during the entire duration of the infusion, a full therapeutic dose is provided with a portion of the therapeutic dose being used as one or more test doses. In this manner, there is no need of interrupting the administration of the therapeutic dose by, for example, providing at a first stage, a test dose contained in a particular pharmaceutical preparation; and then, after having confirmed that the patient will have no negative reaction to the drug, continuing to infuse the pharmaceutical preparation to the patient. Thus, according to the first embodiment of the disclosure only a single pharmaceutical preparation is required to provide the full therapeutic dose including any test doses.

The method and system in accordance with a second embodiment of the disclosure also allows the administration to a patient a single pharmaceutical preparation to provide the full therapeutic dose including the test doses. However, as will be explained below, the method and system in accordance with a second embodiment of the disclosure allows the accuracy with which the pharmaceutical preparation is provided to the patient to be increased. It does so by permitting an increase in the initial flow rate of a pharmaceutical preparation driven by an infusion driver 14, when compared to a flow rate of the pharmaceutical preparation when using the method and system in accordance with the first embodiment of the disclosure (the Tansy Method). In some embodiments, the infusion driver 14 may be, a syringe driver or peristaltic pump or similar drug infusion pump. In some embodiments, the infusion driver is in the form of an infusion device. In some embodiments, an infusion device comprises the infusion driver.

Increasing the flow rate with which the pharmaceutical preparation exits the infusion driver 14 when the flow rate is relatively low increases the accuracy of the administration process of the pharmaceutical preparation because it is known that infusion drivers 14 do not deliver accurately pharmaceutical preparations at relative low rates such as those that occur when using the Tansy function.

However, the methods and systems in accordance with a second embodiment of the disclosure use another function (a Sadleir function) to control a rate at which the pharmaceutical preparation is delivered (infused) to the patient. Infusing the pharmaceutical preparation as dictated by the Sadleir function allows the pharmaceutical preparation to be given at a higher initial flow rate (with respect to the Tansy method) as a consequence of the use of a dilution chamber 32 that is located between an active agent chamber and the patient. The pharmaceutical preparation flows through the dilution chamber 32 prior entering the patient. The dilution chamber 32 comprises a diluent for mixing with the pharmaceutical preparation entering the dilution chamber 32. The dilution chamber 32 is adapted to ensure rapid mixing of the pharmaceutical preparation with the diluent in the dilution chamber 32. The mixing is initially done by varying repeatedly the flow rates between lower and higher values during a second priming step (occurring when the initial mixed pharmaceutical preparation is infused from the dilution chamber 32 via the conduit 30b to the patient intravenous access point). Subsequent mixing and dilution occurs within the dilution chamber 32 during the course of the delivery of the Sadleir function infusion program. This may include the use of an injection catheter within the dilution chamber 32 that includes a flexible sleeve to allow dynamic adjustment to resistance according to the flow rates.

In particular, using the Sadleir method permits reducing the concentration of the pharmaceutical preparation entering the patient at the start of the infusion process when compared to the Tansy method. The Sadleir method therefore requires a higher initial flow rate to give a similar dosing profile as that of the Tansy function, and a higher minimum infusion rate. It is important to note that the pharmaceutical drug dosing profile in accordance with the Sadleir method is the same as that delivered by the Tansy method, except that the dose in the Sadleir method at any point in time during the infusion is reduced by a fixed fraction to compensate for an amount of the drug remaining in the dilution chamber 32 at the end of the infusion process. However, it is important to note that using either the Tansy method or the Sadleir method will result in separation of orders of magnitude of cumulative dose of active ingredient of the pharmaceutical preparation.

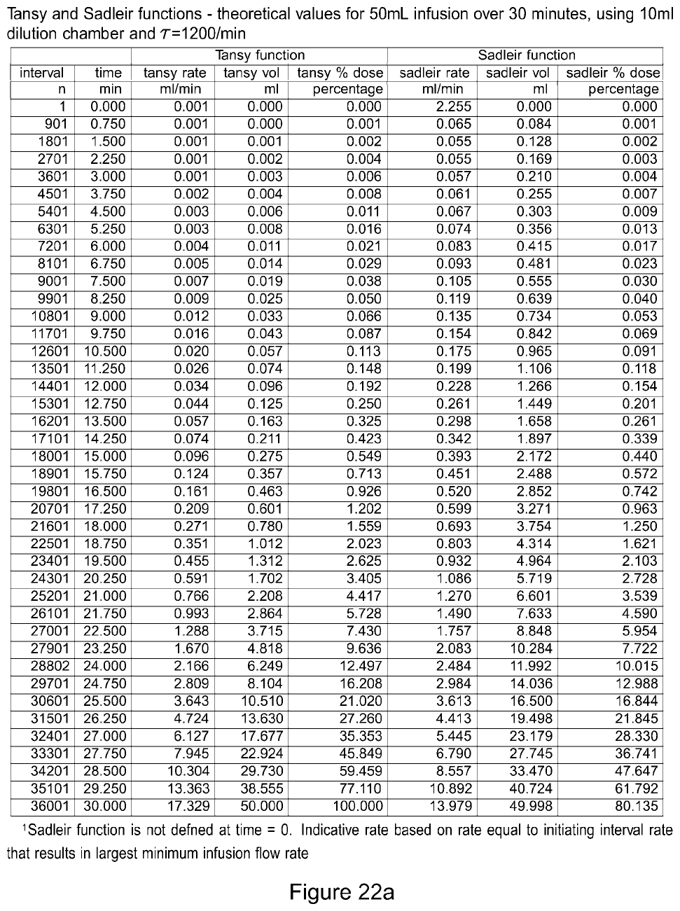

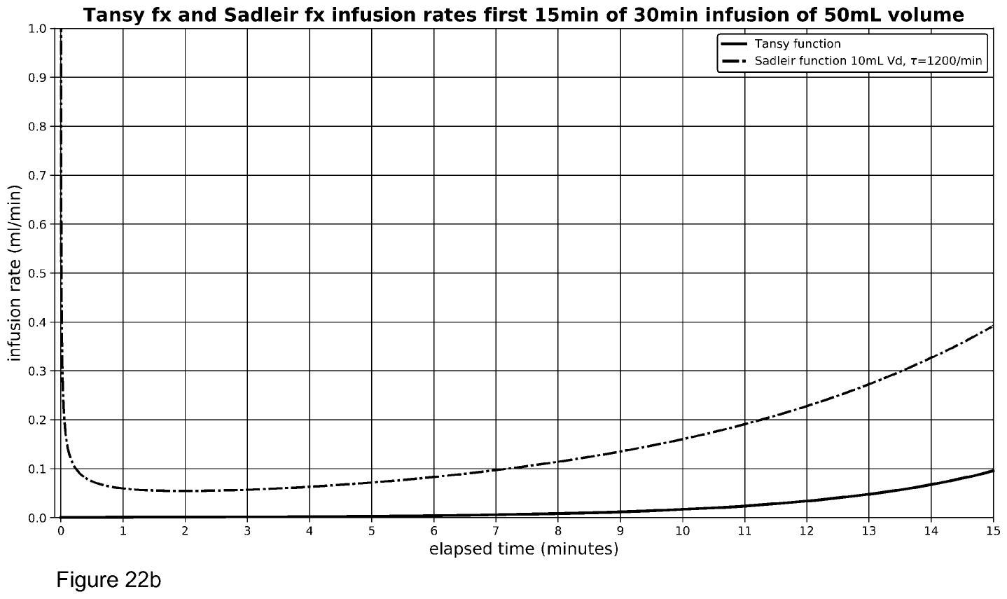

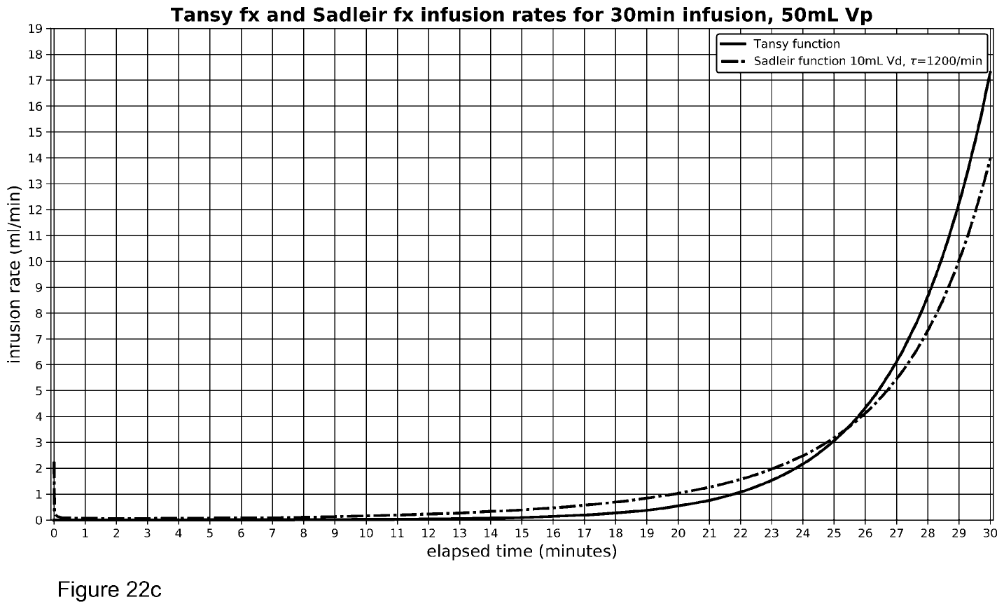

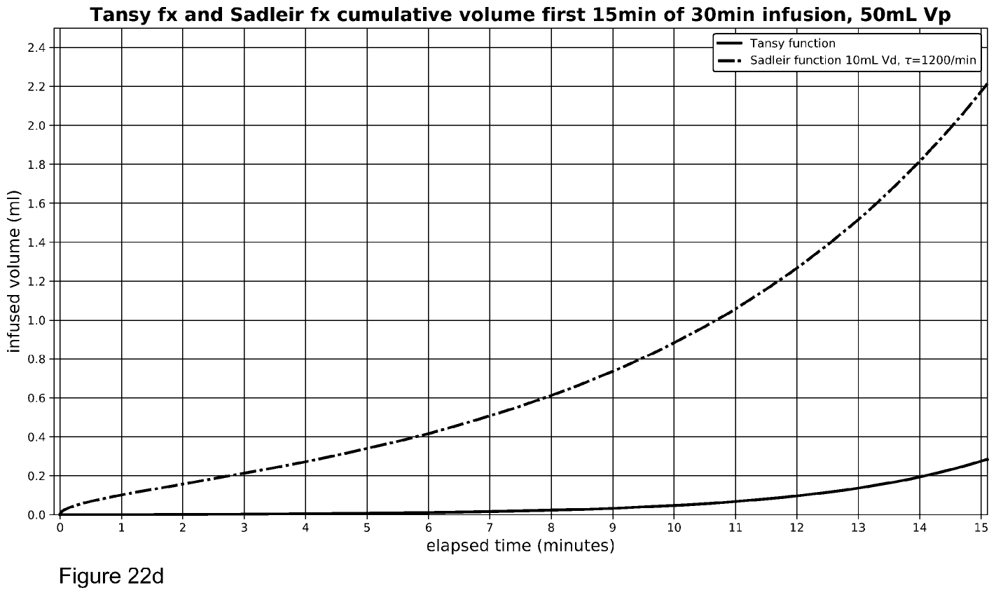

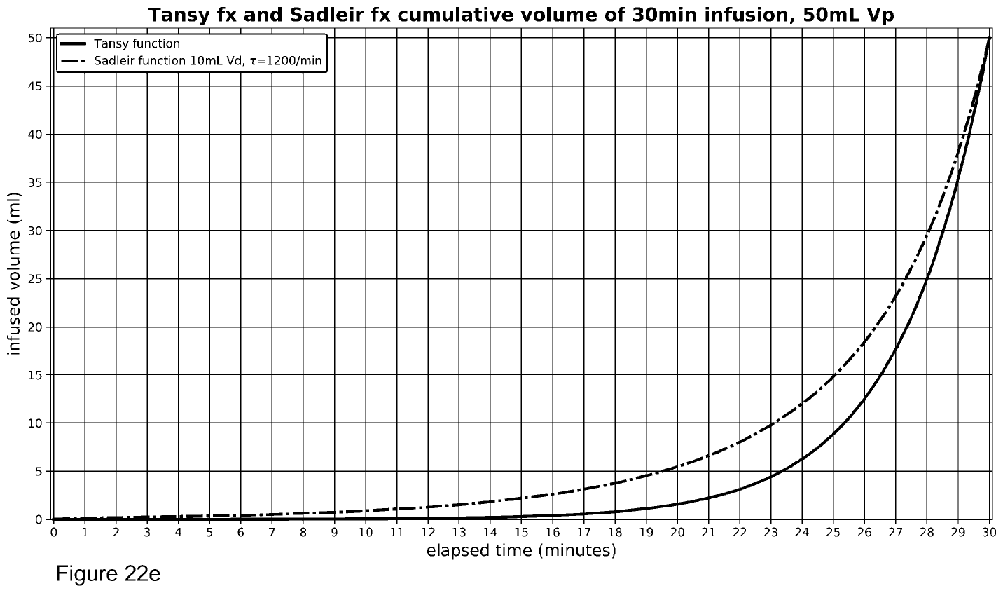

FIGS. 22b and 22c illustrate the difference in pharmaceutical preparation fluid injection or infusion rates (ml/min) when using the first (Tansy) or second (Sadleir with 10 ml dilution chamber) embodiments of the disclosure for a 50 ml infusion over 30 minutes. FIG. 22b illustrates the first 15 minutes of the 30 minute infusion, with the pharmaceutical preparation flow rate (in ml/min) being greater for the Sadleir method early in the infusion, with the Tansy method having a higher flow rate at the end of the infusion.

FIGS. 22d and 22e illustrate the difference in cumulative volume infused from the pharmaceutical preparation fluid syringe or container over the course of a 30 minute infusion when using the first (Tansy) or second (Sadleir with 10 ml dilution chamber) embodiments of the disclosure for a 50 ml infusion. FIG. 22d illustrates the first 15 minutes of a 30 minute infusion. The cumulative volume infused at a point in time is intended to mean the total volume of pharmaceutical preparation that has been infused into the patient since the beginning of the infusion until that point in time.

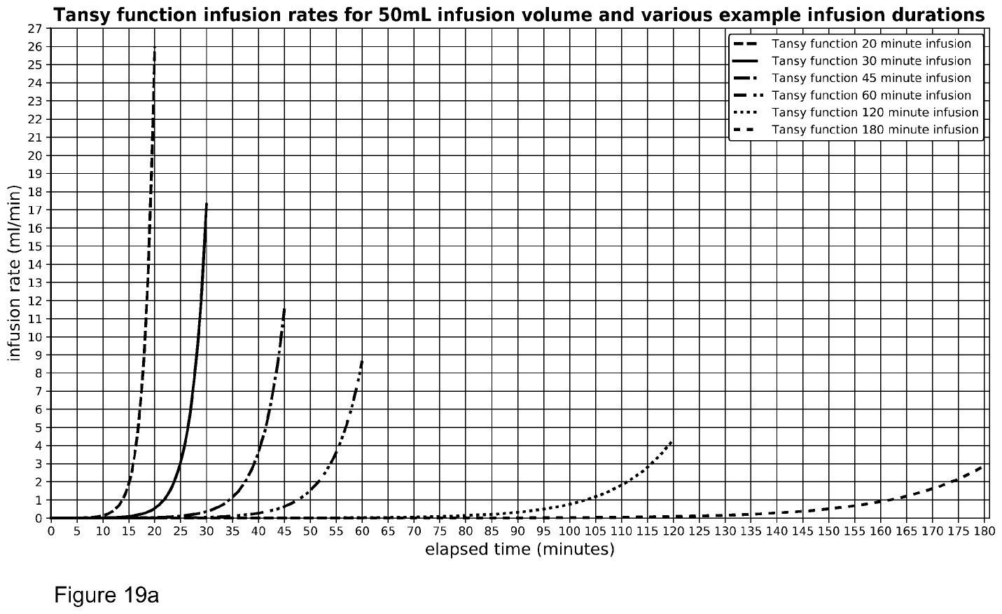

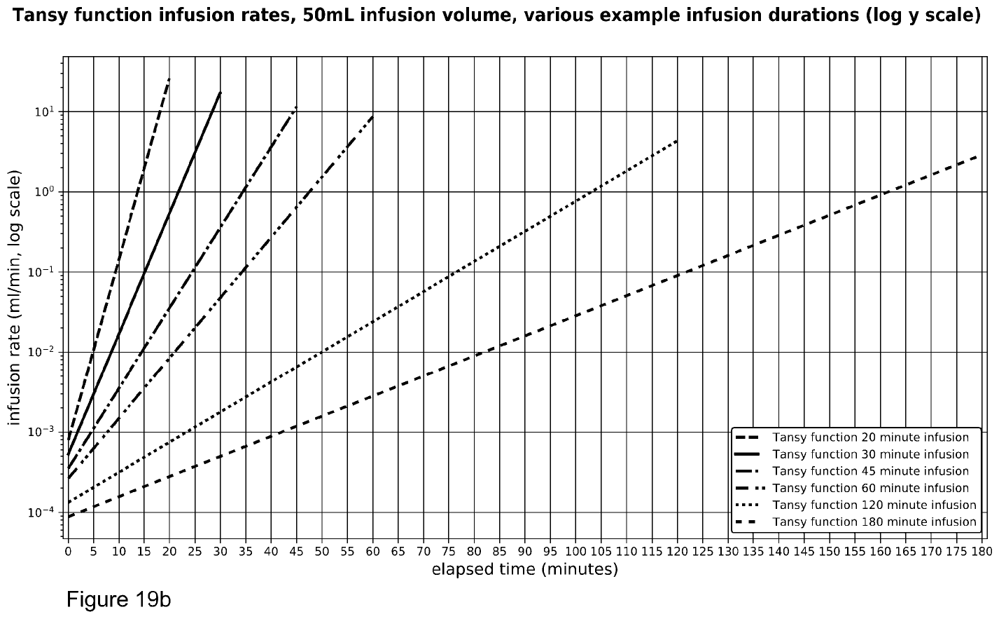

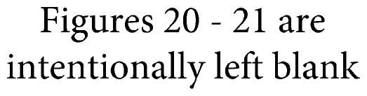

In accordance with the first embodiment of the disclosure, there is provided a method and a system that provide a pharmaceutical preparation to a patient. A flow rate of the pharmaceutical preparation follows a curve of a Tansy function (see FIGS. 19a and 19b). This method (referred to as a Tansy Method) comprises the step of providing the drug at a particular flow rate dictated by the Tansy function.

### Medication Delivery System

The medication delivery system 1 comprises a medication delivery apparatus 10 for the provision of the pharmaceutical preparation. The medication delivery apparatus 10 may be referred to herein as an apparatus 10. The medication delivery apparatus 10 is configured to provide a pharmaceutical preparation at, or approximating the flow rate dictated by the Tansy function.

The medication delivery system 1 comprises an infusion device. The infusion device may be in the form of an infusion driver 14. In some embodiments, the apparatus 10 may comprise the infusion driver 14 (such as syringe driver, peristaltic pump Plum pump or similar drug infusion pump). The infusion device may comprise or be in the form of a vacuum infusion device. The infusion device may apply an infusion pressure (i.e. a vacuum pressure) at a dilution chamber opening 110 of the medication delivery apparatus. The infusion pressure may be a negative pressure.

The infusion driver 14 comprises a control unit for controlling the flow rate at which the infusion driver 14 delivers the drug (pharmaceutical preparation) from a syringe or bag via a generic length of tubing to the patient. The control unit comprises hardware and software for controlling the infusion driver 14 to deliver the drug at the flow rate established by the Tansy function. The software comprises a plurality of instructions for running an algorithm designed to calculate the flow rate as dictated by the Tansy function.

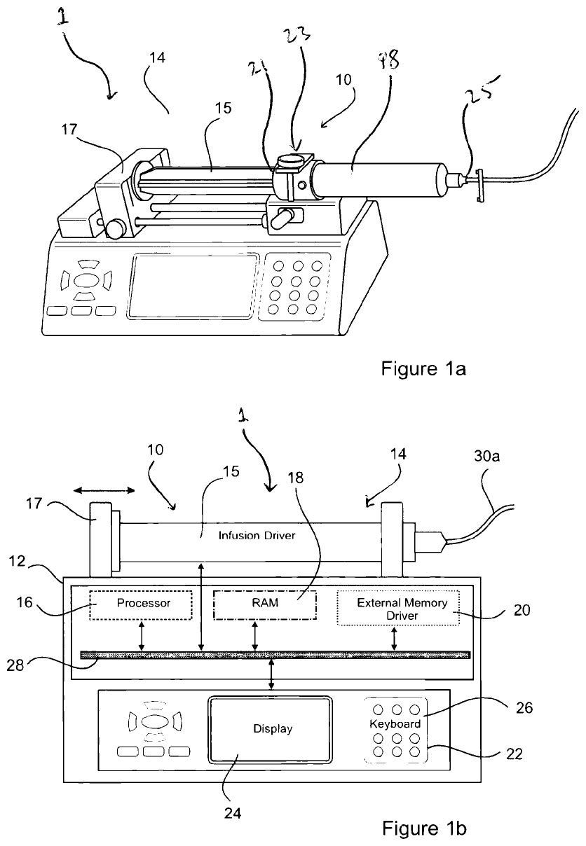

FIG. 1b show a block diagram of the apparatus 10 for controlling the flow rate at which the infusion driver 14 delivers the drug from a syringe or bag via a generic length of tubing to the patient.

The apparatus 10 comprises a computer system 12. The medication delivery apparatus 10 comprises an infusion driver 14. The infusion driver 14 may be referred to as an infusion device. The infusion driver 14 comprises a syringe 15 and a syringe driver 17. The syringe 15 defines an infusion container 19. The syringe 15 comprises a plunger 21. The infusion container is configured to receive at least a portion of the plunger 21. The plunger 21 and the infusion container together define an active agent chamber 98. The active agent chamber 98 may be referred to as a first chamber. The active agent chamber 98 is configured to receive an active agent. In particular, the active agent chamber 98 is configured to receive a pharmaceutical preparation. The pharmaceutical preparation comprises the active agent.

The active agent chamber 98 comprises an active agent chamber opening. The active agent chamber opening 23 is configured to receive the at least a portion of the plunger 21. The active agent chamber opening 23 may be considered an active agent chamber inlet. The active agent chamber 98 comprises an active agent chamber outlet 25.

The plunger 21 is configured to be displaced with respect to a longitudinal axis of the infusion container. Displacement of the plunger 21 along the longitudinal axis of the infusion container displaces the pharmaceutical preparation in the active agent chamber through the active agent chamber outlet 25. The pharmaceutical preparation is displaced into the conduit 30a.

In some embodiments, the infusion driver 14 comprises the computer system 12 and the syringe driver 17. The infusion driver 14 comprises a driving mechanism. In particular, the syringe driver 17 comprises the driving mechanism. The driving mechanism is controlled by the computer system 12 (the control unit 12). In particular, the control unit 12 is adapted to control the driving mechanism of the syringe driver 17 in order to deliver the drug (contained in the syringe 15) to the patient in a specific manner, for example, in accordance to either the Tansy function or the Sadleir function.

The computer system 12 comprises computer components such as a processor 16, a random access memory (RAM) 18, an external memory drive 20, and a user interface 22 such as a display 24 and a keyboard 26. These computer components are interconnected with respect to each other and the infusion driver 14 via a system bus 28.

In some embodiments, the infusion device comprises at least one infusion device processor in communication with infusion device memory. The at least one infusion device processor may comprise, or be in the form of the processor 16. The infusion device memory may comprise one or more of the random access memory 18 and the external memory drive 20. The at least one infusion device processor is configured to execute infusion device program instructions stored in infusion device memory to cause the infusion device to function as described herein. In other words, the infusion device program instructions are accessible by the at least infusion device processor, and are configured to cause the at least one infusion device processor to function as described herein.

In some embodiments, the infusion device program instructions are in the form of program code. The at least one infusion device processor comprises one or more microprocessors, central processing units (CPUs), application specific instruction set processors (ASIPs), application specific integrated circuits (ASICs) or other processors capable of reading and executing program code.

Infusion device memory may comprise one or more volatile or non-volatile memory types. For example, infusion device memory may comprise one or more of random access memory (RAM), read-only memory (ROM), electrically erasable programmable read-only memory (EEPROM) or flash memory. Infusion device memory is configured to store program code accessible by the at least one infusion device processor. The program code may comprise executable program code modules. In other words, infusion device memory is configured to store executable code modules configured to be executable by the at least one infusion device processor. The executable code modules, when executed by the at least one infusion device processor cause the at least one infusion device to perform certain functionality, as described herein.

The computer system 12 may optionally include a drug library, and database which contains the maximum allowable drug administration rate for each particular drug that may be infused to patients. If the drug delivery rate expected during use of the infusion driver 14 (e.g. during execution of the Tansy or Sadleir method), exceeds the maximum allowable drug administration rate, then the infusion rate will be reduced according to the maximum allowed infusion rate such that the concentration of drug leaving the dilution chamber (C_{d}) does not exceed the maximum allowable drug administration rate. This may result in the infusion time being greater than intended for the infusion, but ensures that the maximum permitted or suggested pharmaceutical drug administration rate is not exceeded.

During the method of infusing the pharmaceutical preparation in accordance with the present methods of the disclosure, the drug library may be accessed by the computer system 12 to confirm whether the drug delivery rate exceeds the maximum allowable drug administration rate; and if it does then, the infusion rate will be reduced according to the maximum allowed infusion rate to give the maximum allowable drug administration rate.

The processor 16 may execute instructions to control the driving mechanism of the syringe driver 17 in order to deliver the drug in accordance to, for example, either the Tansy function or the Sadleir function. The code executed by the processor 16 may be stored in the RAM 18 of the computer system 12 or may be provided from external sources through the external memory drive 20. This software will include the instructions to control the driving mechanism of the infusion driver 14 (e.g. the syringe driver 17) such that the pharmaceutical preparation exits the syringe 15 at a particular flow rate to match, or approximate the infusion rate of the pharmaceutical preparation dictated by the Tansy, Sadleir or another function specifying the rate at which the pharmaceutical preparation will be infused into the patient. In accordance with the first embodiment of the disclosure, the infusion driver 14 directly delivers the drug to the patient via conduit 30a (such as a minimal volume tubing with a three-way-tap to allow priming of the tubing with pharmaceutical preparation prior to commencing the program); and, the processor 16 execute codes for driving of the syringe driver 17 in order to deliver the drug (contained in the syringe 15) to the patient in accordance with the Tansy function. The software code (for example, FIG. 27), executed by the processor 16, comprises instructions for running an algorithm for calculating the infusion rates dictated by the Tansy function to control the flow rate using the syringe driver 17.

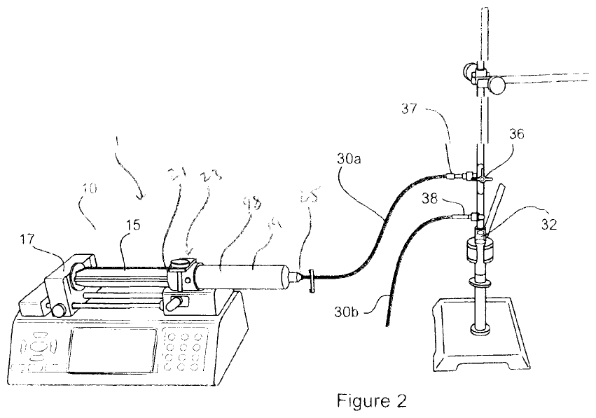

Referring now to FIG. 2, a medication delivery apparatus 10 according to a second embodiment of the disclosure is shown. Again, the medication delivery apparatus 10 may be referred to as an apparatus 10. The apparatus 10 according to the second embodiment is similar to the apparatus 10 according to the first embodiment and similar reference numerals are used to identify similar parts.

As described with reference to FIG. 1, the medication delivery apparatus 10 comprises an infusion container and a plunger 21. The infusion container and the plunger 21 may form at least part of a syringe. The infusion container is configured to receive at least a portion of the plunger 21. The plunger 21 and the infusion container together define an active agent chamber 98. The active agent chamber 98 is configured to receive a pharmaceutical preparation. The pharmaceutical preparation comprises an active agent, as previously described. The active agent chamber 98 comprises an active agent chamber opening 23. The active agent chamber opening 23 is configured to receive at least a portion of the plunger 21. The active agent chamber 98 comprises an active agent chamber outlet 25.

One of the differences of the apparatus 10 of the second embodiment of the disclosure is that the infusion driver 14 delivers the pharmaceutical preparation to a dilution chamber 32, before the pharmaceutical preparation is delivered to the patient. Thus, the medication delivery apparatus 10 comprises the dilution chamber 32. The dilution chamber 32 is fluidly connected to the infusion container. The dilution chamber 32 is configured to receive a diluent. The dilution chamber 32 is configured to receive the pharmaceutical preparation from the active agent chamber 98. In particular, the dilution chamber 32 is configured to receive the pharmaceutical preparation from the active agent chamber outlet 25. The dilution chamber 32 comprises a dilution chamber outlet 27.

The plunger 21 is configured to be displaced with respect to a longitudinal axis of the infusion container. Displacement of the plunger 21 along the longitudinal axis of the infusion container displaces the pharmaceutical preparation in the active agent chamber 98 through the active agent chamber outlet 25. The pharmaceutical preparation is displaced into the conduit 30a. The pharmaceutical preparation is displaced through the conduit 30a into the dilution chamber 32. The pharmaceutical preparation is diluted in the dilution chamber 32. The displacement of the plunger 21 displaces the diluted pharmaceutical preparation from the dilution chamber 32, through a second conduit 30b and to the patient.

The software code, executed by the processor 16, comprises instructions for running an algorithm for calculating the infusion rates dictated by the Sadleir function to control the flow rate of the syringe driver 17. Delivery of the pharmaceutical preparation from the infusion driver 14 (i.e. the active agent chamber 98) to the dilution chamber 32 and subsequently to the patient is conducted via conduits 30a and 30b. The conduits 30a and 30b comprise minimum volume extension tubing. The conduit 30a may be referred to as a first conduit. Conduit 30b may be referred to as a second conduit. The conduit 30a is configured to fluidly connect the active agent chamber outlet 25 and a dilution chamber inlet 29.

As mentioned before, the apparatus 10 in accordance with the second embodiment of the disclosure comprises a dilution chamber 32. An example arrangement of a dilution chamber 32 is shown in operation in FIG. 2.

There are two different disposable consumable systems particularly suited to clinical use, one with a 10 ml dilution chamber 32, and one with a 20 ml dilution chamber 32, although the method includes arrangements with dilution chambers 32 of other volume sizes (and the example of a method with a chamber volume of 0 mL is equivalent to the Tansy Method). The 20 ml dilution chamber 32 allows for a greater minimum infusion rate and a lower maximum infusion rate that the 10 ml chamber 32, but at a cost. This costs is that the fraction of drug delivered to the patient at any point of the infusion is reduced (by

$$\frac{{V}_{p} - {V}_{d} ( 1 - {e}^{- \frac{V p}{V d}} )}{{V}_{p}} )$$

where V_{d} is the volume of the dilution chamber 32 and Vₚ is the primary syringe infusion volume; the intention of this is that the drug remaining in the dilution chamber 32 (upon completion of the infusion process) is delivered to the patient as a bolus by emptying the dilution chamber 32 by, for example, depressing the syringe plunger, or by flushing the system with saline.

Alternatively, (1) the concentration of active ingredient in the pharmaceutical preparation can be increased (‘Increased concentration Sadleir method’) or (2) the volume of the pharmaceutical preparation and infusion rates can be increased (‘Increased volume Sadleir method’); any of (1) or (2) is done to deliver the same dose as the equivalent Tansy method at the end of the infusion period (i). In both of these alternative methods, the drug remaining in the dilution chamber 32 upon completion of the infusion process is discarded.

For infusions of duration greater than 25 minutes, a dilution chamber of ⅕ the volume of the infusion volume (i.e. 10 ml for a 50 ml infusion, 20 ml for a 100 ml infusion) is appropriate because approximately 80% of the total dose is given prior to the final bolus. For infusions over 20-25 minutes, a ⅖ (i.e.: a 20 ml dilution chamber for a 50 ml primary infusion volume) ratio ensures that infusion rates do not exceed 20 ml/min for a 50 ml infusion.

Clinically, the 30 minute infusion with 50 ml volume and 10 ml dilution chamber is appropriate in terms of the competing interests of (1) achieving infusion of the full therapeutic dose in a relative short period of time, but also (2) allowing for detection of submaximal adverse reaction in the patient. For infusions that are not witnessed by a doctor (i.e.: given unattended on the ward), it may be more appropriate to use the Sadleir function over 60 to 120 minutes, and with a 100 ml volume and 20 ml dilution chamber.

However, the infusion duration is likely to be limited by several factors. The first factor is the maximum infusion rate tolerated by typical-sized intravenous cannulas (i.e.: 22g). A second factor is that the maximum infusion rate of 20 ml/hr on most infusion drivers 14 resulting in that the minimum commonly used Sadleir function infusion duration will be 20 minutes for a 50 ml infusion volume and a 20 ml dilution chamber 32.

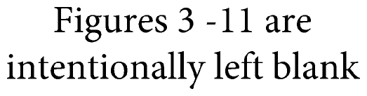

In accordance with the second embodiment of the disclosure, the infusion driver 14 delivers the drug via conduit 30a to the dilution chamber 32 and then to the patient via conduit 30b fluidly connected to the patient (see FIG. 3). And, the processor 16 executes codes running a particular algorithm for driving of the syringe driver 17 in order to deliver the pharmaceutical preparation (contained in the syringe 15) to the patient as dictated by the Sadleir function.

The apparatus 10 may be used for the administration of all therapeutic doses of any drugs (active ingredients such as medications) diluted in a diluent forming diluted pharmaceutical preparations that can be gradually administered to patients with the objective of decreasing the incidence of severe hypersensitivity reactions and avoid death of any hypersensitivity patients.

In particular, the apparatus 10 in accordance with the first and second embodiment of the disclosure is intended to be used, for example, in one of three scenarios:

Drug Test Dose—in a patient who is not suspected to be hypersensitive to the drug to be administered to the patient, in which case the apparatus 10 is used to administer the therapeutic dose of a drug in a particular manner (for example providing sequentially increasing test doses) that increases the chance that any unexpected hypersensitivity is detected, permitting stopping of the infusion process before a dose that will cause a more serious reaction to the patient has been administered. In this particular scenario, patients, who would otherwise have had an unexpected reaction to the drug, with the particular manner in which the therapeutic dose is administered, a tolerance is induced in the patient and no negative reaction will occur. Thus, this particular scenario generates what is typically referred to as unintentional acute desensitization.

Drug Challenge—in a patient who is suspected of having a hypersensitivity reaction due to a particular drug, and in whom it is deemed advantageous to confirm that the particular drug administered was responsible for the reaction, the apparatus 10 is used to administer the therapeutic dose of a drug in a particular manner that increases the capability or probability that, if a hypersensitivity reaction does occur, the infusion can be stopped before a particular quantity of the drug becomes a dose that will cause a more serious reaction in the patient. This scenario is particularly useful for confirming that the drug administered to the patient was responsible for the patient's hypersensitivity reaction.

Drug Desensitisation—in a patient who is known to be hypersensitive to a particular drug, in which case a therapeutic dose of the particular drug is administered in a particular manner (for example, providing relative low doses at the start of the infusions process) using the apparatus 10 such that tolerance is induced to the drug. This scenario is particularly useful for desensitising the patient to the particular drug.

### Methods for Delivering a Pharmaceutical Preparation

### The Tansy Method

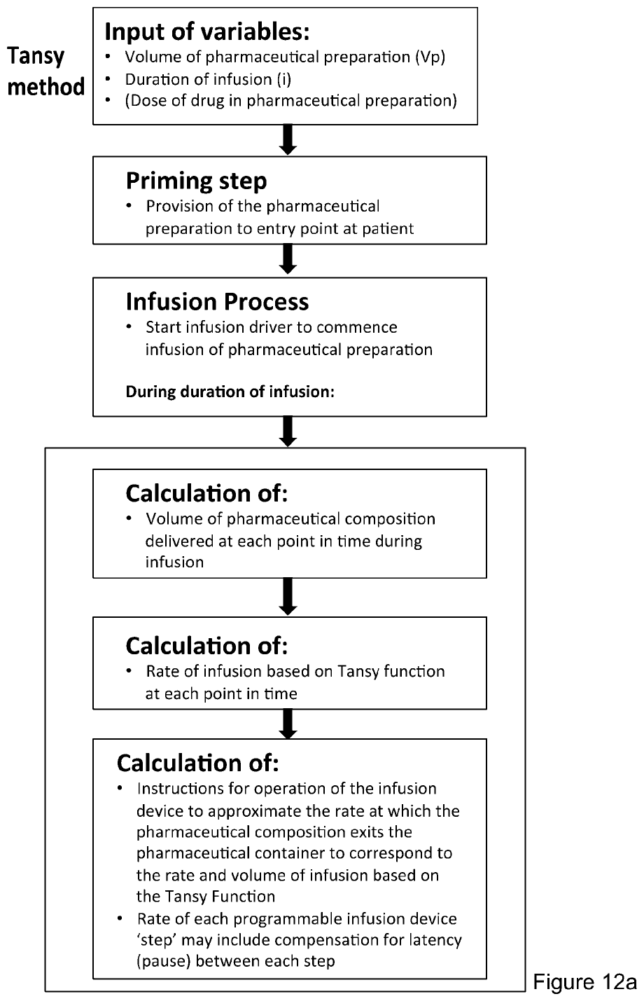

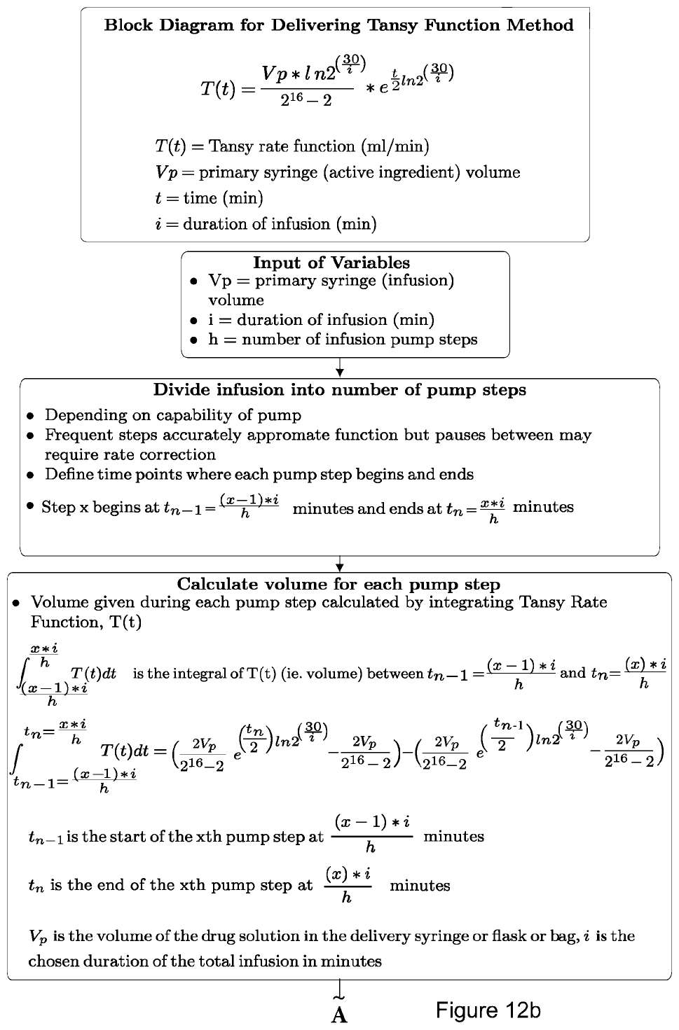

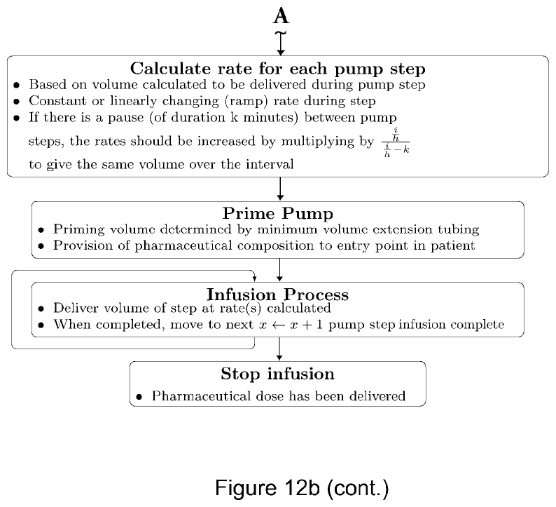

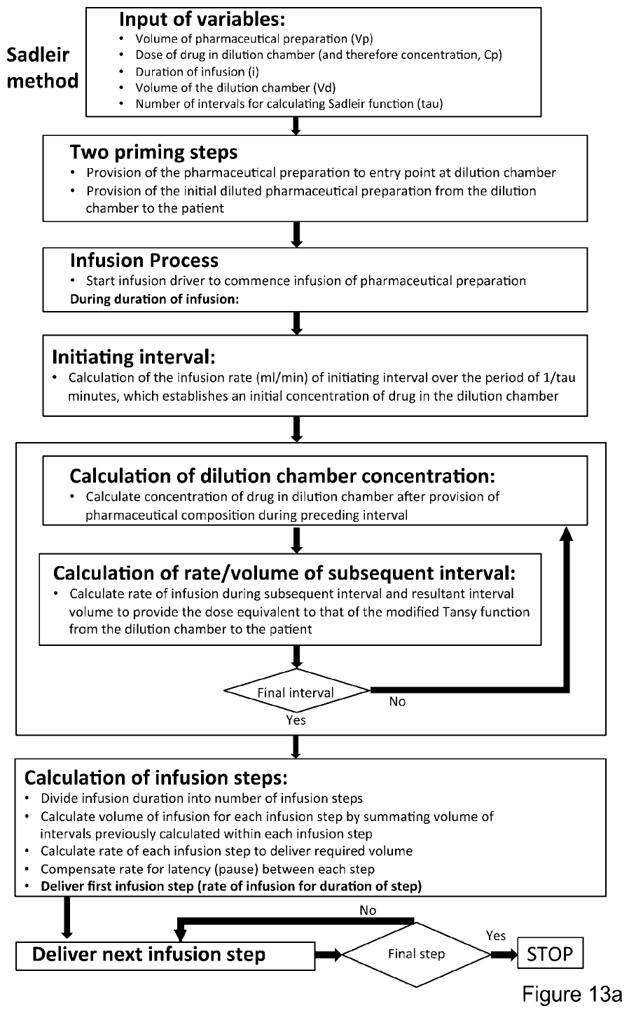

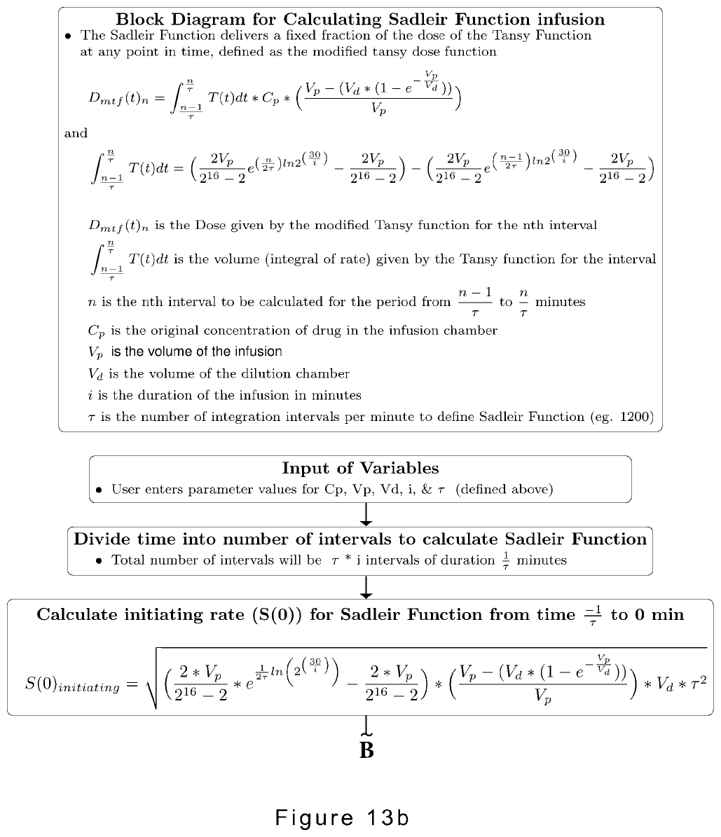

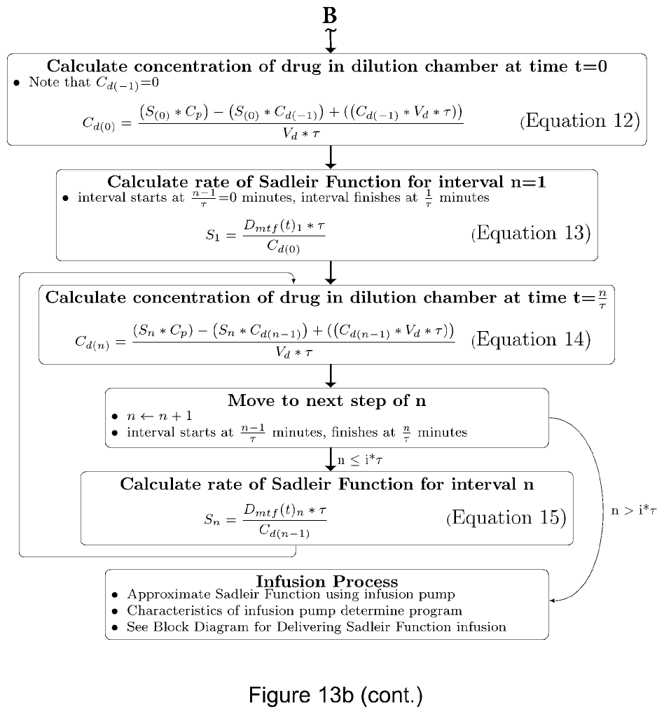

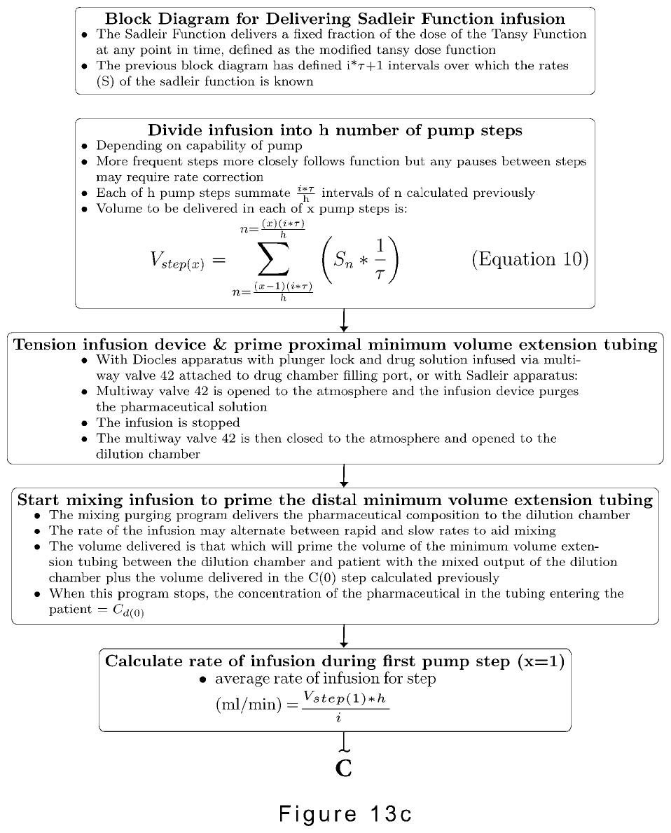

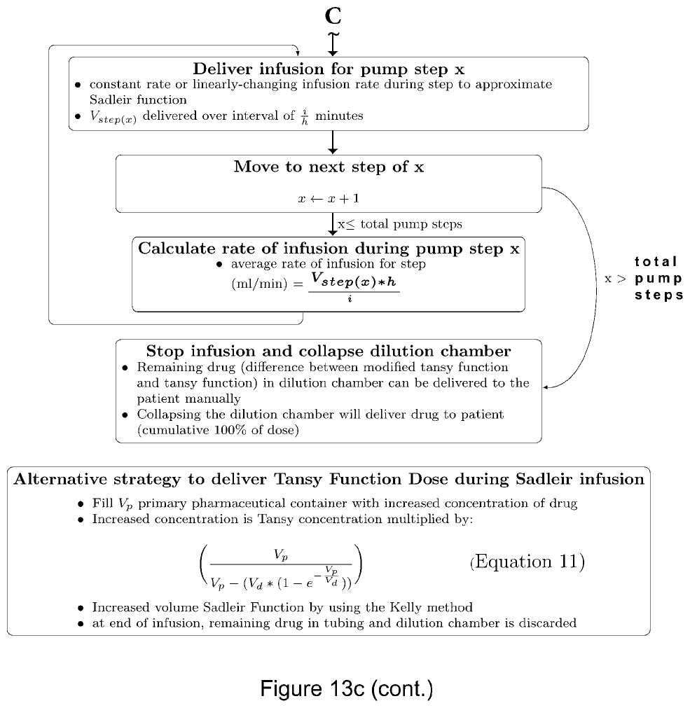

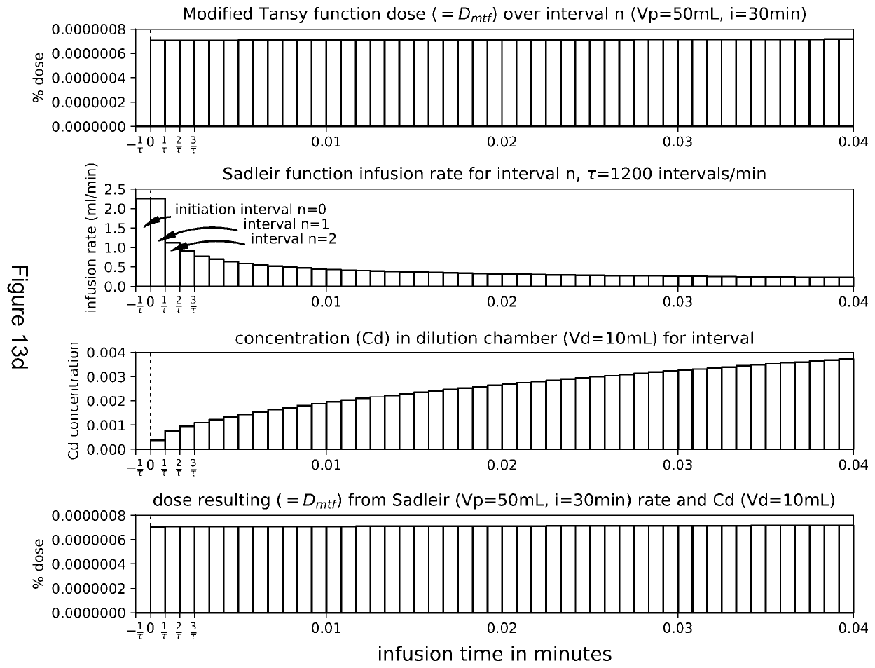

FIGS. 12a and 13a broadly illustrate steps for delivery of a therapeutic dose of the drug contained in the pharmaceutical preparation to be delivered by the infusion driver 14.

FIGS. 12a and 12b illustrate a method in accordance with a first embodiment of the disclosure. In the first embodiment of the disclosure, there is provided a method of delivering a pharmaceutical preparation to a patient. The pharmaceutical preparation is delivered directly to the patient in accordance with a flow rate as dictated by a Tansy function per equation (1) to be introduced below. In some embodiments, the pharmaceutical preparation is delivered in accordance with an infusion modelling function. In some embodiments, the Tansy function is the infusion modelling function.

In accordance with the first embodiment of the disclosure, there is provided a method for delivering a therapeutic dose of a particular drug to a patient using the apparatus 10 in accordance with the first embodiment of the disclosure, and depicted in FIG. 1. This method is referred to as the Tansy Method.

As mentioned before, the apparatus 10 in accordance with the first embodiment of the disclosure uses the Tansy function to control the flow rate to deliver the therapeutic dose of a particular drug directly (without using the dilution chamber 32) to a patient.

The particular drug to be administered is prepared in the syringe 15 containing a solvent (sterile water or saline), and delivered via the infusion driver 14 to the patient.

As shown in FIG. 12a, the operator inputs via the keyboard 26 of the infusion driver 14:
a) volume of pharmaceutical preparation (Vₚ) to be administered to the patient in ml, comprising an amount of drug (active ingredient in units of mass) and volume of solvent for mixing with the drug (the active ingredient); and
b) time over which the pharmaceutical preparation is to be administered in minutes (also referred to as the duration of infusion),
c) optionally, the identity of the particular drug (drug name), dose of drug, and/or maximum drug administration rate (dose/min) for the particular drug to ensure that the maximum drug administration rate is not exceeded during the infusion process.

Subsequently, the operator provides the pharmaceutical preparation to the entry point of the patient. This step is referred to as the priming step.

Then, the operator starts the infusion driver 14 via instructions through the keyboard 26.

The processor 16 of the infusion driver 14 than executes corresponding instructions for calculating the flow rate (ml/min) of the pharmaceutical preparation at each point in time during the duration of the infusion as dictated by the Tansy function per equation (1) below:

$$\begin{matrix}T ( t ) = \frac{Vp * ln ( {2}^{( \frac{30}{i} )} )}{{2}^{16} - 2} * {e}^{\frac{t}{2} ln ( {2}^{( \frac{30}{i} )} )} \\ T ( t ) = Tansy rate function ( ml / min ) \\ Vp = primary syringe ( infusion ) volume \\ t = time ( min ) \\ i = duration of infusion ( min )\end{matrix} \qquad ( 1 )$$

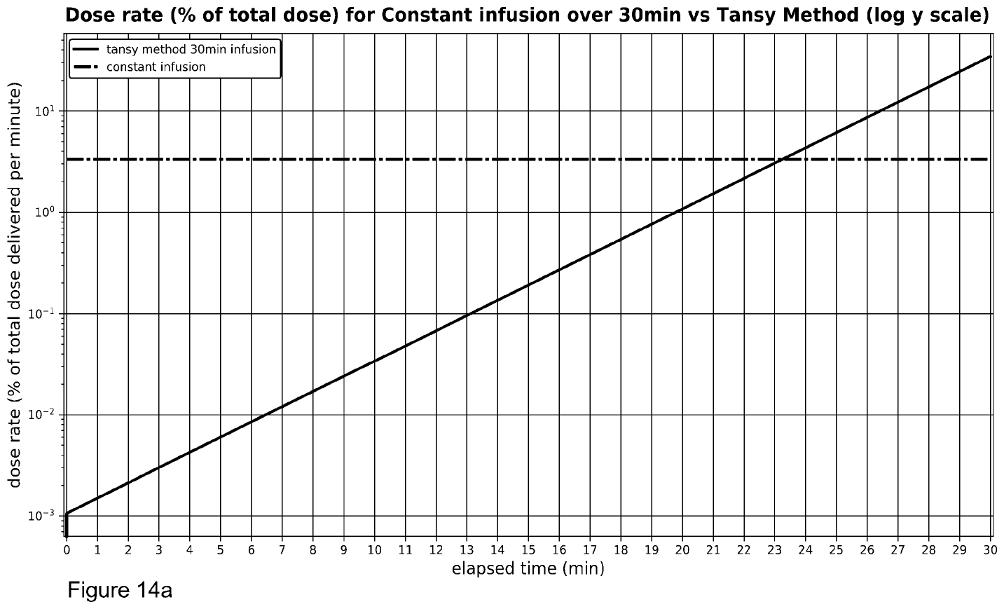

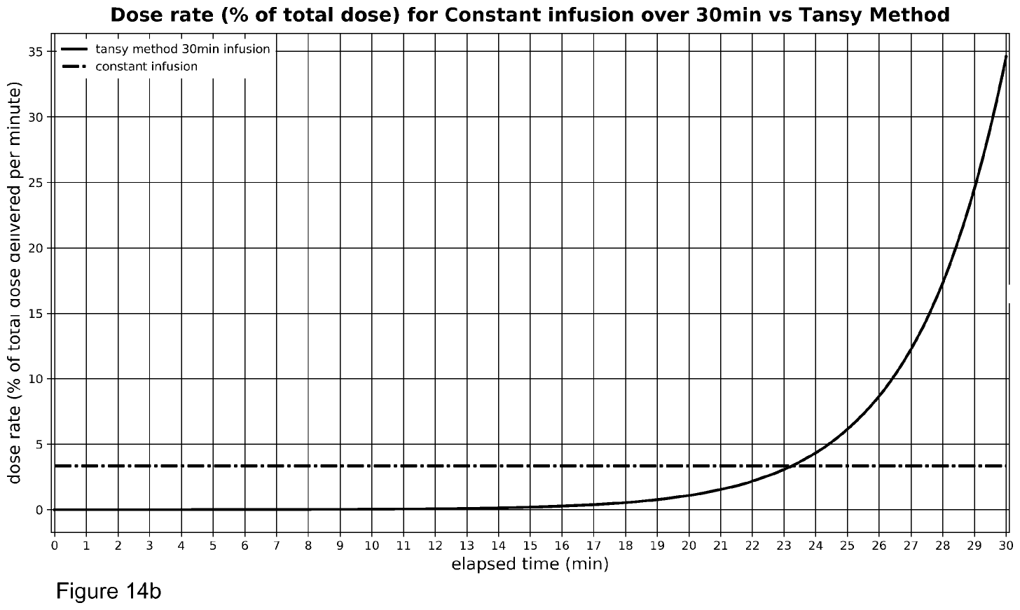

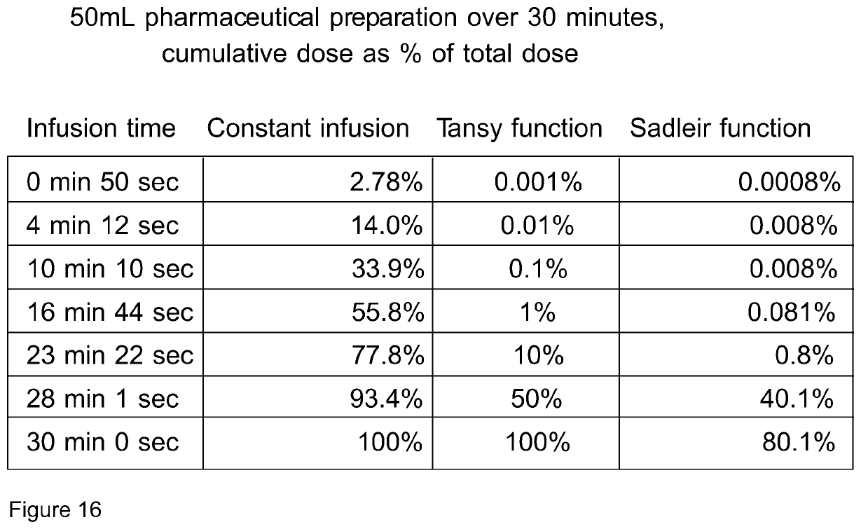

The Tansy method for a duration of infusion of 30 minutes has the following original features:
a) the Tansy method will deliver 0.01% of the dose after 14%, 0.1% after 34%, and 1% after 56% of the time period corresponding to the duration of the infusion process (see FIGS. 15 and 16). This increases the likelihood that a negative reaction will be detected and the infusion process may be stopped before a more serious negative reaction occurs. (In contrast, when using a conventional method based on a constant infusion 0.01%, 0.1% and 1% of the total dose will be all administered within the first 1% of the infusion process).
b) the flow rate increases continuously throughout the infusion, doubling every 2 minutes for a 30 minute infusion—see FIGS. 14a and 14b.

In relation to the original feature (a.) mentioned above, FIG. 15 shows the difference in cumulative dose administered over a period of a 30 minute infusion for the Tansy method versus the conventional Constant Infusion method. The total dose delivered over 30 minutes is the same in both methods (Tansy and Conventional (constant infusion over 30 minutes) methods).

Further, FIGS. 15a and 15b illustrate the clear separation in time of clinically-relevant magnitudes of cumulative drug administration when using the Tansy method.

However, as shown in FIG. 15, using the Constant Infusion method over 30 minutes will result in 0.01%, 0, 1% and 1% of the dose to have been administered over only the first 18 seconds of the infusion. When using the Constant Infusion method, if a patient was to have a minor reaction at 0.01% of the dose, and a maximal reaction at 10× or 100× times the 0.01% dose, the clinician is unlikely to recognize that the patient is hypersensitive to the drug and will not stop the infusion process before the dose that will induce a maximal reaction has been administered resulting in injury and potential death of the patient.

In contrast, the Tansy method starts at a relative low infusion rate and continuously increases the infusion rate. In particular, using the Tansy Method will result in a patient being administered 0.01% of the dose at 4.18 minutes, and 0.1% of the dose 5.97 minutes later. This almost 6-minute interval will increase the ability of a reaction being detected and permit ceasing of the infusion prior to the patient receiving a supramaximal dose, therefore minimizing any complications. Similarly, a cumulative 1% dose is achieved after another 6 minutes, as is the 10% cumulative dose. The approximately 6 minute separation of orders of magnitude of cumulative dose (for a 30 minute infusion) is a particular feature of the apparatus 10 in accordance with the first and second embodiment of the disclosure. This is illustrated in FIGS. 15 and 16.

In relation to the original feature (b.) mentioned above, FIG. 14a illustrates rate of drug administration, comparing the conventional Constant Infusion method and the Tansy method, using a logarithmic scale. This demonstrates that the rate of drug administration varies (in this particular arrangement it doubles) every two minutes when using the Tansy method for a 30-minute infusion. In particular, the Tansy method has the properties that the rate of drug administration is 0.01% of final infusion rate at 3.425 minutes into the infusion, 0.1% of maximal at 10.07 minutes, 1% at 16.71 minutes, 10% at 23.36 minutes, and 100% at 30 minutes. The total drug administered at is 0.01% after 4.18 minutes, 0.1% after 10.15 minutes, 1% after 16.72 minutes, 10% after 23.35 minutes, and 100% after 30 minutes (see FIG. 16).

As mentioned above, for a 30 minutes infusion the flow rate doubles every two minutes. However, the variation in flow rate is adjustable by changing the infusion duration (see FIGS. 19a and 19b). As shown in FIG. 19b, as the duration of infusion increases the variation in rate is reduced and as the duration of infusion decreases the variation in flow rate is increased.

Below is outlined the general equation for the cumulative volume of pharmaceutical preparation provided at each point in time during infusion in accordance with the first embodiment (i.e.: using the Tansy method).

$$\begin{matrix}V ( t ) = \frac{2 * Vp}{{2}^{16} - 2} * {e}^{\frac{t}{2} ln ( {2}^{( \frac{30}{i} )} )} - \frac{2 * Vp}{{2}^{16} - 2} \\ V ( t ) = Tansy volume function , cumulative volume ( ml / min ) at time t ( min ) \\ Vp = primary syringe ( infusion ) volume \\ t = time ( min ) \\ i = duration of infusion ( min )\end{matrix} \qquad ( 2 )$$

As previously described, the medication delivery system 1 may comprise the above described medication delivery apparatus 10. The medication delivery system 1 may also comprise the infusion device. The infusion device comprises the at least one infusion device processor and infusion device memory storing program instructions accessible by the at least one infusion device processor. The program instructions are configured to cause the at least one infusion device processor to actuate an infusion device actuator (e.g. infusion driver 14) to control the medication delivery apparatus 10 to deliver medication in accordance with the Tansy method.

In particular, the program instructions are configured to cause the at least one infusion device processor to receive a volume input (Vₚ) that is indicative of a volume of the pharmaceutical preparation. This may be a volume of the pharmaceutical preparation in the active agent chamber. The volume input (Vₚ) may be received via an input provided by a user. For example, the volume input (Vₚ) may be input using the user interface 22. Alternatively, the volume input (Vₚ) may be retrieved from the infusion device memory. Throughout this disclosure, the volume input (Vₚ) may correspond to the volume of pharmaceutical preparation.

The program instructions are further configured to cause the at least one infusion device processor to receive a time input (i) that is indicative of a time over which the pharmaceutical preparation is to be administered. The time input (i) may be received via an input provided by a user. For example, the time input (i) may be input using the user interface 22. Alternatively, the time input (i) may be retrieved from the infusion device memory.

The program instructions are further configured to cause the at least one infusion device processor to determine a number of infusion steps that are to be executed during the time over which the pharmaceutical preparation is to be administered. Although referred to as “infusion steps” herein, it will be understood that an infusion step may be considered, or referred to as a pump step. Determining the number of infusion steps may comprise receiving an infusion step input that is indicative of the number of infusion steps. Determining the number of infusion steps may comprise retrieving the number of infusion steps from the infusion device memory.

The program instructions are further configured to cause the at least one infusion device processor to determine a pharmaceutical preparation output volume for each of the infusion steps of the number of infusion steps. Each pharmaceutical preparation output volume corresponds to a volume of the pharmaceutical preparation that is to be output by the medication delivery apparatus during the respective infusion step. Determining the pharmaceutical preparation output volume for each of the number of infusion steps may comprise integrating the Tansy function between a first time that corresponds to a start of the relevant infusion step, and a second time that corresponds to an end of the relevant infusion step.

The Tansy function T(t) may be defined by:

$$T ( t ) = \frac{{V}_{p} \times ln {2}^{( \frac{30}{i} )}}{{2}^{16} - 2} {e}^{\frac{t}{2} l n {2}^{( \frac{30}{i} )}}$$

Where Vₚ is the volume input, t is the time and i is the time input.

Determining the pharmaceutical preparation output volume for each of the number of infusion steps comprises calculating:

$${\int }_{n - 1}^{n} T ( t ) d t$$

The program instructions are further configured to cause the at least one infusion device processor to determine a target flow rate of each infusion step. Each target flow rate is indicative of a target flow rate of the pharmaceutical preparation to be output by the medication delivery apparatus during the respective infusion step. Each target flow rate is determined based at least in part on the pharmaceutical preparation output volume of the respective infusion step. Determining the target flow rate of each infusion step may comprise dividing the pharmaceutical preparation output volume of a respective infusion step by a length of that infusion step. Determining the target flow rate of each infusion step may comprise determining an initial target flow rate and a final target flow rate for each infusion step. The initial target flow rate of a respective infusion step may be equal to the final target flow rate of a preceding infusion step. The final target flow rate of the respective infusion step may be equal to the initial target flow rate of the following infusion step.

The program instructions are further configured to cause the at least one infusion device processor to receive a pharmaceutical preparation input. The pharmaceutical preparation input is indicative of one or more of: an identity of the pharmaceutical preparation, a dose of the pharmaceutical preparation, and a maximum pharmaceutical preparation administration rate. The target flow rate may be limited at the maximum pharmaceutical preparation administration rate, such that the target flow rate does not exceed the maximum pharmaceutical preparation administration rate during infusion.

The program instructions are further configured to cause the at least one infusion device processor to actuate an infusion device actuator to displace the plunger 21 within the active agent chamber 98 such that the pharmaceutical preparation is output by the medication delivery apparatus 10 at the respective target flow rate during each infusion step.

### The Sadleir Method

In accordance with the second embodiment of the disclosure there is provided a method for delivering a therapeutic dose of a particular drug to a patient using the apparatus 10 in accordance with the second embodiment of the disclosure.

As mentioned before, the apparatus 10 in accordance with the second embodiment of the disclosure uses the Sadleir function to control the flow rate of pharmaceutical preparation leaving the infusion driver 14 for delivery of the pharmaceutical preparation to the dilution chamber 32 and, from the dilution chamber 32, to the patient.

The method in accordance with the second embodiment of the disclosure improves the accuracy of the manner in which the drug is delivered by delivering the drug at similar variations of rate as the first embodiment of the disclosure but, in contrast with the first embodiment of the disclosure, the drug when using the second embodiment of the disclosure is delivered at (1) a minimum flow rate that is greater than the minimum flow rate of the first embodiment of the disclosure, and (2) at a maximum infusion rate that is lower than the maximum rate of the first embodiment of the disclosure. See FIGS. 22a, 22b and 22c.

The improvement in accuracy (i.e.: being able to deliver a higher flow rate of the pharmaceutical preparation during the early phase of the infusion process) is achieved by delivering the pharmaceutical preparation to the dilution chamber 32. The dilution chamber 32 contains a fixed volume of diluent (saline or similar) to which the pharmaceutical preparation will mix during the course of the infusion. Therefore, by directing the pharmaceutical preparation into the dilution chamber 32, diluted pharmaceutical preparation is provided.

However, the fact that the pharmaceutical preparation is diluted in the dilution chamber 32 results in a reduction in the drug concentration within the dilution chamber 32 as compared to the drug concentration of the pharmaceutical preparation contained in the syringe 15 (i.e. the active agent chamber 98). This results in the pharmaceutical preparation exiting the dilution chamber 32 having a lower concentration than the pharmaceutical preparation contained in the syringe 15 (the active agent chamber 98) of the infusion driver 14. The concentration of pharmaceutical preparation leaving the dilution chamber 32 will be lowest at the beginning of the infusion, and increase throughout the duration of the infusion (see FIG. 26c for an example using a 10 ml dilution chamber with 50 mL infusion over 30 minutes). The flow rate of the pharmaceutical preparation is adjusted to a higher rate in order to compensate for the reduction in pharmaceutical preparation (drug) concentration (due to having been diluted in the dilution chamber 32) compared to that provided by the first embodiment of the disclosure (the Tansy Method),

Further, due to the pharmaceutical preparation being delivered not directly to the patient but instead to the dilution chamber 32, at the end of the process of administering the pharmaceutical preparation, a remainder of the pharmaceutical preparation will remain in the conduits 30 and the dilution chamber 32. The remainder of the pharmaceutical preparation (contained in the dilution chamber 32) may be administered by either, for example, decreasing the volume of the dilution chamber 32 or by flushing conduits 30 and the dilution chamber 32 with saline or other appropriate solution. For this, as described before in accordance with the second embodiment of the disclosure, in the arrangement shown in the figures, the dilution chamber 32 comprises a syringe permitting reduction of the volume of the dilution chamber 32 by pressing the plunger of the syringe. The dilution chamber 32 may comprise a second plunger (i.e. part of the syringe).

The quantity of remainder of the dose (Vᵣ) in the dilution chamber 32 at the end of the infusion process is dependent on the ratio of the volume of the drug to be administered (Vₚ) and volume of the dilution chamber (V_{d}). In particular, the volume of the remainder of the dose (Vᵣ) in dilution chamber 32 at end of the drug administration process is given by:

$$\begin{matrix}\frac{{V}_{p} - {V}_{d} ( 1 - {e}^{- \frac{Vp}{Vd}} )}{{V}_{p}} = {V}_{r} \\ {V}_{p} = volume of drug - containing infusion container \\ {V}_{d} = volume of dilution chamber\end{matrix} \qquad ( 3 )$$

Comparing the Tansy and Sadleir methods, the particular quantity of drug that remains (at the end of the infusion process) in the dilution chamber 32 and is not delivered to the dose delivered via the Sadleir method, is less than the full therapeutic dose or the dose that is delivered by the Tansy method. In particular, at any point in time during the drug administration process the dose delivered using the Sadleir function is obtained using equation 3 below

$$\frac{{V}_{p} - {V}_{d} ( 1 - {e}^{- \frac{{V}_{p}}{{V}_{d}}} )}{{V}_{p}} \qquad ( 3 )$$

by multiplying the dose that is delivered by the Tansy method by equation 3 above. Equation 3 is referred to as the ‘correction factor’.

The variation in the rate of administration of drug (active ingredient) for the Tansy method and the Sadleir method is similar, but the amount per unit time and the total dose (of the drug) delivered to the patient is reduced by a fixed fraction (by multiplying by the ‘correction factor’) that depends on the volume of the dilution chamber 32 relative to that of the total infusion volume, see FIG. 22a.

In particular, for a 10 ml dilution chamber with a 50 ml primary drug infusion (or 20 ml dilution chamber with 100 ml primary drug infusion), 19.865% of the dose remains in the dilution chamber 32 at the end of the infusion, and therefore only 80.135% of the full therapeutic dose is administered to the patient.

The volume of dose remaining in the dilution chamber 32 may be delivered to the patient by reducing the volume of the dilution chamber 32 in order that the final 19.865% of dose can be given to the patient as a push (by depressing the plunger in the dilution chamber), or by flushing the system with saline solution and deliver it to the patient.

The advantage of the Sadleir method, which is used in conjunction with the apparatus 10 incorporating the dilution chamber 32, is that the minimum flow rate of the pharmaceutical preparation exiting the infusion driver 14 is orders of magnitude greater than that of the Tansy method, and so the ability to accurately administer the drug is improved, and the total volume of the pharmaceutical preparation can be reduced. As mentioned before, infusion drivers 14 are not able to provide proper infusion rates at relative low flow rates such as the initial rates of infusion using the Tansy method. The Sadleir method also reduces the maximum flow rates required, reducing the required size of patient intravenous cannula size and improving patient tolerance.

The Sadleir method accomplishes this by using the dilution chamber 32 of the apparatus 10 in accordance with the second embodiment of the disclosure.

The precision of the estimate for the volume administered in the first minute of the Sadleir function achieves 3 significant figures when the algorithm used for calculating the calculate the volume operates on a time interval of 1/600th of a minute or shorter intervals (see FIG. 16 for volume in first minute for a 30 minute infusion from a 50 ml syringe with a 10 ml dilution chamber).

The Sadleir method, when using the same pharmaceutical preparation concentration, delivers a known fraction of the Tansy protocol dose, increasing proportionally at a similar rate. The Sadleir function is calculated by numerical approximation of a nonlinear function and this calculation is detailed below.

FIGS. 13a, 13b and 13c illustrates the method in accordance with the second embodiment of the disclosure where the pharmaceutical preparation is delivered via the dilution chamber 32 to the patient in accordance with the variation of the flow rate as dictated by the Sadleir function per equation (6) to be introduced below. FIG. 13d illustrates for each interval n (with an interval duration of 1/1200 min) the value of: the flow rates of as dictated by the Sadleir function, the concentration in dilution chamber and the % dose.

In accordance with the second embodiment of the disclosure the method for delivering a therapeutic dose of a particular drug to a patient uses the apparatus 10 in accordance with the second embodiment of the disclosure and depicted in FIGS. 2 and 3. This method is referred to as the Sadleir Method.

As mentioned before, the apparatus 10 in accordance with the second embodiment of the disclosure uses the Sadleir function to indicate to the syringe driver 17 at which flow rate the pharmaceutical preparation will be delivered to a patient using the dilution chamber 32.

The particular drug to be administered to the patient is prepared in, the syringe 15 containing a diluent (sterile water or saline), and delivered via the infusion driver 14 to the patient. The diluent may also be referred to as a solvent.

Referring to FIG. 13a, the operator inputs via the keyboard 26 of the infusion driver 14:
a) Volume of the pharmaceutical preparation (Vₚ) in mL to be delivered to the patient, comprising of the volume of solution to give the correct therapeutic dose of drug (active ingredient);
b) Volume of dilution chamber 32;
c) Concentration of drug in primary syringe (e.g. percent of therapeutic dose/ml);
d) Time (i) over which the pharmaceutical preparation is to be administered in minutes (also referred to as the duration of infusion);
e) Number of intervals per minute (t). (As will be explained below, the infusion process is divided into intervals over which the algorithm (run by the processor 16 of the infusion driver 14 and used for calculating the flow rate values as dictated by the Sadleir function) will be iterated.); and
f) Optionally, the identity of the particular drug (drug name), dose of drug, and/or maximum drug administration rate (dose/min) for the particular drug to ensure that the maximum drug administration rate is not exceeded during the infusion process.

Subsequently, the processor 16 of the infusion driver 14 calculates the parameters required for calculating the flow rate at which the infusion driver 14 needs to drive the pharmaceutical preparation with the syringe driver 17 from the syringe 15 (the pharmaceutical preparation) in order to comply with the Sadleir function; these parameters are:
1. the number of intervals during the infusion process over which the values of the dilution chamber concentration is calculated (the number of intervals per minute (t) multiplied by the duration of the infusion in minutes (i)); and
2. the flow rate S(0)_{initiating} of the pharmaceutical preparation that establishes a particular concentration of drug in the dilution chamber 32. This interval occurs prior to the delivery of drug to the patient and it begins at 1/τ minutes prior to the infusion, is of duration 1/τ minutes, and finishes at time 0. The equation below provides the rate of the initiating dose in ml/min:

$$\sqrt{( ( \frac{2 {V}_{p}}{{2}^{16} - 2} {e}^{\frac{1}{2 τ} l n ( {2}^{\frac{30}{i}} )} - \frac{2 {V}_{p}}{{2}^{16} - 2} ) ) * ( \frac{{V}_{p} - ( {V}_{d} * ( 1 - {e}^{- \frac{{V}_{p}}{{V}_{d}}} ) )}{{V}_{p}} ) ) * {τ}^{2} * {V}_{d}} \qquad ( 4 )$$

The processor 16 executes instructions to run an algorithm for calculating the rate or volume of the initiating interval, and that of the τ*i intervals during the infusion process according to the algorithm illustrated in FIG. 13b conducted by the python 3 software instructions (software) shown in FIG. 28.

The processor 16 executes instructions to run an algorithm for calculating the initiating interval rate using the equation (4) above for delivering of the pharmaceutical preparation during the time period from −1/τ to 0 to the dilution chamber 32. Deduction of the equation 4 is shown at a later stage below.

The initiating step occurs during the time period from −1/τ to 0 and during this step a concentration of active ingredient is established within the dilution chamber 32.

To calculate the flow rate that the pharmaceutical preparation needs to exit the syringe driver 17 in accordance with the Sadleir method during each subsequent interval after the initiating interval, it is necessary to calculate the concentration of the dilution chamber 32 prior to each subsequent interval.

For example, at time 0 and prior to commencement of the infusion process, it is necessary to calculate the concentration of the pharmaceutical preparation contained in the dilution chamber 32 in order to calculate the flow rate for the first subsequent interval occurring after the initiating step. Equation 12 shown in FIG. 13b provides the concentration of the dilution chamber 32 at time 0.

Once the concentration of the dilution chamber 32 at time 0 is calculated, the flow rate during the first interval (n=1) is calculated by the processor 16 via equation 13 shown in FIG. 13b. This requires calculation of the dose of drug (active ingredient) that is administered using the Tansy function for the equivalent interval of time for an infusion with the same pharmaceutical preparation characteristics in the infusion driver syringe (concentration of drug, volume of pharmaceutical preparation to be administered (Vₚ), and total duration of the infusion (i)). This particular dose (as it would be administered using the Tansy function) is then reduced by multiplying its value by the correction factor

$$\frac{{V}_{p} - {V}_{d} ( 1 - {e}^{- \frac{{V}_{p}}{{V}_{d}}} )}{{V}_{p}}$$

The dose obtained by this multiplication is referred to as the dose of the modified Tansy function, or D_{mtf}, and is defined in FIG. 13b.

After the flow rate for the interval n=1 is calculated by the processor 16, the concentration of drug in dilution chamber 14 at the end of this interval (at time 1/τ minutes) is calculated using equation 14 of FIG. 13b. This equation estimates the concentration of drug in the dilution chamber 14 at the end of the interval n (in this example n=1) by dividing the amount of drug in the dilution chamber by the volume of the dilution chamber 32. The amount of drug in the dilution chamber 32 is estimated from the amount of drug present in the dilution chamber 32 at the start of the previous interval (n−1, at this point n=0 or the initiating interval), the particular dose that has entered the dilution chamber 32 during the interval n, and the particular dose has exited the dilution chamber 32 during the interval, n.

At this stage, the flow rate during each subsequent interval n, after the first interval that occurred between time 0 to 1/τ minutes, is calculated by the processor 16 by calculating in sequence the flow rate for each interval n via equation 15 shown in FIG. 13b, and then the concentration of drug in the dilution chamber at the end of each interval n via equation 14 shown in FIG. 13b.

In particular, the flow rate (Sn) during each subsequent interval is such that the same dose is given to a patient as when using the Tansy method but modified by reducing the rate of the Tansy function to account for the amount of drug remaining inside the dilution chamber 32 at the end of the infusion. The infusion rate is calculated per equation:

$${S}_{n} = \frac{{D}_{{mtf ( t )}_{n}} * τ}{{C}_{{d}_{( n - 1 )}}}$$

Deduction of the equation of the Starting Rate for Priming Dose

The initial rate of the theoretical Sadleir Function is undefined (as the dilution chamber concentration is zero, the initial rate is equal to the Tansy Function dose (0), divided by the concentration (0), i.e. 0/0).

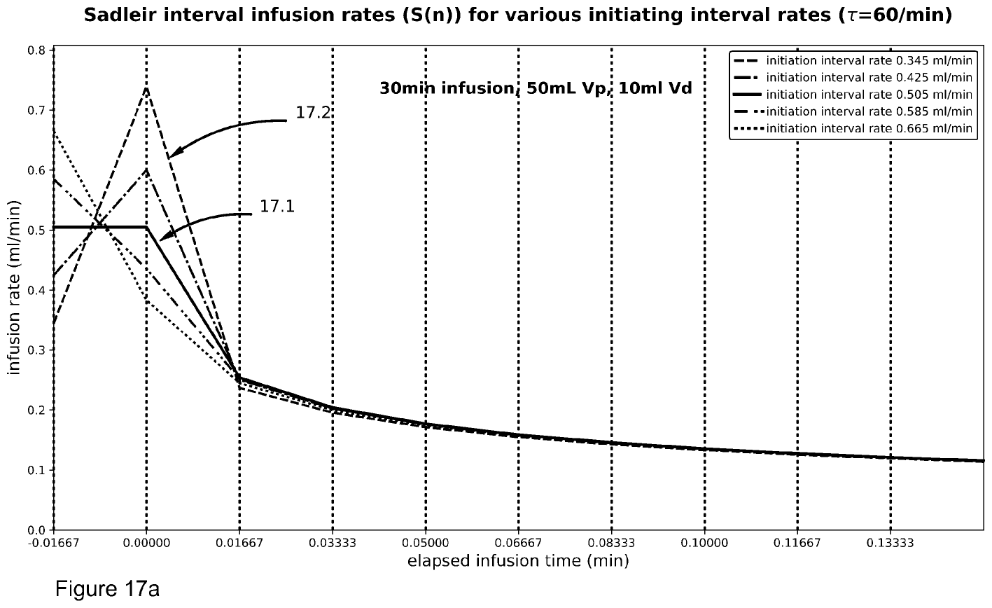

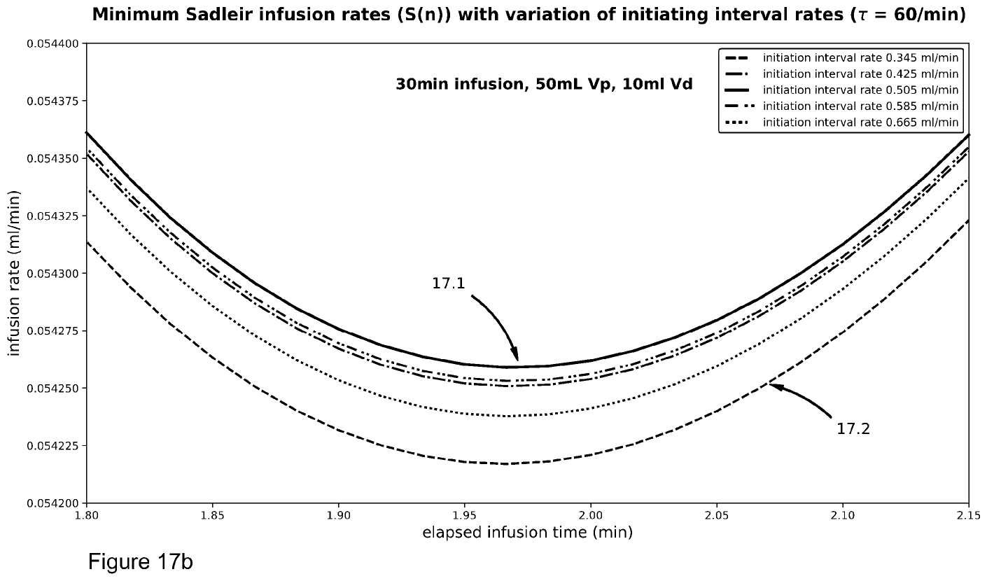

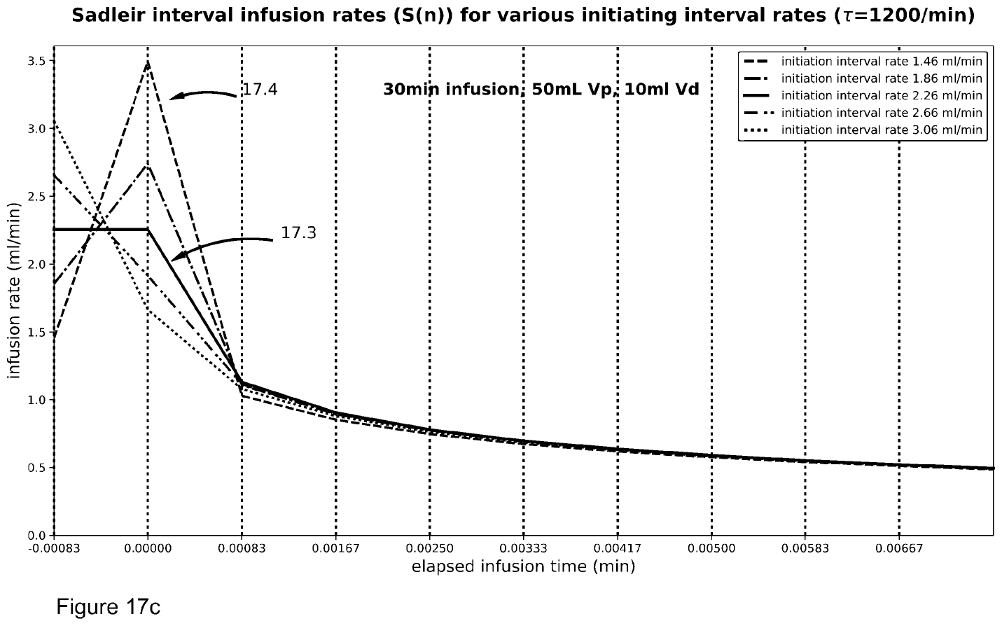

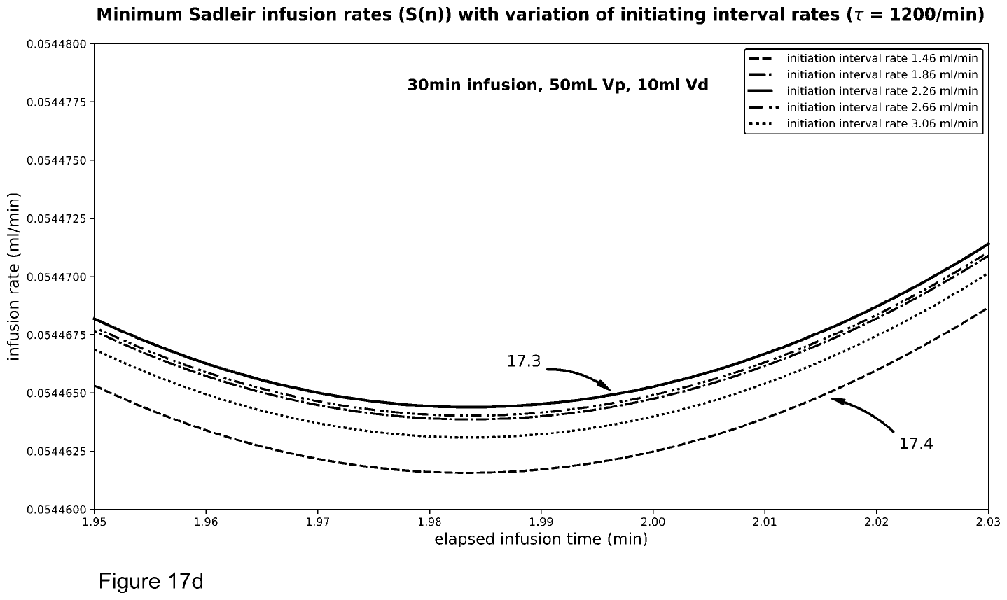

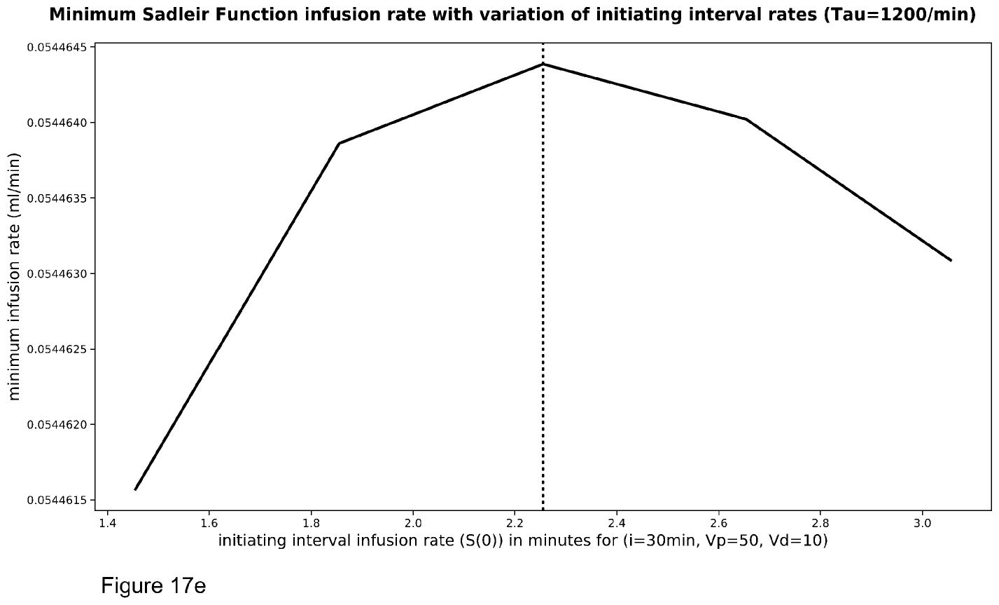

The Sadleir Function follows a concave curve starting from a particular value at t=0, reducing to a minimum value, and, after reaching the minimum value, increasing to a final value. FIGS. 17, in particular FIGS. 17b and 17d, illustrate the flow rate as dictated by the Sadleir function over a particular period of time for a 30 minute infusion duration, for different values of τ (60 and 1200, respectively).

As shown in, for example, FIGS. 17a and 17b, the flow rate starts at a particular flow rate and slows down until reaching a minimum flow rate at which thereafter the flow rate increases continuously until completion of the infusion process.

The optimal initiating flow rate (for infusion processes in accordance with the Sadleir method) is that particular flow rate that will result in the maximum minimum flow rate over the course of the infusion process. The reason that this particular flow rate is the optimal flow rate is that, as mentioned before, increasing the flow rate with which the pharmaceutical preparation exits the infusion driver 14 (i.e. the active agent chamber 98) increases the accuracy of the administration process of the pharmaceutical preparation because it is known that infusion drivers 14 do not deliver accurately pharmaceutical preparations at relative low rates as occurs when using the Tansy function.

As can be seen from FIG. 17a (tau=60, i=30 min, Vₚ=50 mL, V_{d}=10 mL), the lowest initiating interval rate (17.2) results in a lower concentration in the dilution chamber at the end of this interval, resulting in a higher rate for S₁, but lower subsequent rates. It can be seen in FIG. 17b that the initiating rate that results an equal S₁ rate will result in the maximum flow rate minimum (17.1).

FIGS. 17c and 17d show a graph plotting the flow rates as dictated by the Sadleir function over particular periods of time for a multitude of instances having different initiating flow rates as for 17a and 17b, but with tau=1200. As shown in FIG. 17d, line 17.1 has a starting flow rate of approximately 2.26 ml/min and (as shown in FIG. 17d) the maximum minimum flow rate, and line 17.2 has the minimum flow rate compared to all other instances.

FIG. 17e shows a graph plotting the value of minimum flow rates for each particular flow rate of a multitude of flow rates from FIGS. 17c and 17d. As shown in FIG. 17e, the maximum minimum flow rate occurs with an initiating flow rate of approximately 2.26 ml/min. This particular flow rate will be chosen as the starting flow rate due to having maximum minimum flow rate.

The ideal priming (initiating) dose will be the one having as flow rate the starting flow rate of line 17.3; due to the fact that this line 17.3 has the maximum minimum flow rate as can be seen in FIG. 17e. The ideal initiating dose or rate, prior commenced of the infusion process, is that which results in the infusion rate of the initiating step (S(0)initiating) to be equal to the infusion rate of the first interval (S(1st interval)), such that the rate of S(0)=S(1).

The sensitivity of the Sadleir function to the variations in the flow rate of the initiating step is increased when the size of the interval (1/τ) over which, the Sadleir function is iterated, is greater; FIGS. 17a and 17b, and FIGS. 17c and 17d demonstrate the above. In fact, in FIGS. 17a and 17b, a value of τ of 60/min is used and the change in minimum flow rate is greater. And, as shown in FIGS. 17c and 17d, if a value of 1200/min for τ is chosen, the change in minimum flow rate is less. Decreasing the size of the intervals (increasing τ) decreases the sensitivity to changes in the initiating interval rate.

Further, after the initiating interval, the infusion process will commence.

The flow rate during the first interval of the Sadleir Function is such that the dose given during the first interval is D_{mtf}(t)₁ (as defined above) calculated based on the concentration inside the dilution chamber 32 after occurrence of the initiating dose.

The infusion time is divided up into τ*i intervals, where i is the number of minutes over which the infusion is delivered, and τ is the number of intervals per minute. Each interval is of duration 1/τ minutes.

The volume given by the modified Tansy function for interval n (between time=(n−1)/τ and n/τ minutes), is given by the integral of the tansy rate function multiplied by a correction factor which accounts for the amount of drug remaining in the dilution syringe at the end of the Sadleir Infusion (the second embodiment of the disclosure), or:

$${{V}_{mtf} ( t )}_{n} = {\int }_{\frac{n - 1}{τ}}^{\frac{n}{τ}} T ( t ) dt * ( \frac{{V}_{p} - ( {V}_{d} * ( 1 - {e}^{- \frac{{V}_{p}}{{V}_{d}}} ) )}{{V}_{p}} )$$

The rate of the Tansy function at any point in time (t) is defined as:

$$T ( t ) = \frac{{V}_{p} ln ( {2}^{\frac{30}{i}} )}{{2}^{16} - 2} {e}^{\frac{t}{2} ln ( {2}^{\frac{30}{i}} )}$$

therefore, the volume of the modified tansy function for any interval n is:

$${{V}_{mtf} ( t )}_{n} = {\int }_{\frac{n - 1}{τ}}^{\frac{n}{τ}} \frac{{V}_{p} ln ( {2}^{\frac{30}{i}} )}{{2}^{16} - 2} {e}^{\frac{t}{2} ln ( {2}^{\frac{30}{i}} )} d t * ( \frac{{V}_{p} - ( {V}_{d} * ( 1 - {e}^{- \frac{{V}_{p}}{{V}_{d}}} ) )}{{V}_{p}} )$$

or expanded to:

$${{V}_{mtf} ( t )}_{n} = ( ( \frac{2 {V}_{p}}{{2}^{16} - 2} {e}^{\frac{n}{2 τ} ln ( {2}^{\frac{30}{i}} )} ) - \frac{2 {V}_{p}}{{2}^{16} - 2} ) - ( \frac{2 {V}_{p}}{{2}^{16} - 2} {e}^{\frac{n - 1}{2 τ} ln ( {2}^{\frac{30}{i}} )} - \frac{2 {V}_{p}}{{2}^{16} - 2} ) ) * ( \frac{{V}_{p} - ( {V}_{d} * ( 1 - {e}^{- \frac{{V}_{p}}{{V}_{d}}} ) )}{{V}_{p}} )$$

The dose of the modified tansy function (Dmtf(t)n) for interval n is given by multiplying (1) the volume given over the interval (Vmtf(t)n) by (2) the concentration of drug from the primary pharmaceutical container (Cₚ), or:

$${{D}_{mtf} ( t )}_{n} = {{V}_{mtf} ( t )}_{n} * {C}_{p}$$

Therefore, the dose of the modified Tansy function for the interval n can be defined:

$${{D}_{mtf} ( t )}_{n} = {\int }_{\frac{n - 1}{τ}}^{\frac{n}{τ}} \frac{V p * ln ( {2}^{\frac{30}{i}} )}{{2}^{16} - 2} * {e}^{\frac{t}{2} ln {2}^{( \frac{30}{i} )}} d t * ( \frac{{V}_{p} - ( {V}_{d} * ( 1 - {e}^{- \frac{{V}_{p}}{{V}_{d}}} ) )}{{V}_{p}} ) * {C}_{p}$$

Or:

$${{D}_{mtf} ( t )}_{n} = ( ( \frac{2 {V}_{p}}{{2}^{16} - 2} {e}^{\frac{n}{2 τ} ln ( {2}^{\frac{30}{i}} )} - \frac{2 {V}_{p}}{{2}^{16} - 2} ) - ( \frac{2 {V}_{p}}{{2}^{16} - 2} {e}^{\frac{n - 1}{2 τ} ln ( {2}^{\frac{30}{i}} )} - \frac{2 {V}_{p}}{{2}^{16} - 2} ) ) * ( \frac{{V}_{p} - ( {V}_{d} * ( 1 - {e}^{- \frac{{V}_{p}}{{V}_{d}}} ) )}{{V}_{p}} ) * {C}_{p}$$

The rate of the initiating interval (S(0)) should equal that of the first interval, (S(1)), as has been explained previously.

The rate of the first interval (S(1)) is determined by the dose of the modified Tansy function for the equivalent interval of the infusion (from time zero to time 1/τ minutes) and concentration C_{d(0)} in the dilution chamber V_{d} at the start of this interval. The rate is equal to the volume given divided by the interval of time, and the volume is determined by the dose divided by the concentration, or:

$$S ( 1 ) = \frac{volume}{time interval} = \frac{( \frac{d o s e}{c o n c e n t r a t i o n} )}{( \frac{1}{τ} )} = \frac{dose * τ}{c o n c e n t r a t i o n}$$

### Or:

$$S ( 1 ) = \frac{{{D}_{mtf} ( t )}_{1} * τ}{{C}_{d ( 0 )}} \qquad ( 16 )$$

The initial concentration of the dilution chamber is given by the dose given during the initiation step (n=0), divided by the volume of the dilution chamber, V_{d}. The dose given during the initiation step is equal to the volume (V₀) given during the initiation step, multiplied by the concentration in the primary pharmaceutical syringe (Cₚ). The volume given during the initiation step is equal to the initiation step rate (S(0)), multiplied by the duration of the interval (1/τ minutes) or:

$${C}_{d ( 0 )} = \frac{{V}_{0} * {C}_{p}}{{V}_{d}} = \frac{S ( 0 ) * \frac{1}{τ} * {C}_{p}}{{V}_{d}} \qquad ( 17 )$$

from equation 16 above, giving the rate of the interval (S₁), we substitute C_{d(0)} to give:

$$S ( 1 ) = \frac{{{D}_{mtf} ( t )}_{1} * τ}{\frac{S ( 0 ) * \frac{1}{τ} * {C}_{p}}{{V}_{d}}}$$

Rearranged:

$$S ( 1 ) * \frac{S ( 0 ) * \frac{1}{τ} * {C}_{p}}{{V}_{d}} = {{D}_{mtf} ( t )}_{1} * τ$$

$$Or$$

$$S ( 1 ) * S ( 0 ) * \frac{\frac{1}{τ} * {C}_{p}}{{V}_{d}} = {{D}_{mtf} ( t )}_{1} * τ$$

As S(0)=S(1), therefore S(0)*S(1)=S(0)²:

$${S ( 0 )}^{2} = \frac{{{D}_{mtf} ( t )}_{1} * τ}{\frac{\frac{1}{τ} * {C}_{p}}{{V}_{d}}}$$

$$Or$$

$${S ( 0 )}^{2} = \frac{{{D}_{mtf} ( t )}_{1} * τ * {V}_{d}}{\frac{1}{τ} * {C}_{p}}$$

$$Or$$

$${S ( 0 )}^{2} = \frac{{{D}_{mtf} ( t )}_{1} * τ * {V}_{d}}{\frac{1}{τ} * {C}_{p}} and as$$

$${{D}_{mtf} ( t )}_{1} = {V}_{mtf} {T ( 1 )}_{1} * {C}_{p}$$

$${S ( 0 )}^{2} = \frac{{V}_{mtf} T ( t ) * {C}_{p} * τ * {V}_{d}}{\frac{1}{τ} * {C}_{p}}$$

Cancel out the Cₚ values and multiply the right hand side by τ/τ:

$$to give {S ( 0 )}^{2} = {{V}_{mtf} ( t )}_{1} * {τ}^{2} * {V}_{d}$$

### Or:

$$S ( 0 ) = \sqrt{{{V}_{mtf} ( t )}_{1} * {τ}^{2} * {V}_{d}}$$

As V_{mtf}(t)₁ is the integral of the modified tansy (rate) function between 0 minutes and 1/τ minutes:

$$S ( 0 ) = \sqrt{{\int }_{\frac{0}{τ}}^{\frac{1}{τ}} T ( t ) d t * ( \frac{{V}_{p} - ( {V}_{d} * ( 1 - {e}^{- \frac{{V}_{p}}{{V}_{d}}} ) )}{{V}_{p}} ) * {τ}^{2} * {V}_{d}}$$

substitute in tansy rate function:

$$S ( 0 ) = \sqrt{{\int }_{\frac{0}{τ}}^{\frac{1}{τ}} \frac{{V}_{p} ln ( {2}^{\frac{30}{i}} )}{{2}^{16} - 2} {e}^{\frac{t}{2} ln ( {2}^{\frac{30}{i}} )} d t * ( \frac{{V}_{p} - ( {V}_{d} * ( 1 - {e}^{- \frac{{V}_{p}}{{V}_{d}}} ) )}{{V}_{p}} ) * {τ}^{2} * {V}_{d}}$$

which expands to:

$$S ( 0 ) = \sqrt{( ( \frac{2 {V}_{p}}{{2}^{16} - 2} {e}^{\frac{1}{2 τ} ln ( {2}^{\frac{30}{i}} )} - \frac{2 {V}_{p}}{{2}^{16} - 2} ) - ( \frac{2 {V}_{p}}{{2}^{16} - 2} {e}^{\frac{0}{2 τ} ln ( {2}^{\frac{30}{i}} )} - \frac{2 {V}_{p}}{{2}^{16} - 2} ) ) ⋆ ( \frac{{V}_{p} - ( {V}_{d} * ( 1 - {e}^{- \frac{{V}_{p}}{{V}_{d}}} ) )}{{V}_{p}} ) * {τ}^{2} * {V}_{d}}$$

which equals:

$$S ( 0 ) = \sqrt{( \frac{2 {V}_{p}}{{2}^{16} - 2} {e}^{\frac{1}{2 τ} ln ( {2}^{\frac{30}{i}} )} - \frac{2 {V}_{p}}{{2}^{16} - 2} ) * ( \frac{{V}_{p} - ( {V}_{d} * ( 1 - {e}^{- \frac{{V}_{p}}{{V}_{d}}} ) )}{{V}_{p}} ) * {τ}^{2} * {V}_{d}}$$

### Notes

The initiation step or interval (n=0) is the dose that establishes the concentration in the dilution chamber, prior to the patient being administered the pharmaceutical in the first interval of the Sadleir method (n=1)
The initiation step occurs prior to the infusion, from time

$$- \frac{1}{τ}$$

to time 0 minutes
Subsequent intervals span from

$$\frac{n - 1}{τ}$$

minutes to n/τ minutes
The first interval (n=1) spans from 0 minutes to

$$\frac{1}{τ}$$

minutes of the infusion
i is the duration in minutes of the infusion
τ is the number of intervals per minute calculated for the Sadleir function
1/τ is the duration of each interval in minutes
n is the nth interval, spanning from

$$\frac{n - 1}{τ}$$

to n/τ minutes of the infusion
for example, a 30 minute infusion with τ=1200 intervals per minute will have 36,000 intervals in total, and the 1801st interval (n=1801) will start at time= 1800/1200 minutes and finish at time= 1801/1200 minutes

In particular, for a 30 minute infusion from a 50 ml syringe 15, with 10 ml dilution chamber 32 and 1/600 minute steps, the initial infusion rate is:

$$Rate = \sqrt{( \frac{2 {V}_{p}}{{2}^{16} - 2} * {e}^{\frac{1}{2 τ} ln ( 2 {( \frac{30}{i} )}_{)}} - \frac{2 {V}_{p}}{{2}^{16} - 2} ) * ( \frac{{V}_{p} - ( {V}_{d} * ( 1 - {e}^{- \frac{{V}_{p}}{{V}_{d}}} ) )}{{V}_{p}} ) * {V}_{d} * {τ}^{2}}$$

therefore:

$$Rate = \sqrt{( \frac{2 * 50}{{2}^{16} - 2} * {e}^{\frac{1}{2 * 600} l n ( 2 ( \frac{30}{30} ) )} - \frac{2 * 50}{{2}^{16} - 2} ) * ( \frac{50 - ( 10 * ( 1 - {e}^{- \frac{50}{10}} ) )}{{V}_{p}} )} * 10 * {600}^{2}$$

simplified to:

$$Rate = \sqrt{( 0.0015259 * 1.0005778 - 0.0015259 ) * ( 0.8013476 * 10 * 60 {0}^{2} )}$$

simplified to:

$$Rate = 1.5 \; 948 \text{ml/hr}$$

S(0)_{initiating} is the rate of infusion for the initiating interval which is of duration

$$\frac{1}{τ}$$

minutes
τ is the number of iterated intervals per minute
Vₚ is the volume of the drug solution in the delivery syringe or flask or bag
i is the chosen duration of the total infusion in minutes V_{d} is the volume of the dilution chamber

For the same configuration, but τ= 1/1200 minutes, the priming rate (for 1/1200 minutes duration) is =2.25526 ml/min).

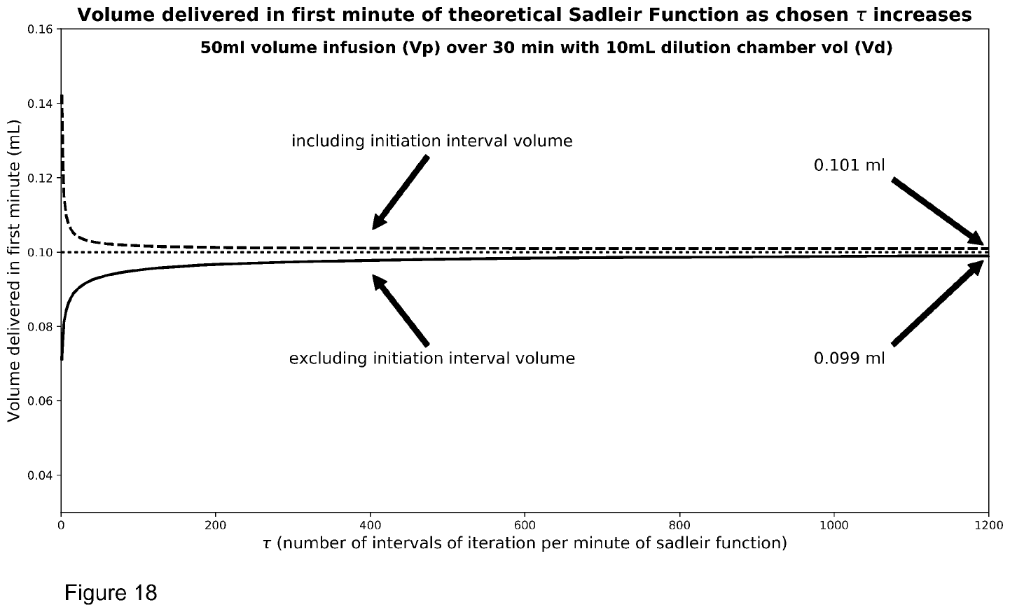

FIG. 18 illustrates the volume administered in the first minute using the Sadleir method using a 30 minute infusion from a 50 ml syringe, with a 10 ml dilution chamber.

The precision of the estimate for the volume administered in the first minute of the Sadleir function achieves 3 significant figures when iterating to a time interval of 1/1200^{th} of a minute (see FIG. 18 for the volume in first minute for a 30 minute infusion from a 50 ml syringe with a 10 ml dilution chamber).

### Calculation of Rate of Subsequent Interval

As mentioned before, to calculate the value of the infusion rate for each subsequent interval occurring after the initiating interval, first requires an estimate of the concentration of drug in the dilution chamber 32 at the end of the interval the occurred prior the particular subsequent interval for which its infusion rate (the subsequent infusion rate) is being calculated. The subsequent infusion rate is calculated as that required to deliver a volume of fluid in the dilution chamber 32 that contains the equivalent dose (D_{mtf}) that would be given by the modified Tansy function (that is, the dose given by the Tansy function in the corresponding interval that is reduced by multiplying by the ‘correction factor’, see FIG. 13b and equation 6a below) assuming the concentration of drug calculated by equation 9 below (equation 14 in FIG. 13b) Thus:

The concentration in the dilution chamber 32 at the end of a particular subsequent interval n is approximated as the amount of drug in the dilution chamber 32 at the end of that subsequent interval n divided by the volume of the dilution chamber 32. The amount of drug in the dilution chamber 32 at the end of the subsequent interval n is approximated by:
the amount of drug in the dilution chamber 32 at the start of the interval (dilution chamber volume multiplied by dilution chamber drug concentration at the end of the previous interval (C_{d(n-1)}));
added to the amount of drug that entered the dilution chamber during the interval (infusion rate (Sₙ) multiplied by interval duration (1/tau)) multiplied by the concentration of drug in the pharmaceutical preparation Cₚ);
and subtracting the amount of drug that exited the dilution chamber 32 during the interval (interval infusion rate (Sₙ) multiplied by interval duration (1/tau)) multiplied by the concentration of drug in the dilution chamber at the end of the previous interval (C_{d(n-1)})).

Thus:

$${C}_{d ( n )} = \frac{( {C}_{d ( n - 1 )} * {V}_{d} ) + ( {S}_{n} * {C}_{p} * \frac{1}{τ} ) - ( {S}_{n} * {C}_{d ( n - 1 )} * \frac{1}{τ} )}{{V}_{d}}$$

The dilution chamber concentration (C_{d(n)}) can be simplified to:

$${C}_{d ( n )} = \frac{( {C}_{d ( n - 1 )} * {V}_{d} * τ ) + ( {S}_{n} * {C}_{p} ) - ( {S}_{n} * {C}_{d ( n - 1 )} )}{{V}_{d} * τ}$$

The infusion rate (Sₙ) of the subsequent interval (n) is then equal to the volume of pharmaceutical preparation to be delivered to the dilution chamber 32 divided by the duration of that interval n. The volume is equal to the dose of active ingredient dictated by the modified Tansy function divided by the concentration in the dilution chamber 32 at the end of the previous interval. The rate of the subsequent interval n is equal to the volume divided by the duration of the interval in minutes, or alternatively the volume multiplied by the number of intervals per minute, or:

$${S}_{n} = \frac{{D}_{m t {f ( t )}_{n}} * τ}{{C}_{{d}_{( n - 1 )}}}$$

As mentioned before, using the Sadleir function instead of the Tansy function, results in administration of a dose that is less than the dose administered at any point in time during the Tansy function. The dose per the Sadleir function is reduced by multiplying the dose as dictated by the Tansy function by the correction factor:

$$\frac{{V}_{p} - {V}_{d} ( 1 - {e}^{- \frac{{V}_{p}}{{V}_{d}}} )}{{V}_{p}}$$

Reducing the dose ensures that the duration of the infusion is equal to that of that provided by the Tansy function for the same volume of infusion and given that at the end of the infusion, an amount of drug remains in the dilution chamber 32.

The number of subsequent intervals is divided by the duration of infusion (in minutes) to give the number of intervals per minute (τ), giving a total of (i*τ) intervals over the infusion period (each interval from a time (n−1)/τ to a time n/τ minutes, see FIG. 13d.

The volume administered by the Tansy function infusion for each interval is calculated by integrating the Tansy function over the time period of each interval; extending from (n−1)/τ to a time n/τ minutes.

The integral of the tansy function is calculated as:

$${\int }_{\frac{n - 1}{τ}}^{\frac{n}{τ}} T ( t ) dt = ( \frac{2 {V}_{p}}{{2}^{16} - 2} {e}^{( \frac{n}{2 τ} ) ln ( {2}^{( \frac{30}{i} )} )} - \frac{2 {V}_{p}}{{2}^{16} - 2} ) - ( \frac{2 {V}_{p}}{{2}^{16} - 2} {e}^{( \frac{n - 1}{2 τ} ) ln ( {2}^{( \frac{30}{i} )} )} - \frac{2 {V}_{p}}{{2}^{16} - 2} ) \qquad ( 5 )$$

$${\int }_{\frac{n - 1}{τ}}^{\frac{n}{τ}} T ( t ) d n$$

is the integral of the Tansy function (ie. volume) between

$$\frac{n - 1}{τ}$$

and n/τ minutes
Vₚ is the volume of the drug solution in the delivery syringe or flask or bag
n is the iteration interval
τ is the number of iterated intervals per minute
i is the chosen duration of the total infusion in minutes

The volume administered for each interval (as calculated above) is converted into a dose by multiplying it by the concentration of the drug in the syringe 15. The calculated numerical value of the dose is then reduced to account for the fact that the total dose administered to the patient using the apparatus 10 using the Sadleir method is less than that infused from the syringe 15 due to the fact that a portion of the drug will remain in the dilution chamber 32 at the end of the infusion. Reduction of the numerical value of the dose is done by multiplying each the dose to be infused during each interval by

$$\frac{{V}_{p} - {V}_{d} ( 1 - {e}^{- \frac{{V}_{p}}{{V}_{d}}} )}{{V}_{p}} ,$$

which is 0.80135 for a 10 ml or 20 ml dilution chamber 32 with 50 ml or 100 ml syringes 15, respectively.

The dose administered by the modified Tansy function (the Sadleir Function) for each interval is therefore given by:

$${{D}_{m T} ( t )}_{n} = {\int }_{\frac{n - 1}{τ}}^{\frac{n}{τ}} T ( t ) d t * {C}_{p} * ( \frac{{V}_{p} - ( {V}_{d} * ( 1 - {e}^{- \frac{{V}_{p}}{{V}_{d}}} ) )}{{V}_{p}} ) \qquad ( 6 a )$$

D_{mT}(t)ₙ is the modified Tansy dose for the interval

$${\int }_{\frac{n - 1}{τ}}^{\frac{n}{τ}} T ( t ) d t$$

is the volume (integral) of the modified tansy function for the interval
Cₚ is the original concentration of drug in the delivery syringe or flask or bag
Vₚ is the volume of the delivery syringe or flask or bag
V_{d} is the volume of the dilution chamber

As mentioned before, prior administering the pharmaceutical preparation to the patient, it is necessary establish a concentration of drug in the dilution chamber 32 by filling the dilution chamber 32 with the pharmaceutical preparation. This is done via the initiating step mentioned earlier and occurs prior to infusion of the pharmaceutical preparation to the patient. As mentioned earlier, the initiating interval (n=0, see FIG. 13d), is of the same duration as the first subsequent interval (interval n=1, see FIG. 13d) and ideally has the same flow rate and volume as the first subsequent interval n=1; using equation (4), the flow rate for the initiating interval (the starting rate S(0)_{initiating}) is given by solving:

$${S ( 0 )}_{initiating} = \sqrt{( \frac{2 {V}_{p}}{{2}^{16} - 2} * {e}^{\frac{1}{2 τ} ln ( {2}^{( \frac{30}{i} )} )} - \frac{2 {V}_{p}}{{2}^{16} - 2} ) * ( \frac{{V}_{p} - ( {V}_{d} * ( 1 - {e}^{- \frac{{V}_{P}}{{V}_{d}}} ) )}{{V}_{p}} ) * {V}_{d} * {τ}^{2}} \qquad ( 6 b )$$

S(0)_{initiating} is the rate of infusion for the initiating period which is of duration

$$\frac{1}{τ}$$

minutes
τ is the number of iterated intervals per minute
Vₚ is the volume of the drug solution in the delivery syringe or flask or bag
i is the chosen duration of the total infusion in minutes V_{d} is the volume of the dilution chamber

This infusion occurring during the initiating interval, results in delivery of a dose to the dilution chamber of volume V_{d}. The resulting concentration in the dilution chamber 32 after the initiating interval is given by:

$${C ( 0 )}_{initiating} = \frac{{S ( 0 )}_{i} * {C}_{p}}{τ * {V}_{d}} \qquad ( 7 )$$

C(0)_{initiating}=concentration of drug in the dilution chamber after the initiating interval
S(0)ᵢ=rate of the sadleir function during the initiating step, in ml/min
τ is the number of iterated intervals per minute, and 1/τ is the duration of each interval
V_{d} is the volume of the dilution chamber
Cₚ is the original concentration of drug in the drug delivery flask or syringe or container

The rate for the first subsequent interval, n=1, after the initiating interval is then calculated using C₍₀₎ as the initial dilution chamber concentration (Cₙ₋₁). This is calculated:

$${S ( t )}_{n} = \frac{{{D}_{m T} ( t )}_{n} * τ}{{C}_{n - 1}} \qquad ( 8 )$$

S(t)ₙ is the Sadleir function rate for interval n (between

$$\frac{n - 1}{τ}$$

and n/τ min) in ml/min
D_{mT}(t)ₙ is the dose given by the modified tansy function between time

$$\frac{n - 1}{τ}$$

and n/τ
Cₙ₋₁ is the concentration of drug in the dilution chamber at the end of interval n−1
τ is the number of intervals per minute

The concentration of drug in the dilution chamber 32 at the end of interval n is then calculated using the equation below:

$${C}_{n} = \frac{( {V}_{d} * τ * {C}_{n - 1} ) + ( {S ( t )}_{n} * {C}_{p} ) - ( {S ( t )}_{n} * {C}_{n - 1} )}{τ * {V}_{d}} \qquad ( 9 )$$

Cₙ is the concentration of drug in the dilution chamber at the end of the nth step
Cₙ₋₁ is the concentration of drug in the dilution chamber at the start of the nth step
V_{d} is the volume of the dilution chamber
Cₚ is the concentration of drug in the delivery syringe or flask or bag
τ is the number of intervals per minute

The flow rate of each particular subsequent interval n is calculated from the last two equations (8) and (9), using the appropriate modified Tansy dose for each particular subsequent interval. In particular, the flow rate of each particular subsequent interval as dictated by the Sadleir function is calculated to give the volume that will result in the same dose as the Tansy function multiplied by the correction factor.

$$\frac{{V}_{p} - {V}_{d} ( 1 - {e}^{- \frac{{V}_{p}}{{V}_{d}}} )}{{V}_{p}} ,$$

which reduces the rate of drug administration at all stages of the Sadleir method by a constant fraction equal to the fraction of drug remaining in the dilution chamber compared to that of the total therapeutic drug dose.

Then, the concentration of the dilution chamber 32 is calculated for the next subsequent intervals based on the amount of pharmaceutical preparation that entered the dilution chamber 32 during the particular subsequent interval preceding each next subsequent intervals.

It is important to note that the above described process (as illustrated in FIGS. 13b and 13d) provides the values of rate as dictated by the Sadleir functions providing a curve (Sadleir theoretical curve) as shown for a particular example (for a 50 mL pharmaceutical preparation, with a 10 mL dilution chamber). Once the Sadleir theoretical curve has been calculated, the apparatus 10 in accordance with the second embodiment of the disclosure is programmed accordingly in order to administer the drug to the patient using the infusion driver 14.

The process for administering the drug using the infusion driver 14 in accordance with the Tansy or Sadleir function requires approximating the Tansy or Sadleir function with a series of ramp infusion steps (linearly changing infusion rate from beginning of the step to end) or constant infusion steps occurring sequentially during the duration of the infusion. Each step need to be adjusted to give the same or approximate volume of pharmaceutical preparation for the summation of corresponding intervals of the infusion driver 14 controlled by the Sadleir function. This particular process of approximation will be described at a later stage.

In operation, the process of setting up the apparatus 10 in accordance with the second embodiment of the disclosure for administering the drug requires two ‘priming’ steps and the drug-dosing infusion sequence for delivering the drug in accordance with the Sadleir function, as follows:
a) a first priming step to ensure the infusion driver 14 is free of slack and primes the conduit 30a, and
b) a second priming step to move the diluted pharmaceutical preparation from the exit of the dilution chamber 38 to the point of intravenous access in the patient.

In the first priming step, the conduit 30a is filled with the pharmaceutical preparation. This is done by opening the multi-way valve 42 to the atmosphere and operating the infusion driver 14 to purge the drug to the multi-way valve 42. The infusion driver 14 is stopped and the multi-way valve 42 is moved to impede contact between the conduit 30a and the atmosphere, and to open the dilution chamber 32 for delivery of the pharmaceutical preparation into the container 34 of the dilution chamber 32.

In the second priming step, the container 34 of the dilution chamber 32 and the catheter 50 plus its distal end 54 are filled with the pharmaceutical preparation. This will result in the pharmaceutical preparation entering the dilution chamber 32. During this second priming step, the infusion driver 14 is programmed to generate alternating rapid and slow flow rates to allow mixing of the drug and diluent contained in the container 34 of the dilution chamber 32. The second priming step continues until the first initial portion of mixed drug and diluent entering the first outlet 38 is advanced the length of conduit 30b and up to the point of entry into the patient. In this step, no drug is administered to the patient; thus, the alternating flow rate does need to be taking into account when calculating patient dosing.

Subsequently, the Sadleir method (for example, using ramp-step or constant-step approximation) is then started, resulting in infusion of the pharmaceutical preparation into the patient at the flow rate as dictated by the Sadleir function.

The functions used in Tansy or Sadleir methods (referred to as Tansy Functions and Sadleir functions) define the flow rate of the pharmaceutical preparation to administer the pharmaceutical preparation active ingredient (drug) to the patient at an initially low rate, with the flow rate varying as the infusion continues.

Approximations of the Tansy or Sadleir function may be used if the infusion driver 14 is only capable of delivering a finite number of infusion steps. The approximation may be done using a constant-infusion profile over each infusion step, or a linearly increasing or decreasing infusion rate over each step.

In fact, typically, programmable infusion devices (such as syringe drivers or peristaltic pumps or similar drug infusion pumps) are not capable of providing in a continuous manner the pharmaceutical preparation (with infinitely small steps). Instead the infusion devices provide either a series of constant steps, or a series of ‘ramp’ steps. The “ramp steps” start at one rate and linearly increase or decrease to another rate over the interval of the step. There may be a finite number of steps, either because of memory limits, or due to the unfavourable effect of latency between each step (an interruption to the infusion between each step). It should be noted that with the Sadleir method, even a series of constant rate or ramp rate infusion steps will result in a continuously changing active ingredient (drug) administration rate due to the continuously changing concentration of pharmaceutical preparation leaving the dilution chamber 32.

In accordance with the present embodiments of the disclosure there are provided several methods of approximating the Tansy or Sadleir function with a series of constant steps or ramp steps, and an improved method for each. FIGS. 25c and 25d illustrate the dose of active ingredient administered to the patient that results from the approximation process for the Sadleir function using a constant infusion method or ramp infusion method for the first 4 minutes of a 30 minute infusion, using 40 steps of 45 seconds duration.

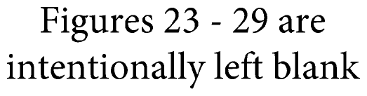

As shown in the FIGS. 12 and 13, each of the Tansy (FIGS. 23b and 23c) Sadlier methods include defining the quantity of infusion steps that will sequentially occur during the duration of the infusion. Each step has a specific duration during which a particular quantity of pharmaceutical preparation will be provided. In a particular arrangement, these steps will deliver a similar volume as the Tansy or Sadleir function over the equivalent time interval of the infusion.

As mentioned above, during each of these steps there will be provided a particular quantity of the pharmaceutical preparation. The particular quantity of the pharmaceutical preparation that will be provided during each particular step will depend on the particular quantity of the pharmaceutical preparation that the Tansy or the Sadleir functions dictate that must be provided during the time interval of the particular infusion interval; in particular, as will be described below, this particular quantity is calculated using the quantity dictated for each particular interval at the corresponding particular moments of time during the infusion process as dictated by the Tansy (see FIG. 12b) or the Sadleir (see FIG. 13c) functions.

FIGS. 12b and 13c respectively illustrate the methods of approximating the Tansy and Sadleir function and delivering the pharmaceutical preparation to the patient.

As shown in FIG. 12b in relation the Tansy method, after having calculated the actual amount (volume) of pharmaceutical preparation that will be delivered at the particular period of time of each step, it is decided whether the flow rate will be kept constant or increased linearly over each infusion step, depending on the capabilities of the infusion driver 14. The volume delivered in each step will be based on the volume of pharmaceutical preparation that has been calculated to be delivered over the corresponding interval of the Tansy function (see FIG. 12b).

Subsequently, the priming step will commence by delivering enough pharmaceutical preparation to the patient to fill the conduits 30a with the pharmaceutical preparation up to the point of the intravenous access point of the patient. At this stage, the infusion process may start by delivering to the patient, during each step, the amount of pharmaceutical preparation calculated for each step. Upon expiry of the infusion period, the infusion process is stopped.

As shown in FIG. 13c in relation to the Sadleir method, after having calculated the actual amount of pharmaceutical preparation that will be infused at the particular period of time of each step, the first priming step will commence by delivering enough pharmaceutical preparation to fill the conduits 30a with the pharmaceutical preparation. Subsequently, the second priming step will commence for filing the dilution chamber 32 and the conduit 30b for the diluted pharmaceutical preparation to reach the patient.

The infusion process then may start by (1) calculating the flow rate during the first step and (2) then delivering the pharmaceutical preparation to the patient at the calculated rate. At this stage, the pharmaceutical preparation may be delivered during each step to the patient until culmination of the infusion process.

With reference to the Sadleir method, as shown in FIG. 13c, after delivery of the pharmaceutical preparation during each step, it is necessary to calculate the flow rate that is required for delivering the required amount of pharmaceutical preparation during the subsequent step. Finally, upon expiry of the infusion period, the infusion process is stopped and the remaining pharmaceutical preparation is delivered to the patient by, for example, collapsing the dilution chamber 32 as was described before in relation to the apparatus 10 depicted in FIGS. 1 to 11.

Alternative arrangements of the Sadleir method to approximate the active ingredient dosing rates of the Tansy method.

In an alternative arrangement of the Sadleir method, the apparatus may comprise a container 34 (comprising the dilution chamber 32) with the container 34 not having the ability to be selectively displaced between an expanded condition and a contracted condition (i.e. is of fixed volume). In order to compensate for the reduced total dose of drug administered compared to the equivalent Tansy function which results as a consequence of drug being present in the dilution chamber 32 at completion of the infusion, the concentration or volume of pharmaceutical preparation can be increased so that the active ingredient dosing rates of the equivalent Tansy method is provided. Particularly we can increase the:
a) concentration of the drug in the syringe 15 of the infusion driver 14 prior to commencement of the infusion process. The concentration will be equal to the original concentration (the concentration that would be required in order to provide the prescribed dose of active ingredient) multiplied by the inverse of the ‘correction factor’, or

$$\frac{{V}_{p}}{{V}_{p} - {V}_{d} ( 1 - {e}^{- \frac{{V}_{p}}{{V}_{d}}} )} .$$

The method (‘Increased concentration Sadleir method’) will deliver the equivalent drug dosing as for the Tansy function, rather than the modified Tansy function, see FIG. 13c. For example, if the Tansy method is used to deliver 2 g of cephazolin as a 50 mL pharmaceutical preparation over 30 minutes (concentration 0.04 g/ml) the Sadleir method that will deliver the same dosing profile will be programmed to deliver 2.496 g of cephazolin in 50 ml of pharmaceutical preparation (concentration approximately 0.05 mg/ml) over 30 minutes, and a pharmaceutical preparation of this concentration (0.05 mg/ml) with sufficient volume to allow for priming the apparatus 10 (total volume of preparation approximately 53 mL) will be required; or
b) volume of pharmaceutical preparation, whereby an increased total volume of pharmaceutical preparation with the same concentration as that of the pharmaceutical preparation for the equivalent Tansy method is delivered over the same duration of the infusion. This increased volume of pharmaceutical preparation is determined by determining the volume that would be delivered in executing the method of the Kelly function over the infusion duration. That is, this is determined by applying the Kelly function to the duration of the infusion interval, and will be an amount less than Vₚ+V_{d}. This total volume is then delivered over the duration of the infusion according to the Kelly function (see FIG. 29a). This alternate version delivers to the patient a dose of active ingredient that is equal to the equivalent Tansy method at each point in time during the infusion, rather than a fraction of the amount as occurs in the Sadleir method. For example, if the Tansy method is used to deliver 2 g of cephazolin as a 50 ml pharmaceutical preparation over 30 minutes (concentration 0.04 g/ml), this method to deliver the same dosing profile will be programmed to deliver 59.98 mL of infusion of pharmaceutical preparation of the same concentration as the Tansy function (2.4 g of cephazolin in 59.98 mL, concentration 0.04 g/ml) and over the same infusion duration, using the algorithm in FIG. 29a. This is illustrated in FIGS. 29b, 29c and 29d and referred to as the ‘Increased volume Sadleir method’. The total volume of pharmaceutical preparation in the syringe in the syringe driver may need to be increased further to allow for the volume required for the priming steps prior to the infusion.

The ‘Increased concentration Sadleir method’ comprises the use of the second embodiment of the disclosure with an increased concentration of active ingredient in the pharmaceutical preparation compared to that of the comparable (same Vₚ and i) Tansy Method. The active ingredient concentration in the pharmaceutical preparation equals the equivalent Tansy method concentration multiplied by:

$$\frac{{V}_{p}}{{V}_{p} - {V}_{d} ( 1 - {e}^{- \frac{{V}_{p}}{{V}_{d}}} )} .$$

For the example of a 50 mL infusion volume with a 10 mL dilution chamber, choosing a pharmaceutical preparation with a drug concentration that is

$$\frac{50}{50 - 10 ( 1 - {e}^{- \frac{50}{10}} )} = 1.2479$$

times the concentration in the equivalent

Tansy Method will result in the same active ingredient (drug) dosing profile. The infusion rates and volumes delivered during the ‘Increased concentration Sadleir method’ are the same as for the previously described Sadleir method. At completion of the infusion, the contents of the dilution chamber are discarded.

If an increased volume of infusion is not contraindicated, a further alternative arrangement of the second embodiment of the disclosure that provides the same active ingredient dosing profile as the equivalent Tansy method is the ‘Increased volume Sadleir method’. In the Increased volume Sadleir method, when compared to the equivalent Tansy method, the same infusion duration and pharmaceutical preparation active ingredient concentration is used. However, a larger volume of pharmaceutical infusion and higher rate of infusion is used to deliver the same dosing of active ingredient as for the equivalent Tansy method. The higher infusion volume is calculated by an iterative function described below, and the higher infusion rates are calculated using a modification of the Sadleir function. Any solution in the dilution chamber 32 at the end of the infusion period is discarded.

The particular infusion driver 14 used for this realisation is capable of linearly-changing rate throughout each infusion step (Ramp-step program) or a constant rate throughout each infusion step (Constant-step program). If there is a period between infusion steps where fluid is not administered (a pause), this is defined as a latent period and the duration of this period is noted and accounted for as was described earlier when explaining the method for approximating the Tansy curve.

As mentioned earlier, the Sadleir and Tansy the infusion rates are relatively low during most of the start of the infusion process. This allows, concurrently with the actual process of infusing the pharmaceutical preparation, administering a wide range of test doses that may recognize a negative reaction in the patient (who was not known to be allergic to the pharmaceutical drug). This may result in the identification that the patient is allergic to the drug that is being infused into the patient and allows the infusion to be stopped before the patient is administered a dose that would result in a more serious or lethal reaction. The present infusion processes are also particularly useful (1) in the circumstances where it is suspected that the patient may be allergic to the drug (a drug challenge), or (2) to induce desensitization in a patient who is allergic to the drug but that which may or may not be suspected prior (a drug desensitization).

### Alternative Medication Delivery System

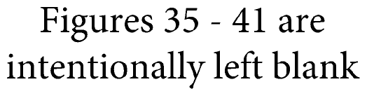

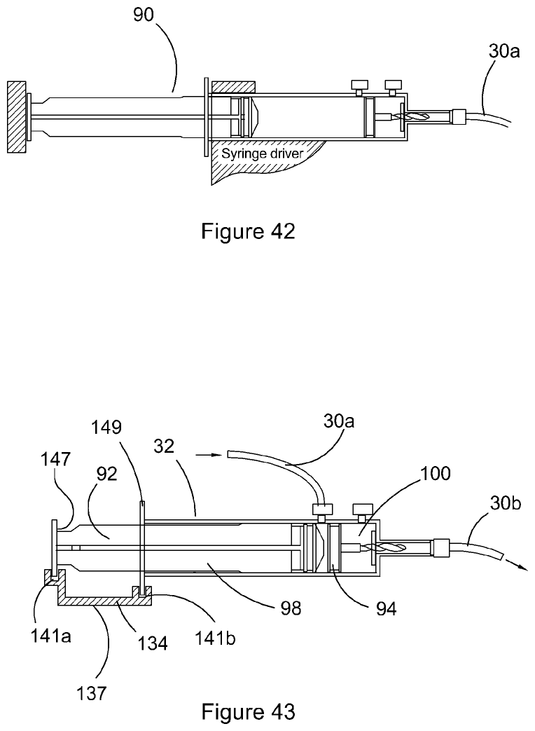

Referring now to FIGS. 30 to 47, FIGS. 30 to 47 show particular arrangements of a medication delivery system 91 comprising a medication delivery apparatus 90 in accordance with particular embodiments of the disclosure.

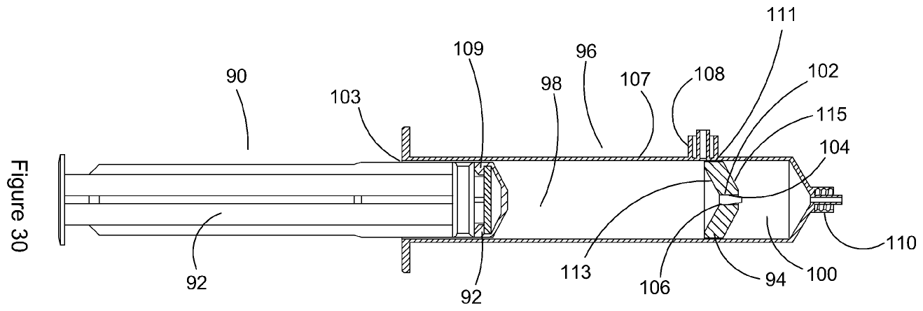

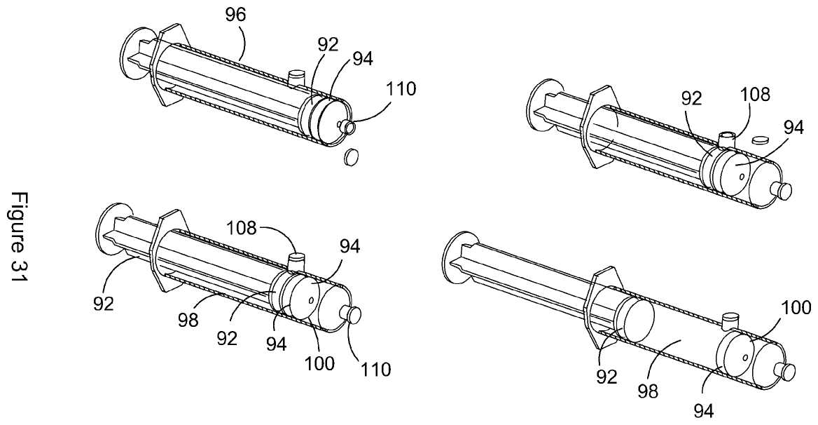

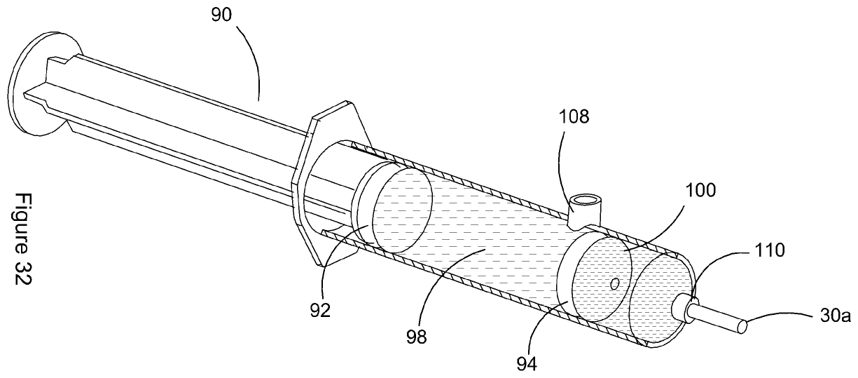

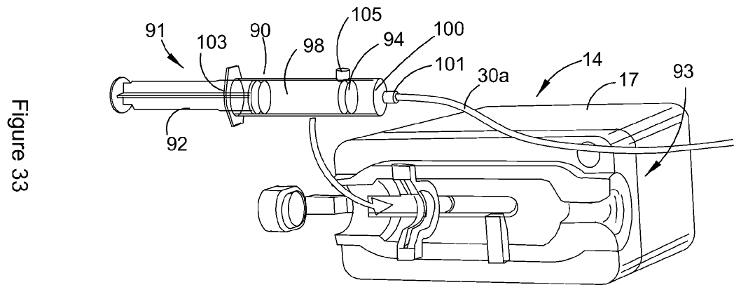

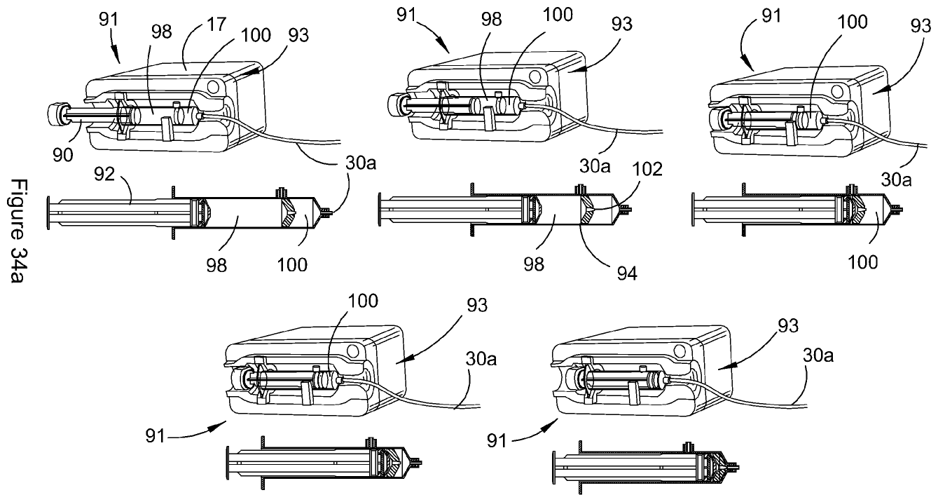

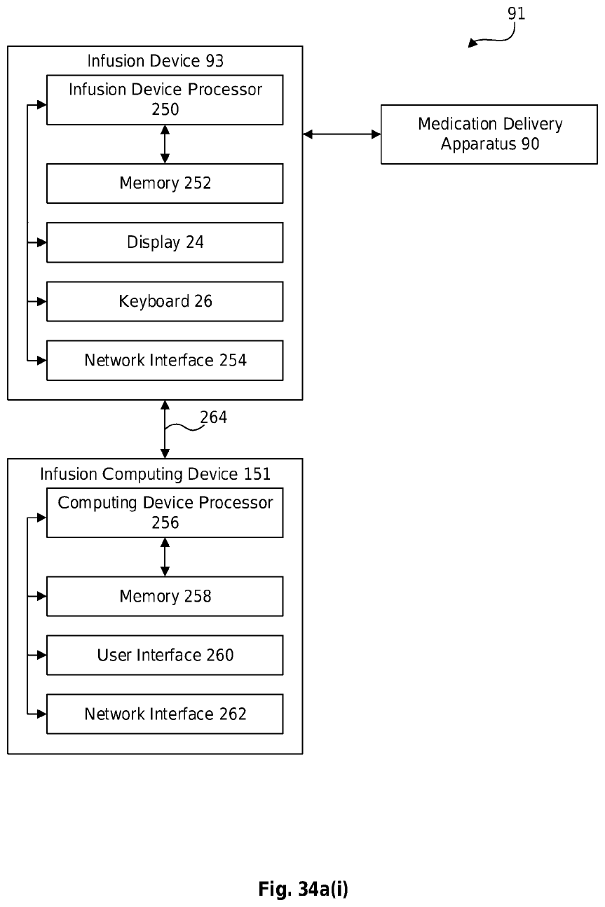

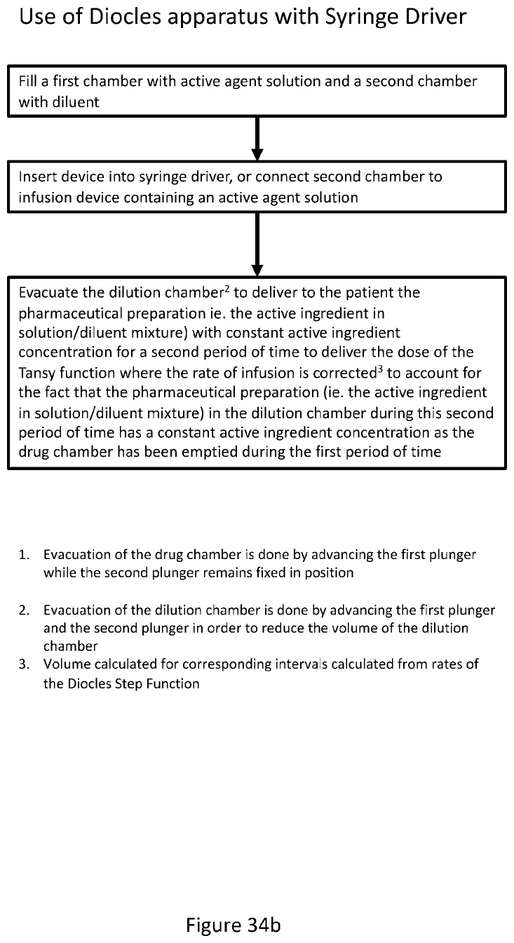

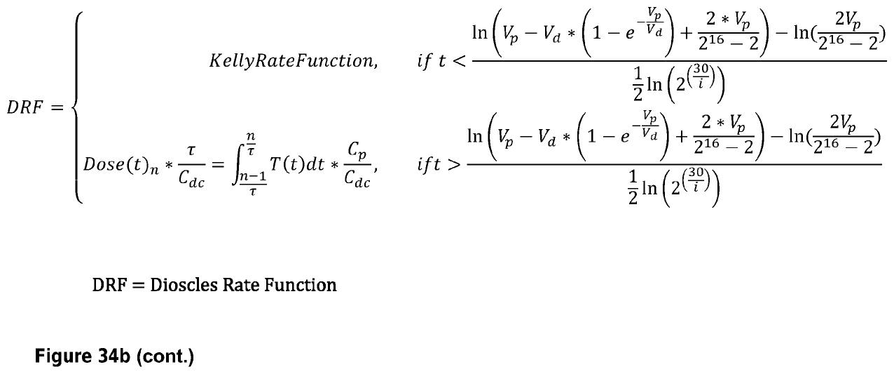

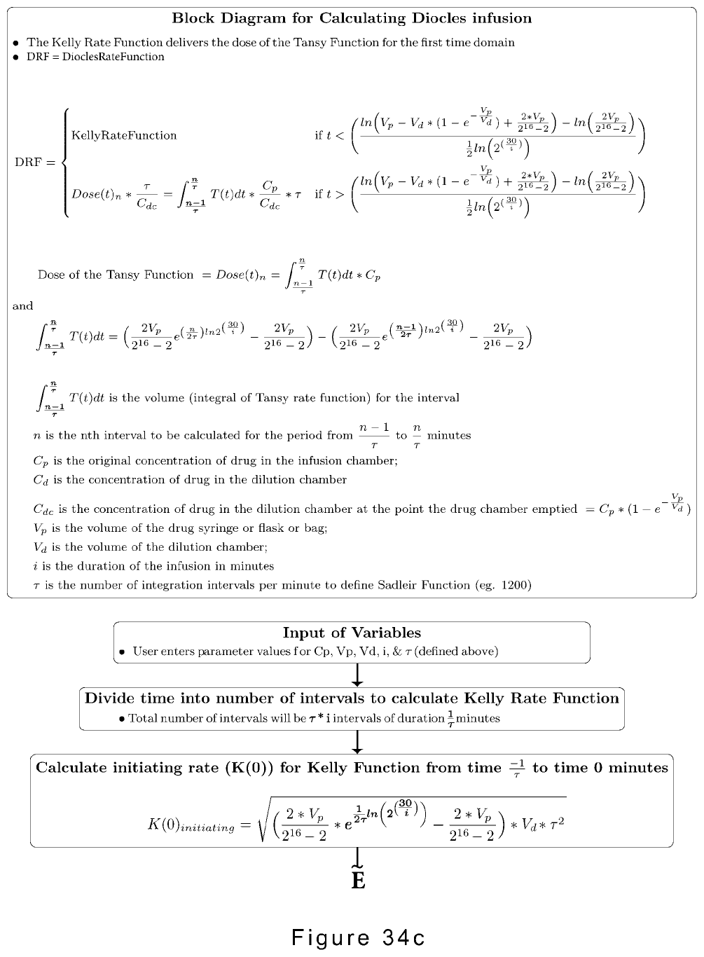

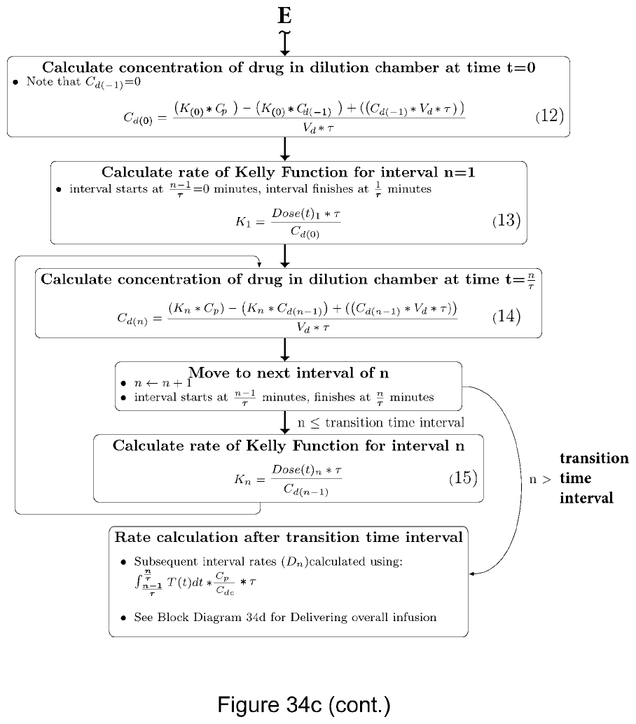

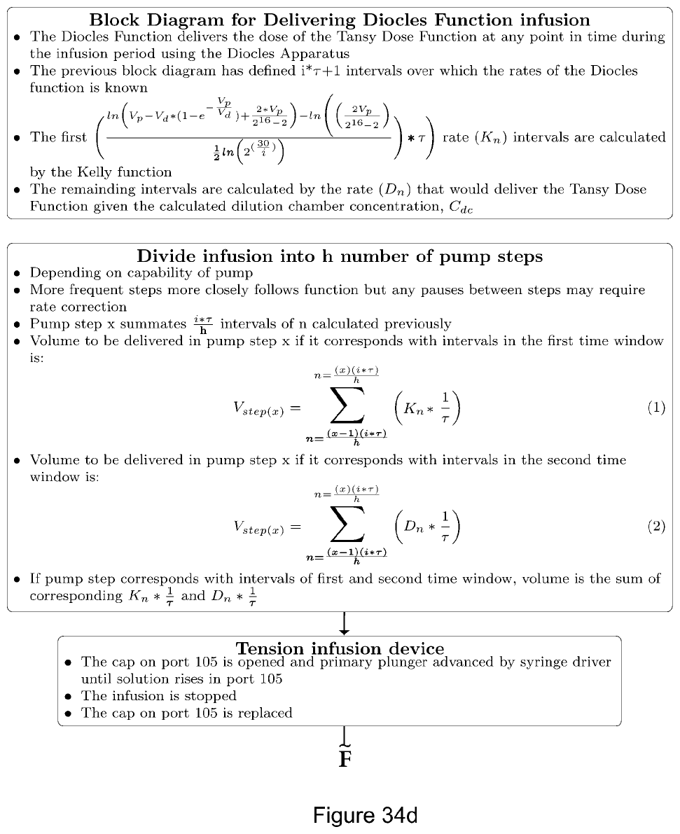

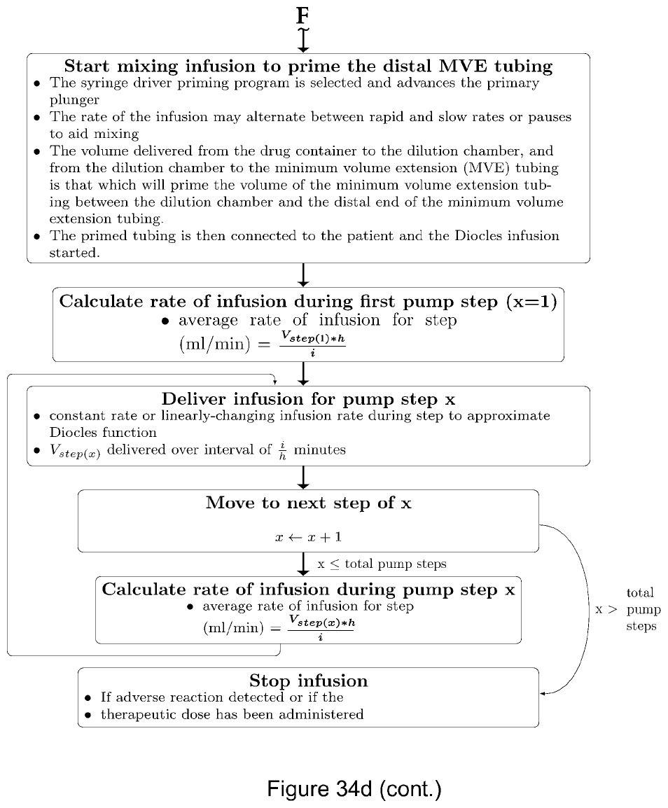

As shown in FIG. 30 and FIG. 34a(i), in some embodiments, the medication delivery system 91 comprises the medication delivery apparatus 90 and an infusion device 93. The infusion device 93 is illustrated in the form of a syringe driver. In some embodiments, the infusion device 93 may comprise or be in the form of a vacuum infusion device. The infusion device 93 may be similar to, or the same as the previously described infusion device 14. The infusion device 93 may apply an infusion pressure (i.e. the vacuum pressure 61) at a dilution chamber opening 53, thereby causing displacement of the first plunger 13 such that the pharmaceutical preparation is output by the medication delivery apparatus 2 at a target flow rate. The infusion pressure may be a negative pressure.

The infusion device 93 comprises an infusion device processor 250. The infusion device 93 comprises infusion device memory 252. The infusion device comprises a user interface. The user interface may comprise a display 24, which may be as described herein. The user interface may comprise a keyboard 26, which may be as described herein. The infusion device 93 is configured to actuate the first plunger 92 of the medication delivery apparatus 90, as is described herein.

The infusion device processor 250 is configured to execute instructions stored in the infusion device memory 252 to cause the infusion device 93 to function according to the described methods. In some embodiments, the instructions are in the form of instruction program code. The infusion device processor 250 may comprise one or more microprocessors, central processing units (CPUs), application specific instruction set processors (ASIPs), application specific integrated circuits (ASICs) or other processors capable of reading and executing instruction code.

The infusion device memory 252 may comprise one or more volatile or non-volatile memory types. For example, the infusion device memory 252 may comprise one or more of random access memory (RAM), read-only memory (ROM), electrically erasable programmable read-only memory (EEPROM) or flash memory. The infusion device memory 252 is configured to store program code accessible by the infusion device processor 250. The program code comprises executable program code modules. In other words, the infusion device memory 252 is configured to store executable code modules configured to be executable by the infusion device processor 250. The executable code modules, when executed by the infusion device processor 250, cause the infusion device 93 to perform certain functionality, as described in more detail herein (e.g. to actuate the first plunger 92).

The infusion device 93 comprises an infusion device network interface 254. The infusion device network interface 254 allows the infusion device 93 to communicate with one or more other computing devices over a communications network 264. The infusion device network interface 254 may comprise a combination of network interface hardware and network interface software suitable for establishing, maintaining and facilitating communication over a relevant communication channel. Examples of a suitable communications network 264 include a cloud server network, wired or wireless internet connection, Bluetooth™ or other near field radio communication, and/or physical media such as USB.

The medication delivery system 91 comprises an infusion computing device 151. The infusion computing device 151 comprises an infusion computing device processor 256. The infusion computing device 151 comprises an infusion computing device memory 258. The infusion computing device 151 comprises an infusion computing device user interface 260. The infusion computing device 151 comprises a network interface 262. The infusion computing device 151 is configured to receive one or more method inputs and to determine an infusion process based at least in part on the one or more method inputs.

The infusion computing device user interface 260 may comprise a display. The display may be as described herein. The infusion computing device user interface may comprise a keyboard. The keyboard may be as described herein. The infusion computing device 151 is configured to receive the one or more method inputs via the infusion computing device user interface 260.

The infusion computing device processor 256 is configured to execute instructions stored in the infusion computing device memory 258 to cause the infusion computing device 151 to function according to the described methods. In some embodiments, the instructions are in the form of instruction program code. The infusion computing device processor 256 may comprise one or more microprocessors, central processing units (CPUs), application specific instruction set processors (ASIPs), application specific integrated circuits (ASICs) or other processors capable of reading and executing instruction code.

The infusion computing device memory 258 may comprise one or more volatile or non-volatile memory types. For example, the infusion computing device memory 258 may comprise one or more of random access memory (RAM), read-only memory (ROM), electrically erasable programmable read-only memory (EEPROM) or flash memory. The infusion device memory 252 is configured to store program code accessible by the infusion computing device processor 256. The program code comprises executable program code modules. In other words, the infusion computing device memory 258 is configured to store executable code modules configured to be executable by the infusion computing device processor 256. The executable code modules, when executed by the infusion computing device processor 256, cause the infusion computing device 151 to perform certain functionality, as described in more detail herein (e.g. to determine an infusion process as described herein).

The network interface 262 allows the infusion commuting device 151 to communicate with one or more other computing devices over the communications network 264. For example, the network interface 262 enables the infusion computing device 151 to communicate with the infusion device 93. The network interface 262 may comprise a combination of network interface hardware and network interface software suitable for establishing, maintaining and facilitating communication over a relevant communication channel.

Although the infusion computing device 151 has been described with reference to the medication delivery system 91, it will be appreciated that in some embodiments, the medication delivery system 1 may comprise an infusion computing device 151 as described herein.

In some embodiments, the medication delivery apparatus 90 comprises a first plunger 92 (which may also be referred to as a primary plunger) and a second plunger 94 (which may also be referred to as a separating plunger). The medication delivery apparatus 90 also comprises a container 96 for receiving the second plunger 94 and at least a portion of the first plunger 92. This may be a distal portion of the first plunger 92.

The presence of the separating plunger 94 within the container 96 defines two chambers within the container 96, in particular: a first chamber 98 (the active agent chamber) and a second chamber 100 (the mixing chamber). In particular, the container 96 and the second plunger 94 together define a dilution chamber 100 that is configured to receive a diluent. The dilution chamber 100 may be similar to, or the same as the dilution chamber 32 previously described. The first plunger 92, the container 96 and the second plunger 94 together define an active agent chamber 98. The active agent chamber 98 is configured to receive a pharmaceutical preparation.

Further, as will be described with the method of operation of the medication delivery apparatus 90, the separating plunger 94 is adapted to permit flow of the fluid (e.g. the active agent) contained in the active agent chamber 98 into the dilution chamber 100. The dilution chamber 100 may also be referred to as a mixing chamber. The mixing chamber 100 comprises the diluent for mixing with the pharmaceutical preparation (or active agent) flowing from the active agent chamber 98 into the mixing chamber 100, for preparation of the pharmaceutical composition (diluted pharmaceutical preparation) to be delivered to the patient.

In accordance with the present embodiments of the disclosure, the second plunger 94 comprises valve means 102 (which may also be referred to as a valve 102) adapted to control flow the active agent entering the mixing chamber 100. In other words, the second plunger 94 comprises a valve 102 configured to control a flow of pharmaceutical preparation from the active agent chamber 98 to the dilution chamber 100. The valve 102 may be configured to control the flow of the pharmaceutical preparation in response to applied pressure. The pressure may be applied by the first plunger 92. Alternatively, the pressure may be applied via the first plunger 92. In the particular arrangement shown in FIGS. 30 to 34a, the valve means 102 comprises a duckbill valve 104. The duckbill valve 104 comprises a plurality of flaps 106 that, as pressure is applied to the first plunger 92, separate with respect to each other opening the duckbill valve 104. Upon removal of the pressure, that is being applied to the first plunger 92, the flaps 106 return to their original condition closing the duckbill valve 104 and impeding backflow of the pharmaceutical preparation back into the active agent chamber 98.

The valve 102 (or valve means 102) comprises an inlet side 113 and an outlet side 115. The valve 102 (or valve means 102) is configured to move from a closed position to an open position upon application of pressure to the inlet side 113. Pressure may be applied to the inlet side 113 of the valve 102 (or valve means 102) by longitudinally displacing (or actuating) the first plunger within the chamber 96 to displace the pharmaceutical preparation. The valve 102 (or valve means 102) is configured to move from the open position to the closed position upon removal of the pressure applied to the inlet side. The valve 102 (or valve means 102) may be configured to move from the closed position to the open position when a pressure applied to the inlet side 113 exceeds a pressure threshold. The valve 102 (or valve means) may be configured to move from the open position to the closed position when the pressure applied to the inlet side 113 is below a pressure threshold. The valve 102 (or valve means 102) is biased towards the closed position. The valve 102 (or valve means 102) comprises the plurality of flaps 106. The plurality of flaps 106 are configured to separate upon application of pressure to the inlet side 113. The first plunger 92 is configured to contact the second plunger 94 once all, or most of, the pharmaceutical preparation in the active agent chamber 98 has been transferred to the dilution chamber 100. Further actuation of the first plunger 92 will also result in movement of the second plunger 94. Thus, actuation of the first plunger 92 causes movement of the second plunger 94, and causes the pharmaceutical preparation in the dilution chamber 100 to be output by the medication delivery apparatus 90.

Further, the container 96 comprises at least one first port 108 (the inlet port) and a second port 110 (the outlet port). The inlet port 108 allows filling of the container 96 with active agent and the second port 110 allows either (1) filing the mixing chamber with diluent or (2) permitting exit of the mixture of the active agent and diluent (the pharmaceutical composition) from the container 96 (in particular, from the mixing chamber 100) for delivery to the patient. The container 96 comprises a first active agent chamber opening 103 that is configured to receive at least a portion of the first plunger 92. In particular, the active agent chamber 98 comprises the active agent chamber opening 103. The inlet port 108 may be considered a second active agent chamber opening that is configured to receive the pharmaceutical preparation. In other words, the active agent chamber 98 may be said to comprise a second active agent chamber opening that is configured to receive the pharmaceutical preparation. The second active agent chamber opening (the inlet port 108) is defined in the wall of the container 96. The active agent chamber 98 may be filled with pharmaceutical preparation by introducing the pharmaceutical preparation into the active agent chamber 98 via the second active agent chamber opening (i.e. the first port 108). The first port 108 may therefore be referred to as an active agent chamber inlet. The dilution chamber 100 comprises a dilution chamber opening 110 that is defined by the container 96. The dilution chamber opening 110 may be referred to as the outlet port of the container 96.

In the arrangement shown in figures, the inlet and outlet ports 108 and 110 (as well as inlet and outlet ports 118 and 120) are shown as male Luer-lock connector; however, in alterative arrangements, for example, the inlet ports, such as 108 and 118, may comprise female Luer-lock connectors.

The first plunger 92 and the second plunger 94 are each configured to be displaced with respect to a longitudinal axis of the container 96. The second plunger 94 is disposed between the first plunger 92 and the dilution chamber opening 110 (i.e. the outlet port 110). The second plunger 94 is disposed between the inlet port 108 (the second active agent chamber opening) and the dilution chamber opening 110.

The container 96 defines a inner container surface 107. The first plunger 92 comprises a first plunger sealing surface 109. The first plunger 92 is configured to seal with the inner container surface 107. In particular, the first plunger sealing surface 109 is configured to seal with the inner container surface 107 to inhibit fluid flow between the inner container surface 107 and the first plunger sealing surface 109.

The second plunger 94 comprises a second plunger sealing surface 111. The second plunger 94 is configured to seal with the inner container surface 107. In particular, the second plunger sealing surface 111 is configured to seal with the inner container surface 107 to inhibit fluid flow between the inner container surface 107 and the second plunger sealing surface 111.

The medication delivery apparatus 91 comprises a conduit 30a. The conduit 30a is configured to be fluidly connected to the dilution chamber opening 110. The conduit 30a is of a predetermined volume. That is, a length and an internal surface area of the conduit 30a are sized so that the conduit 30a defines a predetermined volume. The conduit 30a can therefore hold or store a volume of the diluted pharmaceutical preparation prior to the diluted pharmaceutical preparation being delivered to the patient. The conduit 30a may be referred to as a minimum volume extension tube. The conduit 30a is configured to retain a first volume of infusion to be delivered to the patient. The first volume of infusion can be prepared by the priming process at a rate that will result in effective mixing in the dilution chamber 110. This is possible because during this time, no pharmaceutical preparation is delivered to the patient. Thus, a different flow rate can be used for the first volume when priming, while driving the mixed fluid exiting the dilution chamber 100 to the end of the conduit 30a. Although the conduit 30a of the medication delivery apparatus 91 is described to be of a predetermined volume, it will be understood that a conduit of a predetermined volume could be used with any of the medication delivery apparatuses disclosed herein to achieve similar functionality and benefits.

FIG. 31 shows the process for filing the container 96 of the medication delivery apparatus 90 with active agent and diluent.

As shown in FIG. 31, the process for filing the container 96 comprises the step of delivering the diluent into the mixing chamber 100 by opening the outlet 110 and delivering the diluent into the mixing chamber 100. Due to the entrance of the diluent into the mixing chamber 100, the separating plunger 94 is displaced away from the outlet 110 permitting entrance of the diluent and carrying with it the primary plunger 92.

Once the mixing chamber 100 is filled with the corresponding quantity of diluent, the outlet 110 is closed permitting filling of the active agent chamber 98.

Filling the active agent chamber 98 comprises the step of opening the inlet port 108 for delivery of the pharmaceutical preparation into the active agent chamber 98. Filling of the active agent chamber 98 displaces the primary plunger 92 further from the outlet 110 until all of the corresponding quantity of pharmaceutical preparation is delivered into the active agent chamber 98.

At this stage, the inlet 108 is closed and the medication delivery apparatus 90 may be prepared for delivery of the pharmaceutical composition to the patient.

Preparation of the medication delivery apparatus 90 comprises the step of attaching the conduit 30a to the outlet 110 as is shown in FIG. 32. The conduit 30a comprises a minimal volume tubing adapted to be attached to the outlet 110 and to an infusion device for delivering the pharmaceutical composition into the patient's blood stream.

Subsequently, as shown in FIG. 33, the medication delivery apparatus 90 is mounted on the infusion device 14, thereby forming a medication delivery system 91. The infusion device of FIG. 33 is in the form of a syringe driver 17. The medication delivery apparatus 90 is mounted to the syringe driver 17 in order to: (1) prepare the pharmaceutical composition by mixing the pharmaceutical preparation and the diluent, and (2) deliver the pharmaceutical composition (i.e. the diluted pharmaceutical preparation, or the pharmaceutical preparation—when the diluent is consumed) into the conduit 30a for infusion into the patient.

As shown in FIG. 34a, preparation of the pharmaceutical composition comprises the step of pushing the primary plunger 92 in order that the pharmaceutical preparation contained in the active agent chamber 98 is delivered into the dilution chamber 100 for mixing with the diluent contained in the diluent chamber 100. The primary plunger 92 is pushed by the syringe driver 17 in such a manner that the pharmaceutical preparation is delivered into the mixing chamber 100 in order to provide, in conjunction with the valve means 102, a particular mixing profile within the mixing chamber 100 to allow proper mixing of the pharmaceutical preparation with the diluent.

As the pharmaceutical preparation contained in the active agent chamber 98 is delivered into the dilution chamber 100, mixing occurs for generating the pharmaceutical composition (in this case, the diluted pharmaceutical preparation), which is then delivered into the conduit 30a for infusion into the patient. As the pharmaceutical composition is delivered into the conduit 30a, the concentration of active agent within the dilution chamber 100 will increase as the active agent is delivered into the dilution chamber 100 during the infusion. For delivery of the pharmaceutical composition to the patient, the primary plunger 92 (with the separating plunger 94 abutting the primary plunger 92) is pushed in such a manner that the pharmaceutical composition is delivered in accordance with a particular profile. In particular, the primary plunger 92 is driven based on particular algorithms.

Initially, before the primary plunger 92 is driven based on the particular algorithms and the conduit 30a is fluidly connected to the patient, the syringe driver 17 is operated to drive the primary plunger 90 in such a manner to fill (i.e. to prime) the conduit 30a to be fluidly connected to the patient for delivery of the pharmaceutical composition.

One advantage of priming the conduit 30a (as described in the previous paragraph) is that the conduit 30a will be filled with pharmaceutical composition (i.e. diluted active agent) prior delivering the pharmaceutical composition to the patient; thus, ensuring that the patient will immediately receive the pharmaceutical composition comprising diluted active agent.

Another advantage of priming the conduit 30a is that during priming of the conduit 30a (prior delivering any pharmaceutical composition to the patient) the active agent may be driven at an arbitrarily fast rate into the dilution chamber 100 to allow good mixing before any of the pharmaceutical composition is delivered to the patient; this ensures proper mixing of the pharmaceutical preparation and the diluent within the dilution chamber 100 prior delivering any pharmaceutical composition to the patient.

The infusion device actuator (e.g. the syringe driver 17) is adapted to drive the primary plunger 92 in a particular manner. For example, the syringe driver 17 may comprise processing means for running of algorithms for driving the primary plunger 92 in a particular manner to obtain a particular mixing profile as well as a delivery profile of the pharmaceutical composition.

The infusion device 93 is configured to control the medication delivery apparatus 90 to deliver medication in accordance with one or more of the methods described herein. In particular, the infusion device 93 is configured to actuate the first plunger 92. The infusion device 93 is configured to actuate the first plunger 92 such that the fluid stored in the dilution chamber 100 is expelled from the mediation delivery apparatus 90 during the infusion process.

The infusion device 93 may actuate the first plunger 92 in one of a number of ways. The infusion device 93 may directly actuate the first plunger 92. That is, the infusion device 93 may contact the first plunger 92 as part of the actuation. For example, where the infusion device 93 comprises a syringe driver 17 or another physical infusion device actuator, the infusion device may apply a force to the first plunger 92. The force may be applied in a direction that is parallel to the longitudinal axis of the container 96. The force may move the first plunger 92 towards the dilution chamber opening 110.

As described herein, in some embodiments, the infusion device 93 comprises or is in the form of a vacuum infusion device. In such cases, the infusion device 93 is configured to apply the vacuum pressure to the dilution chamber opening 110 (and/or the port 110). The vacuum pressure applies a vacuum force to the fluid in the dilution chamber 100. This can draw the fluid out of the dilution chamber 100 via the dilution chamber opening 110. The fluid may be drawn into the conduit 30A.

As the fluid in the dilution chamber 100 is generally incompressible, the vacuum force is also applied to the second plunger 94. The vacuum force may be transmitted to the fluid in the active agent chamber 98 (i.e. the pharmaceutical preparation) via the valve 102 of the second plunger 94 if the vacuum force exceeds a valve force threshold. The valve 102 is configured to open if a force exceeding the valve force threshold is applied to the inlet side 113 of the valve 102. Similarly, the valve 102 is configured to open if a magnitude of a negative force applied to the outlet side 115 of the valve 102 is greater than the valve force threshold. In other words, the valve 102 is configured to open if a pressure differential between the inlet side 113 of the valve 102 and the outlet side 115 of the valve 102 is greater than a pressure differential threshold.

As the vacuum force is applied to the outlet side 115 of the valve 102, the valve 102 can open and the vacuum force can draw the pharmaceutical preparation through the valve 102. The vacuum force can therefore be transmitted to the pharmaceutical preparation in the active agent chamber 98. As the pharmaceutical preparation is a fluid, it is also generally incompressible. Thus, the vacuum force is also applied to the first plunger 92.

In cases where the valve threshold force is greater than a break loose force of the first plunger 92, the first plunger 92 will be moved by the vacuum force. Therefore, the infusion device 93 is configured to actuate the first plunger 92, at least by using the vacuum pressure to cause motion of the first plunger 92.

### The Sadleir Method

The medication delivery system 91 previously described may be controlled to deliver a pharmaceutical preparation to a patient according to the Sadleir method. As previously described, the medication delivery system 91 comprises the medication delivery apparatus 90 and the infusion device 93. The infusion device 93 comprises the at least one infusion device processor and infusion device memory as previously described. The infusion device memory stores program instructions accessible by the at least one infusion device processor. The program instructions are configured to cause the at least one infusion device processor to actuate an infusion device actuator (e.g. syringe driver 17) to control the medication delivery apparatus 90 to deliver medication in accordance with the Sadleir method.

In particular, the program instructions are configured to cause the at least one infusion device processor to receive a concentration input (Cₚ) that is indicative of a concentration of the pharmaceutical preparation in the active agent chamber. The concentration may be a concentration of active agent in the pharmaceutical preparation. The concentration input (Cₚ) may be received via an input provided by a user. For example, the concentration input (Cₚ) may be input using the user interface 22. Alternatively, the concentration input (Cₚ) may be retrieved from the infusion device memory. Throughout this description, the concentration input (Cₚ) may be a concentration of a drug in, or delivered from the active agent chamber.

The program instructions are further configured to cause the at least one infusion device processor to receive a volume input (Vₚ) that is indicative of a volume of the pharmaceutical preparation. This may be a volume of the pharmaceutical preparation in the active agent chamber. The volume input (Vₚ) may be received via an input provided by a user. For example, the volume input (Vₚ) may be input using the user interface 22. Alternatively, the volume input (Vₚ) may be retrieved from the infusion device memory.

The program instructions are further configured to cause the at least one infusion device processor to receive a dilution chamber volume input (V_{d}) that is indicative of a volume of the dilution chamber 100. The dilution chamber volume input (V_{d}) may be received via an input provided by a user. For example, the dilution chamber volume input (V_{d}) may be input using the user interface 22. Alternatively, the dilution chamber volume input (V_{d}) may be retrieved from the infusion device memory. Throughout this disclosure, the dilution chamber volume input (V_{d}) may correspond to volume of the relevant dilution chamber.

The program instructions are further configured to cause the at least one infusion device processor to receive a time input (i) that is indicative of a time window over which the pharmaceutical preparation is to be administered. The time input (i) may be received via an input provided by a user. For example, the time input (i) may be input using the user interface 22. Alternatively, the time input (i) may be retrieved from the infusion device memory.

The program instructions are further configured to cause the at least one infusion device processor to receive an infusion number input (τ) that is indicative of a number of infusion intervals per minute over which an infusion modelling function is to be numerically approximated over the time window. The infusion number input (τ) may be received via an input provided by a user. For example, the infusion number input (τ) may be input using the user interface 22. Alternatively, the infusion number input (τ) may be retrieved from the infusion device memory. Throughout this disclosure, the infusion number input (τ) may correspond to the number of infusion intervals per minute over which the relevant function (e.g. the Sadleir function) is calculated.

Throughout this disclosure, it will be understood that an infusion interval is an interval over which the infusion is approximated via a numerical approximation. This may differ from infusion steps. Infusion steps are the actual infusion steps delivered by the relevant infusion device. The number of infusion intervals may exceed the number of pump steps for a given period of time. For example, a 30 s pump step may be numerically approximated by 600 infusion intervals. These infusion intervals are used to improve the accuracy of numerical approximations when using infusion modelling functions. The volumes, concentrations and/or flow rates determined with respect to infusion intervals during the numerical approximations are targeted when executing the lower resolution infusion steps that are actually executed by the infusion devices disclosed herein.

The program instructions are further configured to cause the at least one infusion device processor to receive a number of infusion steps (h) that are to be executed during the time window. Receiving the number of infusion steps (h) that are to be executed during the time over which the pharmaceutical preparation is to be administered may comprise receiving an infusion step input that is indicative of the number of infusion steps. Alternatively, receiving the number of infusion steps that are to be executed during the time over which the pharmaceutical preparation is to be administered may comprise retrieving the number of infusion steps from the infusion device memory. Receiving the number of infusion steps that are to be executed during the time over which the pharmaceutical preparation is to be administered may comprise multiplying the time input (i) and the infusion number input (τ). There are h infusion steps of i/h duration during the infusion process.

The program instructions are further configured to cause the at least one infusion device processor to receive a pharmaceutical preparation input. The pharmaceutical preparation input is indicative of one or more of an identity of the pharmaceutical preparation, a dose of the pharmaceutical preparation and a maximum pharmaceutical preparation administration rate.

The program instructions are further configured to cause the at least one infusion device processor to numerically approximate the infusion modelling function over the time window. The at least one infusion device processor may approximate the infusion modelling function over the time window as described in FIGS. 13a to 13c.

The program instructions are further configured to cause the at least one infusion device processor to determine the infusion rate of an infusion step. This is determined by summating a plurality of infusion interval volumes calculated by numerical approximation over which the infusion step will occur, and then determining the infusion rate that will deliver this volume across the duration of the infusion step.

The program instructions are configured to cause the at least one infusion device processor to take the user inputs and create a theoretical program of infusion rate versus time or infusion cumulative volume versus time where time is the duration over which the pharmaceutical preparation is to be administered. Alternatively, the program instructions may be configured to cause the at least one infusion device processor to look up a theoretical program stored in the device memory. The theoretical program may be the numerical approximation described herein.

Numerically approximating the infusion modelling function comprises determining a number of infusion intervals within the time window. That is, the at least one infusion device processor determines a number of infusion intervals within the time window.

Numerically approximating the infusion modelling function comprises determining an initiating target flow rate parameter (S(0)_{initiating}). The initiating target flow rate parameter (S(0)_{initiating}) is indicative of a target flow rate of the pharmaceutical preparation to be output by the medication delivery apparatus 90 during an initiating infusion interval of the numerical approximation.

Determining the initiating target flow rate (S(0)_{initiating}) comprises calculating:

$$\sqrt{( ( \frac{2 {V}_{p}}{{2}^{16} - 2} {e}^{\frac{1}{2 τ} ln ( {2}^{\frac{30}{i}} )} - \frac{2 {V}_{p}}{{2}^{16} - 2} ) ) * ( \frac{{V}_{p} - ( {V}_{d} * ( 1 - {e}^{- \frac{{V}_{p}}{{V}_{d}}} ) )}{{V}_{p}} ) ) * {τ}^{2} * {V}_{d}}$$

The program instructions are further configured to cause the at least one infusion device processor to determine an initiating pharmaceutical preparation concentration. The initiating pharmaceutical preparation concentration is indicative of an approximated concentration of the pharmaceutical preparation in the dilution chamber after the initiating infusion interval of the numerical approximation. The at least one infusion device processor determines the initiating pharmaceutical preparation concentration by calculating:

$${C}_{{d}_{( o )}} = \frac{( {S ( 0 )}_{initiating} \times {C}_{p} ) - ( {S ( 0 )}_{initiating} \times {C}_{{d}_{( - 1 )}} ) + ( {C}_{{d}_{( - 1 )}} \times {V}_{d} \times τ )}{{V}_{d} \times τ}$$

where C_{d}_{₍₋₁₎}=0 and C_{d}_{₍ₒ₎} is the initiating pharmaceutical preparation concentration.

The program instructions are further configured to cause the at least one infusion device processor to determine a subsequent target flow rate and a subsequent pharmaceutical preparation concentration for each of a plurality of subsequent infusion intervals of the numerical approximation. The subsequent target flow rates are each indicative of a target flow rate of the pharmaceutical preparation to be output by the medication delivery apparatus 90 during a respective subsequent infusion interval of the numerical approximation. The subsequent pharmaceutical preparation concentrations are each indicative of a subsequent approximated concentration of the pharmaceutical preparation in the dilution chamber after the respective subsequent infusion interval.

Each of the subsequent target flow rates is determined based at least in part on the subsequent pharmaceutical preparation concentration of a previous infusion interval of the respective infusion interval. That is, each of the subsequent target flow rates is determined based at least in part on the subsequent pharmaceutical preparation concentration of the infusion interval that occurred immediately before the infusion interval of that subsequent target flow rate. Each of the subsequent pharmaceutical preparation concentrations is determined based at least in part on the subsequent target flow rate of the respective subsequent infusion interval.

Determining a subsequent target flow rate for one of the plurality of subsequent infusion intervals of the numerical approximation comprises determining a flow rate parameter Sₙ where n is the number of the relevant infusion interval. Determining the flow rate parameter Sₙ comprises determining a dose parameter D_{mtf}(t)ₙ. Determining the dose parameter D_{mtf}(t)ₙ comprises calculating:

$${{D}_{mtf} ( t )}_{n} = {\int }_{\frac{n - I}{τ}}^{\frac{n}{τ}} T ( t ) d t \times {C}_{p} \times ( \frac{{V}_{p} - ( {V}_{d} \times ( 1 - {e}^{\frac{{V}_{p}}{{V}_{d}}} ) )}{{V}_{p}} )$$

where:
T(t) is a Tansy rate function;
Cₚ is the concentration input;
Vₚ is the volume input;
V_{d} is the dilution chamber volume input;
n is the number of the relevant infusion interval; and
t is the infusion number input.

Determining the flow rate parameter Sₙ comprises calculating:

$${S}_{n} = \frac{{{D}_{mtf} ( t )}_{n} \times τ}{{C}_{d ( n - 1 )}}$$

where n is the number of the relevant infusion interval, C_{d(n-1)} is a subsequent pharmaceutical preparation concentration of a previous infusion interval of the nth infusion interval and D_{mtf}(t)ₙ is the dose parameter.

In some embodiments, determining the subsequent pharmaceutical preparation concentrations of the numerical approximation comprises calculating:

$${C}_{d ( n )} = \frac{( {S}_{n} \times {C}_{p} ) - ( {S}_{n} \times {C}_{d ( n - 1 )} ) + ( {C}_{d ( n - 1 )} \times {V}_{d} \times τ )}{{V}_{d} \times τ}$$

where C_{d(n)} is the subsequent pharmaceutical preparation concentration for the nth infusion interval of the numerical approximation and C_{d(n-1)} is the subsequent pharmaceutical preparation concentration for the n−1 th infusion interval of the numerical approximation. In other words, n is the number of the relevant infusion interval and C_{d(n-1)} is a subsequent pharmaceutical preparation concentration of a previous infusion interval of the nth infusion interval.

This calculation may be performed for each subsequent pharmaceutical preparation concentration of the iteration.

In some embodiments, determining the initiating target flow rate (S(0)_{initiating}) comprises calculating:

$$\sqrt{( \frac{2 {V}_{p}}{{2}^{16} - 2} {e}^{\frac{1}{2 τ} ln ( {2}^{\frac{30}{i}} )} - \frac{2 {V}_{p}}{{2}^{16} - 2} )} \times {V}_{d} \times {τ}^{2}$$

In some embodiments, determining the dose parameter comprises determining a dose of the Tansy function, by calculating

$${\int }_{\frac{n - 1}{τ}}^{\frac{n}{τ}} T ( t ) d t \times {C}_{p} .$$

Refer, for example, to FIG. 29a.

In some embodiments,

$${\int }_{\frac{n - 1}{τ}}^{\frac{n}{τ}} T ( t ) dt$$

is equal to:

$$( \frac{2 {V}_{p}}{{2}^{16} - 2} {e}^{( \frac{n}{2 τ} ) ln {2}^{( \frac{30}{i} )}} - \frac{2 {V}_{p}}{{2}^{16} - 2} ) - ( \frac{2 {V}_{p}}{{2}^{16} - 2} {e}^{( \frac{n - 1}{2 τ} ) ln {2}^{( \frac{30}{i} )}} - \frac{2 {V}_{p}}{{2}^{16} - 2} )$$

The subsequent target flow rate is indicative of a target flow rate of the pharmaceutical preparation to be output by the medication delivery apparatus 90 during a subsequent infusion step. The subsequent target flow rate is determined based at least in part on the subsequent pharmaceutical preparation concentration. The subsequent target flow rate is limited at the maximum pharmaceutical preparation administration rate. Therefore, the subsequent target flow rate does not exceed the maximum pharmaceutical preparation administration rate during infusion. Determining the subsequent target flow rate comprises determining a flow rate parameter Sₙ where n is the number of the relevant infusion step. Determining the flow rate parameter Sₙ comprises determining a dose parameter D_{mtf}(t)ₙ. Determining the dose parameter D_{mtf}(t)ₙ comprises calculating:

$${{D}_{mtf} ( t )}_{n} = {\int }_{\frac{n - 1}{τ}}^{\frac{n}{τ}} T ( t ) d t \times {C}_{p} \times ( \frac{{V}_{p} - ( {V}_{d} \times ( 1 - {e}^{- \frac{{V}_{p}}{{V}_{d}}} ) )}{{V}_{p}} )$$

where:
T(t) is a Tansy function;
Cₚ is the concentration input;
Vₚ is the volume input;
V_{d} is the dilution chamber volume input;
n is the number of the relevant infusion step; and
τ is the infusion number input.

Determining the initiating target flow rate (S(0)initiating) may comprise calculating:

$$\sqrt{( \frac{2 {V}_{p}}{{2}^{16} - 2} {e}^{( \frac{1}{2 τ} ) ln ( {2}^{\frac{30}{i}} )} - \frac{2 {V}_{p}}{{2}^{16} - 2} ) \times {V}_{d} \times {τ}^{2}}$$

Determining the dose parameter may comprise determining a dose of the Tansy function, by calculating:

$${\int }_{\frac{n - 1}{τ}}^{\frac{n}{τ}} T ( t ) dt$$

Which may be equal to:

$$( \frac{2 {V}_{p}}{{2}^{16} - 2} {e}^{( \frac{n}{2 τ} ) ln {2}^{( \frac{30}{i} )}} - \frac{2 {V}_{p}}{{2}^{16} - 2} ) - ( \frac{2 {V}_{p}}{{2}^{16} - 2} {e}^{( \frac{n - 1}{2 τ} ) ln {2}^{( \frac{30}{i} )}} - \frac{2 {V}_{p}}{{2}^{16} - 2} )$$

The program instructions are further configured to cause the at least one infusion device processor to determine an infusion volume for each of the number of infusion steps (h). The at least one processor determines the infusion volume for each of the number of infusion steps (h) based at least in part on the numerical approximation. Each infusion volume is indicative of a volume of the pharmaceutical preparation that is to be output by the medication delivery apparatus 90 during the respective infusion step.

In some embodiments, determining the infusion volume for one of the infusion steps comprises calculating:

$${V}_{step ( x )} = {\sum }_{n = \frac{( x - 1 ) ( i \times τ )}{h}}^{n = \frac{( x ) ( i \times τ )}{h}} ( {S}_{n} \times \frac{1}{τ} )$$

where Vₛₜₑₚ₍ₓ₎ is the infusion volume of the xth infusion step.

In some embodiments, the program instructions are further configured to cause the at least one infusion device processor to determine an infusion rate for each of the infusion steps. Determining the infusion rate for one of the infusion steps comprises calculating

$$\frac{{V}_{step ( x )} \times h}{i} ,$$

where Vₛₜₑₚ₍ₓ₎ is the infusion volume of the xth infusion step.

In some embodiments, the program instructions are further configured to cause the at least one infusion device processor to actuate the infusion device actuator to displace the first plunger within the chamber such that the determined infusion volume for each infusion step is output by the medication delivery apparatus 90 during the respective infusion step.

In some embodiments, the program instructions are further configured to cause the at least one infusion device processor to actuate the infusion device actuator such that the determined infusion volume for each infusion step is output by the medication delivery apparatus 90 during the respective infusion step at the determined infusion rate.

In some embodiments, the program instructions are further configured to cause the at least one infusion device processor to actuate the infusion device actuator such that the determined infusion volume for each infusion step is delivered according to a constant-rate profile or a linearly-changing rate profile. The constant-rate profile may be as described herein. The linearly-changing rate profile may be as described herein.

In some embodiments, the program instructions are further configured to cause the at least one infusion device processor to actuate the infusion device actuator such that the determined infusion volume for each infusion step is output by the medication delivery apparatus 90 during the respective subsequent infusion step in bursts. The bursts may be as described herein, for example, with reference to FIG. 48. The volume of infusion given during any infusion step in the Sadleir method may be given by constant infusion or linearly-varying infusion rates (‘ramp’). It may also be given by a single brief injection at a higher injection rate but lower duration such that the same volume is given but that the velocity of injection is greater and there is also a period where there is no advancement of the first plunger. There may be more than one cycle of advancement and no advancement during an infusion step (eg. ‘double burst’). The period of no advancement of the first plunger may allow the valve means 102 to close and resumption of advancement may result in opening and enhanced mixing.

In some embodiments, the concentration input Cₚ is increased by a factor of

$$( \frac{{V}_{p}}{{V}_{p} - ( {V}_{d} ( 1 - {e}^{- \frac{{V}_{p}}{{V}_{d}}} ) )} ) .$$

In some embodiments, the infusion modelling function is a Sadleir function.

The program instructions are further configured to cause the at least one infusion device processor to actuate the infusion device actuator to displace the first plunger 92 within chamber 96 such that the pharmaceutical preparation is output by the medication delivery apparatus 90 at subsequent flow rates for the remaining infusion steps until a total of h infusion steps have been delivered and the infusion is complete.

The volume of infusion given during any pump step in the Diocles method may be given by constant infusion or linearly-varying infusion rates (‘ramp’). It may also be given by a single brief injection at a higher injection rate but lower duration such that the same volume is given but that the velocity of injection is greater and there is also a period where there is no advancement of the first plunger. There may be more than one cycle of advancement and no advancement during an infusion pump step (e.g. ‘double burst’). The period of no advancement of the first plunger 92 may allow the valve means 102 to close and resumption of advancement may result in opening and enhanced mixing.

After completion of the infusion, the active ingredient remaining in the dilution chamber can be administered to the patient by collapsing the dilution chamber.

### The Diocles Method

FIGS. 34b, 34c and 34d show a particular arrangement of the method of operation of the medication delivery system 91 depicted in FIGS. 30 to 41. That is, FIGS. 34b, 34c and 34d show a particular arrangement of the method of operation of the medication delivery apparatus 90 while being mounted on the syringe driver 17 (which may also be referred to as an infusion driver or an infusion device).

In particular, the rate of drug administration is controlled by a particular function (referred to as the Diocles function) in accordance with the present embodiments of the disclosure. The Diocles function may be referred to as an infusion modelling function. The Diocles is a piecemeal function with two time periods to deliver to the patient the same dose of drug over time as the Tansy function using the medication delivery apparatus 90 depicted in FIGS. 30 to 41. The first time period (when the volume of the active agent chamber 98 is greater than zero and is reducing) uses a Kelly function (see FIG. 34c). The Kelly function is a numerically integrated algorithm to determine the volumes over time to be delivered to the patient so that the dose delivered to the patient after mixing in the dilution chamber 100 approximates that of the Tansy Function. The second time period is controlled by the Tansy function corrected for the concentration of active agent in the dilution chamber 100, which is constant once the active agent chamber 98 has been emptied.

The Diocles method is used to actuate the infusion device in order to deliver the pharmaceutical preparation to the patient by means of a medication delivery apparatus, in order to give the patient the dose of pharmaceutical preparation over time that is defined by a Tansy Dose Function. The Diocles method provides the pharmaceutical preparation in accordance with a step function with two time windows, as there are two physically different stages in the use of the medication delivery apparatus (changing drug chamber volume, constant dilution chamber volume vs constant (empty) drug chamber volume, changing dilution chamber volume).

The dilution chamber 100 is filled with diluent and a cap placed on the exit from the dilution chamber 100. The active agent chamber 100 is filled with the pharmaceutical preparation as a solution and cap placed on the filling port. The medication delivery apparatus 90 is placed in the syringe driver (i.e. the infusion driver). The cap is removed from the filling port and the syringe driver advances the first plunger 92 until fluid rises up the filling port (slack removed from system). The cap is replaced on the filling port.

The cap is removed from the exit of the dilution chamber 100. A minimum volume extension tubing is attached to the exit of the dilution chamber 100. The infusion driver advances the first plunger 92, injecting the pharmaceutical preparation into the dilution chamber 100 and fluid from the dilution chamber 100 into the minimum volume extension tubing until the mixed fluid reaches the end of the tubing, then the infusion is stopped.

The tubing is attached to a patient intravenous access. The program is started and the first of h infusion steps begins. Once the first infusion step has completed, the subsequent infusion step begins. Once the final infusion step has completed, the infusion stops.

FIGS. 34c and 34d are a flow diagrams that illustrates a method for delivering a pharmaceutical preparation to a patient. The method is the Diocles method.

As previously described, a medication delivery system 91 comprises the medication delivery apparatus 90 and the infusion device 93. The infusion device 93 may be as previously described. That is, the infusion device 93 comprises at least one infusion device processor and infusion device memory. The infusion device memory stores program instructions accessible by the at least one infusion device processor.

The program instructions are configured to cause the at least one infusion device processor to receive a concentration input (Cₚ) that is indicative of a concentration of the pharmaceutical preparation in the active agent chamber 98. The concentration may be a concentration of active agent in the pharmaceutical preparation. The concentration input (Cₚ) may be received via an input provided by a user. For example, the concentration input (Cₚ) may be input using the user interface 22. Alternatively, the concentration input (Cₚ) may be retrieved from the infusion device memory.

The program instructions are further configured to cause the at least one infusion device processor to receive a volume input (Vₚ) that is indicative of a volume of the pharmaceutical preparation. This may be a volume of the pharmaceutical preparation in the active agent chamber 98. The volume input (Vₚ) may be received via an input provided by a user. For example, the volume input (Vₚ) may be input using the user interface 22. Alternatively, the volume input (Vₚ) may be retrieved from the infusion device memory.

The program instructions are further configured to cause the at least one infusion device processor to receive a dilution chamber volume input (V_{d}). The dilution chamber volume input (V_{d}) is indicative of a volume of the dilution chamber 100. The dilution chamber volume input (V_{d}) may be received via an input provided by a user. For example, the dilution chamber volume input (V_{d}) may be input using the user interface 22. Alternatively, the dilution chamber volume input (V_{d}) may be retrieved from the infusion device memory.

The program instructions are further configured to cause the at least one infusion device processor to receive a time input (i). The time input (i) is indicative of a time window over which the pharmaceutical preparation is to be administered. The time input (i) may be received via an input provided by a user. For example, the time input (i) may be input using the user interface 22. Alternatively, the time input (i) may be retrieved from the infusion device memory. The time window comprises a first time window and a second time window.

The program instructions are further configured to cause the at least one infusion device processor to receive an infusion number input (τ). The infusion number input (τ) is indicative of a number of infusion intervals per minute over which an infusion modelling function is to be numerically approximated over the first time window. The infusion modelling function may be the Kelly function. The infusion number input (τ) may be received via an input provided by a user. For example, the infusion number input (τ) may be input using the user interface 22. Alternatively, the infusion number input (τ) may be retrieved from the infusion device memory.

The program instructions are further configured to cause the at least one infusion device processor to receive a number of infusion steps (h) that are to be executed during the time window. A first number of infusion steps (h₁) are to be executed during the first time window. A second number of infusion steps (h₂) are to be executed during the second time window. Receiving the number of infusion steps (h) that are to be executed during the time window may comprise receiving an infusion step input that is indicative of the number of infusion steps (h). Alternatively, determining the number of infusion steps (h) that are to be executed during the time window may comprise retrieving the number of infusion steps (h) from the infusion device memory. Receiving the number of infusion steps (h) that are to be executed during the time window may comprise multiplying the time input (i) and the infusion number input (τ).

The program instructions may further be configured to cause the at least one infusion device processor to determine a current time (t). The current time (t) may indicate the time within the time window.

The at least one infusion device processor numerically approximates the infusion modelling function. In particular, the at least one infusion device processor numerically approximates the infusion modelling function over the first time window. To numerically approximate the infusion modelling function over the first time window, the at least one infusion device processor may perform the functionality described below. That is, numerically approximating the infusion modelling function may comprise the functionality described below.

The at least one processor determines a number of infusion intervals of the first time window. Determining the number of infusion intervals within the first time window of the numerical approximation comprises multiplying the time input (i) and the infusion number input (τ). As previously described, the infusion device is capable of executing a certain number of infusion ‘events’ per minute (i.e. infusion steps). It might be that the infusion device, for example, can deliver an infusion at a particular rate for an interval of 20 seconds at a certain constant rate, then 20 seconds at another constant rate, then 20 seconds for another constant rate. So there would be three infusion ‘events’ per minute (i.e. three infusion steps per minute). Some infusion devices, for example are limited to 99 programmable ‘events’ during the course of the infusion and so a 30 minute infusion with 3 events per minute would be close to the limit of programmability of this infusion device. The particular characteristics of the infusion device will vary, what is important is that they can be programmed and that the infusion device is able to approximating the ‘ideal’ infusion program by means of a series of ‘steps’ of infusion at a particular rate.

The at least one processor determines an initiating target flow rate parameter (K(O)_{initiating}). The initiating target flow rate parameter is indicative of a target flow rate of the pharmaceutical preparation to be output into the dilution chamber 100 during an initiating infusion interval of the numerical approximation.

Determining the initiating target flow rate parameter (K(0)_{initiating}) comprises calculating:

$${K ( 0 )}_{initiating} = \sqrt{( \frac{2 {V}_{p}}{{2}^{16} - 2} {e}^{\frac{1}{2 τ} ln ( {2}^{\frac{30}{i}} )} - \frac{2 {V}_{p}}{{2}^{16} - 2} ) \times {V}_{d} \times {τ}^{2}}$$

The at least one processor determines an initiating pharmaceutical preparation concentration. The initiating pharmaceutical preparation concentration is indicative of an approximated concentration of the pharmaceutical preparation in the dilution chamber 100 after the initiating infusion interval of the numerical approximation. Determining the initiating pharmaceutical preparation concentration comprises calculating:

$${C}_{{d}_{( o )}} = \frac{( {K ( 0 )}_{initiating} \times {C}_{p} ) - ( {K ( 0 )}_{initiating} \times {C}_{{d}_{( - 1 )}} ) + ( {C}_{{d}_{( - 1 )}} \times {V}_{d} \times τ )}{{V}_{d} \times τ}$$

where C_{d(-1)}=0 and C_{d(0)} is the initiating pharmaceutical preparation concentration.

The at least one processor iteratively determines a subsequent target flow rate and a subsequent pharmaceutical preparation concentration for each of a plurality of subsequent infusion intervals of the numerical approximation. The subsequent target flow rates are each indicative of a target flow rate of the pharmaceutical preparation to be output by the medication delivery apparatus during a respective subsequent infusion interval of the numerical approximation. The subsequent pharmaceutical preparation concentrations are each indicative of a subsequent approximated concentration of the pharmaceutical preparation in the dilution chamber 100 after the respective subsequent infusion interval. Each of the subsequent target flow rates is determined based at least in part on the subsequent pharmaceutical preparation concentration of a previous infusion interval of the respective infusion interval. Each of the subsequent pharmaceutical preparation concentrations is determined based at least in part on the subsequent target flow rate of the respective subsequent infusion interval.

Determining the subsequent target flow rates comprises determining a flow rate parameter Kₙ for each of the subsequent target flow rates. The at least one infusion device processor determines Kₙ by calculating:

$${K}_{n} = \frac{Dose {( t )}_{n} * τ}{{C}_{d ( n - 1 )}}$$

where n is the number of the relevant infusion interval, C_{d(n-1)} is a subsequent pharmaceutical preparation concentration of a previous infusion interval of the nth infusion interval and Dose(t)ₙ is a target dose of the respective infusion interval of the first time window. The target dose is described in more detail herein. For example, the target dose Dose(t)ₙ may be similar to the previously described dose parameter.

In particular, determining the target dose Dose(t)ₙ comprises determining a dose of a Tansy function T(t). That is, determining the target dose Dose(t)ₙ comprises calculating:

$${\int }_{\frac{n - 1}{τ}}^{\frac{n}{τ}} T ( t ) dt \times {C}_{p}$$

where T(t) is the Tansy function.

In some embodiments,

$${\int }_{\frac{n - 1}{τ}}^{\frac{n}{τ}} T ( t ) dt$$

is equal to:

$$( \frac{2 {V}_{p}}{{2}^{16} - 2} {e}^{( \frac{n}{2 τ} ) ln {2}^{( \frac{30}{i} )}} - \frac{2 {V}_{p}}{{2}^{16} - 2} ) - ( \frac{2 {V}_{p}}{{2}^{16} - 2} {e}^{( \frac{n - 1}{2 τ} ) ln {2}^{( \frac{30}{i} )}} - \frac{2 {V}_{p}}{{2}^{16} - 2} )$$

In some embodiments, determining the subsequent pharmaceutical preparation concentrations of the first numerical approximation comprises calculating:

$${C}_{d ( n )} = \frac{( {K}_{n} \times {C}_{p} ) - ( {K}_{n} \times {C}_{d ( n - 1 )} ) + ( {C}_{d ( n - 1 )} \times {V}_{d} \times τ )}{{V}_{d} \times τ}$$

where C_{d(n)} is the subsequent pharmaceutical preparation concentration for the nth infusion interval and C_{d(n-1)} is the subsequent pharmaceutical preparation concentration for the n−1 th infusion interval. In other words, n is the number of the relevant infusion interval and C_{d(n-1)} is a subsequent pharmaceutical preparation concentration of a previous infusion interval of the nth infusion interval.

This calculation may be performed for each subsequent pharmaceutical preparation concentration of the iteration.

The at least one infusion device processor determines a first infusion volume for each of the first number of the infusion steps (h₁). In particular, the at least one infusion device processor determines the first infusion volume for each of the first number of the infusion steps (h₁) based at least in part on the numerical approximation. The infusion volume is indicative of a volume of the pharmaceutical preparation that is to be output by the medication delivery apparatus during the respective infusion step.

The at least one infusion device processor determines the first infusion volume f or one of the first number of the infusion steps (h₁) by calculating:

$${V}_{step ( x )} = {{\sum }^{n = \frac{( x ) ( i \times τ )}{h}}}_{n = \frac{( x - 1 ) ( i \times τ )}{h}} ( {K}_{n} \times \frac{1}{τ} )$$

where Vₛₜₑₚ₍ₓ₎ is the infusion volume of the xth infusion step of the first number of the infusion steps (h₁).

The at least one infusion device processor determines a number of infusion intervals of the second time window.

The at least one infusion device processor determines a target dose Dose(t)ₙ for each of the number of infusion intervals of the second time window. This may be as described in FIGS. 34a to 34c.

The at least one infusion device processor determines a target flow rate Dₙ for each of the number of infusion intervals of the second time window, based at least in part on the target dose for the respective infusion interval. Determining the target flow rate for each of the number of infusion intervals of the second time window comprises calculating:

$${\int }_{\frac{n - 1}{τ}}^{\frac{n}{τ}} T ( t ) dt \times \frac{{C}_{p}}{{C}_{dc}} .$$

where C_{dc} is a concentration of the pharmaceutical preparation in the dilution chamber at a point when the active agent chamber is empty.

The at least one infusion device processor determines determine a second infusion volume for each of the second number of infusion steps (h₂) based at least in part on the target flow rate. Determining the second infusion volume for one of the second number of the infusion steps (h₂) comprises calculating:

$${V}_{step ( x )} = {{\sum }^{n = \frac{( x ) ( i \times τ )}{h}}}_{n = \frac{( x - 1 ) ( i \times τ )}{h}} ( {D}_{n} \times \frac{1}{τ} )$$

where Vₛₜₑₚ₍ₓ₎ is the infusion volume of the xth infusion step of the second number of the infusion steps (h₂) and Dₙ is the target flow rate for one of the number of infusion intervals of the second time window.

The at least one infusion device processor actuates an infusion device actuator to displace the first plunger such that the determined infusion volume for each infusion step (h) is output by the medication delivery apparatus 90 during the respective infusion step.

In some embodiments, the program instructions are further configured to cause the at least one infusion device processor to determine an infusion rate for each of the infusion steps (h), and wherein determining the infusion rate for one of the infusion steps comprises calculating

$$\frac{{V}_{step ( x )} \times h}{i} ,$$

where Vₛₜₑₚ₍ₓ₎ is the infusion volume of the xth infusion step.

In some embodiments, the program instructions are further configured to cause the at least one infusion device processor to actuate the infusion device actuator such that the determined infusion volume for each infusion step is output by the medication delivery apparatus 90 during the respective infusion step at the determined infusion rate.

In some embodiments, the program instructions are further configured to cause the at least one infusion device processor to actuate the infusion device actuator such that the determined infusion volume for each infusion step is delivered according to a constant-rate profile or a linearly-changing rate profile.

In some embodiments, the program instructions are further configured to cause the at least one infusion device processor to actuate the infusion device actuator such that the determined infusion volume for each infusion step is output by the medication delivery apparatus during the respective subsequent infusion step in bursts.

In some embodiments, receiving the number of infusion steps that are to be executed during the time window comprises receiving an infusion step input that is indicative of the number of infusion steps. In some embodiments, receiving the number of infusion steps that are to be executed during the time window comprises retrieving the number of infusion steps from the infusion device memory

In some embodiments, the program instructions are further configured to cause the at least one infusion device processor to receive a pharmaceutical preparation input. The pharmaceutical preparation input may be indicative of one or more of an identity of the pharmaceutical preparation; a dose of the pharmaceutical preparation; and a maximum pharmaceutical preparation administration rate. In some embodiments, the subsequent target flow rates are limited at the maximum pharmaceutical preparation administration rate, such that the subsequent target flow rates do not exceed the maximum pharmaceutical preparation administration rate.

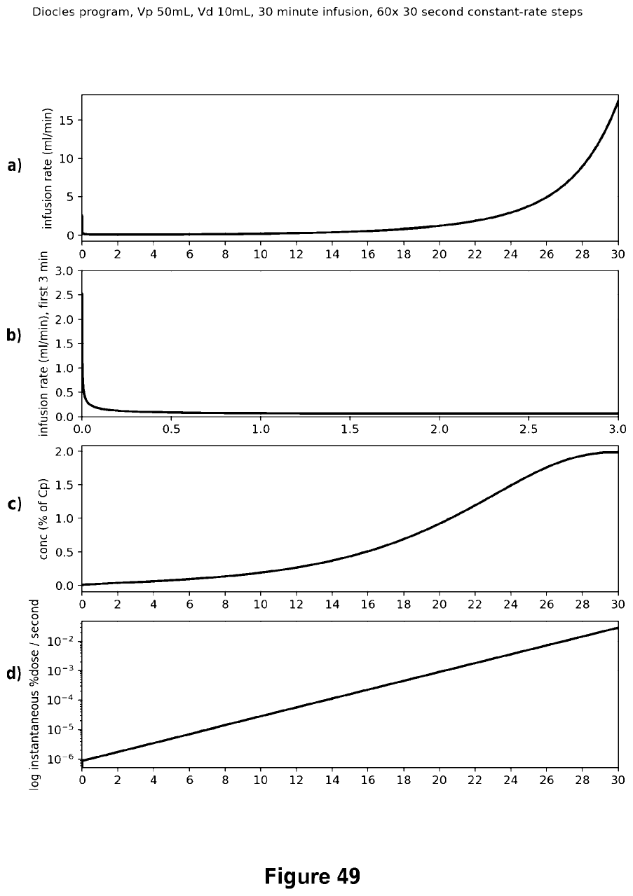

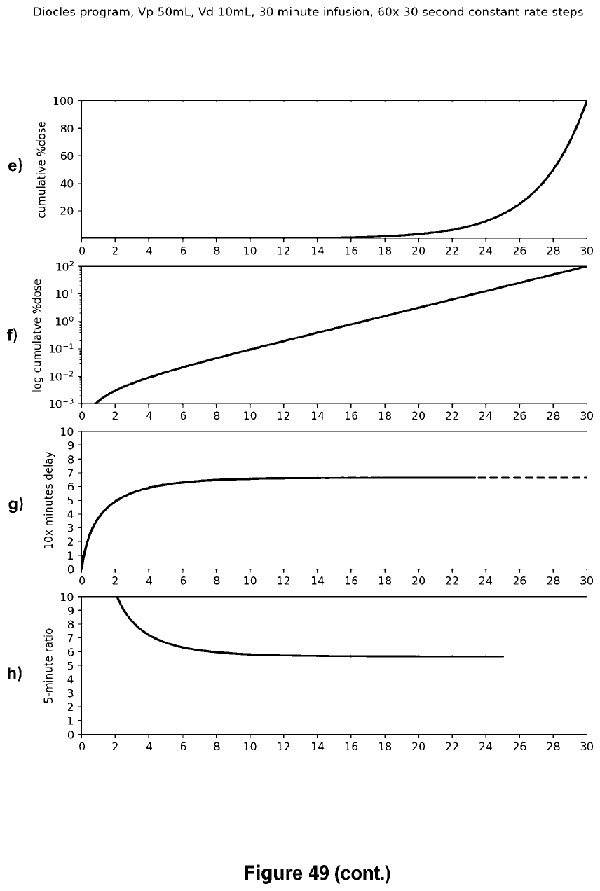

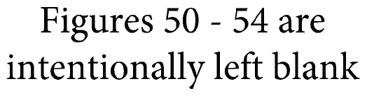

FIGS. 49a to 49g show theoretical results provided by implementation of the Diocles method for a particular value of Vₚ, V_{d} and i. FIG. 49a is a chart illustrating a fluid injection rate (y-axis) in mL/min of the Diocles method against an infusion time (x-axis) in minutes. FIGS. 49a and 49b (first 3 minutes of a 30 minute infusion) are charts illustrating the infusion flow rate vs time. FIG. 49c is a chart illustrating a concentration of the pharmaceutical preparation delivered to the patient (x-axis), with the units of the x-axis being percentage of total dose in the active agent chamber 98 (or therapeutic dose per mL) against an infusion time (x-axis) in minutes. FIG. 49d is a log chart of the instantaneous percentage dose of the pharmaceutical preparation delivered per second (y-axis) against infusion time (x-axis) in minutes. FIG. 49e is a chart illustrating the cumulative percentage dose (y-axis) against infusion time (x-axis) in minutes. FIG. 49f is a log chart illustrating the cumulative percentage dose (y-axis) against infusion time (x-axis) in minutes. FIG. 49g is a chart showing the number of minutes until the cumulative dose delivered is 10 times that at the time indicated on the x-axis. For example, at the point in time 2 minutes in the infusion, it will be another 5 minutes before the cumulative dose is 10 times that which the cumulative dose was at 2 minutes, and at the point in time 14 minutes into the infusion, it will be another 6.7 minutes before the cumulative dose administered is 10× what the cumulative dose was at 14 minutes. FIG. 49h is a chart showing the ratio of cumulative dose at each point in time during the infusion at that point compared to the cumulative dose 5 minutes later in the infusion. For example, at 2 minutes into the infusion the cumulative dose 5 minutes later will be approximately 10 times the cumulative dose at 2 minutes, and at 14 minutes into the infusion the ratio of cumulative dose 5 minutes later will be approximately 5.7 times greater than it was at 14 minutes. These graphs indicate the interval likely to be available to wait for the emergence of an adverse reaction before a dose that would cause a more severe reaction is given.

The volume of infusion given during any pump step in the Diocles method may be given by constant infusion or linearly-varying infusion rates (‘ramp’). It may also be given by a single brief injection at a higher injection rate but lower duration such that the same volume is given but that the velocity of injection is greater and there is also a period where there is no advancement of the first plunger 92. There may be more than one cycle of advancement and no advancement during an infusion step (e.g. ‘double burst’). The period of no advancement of the first plunger 92 may allow the valve means 102 to close and resumption of advancement may result in opening and enhanced mixing.

Method of Delivery where a Maximum Delivery Rate is Exceeded or a Maximum Tolerable Dose is Exceeded

The maximum rate of delivery of drug may be exceeded during the infusion process as a consequence of user settings. In order to ensure this does not happen, the medication delivery system can check that each infusion step does not exceed the maximum allowable dosing rate by estimating the dilution chamber drug concentration and the fluid infusion rate. The dilution chamber drug concentration as a function of cumulative drug volume infused (V) is given by the following equation:

$${C}_{d} = {C}_{p} * ( 1 - {e}^{- \frac{V}{{V}_{d}}} )$$

C_{d} is the concentration of drug in the dilution chamber
Cₚ is the original concentration of drug in the drug delivery flask or syringe or container
V_{d} is the volume of the dilution chamber
V is cumulative volume infused into the dilution chamber or patient

### Cisplatin Dosing

For example, current dosing for a man is 40 mg/m2 over 1 hour in 1000 ml of diluent. This protocol (i.e. the medication delivery system) will deliver 72 mg of Cisplatin in 1000 mL over 60 minutes, which is a fluid injection rate of 16.7 ml/min, and a dose rate of 1.2 mg/min.

If this is delivered using the previously disclosed medication delivery apparatus 90, one can prepare the 72 mg of Cisplatin in 1000 mL of diluent in a flask, connected to the medication delivery apparatus 90 by a peristaltic fluid pump. The dilution chamber 100 can be set at 50 mL volume. The Diocles algorithm can be used as the infusion duration will be limited by the maximum dosing rate in both described instances (rather than using the Sadleir algorithm which is chosen when the dilution chamber 100 is not automatically collapsed and where the automatic program prior to manually collapsing the dilution chamber is desired to be of the duration set).

The duration of infusion can be set to 60 minutes using 120 constant infusion steps of 30 seconds. Using this arrangement, the dose rate increases exponentially over the duration of the infusion. The minimum infusion flow rate will be 0.306 ml/min (18.4 ml/hr). The maximal allowable dose rate (1.2 mg/min) is reached at 46 minutes and 29 seconds, when the cumulative volume administered has been 143 mL, the dilution chamber concentration is 0.0.679 mg/mL, and the infusion rate is 17.7 mL/min. For the subsequent infusion step, the infusion rate is limited to 17.7 ml/min and the cumulative volume at the end of that step is 161 mL. The dilution chamber concentration is then estimated at 0.0691 mg/mL. The following step will have the infusion rate reduced to 17.4 mL/min to ensure that the maximal allowable dose rate (1.2 mg/min is not exceeded). This adjustment of each step infusion rate will continue until the infusion is completed. The duration of the infusion will be extended to a total infusion duration of approximately 98 minutes. The final step infusion rate will be approximately 16.7 mL/min and the dilution chamber concentration 0.072 mg/ml. After completion of the infusion, either the dilution chamber can be collapsed to deliver the final 50 ml of solution, or an additional 50 mL of drug infusion can be delivered from the drug flask via the dilution chamber.

The duration of infusion can be set to 180 minutes using 360 constant infusion steps of 30 seconds. Using this arrangement, the dose rate increases exponentially over the duration of the infusion. The minimum infusion flow rate will be 0.1 mL/min (6 ml/hr). The maximum allowable infusion dose rate (1.2 mg/min) is exceeded at 158 minutes and 29 seconds, therefore the infusion will be limited to the infusion rate for the subsequent interval (starting at 158 minutes and 30 seconds). The cumulative volume delivered is 338.5 mL and the infusion rate 16.7 ml/min. The dilution chamber concentration is estimated at 0.0719 mg/mL and so the allowable infusion rate is 16.7 mL/min for all subsequent intervals. The remaining 661.5 mL infusion will complete in a further 40 minutes (total infusion duration approximately 198 minutes). After 1000 mL of the drug infusion has been infused, the dilution chamber can be collapsed to deliver the final 50 mL of solution, or an additional 50 mL of drug infusion can be delivered from the drug flask via the dilution chamber.

### Rocuronium Dosing

Rocuronium is a non-depolarising neuromuscular blocking agent and is chosen as an example of a drug that can only have a part of its therapeutic dose administered slowly (the rest having to be administered either quickly or slowly when anaesthetized). It is administered intravenously in a dose of 0.6 mg/kg (50 mg for an 80 kg patient). This is usually administered as a push after anaesthesia is induced.

Rocuronium may be administered to an awake patient up to a dose of approximately 0.03 mg/kg (2.4 mg in an 80 kg patient). This will cause minor, tolerable side-effects (blurred vision).

Test doses or desensitisation may be administered by diluting 50 mg of rocuronium in an infusion volume Vₚ of 50 mL, with a 10 mL dilution chamber, infused over 30 minutes, but pausing the infusion for induction of anaesthesia once 0.03 mg/kg has been administered. Then the remainder of the infusion can either be given as a push (if relaxation is required immediately at induction) or by continuing the remainder of the infusion.

Using the medication delivery system 91 with this protocol, 2.4 mg is administered after 21 minutes and 14 seconds. The infusion rate at this point is 1.43 ml/min and 7.83 ml of solution has been infused.

### Method of Calculating Infusion Rate and Cumulative Volume Delivered Using the Medication Delivery System 91

As described, the dilution chamber drug concentration as a function of cumulative drug volume infused (V) is given by the following equation:

$${C}_{d} = {C}_{p} * ( 1 - {e}^{- \frac{V}{{V}_{d}}} )$$

C_{d} is the concentration of drug in the dilution chamber
Cₚ is the original concentration of drug in the drug delivery flask or syringe or container V_{d} is the volume of the dilution chamber
V is cumulative volume infused into the dilution chamber or patient

This relationship can hold until the dilution chamber volume is reduced by the advancing plunger, beyond which point the dilution chamber concentration remains constant.

Example Infusions in Accordance with the Diocles Method and the Medication Delivery System 91

FIGS. 30 to 34d and 42 show the medication delivery apparatus 90 operating as a syringe for mounting on a syringe driver 17 for delivering the pharmaceutical composition (i.e. mixture of active agent and diluent). This particular arrangement of medication delivery apparatus 90 is particularly useful (when compared against the dilution chamber 32 described with reference to FIG. 2) because it permits omitting the dilution chamber 32 (located at a remote location of the syringe driver 17) which is used for mixing the active agent and diluent prior delivering the pharmaceutical composition (containing active agent and diluent) to the patient.

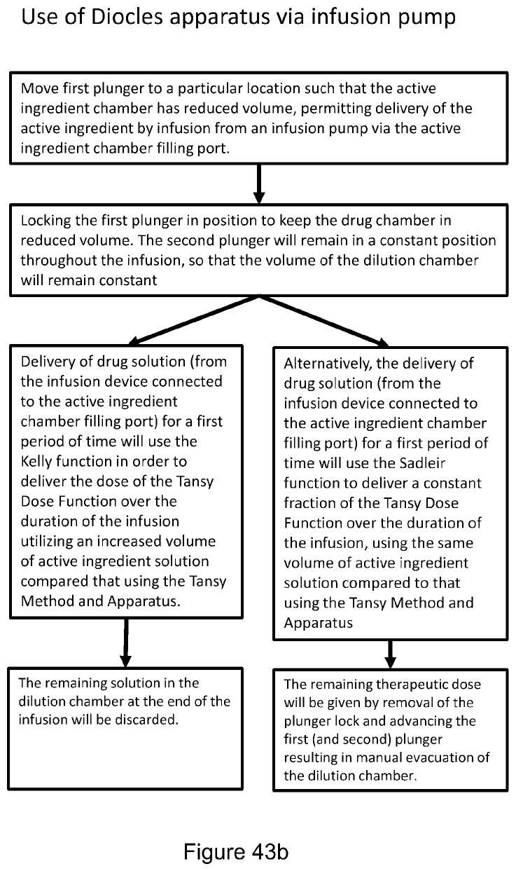

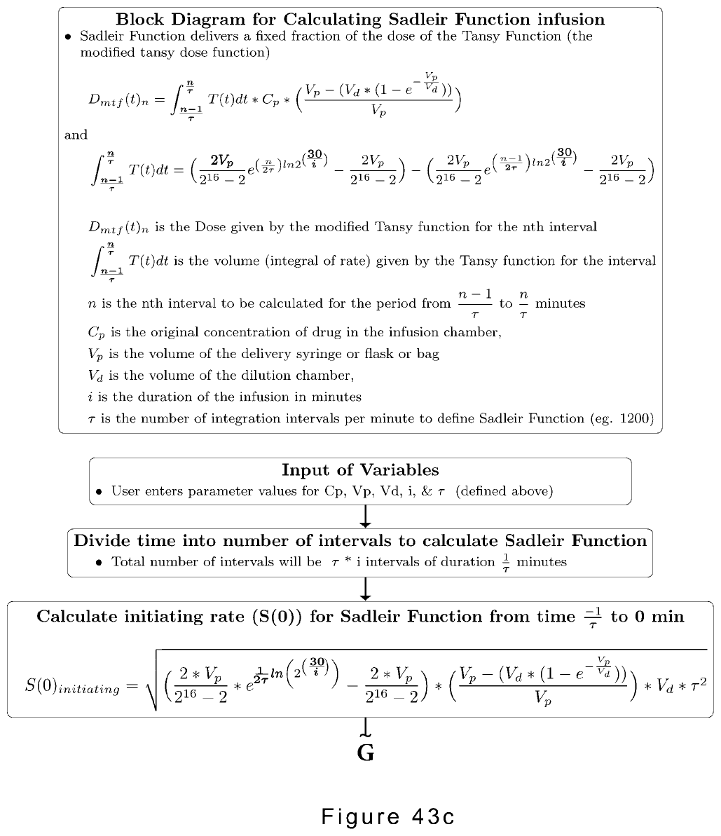

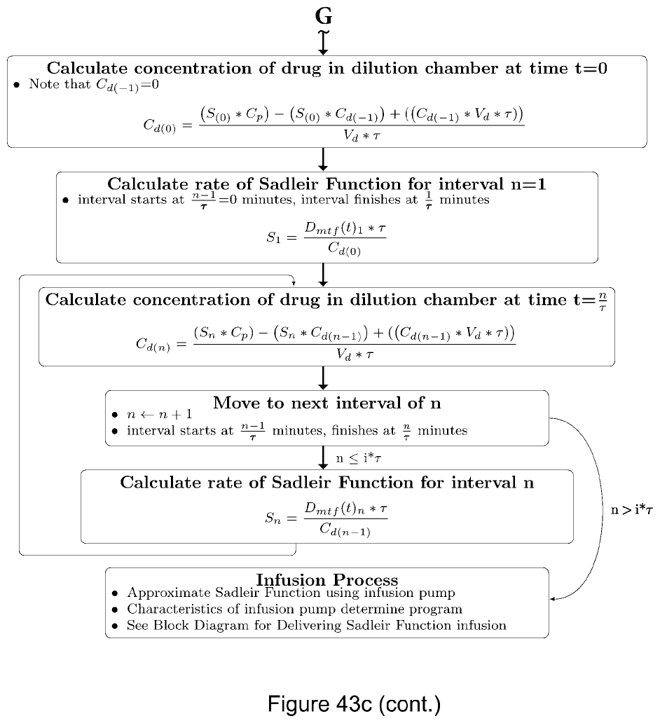

However, in alternative arrangements (see FIG. 43), the dilution chambers 100 may function as a dilution chamber 32 being located at a remote location of the syringe driver 17 as depicted in FIG. 2.

As shown in FIG. 43, the medication delivery apparatus 90 comprises a plunger lock 134 in order to fix the primary plunger 92 at a particular location permitting to deliver the active agent (coming from the syringe driver 17) into the active agent chamber 98 for delivery of the drug, through the separation plunger, into the mixing chamber 100 for delivery into the patient via the conduit 30b.

The plunger lock 134 shown in FIG. 43 comprises a body having a lower surface 137 for resting on a support surface and an upper surface 139 having spaced apart grooves 141a and 141b for receiving the flanges 145 and 147 of the primary plunger 92 and the active agent chamber 98. In this manner, the primary plunger 92 is fixed at a particular location not being able to move within the active agent chamber 98.

As shown in FIG. 43, the primary plunger 92 is located in a particular location such that the active agent chamber 98 has a relatively small volume. The fact that the primary plunger 92 is locked in position due to the plunger lock 134, impedes the primary plunger 92 from moving, thus maintaining the relative small volume of the active agent chamber 98 constant as active agent is delivered from the syringe driver 17 via conduit 30a into the active agent chamber 98.

In operation, as active agent is delivered into the active agent chamber 98 of constant volume, the pharmaceutical preparation is forced to flow through the separating plunger 94 into the mixing chamber 100 for mixing of the pharmaceutical preparation and the diluent to prepare the pharmaceutical composition for delivery into the blood stream of the patient via conduit 30a. FIGS. 43b and 43c illustrates the method of operation of the medication delivery apparatus 90 when operating remotely from the syringe driver 17 comprising a syringe 15 solely filled with active agent.

In particular, the rate of active agent administration is governed by the Sadleir function. The Sadleir function is a numerically-integrated function to determine the volumes over time to be delivered to the patient so that the dose delivered to the patient after mixing in the medication delivery apparatus 90 approximates that of a fixed fraction of the Tansy Function at that time using the Sadleir embodiment. This is because at the end of the infusion some pharmaceutical composition still remains in the mixing chamber 100 that will be delivered to the patient by moving the primary plunger 92 towards the outlet 110.

In an alternative arrangement and as mentioned before, it is also possible to increase the concentration of the active agent in the dilution chamber 100 to deliver the same dose as the Tansy function over time, and then discard the remaining pharmaceutical composition in the dilution chamber 100 instead than delivering it to the patient.

### Controlling the Infusion Device Using Analytical Solutions to Cumulative Volume and Rate Functions

A number of the methods for delivering pharmaceutical preparations to patients described herein involve the use of numerical approximations of infusion modelling functions to control actuation of the infusion device actuator and the flow rate of the fluid that is expelled from the medication delivery apparatus 1, 90. Using numerical approximations in this way can increase the computational requirements of the hardware on which the methods are performed. For example, the infusion computing device 151 and or the infusion device 93 may require a more powerful processor and/or more memory to compute the numerical approximations of the infusion modelling functions. It may therefore be advantageous to be able to deterministically compute solutions to the infusion modelling functions which can be used to control the medication delivery system 1, 91. That is, using analytical solutions of the infusion modelling functions to control the medication delivery system 1, 91 can reduce the requirements of the infusion computing device 151 and/or the infusion device 93, thereby reducing its cost. In some embodiments, using analytical solutions of the infusion modelling functions to control the medication delivery system 1, 91 can decrease pre-infusion delays associated with the infusion computing device 151 and/or the infusion device 93 preparing for the infusion (i.e. the necessary computations can be performed on existing hardware faster than alternative methods).

A number of methods of delivering a pharmaceutical preparation to a patient are disclosed herein. One or more of these methods comprises determining an infusion process that is performed by an infusion device. The infusion process is determined by the infusion computing device 151. The infusion process is stored as an infusion process file. For example, the infusion process may be stored as an infusion process file in the infusion computing device memory 258. The infusion process file may be transmitted to the infusion device 93 to be performed.

### Method 5500 for Delivering a Pharmaceutical Preparation to a Patient

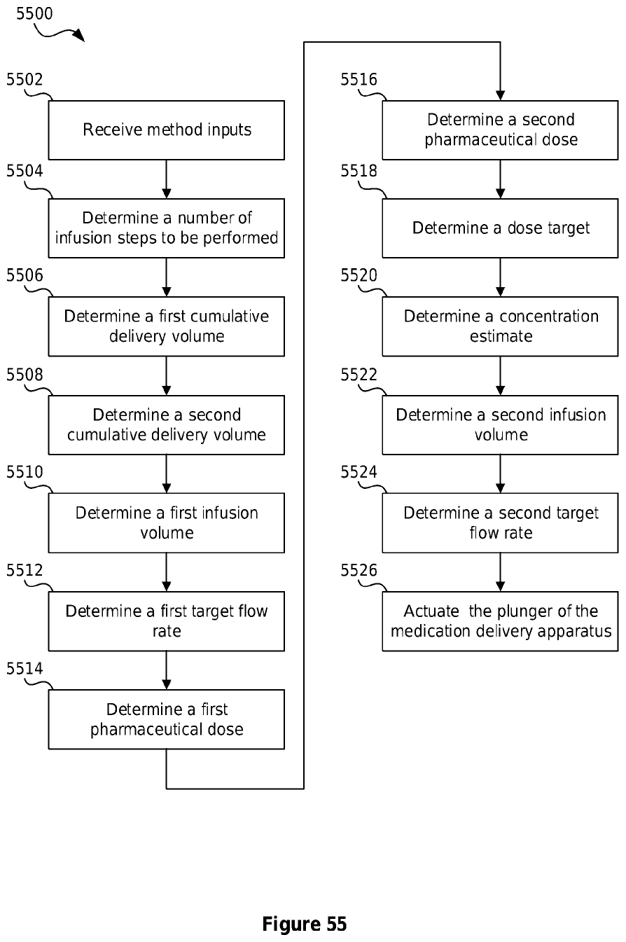

FIG. 55 is a process flow diagram of a method 5500 for delivering a pharmaceutical preparation to a patient, according to some embodiments. The method 5500 may be performed by the medication delivery system 1 and/or the medication delivery apparatus 10 described with reference to FIGS. 1 to 2. The method 5500 may be performed by the medication delivery system 91 and/or the medication delivery apparatus 90 described with reference to FIG. 30 to 34a or 42 to 43. The medication delivery system 91 comprises the medication delivery apparatus 90 and the infusion device 93, as is described herein. The pharmaceutical preparation is delivered to the patient in accordance with an infusion process.

The infusion process commences at an initial time. Although the method 5500 is described with reference to the medication delivery system 91 of FIGS. 30 to 34A and 42 to 43, it will be understood that the description is also applicable to the medication delivery system and/or the medication delivery apparatus 10 described with reference to FIGS. 1 to 2 and the medication delivery system.

Some or all of the method 5500 may be performed by the infusion device processor 250 (i.e. the processor of the infusion device 93). Some or all of the method may be performed by the infusion computing device 151. Some or all of the method may be performed by another computing device. The method 5500 may therefore be considered a computer implemented method.

As described herein, the medication delivery apparatus 90 is configured to store at least one fluid. Specifically, the medication delivery apparatus 90 comprises an active agent chamber 98 that is configured to store a fluid comprising an active agent. The fluid comprising the active agent may be referred to as a first fluid. The fluid comprising the active agent may be referred to as a pharmaceutical preparation, as described herein. The medication delivery apparatus 90 comprises a dilution chamber 100 that is configured to store a diluent. The diluent may be referred to as a second fluid.

The medication delivery apparatus 90 is configured to output a fluid. When the active agent chamber 98 stores fluid, actuation of the first plunger 92 (i.e. movement of the first plunger towards the dilution chamber outlet 27) applies a pushing force to the fluid. This pushes the fluid stored in the active agent chamber 98 through the valve 39 of the second plunger 94 into the dilution chamber 100. The fluid in the dilution chamber 100 (i.e. diluent or diluted pharmaceutical preparation) is thereby pushed out of the dilution chamber outlet 110 and to the patient (via the conduit 30a). That is, the fluid in the dilution chamber 100 is expelled from the dilution chamber 100.

When the active agent chamber 98 has been emptied, actuation of the first plunger 92 (i.e. movement of the first plunger 92 towards the dilution chamber outlet 110) applies a pushing force to second plunger 94 that causes movement of the second plunger 94. This pushes the fluid stored in the dilution chamber 100 out of the dilution chamber outlet 110 and to the patient. That is, the fluid in the dilution chamber 100 is expelled from the dilution chamber 100. For the purposes of at least one of the methods disclosed herein, the fluid that is expelled from the medication delivery apparatus is the fluid stored in the dilution chamber 100 (i.e. the diluted pharmaceutical preparation).

In some embodiments, the fluid pushed out of the dilution chamber outlet 110 may be referred to as the pharmaceutical preparation. In some embodiments, the fluid pushed out of the dilution chamber outlet 110 is referred to as a diluted pharmaceutical preparation. In some embodiments, the fluid pushed out of the dilution chamber outlet 110 is referred to as a pharmaceutical composition.

In some embodiments, method 5500 may be referred to as an analytical Diocles method or a Diocles method.

At 5502, the infusion computing device 151 receives one or more method inputs. In particular, the infusion computing device processor 256 receives the one or more method inputs. In some embodiments, the one or more method inputs comprises a plurality of method inputs. The one or more method inputs may be received via the infusion computing device user interface 260 (e.g. via the display and/or the keyboard). In some embodiments, at least one of the one or more method inputs is an input of a cumulative delivery volume function. In some embodiments, at least one of the one or more method inputs is an input of a dose function. The clinician may input the one or more method inputs.

In some embodiments, the one or more method inputs comprises a concentration input (Cₚ). The concentration input (Cₚ) may be as described herein. The concentration input (Cₚ) is indicative of a concentration of the pharmaceutical preparation in the active agent chamber 98. That is, the concentration input (Cₚ) may be indicative of a concentration of a drug dissolved in a solvent, where the solvent comprising the dissolved drug is the pharmaceutical preparation.

In some embodiments, the one or more method inputs comprises a volume input (Vₚ). The volume input (Vₚ) may be as is described herein. The volume input (Vₚ) is indicative of a volume of the pharmaceutical preparation in the active agent chamber 98. The volume input (Vₚ) may correspond to a volume of the active agent chamber 98.

In some embodiments, the one or more method inputs comprises a dilution chamber volume input (V_{d}). The dilution chamber volume input (V_{d}) may be as is described herein. The dilution chamber volume input (V_{d}) is indicative of a volume of the dilution chamber 100. The dilution chamber volume input (V_{d}) may correspond to a volume of the diluent.

In some embodiments, the one or more method inputs comprises a time input (i). The time input (i) may be as is described herein. The time input (i) is indicative of at least a portion of a time window over which the pharmaceutical preparation is to be delivered. In some embodiments, the time input (i) is indicative of a total length of the time window. The time window comprises a first time window and a second time window.

In some embodiments, the first time window spans a time period during which the first plunger 92 is displaced towards the second plunger 94 without having contacted the second plunger 94. This may be the case, for example, where the method 550 is performed by the medication delivery system 91 described with reference to FIG. 30 to 34A or 42 to 43. During the first time window, actuation of the first plunger 92 displaces pharmaceutical preparation from the active agent chamber 98 to the dilution chamber 100.

The second time window spans a time period during which the first plunger 92 and the second plunger 94 moved simultaneously. In some embodiments, this is when the first plunger 92 and the second plunger 94 are in contact. This may be the case, for example, where the method 550 is performed by the medication delivery system 93 described with reference to FIG. 30 to 34A or 42 to 43. During the first time window, actuation of the first plunger 92 displaces pharmaceutical preparation from the active agent chamber 98 to the dilution chamber 100.

During the second time window, displacement of the first plunger 92 causes a corresponding displacement of the second plunger 94. During the second time window, a concentration of the pharmaceutical preparation within the dilution chamber 100 is constant, as the active agent chamber 98 has been emptied into the dilution chamber 100.

The method 5500 comprises performing a number of infusion steps (h) within the time window. A first number of infusion steps (h₁) are performed within the first time window. A second number of infusion steps (h₂) are performed within the second time window.

At 5504, the infusion computing device 151 determines the number of infusion steps (h) that are to be performed within the time window. In particular, the infusion computing device processor 256 determines the number of infusion steps (h) that are to be performed within the time window. The infusion computing device processor 256 determines the first number of infusion steps (h₁) that are to be performed within the first time window. The infusion computing device processor 256 determines the second number of infusion steps (h₂) that are to be performed within the second time window. The time window, first time window, second time window, number of infusion steps (h), first number of infusion steps (h₁) and/or second number of infusion steps (h₂) may be as described herein.

In some embodiments, determining the number of infusion steps (h) comprises receiving the number of infusion steps (h). The number of infusion steps (h) may be received (e.g. by the infusion computing device processor 256) as an infusion step input. Alternatively, the one or more method inputs may comprise the number of infusion steps (h).

In some embodiments, determining the number of infusion steps (h) comprises calculating a product of a number of infusion steps (per minute) that are to be performed within the time window (g) and the time input (i). That is, to determine the number of infusion steps (h), the infusion computing device processor 256 calculates:

$$h = g \times i$$

where h is the number of infusion steps that are to be performed within the time window, g is the number of infusion steps per minute that are to be performed during the infusion process and (i) is the time input. The time input (i) may therefore indicate a length of the infusion process in minutes. In these embodiments, the infusion computing device processor 256 may store the number of infusion steps (h) in the infusion computing device memory 258. The infusion computing device processor 256 may subsequently issue a read transaction to the infusion computing device memory 258 relating to the number of infusion steps (h), and may receive the number of infusion steps (h) in response. Thus, receiving the number of infusion steps (h) may comprise determining the number of infusion steps (h).

In some embodiments, the infusion computing device processor 256 determines the first number of infusion steps (h₁) based at least in part on a determined transitional time. For example, the infusion computing device processor 256 may divide a difference between the transitional time and an initial time of the infusion process by an infusion step duration. The infusion computing device processor 256 may determine the second number of infusion steps (h₂) based at least in part on the transitional time. For example, the infusion computing device processor 256 may divide a difference between an infusion conclusion time (i.e. the time at the end of the infusion process) and the initial time by the infusion step duration. The infusion computing device processor 256 may subtract this from the first number of infusion steps (h₁) to determine the second number of infusion steps (h₂). The transitional time may be as is described herein.

At 5506, the infusion computing device 151 determines a first cumulative delivery volume (KV₁) for an infusion step of the first number of infusion steps (h₁). In particular, the infusion computing device processor 256 determines the first cumulative delivery volume (KV₁) for the infusion step of the first number of infusion steps (h₁). The first cumulative delivery volume (KV₁) is indicative of a cumulative volume of a fluid that is to be expelled from the medication delivery apparatus 90 between an initial time and an initial infusion step time. The fluid that is expelled from the medication delivery apparatus 90 is the fluid that is stored in the dilution chamber 100 at the time of the infusion step of the first number of infusion steps (h₁). That is, the fluid is the diluted pharmaceutical preparation. As described herein, the initial time corresponds to a start of the infusion process. For example, the initial time may be 0. The initial infusion step time corresponds to a start of the infusion step of the first number of infusion steps (h₁). The initial infusion step time may be indexed with respect to the initial time. For example, the initial infusion step time may be the number of milliseconds or seconds that the infusion step of the first number of infusion steps (h₁) starts at after the initial time.

The infusion computing device processor 256 determines the first cumulative delivery volume (KV₁) using a cumulative delivery volume function. The cumulative delivery volume function may be referred to as a Kelly Cumulative Volume Function. The cumulative delivery volume function has one or more inputs. The one or more inputs of the cumulative delivery volume function comprise one or more of the initial infusion step time of the infusion step of the first number of infusion steps (h₁), the time input (i), the dilution chamber volume input (V_{d}) and the volume input (Vₚ). Thus, the infusion computing device processor 256 determines the first cumulative delivery volume (KV₁) based at least in part on one or more of the initial infusion step time of the infusion step of the first number of infusion steps (h₁), the time input (i), the dilution chamber volume input (V_{d}) and the volume input (Vₚ).

The cumulative delivery volume function can be expressed as:

$$KV = {V}_{d} + {V}_{d} {W}_{0} ( - {e}^{- \frac{( {( {2}^{\frac{30}{i}} )}^{\frac{t}{2}} - 1 ) {V}_{p}}{32767 \times {V}_{d}} - 1} ) + \frac{{V}_{p} ( {( {2}^{\frac{30}{i}} )}^{\frac{t}{2}} - 1 )}{32767}$$

where:
KV is indicative of the cumulative volume of fluid expelled at time t;
V_{d} is the dilution chamber volume input;
W₀ is the principle branch of the Lambert W function;
i is the time input;
t is a relevant time that is indicative of a time at which the cumulative delivery volume function is being solved; and
Vₚ is the volume input.

This may be referred to as a Kelly Cumulative Volume Function.

Solving for KV in the cumulative delivery volume function provides an indication of the cumulative volume of a fluid that is to be expelled from the medication delivery apparatus 90 between the initial time and the relevant time (t).

Therefore, the infusion computing device processor 256 determines the first cumulative delivery volume (KV₁) by calculating:

$${KV}_{1} = {V}_{d} + {V}_{d} {W}_{0} ( - {e}^{- \frac{( {( {2}^{\frac{30}{i}} )}^{\frac{{t}_{i}}{2}} - 1 ) {V}_{p}}{32767 \times {V}_{d}} - 1} ) + \frac{{V}_{p} ( {( {2}^{\frac{30}{i}} )}^{\frac{{t}_{i}}{2}} - 1 )}{32767}$$

### Where:

KV₁ is indicative of the first cumulative delivery volume; and
tᵢ is indicative of the initial infusion step time of the infusion step of the first number of infusion steps (h₁).

The first cumulative delivery volume KV₁ provides an indication of the cumulative volume of the fluid that is to be expelled from the medication delivery apparatus 90 between the initial time and the initial infusion step time (tᵢ).

At 5508, the infusion computing device 151 determines a second cumulative delivery volume (KV₂) for the infusion step of the first number of infusion steps (h₁). In particular, the infusion computing device processor 256 determines the second cumulative delivery volume (KV₂) for the infusion step of the second number of infusion steps (h₂). The second cumulative delivery volume (KV₂) is indicative of a cumulative volume of a fluid that is to be expelled from the medication delivery apparatus 90 between the initial time and a subsequent infusion step time. The cumulative volume of the fluid that is to be expelled from the medication delivery apparatus 90 between the initial time and the subsequent infusion step time may be referred to as a second cumulative volume. The fluid that is expelled from the medication delivery apparatus 90 is the fluid that is stored in the dilution chamber 100 at the time of the infusion step of the first number of infusion steps (h₁). That is, the fluid is the diluted pharmaceutical preparation. As described herein, the initial time corresponds to a start of the infusion process. The subsequent infusion step time corresponds to an end of the infusion step of the first number of infusion steps (h₁). The subsequent infusion step time may be indexed with respect to the initial time. For example, the subsequent infusion step time may be the number of milliseconds or seconds at which the infusion step of the first number of infusion steps (h₁) ends after the initial time.

The infusion computing device processor 256 determines the second cumulative delivery volume (KV₂) using the cumulative delivery volume function.

In particular, the infusion computing device processor 256 determines the second cumulative delivery volume (KV₂) by calculating:

$${KV}_{2} = {V}_{d} + {V}_{d} {W}_{0} ( - {e}^{- \frac{( {( {2}^{\frac{30}{i}} )}^{\frac{{t}_{s}}{2}} - 1 ) {V}_{p}}{32767 \times {V}_{d}} - 1} ) + \frac{{V}_{p} ( {( {2}^{\frac{30}{i}} )}^{\frac{{t}_{s}}{2}} - 1 )}{32767}$$

where:
KV₂ is indicative of the second cumulative delivery volume; and
tₛ is indicative of the subsequent infusion step time of the infusion step of the first number of infusion steps (h₁).

The second cumulative delivery volume KV₂ provides an indication of the cumulative volume of the fluid that is to be expelled from the medication delivery apparatus 90 between the initial time and the subsequent infusion step time (tₛ).

At 5510, the infusion computing device 151 determines a first infusion volume for the infusion step of the first number of infusion steps (h₁). In particular, the infusion computing device processor 256 determines the first infusion volume. The first infusion volume is indicative of a volume of the fluid that is to be expelled from the medication delivery apparatus 90 during the infusion step of the first number of infusion steps (h₁). The infusion computing device processor 256 determines the first infusion volume based at least in part on the first cumulative delivery volume (KV₁) and the second cumulative delivery volume (KV₂).

In some embodiments, the infusion computing device processor 256 determines the first infusion volume by determining a difference between the second cumulative delivery volume (KV₂) and the first cumulative delivery volume (KV₁). That is, the infusion computing device processor 256 subtracts the first cumulative delivery volume (KV₁) from the second cumulative delivery volume (KV₂). The result of this subtraction is the first infusion volume.

At 5512, the infusion computing device 151 determines a first target flow rate for the infusion step of the first number of infusion steps (h₁). In particular, the infusion computing device processor 256 determines the first target flow rate. The first target flow rate is a target flow rate of the infusion step of the first number of infusion steps (h₁). The infusion computing device processor 256 determines the first target flow rate based at least in part on the first infusion volume of the infusion step of the first number of infusion steps (h₁).

The medication delivery system 91 is configured to deliver the infusion step of the first number of infusion steps (h₁) over a first infusion step duration. The first infusion step duration is a time period (e.g. 20 s). In some embodiments, determining the first target flow rate comprises dividing the first infusion volume by the first infusion step duration.

In some embodiments, the first target flow rate is determined using a target flow rate function. The target flow rate function may be the derivative of the cumulative volume function.

$$Rate = \frac{{V}_{p} ln ( {2}^{30 / i} ) {( {2}^{30 / i} )}^{t / 2}}{65534 ( {W}_{0} ( - {e}^{- \frac{( {( {2}^{30 / i} )}^{t / 2} - 1 ) {V}_{p}}{32767 {V}_{d}} - 1} ) + 1 )}$$

Where Rate is the target flow rate and t is the relevant time.

It should be noted that in some embodiments, the infusion step durations of each of the infusion steps of the first number of infusion steps (h₁) may be the same. Alternatively, the infusion step durations of at least some of the infusion steps of the first number of infusion steps (h₁) may be different. For example, the first infusion step duration may be different to an infusion step duration of another infusion step of the first number of infusion steps (h₁).

In some embodiments, the first infusion step duration is less than an infusion step duration of another infusion step of the first number of infusion steps (h₁). In such cases, the infusion step of the first number of infusion steps (h₁) may be delivered closer to the initial time than the another infusion step of the first number of infusion steps (h₁). Alternatively, the infusion step of the first number of infusion steps (h₁) may be delivered further away from the initial time than the another infusion step of the first number of infusion steps (h₁).

At 5514, the infusion computing device 151 determines a first pharmaceutical dose (Dose_{c1}) for an infusion step of the second number of infusion steps (h₂). In particular, the infusion computing device processor 256 determines the first pharmaceutical dose (Dose_{c1}). The first pharmaceutical dose (Dose_{c1}) is indicative of a cumulative pharmaceutical preparation dose that is to be output by the medication delivery apparatus between the initial time and an initial infusion dose time. This may be a cumulative dose of the active agent of the pharmaceutical preparation. The initial infusion dose time corresponds to a start of the infusion step of the second number of infusion steps (h₂). The fluid that is expelled from the medication delivery apparatus 90 is the fluid that is stored in the dilution chamber 100 at the time of the infusion step of the second number of infusion steps (h₂). That is, the fluid is the diluted pharmaceutical preparation. The cumulative pharmaceutical preparation dose is the dose (i.e. amount) of the active agent output by the medication delivery apparatus 90 in this fluid. As described herein, the initial time corresponds to a start of the infusion process. For example, the initial time may be 0. The initial infusion dose time corresponds to a start of the infusion step of the second number of infusion steps (h₂). The initial infusion dose time may be indexed with respect to the initial time. For example, the initial infusion dose time may be the number of milliseconds or seconds that the infusion step of the second number of infusion steps (h₂) starts at after the initial time.

The infusion computing device processor 256 determines the first pharmaceutical dose (Dose_{c1}) using a dose function. The dose function has one or more inputs. The one or more inputs of the dose function comprise one or more of the initial infusion dose time of the infusion step of the second number of infusion steps (h₂), the concentration input (Cₚ), the time input (i), the dilution chamber volume input (V_{d}) and the volume input (Vₚ). The dose function involves a flow rate function. In particular, the dose function involves the flow rate function at the initial infusion dose time. The flow rate function may be the Tansy function described herein. Therefore, the infusion computing device processor 256 determines the first pharmaceutical dose (Dose_{c1}) based at least in part on the concentration input (Cₚ) and a value of the flow rate function at the initial infusion dose time.

The dose function can be expressed as:

$${Dose}_{c} = {C}_{p} \times {\int }_{0}^{t} T dt$$

where:
a. t is a relevant time that is indicative of a time at which the dose function is being solved;
b. Dose_{c} is indicative of the delivered pharmaceutical dose as of the relevant time t;
c. Cₚ is the concentration input;

$$T = \frac{2 {V}_{p}}{{2}^{16} - 2} {e}^{\frac{t}{2} \times ln ( {2}^{\frac{30}{i}} )} - \frac{2 {V}_{p}}{{2}^{16} - 2} ; \qquad d$$

e. i is the time input; and
f. Vₚ is the volume input.

Solving for Dose_{c} in the dose function provides an indication of the cumulative dose of the active agent (i.e. the pharmaceutical preparation) that is to be output by the medication delivery apparatus 90 between the initial time and the relevant time (t).

Therefore, the infusion computing device processor 256 determines the first pharmaceutical dose (Dose_{c1}) by calculating:

$${Dose}_{c 1} = {C}_{p} \times {\int }_{0}^{{t}_{i 2}} {T}_{i 2} dt$$

where:
a. Dose_{c1} is indicative of the first pharmaceutical dose;
b. tᵢ₂ is indicative of the initial infusion dose time; and

$${T}_{i 2} = \frac{2 {V}_{p}}{{2}^{16} - 2} {e}^{\frac{{t}_{i 2}}{2} \times ln ( {2}^{\frac{30}{i}} )} - \frac{2 {V}_{p}}{{2}^{16} - 2} . \qquad c$$

The first pharmaceutical dose (Dose_{c1}) provides an indication of the cumulative dose of the active agent (i.e. the pharmaceutical preparation) that is to be output by the medication delivery apparatus 90 between the initial time and the initial infusion dose time (tᵢ₂).

At 5516, the infusion computing device 151 determines a second pharmaceutical dose (Dose_{c2}) for the infusion step of the second number of infusion steps (h₂). In particular, the infusion computing device processor 256 determines the second pharmaceutical dose (Dose_{c2}). The second pharmaceutical dose (Dose_{c2}) is indicative of a cumulative pharmaceutical preparation dose that is to be output by the medication delivery apparatus between the initial time and a subsequent infusion dose time. This may be a cumulative dose of the active agent of the pharmaceutical preparation. The cumulative pharmaceutical preparation dose that is to be output by the medication delivery apparatus between the initial time and a subsequent infusion dose time may be referred to as a second cumulative pharmaceutical preparation dose. The fluid that is expelled from the medication delivery apparatus 90 is the fluid that is stored in the dilution chamber 100 at the time of the infusion step of the second number of infusion steps (h₂). That is, the fluid is the diluted pharmaceutical preparation. The cumulative pharmaceutical preparation dose is the dose (i.e. amount) of the active agent output by the medication delivery apparatus 90 in this fluid. As described herein, the initial time corresponds to a start of the infusion process. The subsequent infusion dose time corresponds to an end of the infusion step of the second number of infusion steps (h₂). The subsequent infusion dose time may be indexed with respect to the initial time. For example, the subsequent infusion dose time may be the number of milliseconds or seconds at which the infusion step of the second number of infusion steps (h₂) ends after the initial time.

The infusion computing device processor 256 determines the second pharmaceutical dose (Dose_{c2}) using the dose function.

In particular, the infusion computing device processor 256 determines the second pharmaceutical dose (Dose_{c2}) by calculating:

$$D o s {e}_{c 2} = {C}_{p} \times {\int }_{0}^{{t}_{s 2}} {T}_{s 2} d t$$

where:
a. Dose_{c2} is indicative of the second pharmaceutical dose;
b. tₛ₂ is indicative of the subsequent infusion dose time; and

$$c .$$

$${T}_{s 2} = \frac{2 {V}_{p}}{{2}^{16} - 2} {e}^{\frac{{t}_{s 2}}{2} \times ln ( {2}^{\frac{30}{i}} )} - \frac{2 {V}_{p}}{{2}^{16} - 2} .$$

The second pharmaceutical dose (Dose_{c2}) provides an indication of the cumulative dose of the active agent (i.e. the pharmaceutical preparation) that is to be output by the medication delivery apparatus 90 between the initial time and the subsequent infusion dose time (tₛ₂).

At 5518, the infusion computing device 151 determines a dose target for the infusion step of the second number of infusion steps (h₂). In particular, the infusion computing device processor 256 determines the dose target. The dose target is indicative of a pharmaceutical preparation dose that is to be output by the medication delivery apparatus during the infusion step of the second number of infusion steps (h₂). The pharmaceutical preparation dose may be a dose of the active agent that is to be output by the medication delivery apparatus during the infusion step of the second number of infusion steps (h₂). The infusion computing device processor 256 determines the dose target based at least in part on the first pharmaceutical dose (Dose_{c1}) and the second pharmaceutical dose (Dose_{c2}).

In some embodiments, the infusion computing device processor 256 determines the dose target by determining a difference between the second pharmaceutical dose (Dose_{c2}) and the first pharmaceutical dose (Dose_{c1}). That is, the infusion computing device processor 256 subtracts the first pharmaceutical dose (Dose_{c1}) from the second pharmaceutical dose (Dose_{c2}). The result of this subtraction is the dose target.

At 5520, the infusion computing device 151 determines a concentration estimate. In particular, the infusion computing device processor 256 determines the concentration estimate. The concentration estimate is for the infusion step of the second number of infusion steps (h₂). The concentration estimate is indicative of a pharmaceutical preparation concentration of the fluid expelled from the medication delivery apparatus 90 during the infusion step of the second number of infusion steps (h₂). This may be a concentration of the active agent in the fluid stored in the dilution chamber 100. The concentration estimate is indicative of a pharmaceutical preparation concentration of the fluid expelled from the medication delivery apparatus 90 during each of the infusion steps of the second number of infusion steps (h₂). This is because the concentration of the pharmaceutical preparation in the fluid of the dilution chamber 100 during the second time window is constant.

The infusion computing device processor 256 determines the concentration estimate using a concentration estimate function. The concentration estimate function may be as is described herein. The concentration estimate function has one or more inputs. The one or more inputs of the concentration estimate function comprise one or more of the concentration input (Cₚ), the dilution chamber volume input (V_{d}) and the volume input (Vₚ). Thus, the infusion computing device processor 256 determines the concentration estimate based at least in part on one or more of the concentration input (Cₚ), the dilution chamber volume input (V_{d}) and the volume input (Vₚ).

The concentration function can be expressed as:

$${C}_{d c} = {C}_{p} \times ( 1 - {e}^{\frac{{V}_{p}}{{V}_{d}}} )$$

where:
C_{dc} is indicative of the concentration estimate;
Cₚ is the concentration input;
Vₚ is the volume input; and
V_{d} is the dilution chamber volume input.

At 5522, the infusion computing device 151 determines a second infusion volume for the infusion step of the second number of infusion steps (h₂). In particular, the infusion computing device processor 256 determines the second infusion volume. The second infusion volume is indicative of a volume of the fluid that is to be expelled from the medication delivery apparatus 90 during the infusion step of the second number of infusion steps (h₂). The infusion computing device processor 256 determines the second infusion volume based at least in part on the dose target and the concentration estimate.

In some embodiments, the infusion computing device processor 256 determines the second infusion volume by dividing the dose target by the concentration estimate.

At 5524, the infusion computing device 151 determines a second target flow rate for the infusion step of the second number of infusion steps (h₂). In particular, the infusion computing device processor 256 determines the second target flow rate. The second target flow rate is a target flow rate of the infusion step of the second number of infusion steps (h₁). The infusion computing device processor 256 determines the second target flow rate based at least in part on the second infusion volume of the infusion step of the second number of infusion steps (h₂).

In some embodiments, the second target flow rate is determined using the target flow rate function described herein.

The medication delivery system 91 is configured to deliver the infusion step of the second number of infusion steps (h₂) over a second infusion step duration. The second infusion step duration is a time period (e.g. 20 s). In some embodiments, determining the second target flow rate comprises dividing the second infusion volume by the second infusion step duration.

It should be noted that in some embodiments, the infusion step durations of each of the infusion steps of the second number of infusion steps (h₂) may be the same. Alternatively, the infusion step durations of at least some of the infusion steps of the second number of infusion steps (h₂) may be different. For example, the second infusion step duration may be different to an infusion step duration of another infusion step of the second number of infusion steps (h₂).

In some embodiments, the second infusion step duration is less than an infusion step duration of another infusion step of the second number of infusion steps (h₂). In such cases, the infusion step of the second number of infusion steps (h₂) may be delivered closer to the initial time than the another infusion step of the second number of infusion steps (h₂). Alternatively, the infusion step of the second number of infusion steps (h₂) may be delivered further away from the initial time than the another infusion step of the second number of infusion steps (h₂).

The infusion computing device processor 256 generates an infusion process based at least in part on the first infusion volume. The infusion computing device processor 256 generates the infusion process based at least in part on the second infusion volume. The infusion process may be referred to as an infusion program. The infusion process defines the rate at which the first plunger is to be actuated during the infusion to deliver the active agent as intended. That is, the infusion process defines the volume of fluid that is to be expelled by the medication delivery apparatus 90 over predetermined units of time. The infusion computing device processor 256 stores the infusion process in the infusion computing device memory 258. In particular, the infusion computing device processor 256 stores the infusion process in the infusion computing device memory 258 in the form of an infusion process file.

In some embodiments, the clinician saves the infusion process file on a removable storage medium such as a USB memory module. The clinician may transfer the infusion process file to the infusion device 93. The infusion device 93 may then perform the infusion process using the infusion process file. In some embodiments, the infusion computing device processor 256 transmits the infusion process file to the infusion device 93. For example, the communications network 264 may be in the form of a network (e.g. the Internet or a Local Area Network). In these cases, the infusion computing device processor 256 may transmit the infusion process file to the infusion device 93 via the communications network 264.

At 5526, the infusion device 93 performs the infusion process. In particular, at 5526, the infusion device 93 actuates a plunger of the medication delivery apparatus. In particular, the infusion device 93 actuates the first plunger 92. The infusion device processor sends a control signal to the infusion device actuator to actuate the first plunger 92. The infusion device 93 may therefore be said to actuate the infusion device actuator to actuate the first plunger 92, in some embodiments.

Where the infusion device actuator is in the form of a syringe driver (or another contact-based actuator), the infusion device 93 may actuate the syringe driver (or the other actuator) to actuate the first plunger 92. The infusion device 93 may move the infusion device actuator such that it contacts the first plunger 92. The infusion device 93 may continue to move the infusion device actuator to cause movement of the first plunger 92 within the container 96. In particular, the infusion device actuator may be actuated to move the first plunger 92 towards the dilution chamber outlet 110. This movement of the first plunger 92 can be considered actuation of the first plunger 92.

As described herein, in some embodiments, the infusion device 93 may be in the form of a vacuum infusion device. The vacuum infusion device may apply a vacuum pressure to the dilution chamber outlet 110, which can cause motion of the first plunger 92. This motion of the first plunger 92 is considered actuation of the first plunger 92. Therefore, in some embodiments, the infusion device 93 actuates the first plunger 92 at least in part by way of applying the vacuum pressure to the dilution chamber outlet 110.

The infusion device 93 actuates the first plunger 92 such that the first infusion volume of the fluid is expelled from the medication delivery apparatus 90 during the infusion step of the first number of infusion steps (h₁). In other words, the infusion device 93 actuates the first plunger 92 to expel the first infusion volume of the fluid from the medication delivery apparatus 90 during the infusion step of the first number of infusion steps (h₁). The infusion device 93 actuates the first plunger 92 such that the first infusion volume of the fluid is expelled from the medication delivery apparatus 90 during the infusion step of the first number of infusion steps (h₁) at the first target flow rate.

The infusion device 93 actuates the first plunger 92 such that the second infusion volume of the fluid is expelled from the medication delivery apparatus 90 during the infusion step of the second number of infusion steps (h₂). In other words, the infusion device 93 actuates the first plunger 92 to expel the second infusion volume of the fluid from the medication delivery apparatus 90 during the infusion step of the second number of infusion steps (h₂). The infusion device 93 actuates the first plunger 92 such that the second infusion volume of the fluid is expelled from the medication delivery apparatus 90 during the infusion step of the second number of infusion steps (h₂) at the second target flow rate.

In some embodiments, the concentration of the active agent in the fluid that is to be expelled from the medication delivery apparatus 90 during the infusion step of the first number of infusion steps (h₁) is a first concentration. The concentration of the active agent in the fluid that is to be expelled from the medication delivery apparatus 90 during the infusion step of the second number of infusion steps (h₂) is a second concentration. In some embodiments, the first concentration is at least one order of magnitude lower than the second concentration. That is, in some embodiments, a concentration of the active agent in the infusion volume of the fluid that is to be expelled from the medication delivery apparatus 90 during the infusion step of the first number of infusion steps (h₁) is at least one order of magnitude lower than a concentration of the active agent in second infusion volume of the fluid that is to be expelled from the medication delivery apparatus 90 during the infusion step of the second number of infusion steps (h₂). In some embodiments, the first concentration is at least one order of magnitude greater than the second concentration. In some embodiments, the first concentration is at least one order of magnitude less than the second concentration. In some embodiments, the first concentration is equal to the second concentration.

### Transitional Infusion Step

In some embodiments, the first plunger 92 contacts the second plunger 94 during an infusion step of the infusion process. That is, the first time window transitions to the second time window during an infusion step of the infusion process. This infusion step may be referred to as a transitional infusion step (hₜ). The transitional time is indicative of a temporal point that divides the first time window and the second time window.

In some embodiments, the method 5500 comprises determining the transitional time. The infusion computing device 151 determines the transitional time. In particular, the infusion computing device processor 256 determines the transitional time. The infusion computing device processor 256 may determine the transitional time by equating a transitional cumulative delivery volume (KV_{T}) to the volume input (Vₚ). The infusion computing device processor 256 may use the cumulative volume function to determine the transitional time. For example, the infusion computing device processor 256 may solve for the transitional time (tₜ) in the below equation:

$${KV}_{t} = {V}_{d} + {V}_{d} {W}_{0} ( - {e}^{- \frac{( {( {2}^{\frac{30}{i}} )}^{\frac{{t}_{t}}{2}} - 1 ) {V}_{p}}{32767 \times {V}_{d}} - 1} ) + \frac{{V}_{p} ( {( {2}^{\frac{30}{i}} )}^{\frac{{t}_{t}}{2}} - 1 )}{32767}$$

Where KV_{T} is indicative of the transitional cumulative delivery volume and tₜ is indicative of the transitional time.

In some embodiments, the infusion computing device processor 256 uses a transitional time function to determine the transitional time. The transitional time function is reproduced below:

$${t}_{t} = \frac{ln ( {V}_{p} - ( {V}_{d} \times ( 1 - {e}^{\frac{{V}_{p}}{{V}_{d}}} ) ) + \frac{2 \times {V}_{p}}{{2}^{16} - 2} ) - ln ( \frac{2 \times {V}_{p}}{{2}^{16} - 2} )}{\frac{1}{2} ln ( {2}^{\frac{30}{i}} )}$$

where tₜ is the transitional time.

In some embodiments, the method 5500 comprises determining a transitional infusion step volume for the transitional infusion step (hₜ). The infusion computing device processor 256 determines the transitional infusion step volume for the transitional infusion step (hₜ). A first transitional infusion volume is indicative of a volume of the fluid that is to be expelled from the medication delivery apparatus 90 during a first portion of the transitional infusion step (hₜ) (i.e. during the first time window). A second transitional infusion volume is indicative of a volume of the fluid that is to be expelled from the medication delivery apparatus 90 during a second portion of the transitional infusion step (hₜ) (i.e. during the second time window). The infusion computing device processor 256 determines the transitional infusion step volume by determining the sum of the first transitional infusion volume and the second transitional infusion volume.

In some embodiments, the infusion computing device processor 256 determines the first transitional infusion volume for the transitional infusion step (hₜ) using the cumulative delivery volume function as described with reference to 5506. The infusion computing device processor 256 calculates the volume of the fluid that is to be delivered from the initial time to an initial transitional infusion step time. The initial transitional infusion step time corresponds to the start of the transitional infusion step (hₜ). The infusion computing device processor 256 calculates the volume of the fluid that is to be delivered from the initial time to the transitional time (tₜ). The infusion computing device processor 256 subtracts the volume of the fluid that is to be delivered from the initial time to the initial transitional infusion step time from the volume of the fluid that is to be delivered from the initial time to the transitional time (tₜ).

In some embodiments, the infusion computing device processor 256 determines the second transitional infusion volume for the transitional infusion step (hₜ) as described with reference to 5514, 5516, 5518, 5520 and 5522.

In some embodiments, the infusion computing device processor 256 determines a transitional target flow rate. The transitional target flow rate is indicative of a flow rate at which the fluid is to be expelled from the medication delivery apparatus 90 during the transitional infusion step (hₜ). The infusion computing device processor 256 determines the transitional target flow rate based at least in part on the transitional step volume. In particular, the infusion computing device processor 256 determines the transitional target flow rate by dividing the transitional step volume by a transitional step duration. The transitional step duration corresponds to a duration of time over which the transitional infusion step (hₜ) is delivered.

The transitional infusion step (hₜ) is performed over the transitional infusion step duration. The transitional infusion step duration comprises the transitional time.

The infusion device 93 actuates the first plunger 92 such that the transitional step infusion volume of the fluid is expelled from the medication delivery apparatus 90 during the transitional infusion step (hₜ). In other words, the infusion device 93 actuates the first plunger 92 to expel the transitional infusion volume of the fluid from the medication delivery apparatus 90 during the transitional infusion step (hₜ). The infusion device 93 actuates the first plunger 92 such that the transitional infusion volume of the fluid is expelled from the medication delivery apparatus 90 during the transitional infusion step (hₜ) at the transitional target flow rate.

The transitional infusion step (hₜ) is between the infusion step of the first number of infusion steps (h₁) and the infusion step of the second number of infusion steps (h₂).

As described herein, in some cases, the infusion process, if left unabated, may deliver the active agent at a rate that exceeds a maximum infusion rate. The maximum infusion rate may vary by active agent. In some embodiments, when determining the infusion process, the infusion computing device processor 256 determines a maximum dose time. The maximum dose time is indicative of a dose temporal point at which a maximum infusion rate threshold is reached. The maximum infusion rate threshold corresponds to a maximum rate at which the active agent can be safely provided to the patient. The infusion computing device processor 256 uses the dose rate function to determine a time at which the infusion process will be delivering the active agent at the maximum infusion rate. The dose rate function is as is described herein, and shown below:

$${D}_{r} = {C}_{p} \times T ( t )$$

Where Dᵣ is the dose rate at a particular time (t) of the infusion process and T(t) is the Tansy rate function.

The clinician can look up the maximum infusion rate for the particular active agent, or alternatively, the infusion device can refer to a database to retrieve the maximum infusion rate. The maximum infusion rate can be set to equal the dose rate (Dᵣ.) of the dose rate function. The time at which this maximum infusion rate is delivered can then be determined. This is the maximum dose time.

The infusion computing device 151 determines whether this time is in the first time window or the second time window. Where it is in the first time window, the infusion computing device 151 determines a cumulative volume of fluid delivered to this point. This may be done using the cumulative volume function described herein. The infusion computing device 151 then uses a constant volume function to ensure that the infusion proceeds with providing the active agent at or below the maximum infusion rate.

Where the maximum dose time is in the second time window, the infusion computing device 151 uses a constant infusion rate function to ensure that the infusion proceeds with providing the active agent at or below the maximum infusion rate. This may be for the remainder of the second time window and until the infusion process concludes.

### Derivation of the Cumulative Volume Function (the Kelly Cumulative Volume Function)

A derivation of the cumulative volume function is detailed herein. The cumulative volume function is the Kelly Cumulative Volume Function. The cumulative volume function is used to control the medication delivery system 91 in cases where the dilution chamber 100 is emptied during the infusion process. The cumulative volume function is only used however, during times when the volume of the dilution chamber 100 remains constant. That is, the cumulative volume function is only used during the first time window. The cumulative volume function determines the rate that the active agent chamber 98 is emptied by actuation of the first plunger 92. This actuation of the first plunger 92 may be either by contacting and moving the first plunger 92 with a directly applied force, or by aspirating the fluid stored in the dilution chamber 100 out of the dilution chamber opening 110.

The rate of change of the amount of the pharmaceutical preparation in the dilution chamber 100 may be represented by A_{d}′(v). This may be with respect to the cumulative volume of the fluid delivered into or out of the dilution chamber (v). The cumulative volume (v) of the fluid delivered into or out of the dilution chamber 100 is equal to the concentration (Cₚ) of the active agent in the active agent chamber 98 minus the concentration C_{d}(v) of the active agent in the dilution chamber 100 at the point that (v) mL has been infused.

$${A}_{d}^{′} ( v ) = {C}_{p} - {C}_{d} ( v )$$

The concentration C_{d}(v) of the active agent in the dilution chamber 100 is equal to the amount of active agent in the dilution chamber A_{d}(v) divided by the volume of the dilution chamber (V_{d}). Therefore:

$${A}_{d}^{′} ( v ) = {C}_{p} - \frac{{A}_{d} ( v )}{{V}_{d}}$$

### Therefore:

$${A}_{d}^{′} ( v ) + \frac{{A}_{d} ( v )}{{V}_{d}} = {C}_{p}$$

### Or:

$${A}_{d}^{′} ( v ) + \frac{1}{{V}_{d}} {A}_{d} ( v ) = {C}_{p}$$

This first order liner equation represents the amount of drug in the dilution chamber with respect to the cumulative volume (v) of fluid entering or leaving the dilution chamber, and has the integrating factor, I:

$$I = {e}^{\int Pdv} = {e}^{\int \frac{I}{{V}_{d}} d v}$$

### Where:

$$P = \frac{1}{{V}_{d}}$$

And the following identity is known:

$$\frac{d}{d v} ( I \times {A}_{d} ( v ) ) = I \times {A}_{d}^{′} ( v ) + I \times P \times {A}_{d} ( v )$$

If we multiply our first order linear equation by I:

$$I \times {A}_{d}^{′} ( v ) + I \times \frac{1}{{V}_{d}} {A}_{d} ( v ) = I \times {C}_{p}$$

Consequently, this can be written in the equivalent form:

$$\frac{d}{d v} ( {e}^{\frac{v}{{V}_{d}}} \times {A}_{d} ( v ) ) = {e}^{\frac{v}{{V}_{d}}} \times {C}_{p}$$

Integrate both sides:

$$\int \frac{d}{d v} ( {e}^{\frac{v}{{V}_{d}}} \times {A}_{d} ( v ) ) d v = \int {e}^{\frac{v}{{V}_{d}}} \times {C}_{p} d v$$

### Therefore:

$$\begin{matrix}{e}^{\frac{v}{{V}_{d}}} \times {A}_{d} ( v ) = {V}_{d} \times {e}^{\frac{v}{{V}_{d}}} \times {C}_{p} + {C}_{1} \\ {A}_{d} ( v ) = \frac{{C}_{p} {V}_{d} \times {e}^{\frac{v}{{V}_{d}}} + {C}_{1}}{{e}^{\frac{v}{{V}_{d}}}} \\ {A}_{d} ( v ) = {C}_{p} {V}_{d} + \frac{{C}_{1}}{{e}^{\frac{v}{{V}_{d}}}}\end{matrix}$$

### Or:

$${A}_{d} ( v ) = {V}_{d} {C}_{p} + {C}_{1} {e}^{- \frac{v}{{V}_{d}}}$$

Where C₁ is a constant.

At V=0, A(V_{d})=0, therefore:

$${C}_{1} = - {V}_{d} {C}_{p}$$

### So:

$${A}_{d} ( v ) = {V}_{d} {C}_{p} - {V}_{d} {C}_{p} {e}^{\frac{v}{{V}_{d}}}$$

### Or:

$${A}_{d} ( v ) = {V}_{d} {C}_{p} ( 1 - {e}^{- \frac{v}{{V}_{d}}} )$$

The concentration of the active agent in the dilution chamber 100 when the cumulative volume injected into or drawn out of the dilution chamber (C(V_{d})) is equal to:

$${C}_{d} ( v ) = \frac{{A}_{d} ( v )}{{V}_{d}} = {C}_{p} ( 1 - {e}^{- \frac{v}{{V}_{d}}} )$$

The integral of C(V_{d}) is the cumulative dose leaving the dilution chamber 100 with respect to the cumulative volume that has left the dilution chamber at that point (V).

$$Dose ( V ) = {\int }_{0}^{V} C ( V ) d V = {\int }_{0}^{v} {C}_{p} ( 1 - {e}^{- \frac{v}{{V}_{d}}} ) d v = {C}_{p} {\int }_{0}^{v} ( 1 - {e}^{- \frac{v}{{V}_{d}}} ) d v$$

### Therefore:

$$Dose ( v ) = {C}_{p} ( ( {V}_{d} {e}^{\frac{v}{{V}_{d}}} ) + v + {C}_{2} )$$

Where C₂ is a constant.

At dose=0, V=0:

$$0 = {C}_{p} ( ( {V}_{d} {e}^{- \frac{0}{{V}_{d}}} ) + 0 + {C}_{2} )$$

### Therefore:

$$0 = {V}_{d} {e}^{- \frac{0}{{V}_{d}}} + 0 + {C}_{2}$$

### Therefore:

$${C}_{2} = - {V}_{d}$$

### Therefore:

$$Dose ( V ) = {C}_{p} ( ( {V}_{d} {e}^{- \frac{v}{{V}_{d}}} ) + v - {V}_{d} )$$

We also know that the dose at any time during the infusion process is given by the Tansy Dose Function (as described herein), which is Cₚ×Tansy Volume Function, which is Cₚ×the integral of the Tansy Rate Function:

$$Dose ( t ) = {C}_{p} ( \frac{2 {V}_{p}}{{2}^{16} - 2} {e}^{\frac{t}{2} ln ( {2}^{30 / i} )} - \frac{2 {V}_{p}}{{2}^{16} - 2} )$$

So for any cumulative dose, we can calculate both the cumulative volume and the cumulative time elapsed. Therefore we can express cumulative volume delivered with respect to time:

$${C}_{p} ( ( {V}_{d} {e}^{- \frac{v}{{V}_{d}}} ) + v - {V}_{d} ) = {C}_{p} ( \frac{2 {V}_{p}}{{2}^{16} - 2} {e}^{\frac{t}{2} ln ( {2}^{30 / i} )} - \frac{2 {V}_{p}}{{2}^{16} - 2} )$$

### Simplified to:

$$( {V}_{d} {e}^{- \frac{v}{{V}_{d}}} ) + v - {V}_{d} = \frac{2 {V}_{p}}{{2}^{16} - 2} {e}^{\frac{t}{2} ln ( {2}^{30 / i} )} - \frac{2 {V}_{p}}{{2}^{16} - 2}$$

Which can be solved for cumulative volume (v) with respect to time (t), giving an exact form of the cumulative volume function (an exact form of the Kelly Cumulative Volume Function):

$$v = {V}_{d} + {V}_{d} {W}_{0} ( - {e}^{- \frac{( ( {( {2}^{30 / i} )}^{t / 2} ) - 1 ) {V}_{p}}{32767 \times {V}_{d}} - 1} ) + \frac{{V}_{p} ( {( {2}^{30 / i} )}^{t / 2} ) - 1}{32767}$$

In this case, v is equivalent to the cumulative delivery volume KV as described herein. W₀ is the principle branch (branch 0) of the Lambert W function. The Lambert W function is defined as the inverse function of we^{w}.

The derivative of the cumulative volume is rate (volume per time), giving an exact form of a rate function (an exact form of the Kelly Rate Function):

$$Rate = \frac{{V}_{p} ln ( {2}^{30 / i} ) {( {2}^{30 / i} )}^{t / 2}}{65534 ( {W}_{0} ( - {e}^{- \frac{( {( {2}^{30 / i} )}^{t / 2} - 1 ) {V}_{p}}{32767 {V}_{d}} - 1} ) + 1 )}$$

### Constant Dose Infusion Cumulative Volume Function

In some embodiments, the infusion device processor is configured to perform the infusion as per the method 5500 for a first infusion portion, and is configured to perform the method according to a constant dose process for a second infusion portion. The infusion computing device processor 256 is configured to determine a maximum dose time. The maximum dose time is a time within the time window at which the dose rate of the infusion process reaches a maximum dose threshold. Where the maximum dose time is within the first time window, rather than using the cumulative volume function described above, the infusion device processor (via the infusion process file) is governed by a constant dose cumulative volume function. For the method 5500, the constant dose cumulative volume function may be referred to as a Kelly Constant Dose Cumulative Volume Function. The Kelly Constant Dose Cumulative Volume Function may be:

$$KV = \frac{1}{i} ( {V}_{d} i + {V}_{d} {iW}_{o} ( - {e}^{- ( \frac{{V}_{p} t}{{V}_{d} i} ) - 1} ) + {V}_{p} t )$$

### Method 5600 for Delivering a Pharmaceutical Preparation to a Patient

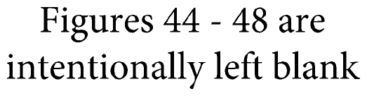

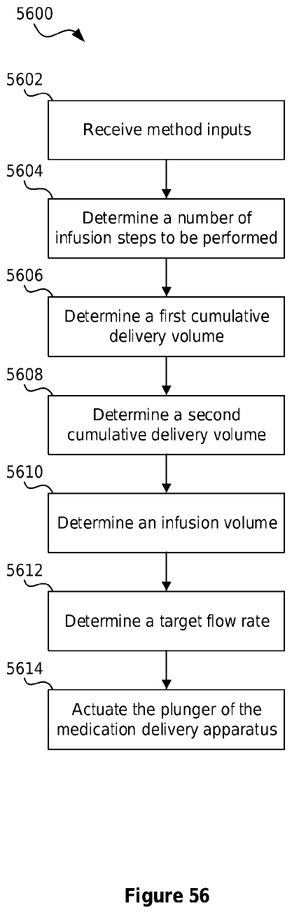

FIG. 56 is a process flow diagram of a method 5600 for delivering a pharmaceutical preparation to a patient, according to some embodiments. The method 5600 may be performed by the medication delivery system 1 and/or the medication delivery apparatus 10 described with reference to FIGS. 1 to 11e. The method 5600 may be performed by the medication delivery system 91 and/or the medication delivery apparatus 90 described with reference to FIG. 30 to 34a or 35 to 43. The method 5600 may be performed by the medication delivery system and/or the medication delivery apparatus 136 described with respect to FIGS. 44 to 47A. The pharmaceutical preparation is delivered to the patient by an infusion process that commences at an initial time. Although the method 5600 is described with reference to the medication delivery system 1 of FIGS. 1 to 11e, it will be understood that the description is also applicable to the medication delivery system 91 and/or the medication delivery apparatus 90 described with reference to FIG. 30 to 34a or 35 to 43 and the medication delivery system and/or medication delivery apparatus 136 described with reference to FIGS. 44 to 47A.

Some or all of the method 5600 may be performed by the infusion device processor (i.e. the processor of the infusion device). Some or all of the method may be performed by the infusion computing device 151. Some or all of the method may be performed by another computing device. The method 5600 may therefore be considered a computer-implemented method.

At 5602, the infusion computing device receives one or more method inputs. In particular, the infusion computing device processor receives the one or more method inputs. In some embodiments, the one or more method inputs comprises a plurality of method inputs. The method inputs may be received via the infusion computing device user interface 260 (e.g. via the display and/or the keyboard). In some embodiments, at least one of the method inputs is an input of a cumulative delivery volume function. In some embodiments, at least one of the method inputs is an input of a dose function. The clinician may input the one or more method inputs.

In some embodiments, the one or more method inputs comprises a concentration input (Cₚ). The concentration input (Cₚ) may be as described herein. In some embodiments, the one or more method inputs comprises a volume input (Vₚ). The volume input (Vₚ) may be as is described herein. In some embodiments, the one or more method inputs comprises a dilution chamber volume input (V_{d}). The dilution chamber volume input (V_{d}) may be as is described herein. In some embodiments, the one or more method inputs comprises a time input (i). The time input (i) may be as is described herein. The time input (i) is indicative of a time window over which the pharmaceutical preparation is to be delivered.

At 5604, the infusion computing device determines the number of infusion steps (h) that are to be performed within the time window. In particular, the infusion computing device processor determines the number of infusion steps (h) that are to be performed within the time window. The time window and the number of infusion steps (h) may be as described herein.

In some embodiments, determining the number of infusion steps (h) comprises receiving the number of infusion steps (h). The number of infusion steps (h) may be received (e.g. by the infusion computing device processor) as an infusion step input. Alternatively, the one or more method inputs may comprise the number of infusion steps (h).

In some embodiments, determining the number of infusion steps (h) comprises calculating a product of a number of infusion steps (per minute) that are to be performed within the time window (g) and the time input (i). That is, to determine the number of infusion steps (h), the infusion computing device processor calculates:

$$h = g \times i$$

where h is the number of infusion steps that are to be performed within the time window, g is the number of infusion steps per minute that are to be performed during the infusion process and (i) is the time input. The time input (i) may therefore indicate a length of the infusion process in minutes. In these embodiments, the infusion computing device processor may store the number of infusion steps (h) in the infusion computing device memory. The infusion computing device processor may subsequently issue a read transaction to the infusion computing device memory relating to the number of infusion steps (h), and may receive the number of infusion steps (h) in response. Thus, receiving the number of infusion steps (h) may comprise determining the number of infusion steps (h).

At 5606, the infusion computing device determines a first cumulative delivery volume (KV₁) for a target infusion step. The target infusion step is an infusion step of the number of infusion steps (h). In particular, the infusion computing device processor determines the first cumulative delivery volume (KV₁) for the target infusion step. The first cumulative delivery volume (KV₁) is indicative of a cumulative volume of a fluid that is to be expelled from the medication delivery apparatus 10 between an initial time and an initial infusion step time. The fluid that is expelled from the medication delivery apparatus 10 is the fluid that is stored in the dilution chamber 32 at the time of the target infusion step. That is, the fluid is the diluted pharmaceutical preparation. As described herein, the initial time corresponds to a start of the infusion process. For example, the initial time may be 0. The initial infusion step time corresponds to a start of the target infusion step. The initial infusion step time may be indexed with respect to the initial time. For example, the initial infusion step time may be the number of milliseconds or seconds that the target infusion step starts at after the initial time.

The infusion computing device processor determines the first cumulative delivery volume (KV₁) using a cumulative delivery volume function.

Determining an infusion volume using a Constant Cumulative Volume Function

The cumulative delivery volume function may be referred to as a second cumulative delivery volume function. The cumulative delivery volume function may be referred to as a Constant Cumulative Volume Function. The cumulative delivery volume function has one or more inputs. The one or more inputs of the cumulative delivery volume function comprise one or more of the initial infusion step time, the time input (i), the dilution chamber volume input (V_{d}) and the volume input (Vₚ). Thus, infusion computing device processor determines the first cumulative delivery volume (KV₁) based at least in part on one or more of the initial infusion step time of the infusion step of the first number of infusion steps (h₁), the time input (i), the dilution chamber volume input (V_{d}) and the volume input (Vₚ).

The cumulative delivery volume function can be expressed as:

$$KV = \frac{1}{i} ( {V}_{d} i + {V}_{d} {iW}_{o} ( - {e}^{- ( \frac{{V}_{p} t}{{V}_{d} i} ) - 1} ) + {V}_{p} t )$$

where:
KV is indicative of the cumulative volume delivered at time t (i.e. the cumulative delivery volume);
V_{d} is the dilution chamber volume input;
W₀ is the principle branch of the Lambert W function;
i is the time input;
t is a relevant time that is indicative of a time at which the cumulative delivery volume function is being solved; and
Vₚ is the volume input.

Solving for KV in the cumulative delivery volume function provides an indication of the cumulative volume of a fluid that is to be expelled from the medication delivery apparatus 10 between the initial time and the relevant time (t).

Therefore, the infusion computing device processor determines the first cumulative delivery volume (KV₁) by calculating:

$${KV}_{1} = \frac{1}{i} ( {V}_{d} i + {V}_{d} {iW}_{o} ( - {e}^{- ( \frac{{V}_{p} {t}_{i}}{{V}_{d} i} ) - 1} ) + {V}_{p} {t}_{i} )$$

where:

KV₁ is indicative of the first cumulative delivery volume; and
tᵢ is indicative of the initial infusion step time of the target infusion step.

The first cumulative delivery volume KV₁ provides an indication of the cumulative volume of the fluid that is to be expelled from the medication delivery apparatus 10 between the initial time and the initial infusion step time (tᵢ).

At 5608, the infusion computing device determines a second cumulative delivery volume (KV₂) for the target infusion step. In particular, the infusion computing device processor determines the second cumulative delivery volume (KV₂) for the target infusion step. The second cumulative delivery volume (KV₂) is indicative of a cumulative volume of a fluid that is to be expelled from the medication delivery apparatus 10 between the initial time and a subsequent infusion step time. The cumulative volume of the fluid that is to be expelled from the medication delivery apparatus 10 between the initial time and the subsequent infusion step time may be referred to as a second cumulative volume. The fluid that is expelled from the medication delivery apparatus 10 is the fluid that is stored in the dilution chamber 32 at the time of the infusion step of the first number of infusion steps (h₁). That is, the fluid is the diluted pharmaceutical preparation. As described herein, the initial time corresponds to a start of the infusion process. The subsequent infusion step time corresponds to an end of the target infusion step. The subsequent infusion step time may be indexed with respect to the initial time. For example, the subsequent infusion step time may be the number of milliseconds or seconds at which the target infusion step ends after the initial time.

The infusion computing device processor determines the second cumulative delivery volume (KV₂) using the cumulative delivery volume function.

In particular, the infusion computing device processor determines the second cumulative delivery volume (KV₂) by calculating:

$${KV}_{2} = \frac{1}{i} ( {V}_{d} i + {V}_{d} {iW}_{o} ( - {e}^{- ( \frac{{V}_{p} {t}_{s}}{{V}_{d} i} ) - 1} ) + {V}_{p} {t}_{s} )$$

where:
KV₂ is indicative of the second cumulative delivery volume; and
tₛ is indicative of the subsequent infusion step time of the target infusion step.

The second cumulative delivery volume KV₂ provides an indication of the cumulative volume of the fluid that is to be expelled from the medication delivery apparatus 10 between the initial time and the subsequent infusion step time (tₛ).

At 5610, the infusion computing device determines an infusion volume for the target infusion step. In particular, the infusion computing device processor determines the infusion volume. The infusion volume is indicative of a volume of the fluid that is to be expelled from the medication delivery apparatus 10 during the target infusion step. The infusion computing device processor determines the infusion volume based at least in part on the first cumulative delivery volume (KV₁) and the second cumulative delivery volume (KV₂).

In some embodiments, the infusion computing device processor determines the first infusion volume by determining a difference between the second cumulative delivery volume (KV₂) and the first cumulative delivery volume (KV₁). That is, the infusion computing device processor subtracts the first cumulative delivery volume (KV₁) from the second cumulative delivery volume (KV₂). The result of this subtraction is the first infusion volume.

### Determining an Infusion Volume Using a Varying Cumulative Volume Function

In some embodiments, the cumulative delivery volume function is in the form of a Varying Cumulative Volume Function. That is, the volume of the fluid that is expelled by the medication delivery apparatus 10 when controlled in accordance with the varying cumulative volume function varies over time (e.g. increases over successive infusion steps).

As described herein, the cumulative delivery volume function has one or more inputs. The one or more inputs of the cumulative delivery volume function comprise one or more of the initial infusion step time, the time input (i), the dilution chamber volume input (V_{d}), the volume input (Vₚ), and a principle branch (W₀) of a Lambert W function. Thus, infusion computing device processor determines the first cumulative delivery volume (KV₁) based at least in part on one or more of the initial infusion step time of the infusion step of the first number of infusion steps (h₁), the time input (i), the dilution chamber volume input (V_{d}) and the volume input (Vₚ).

The cumulative delivery volume function (i.e. the Varying Cumulative Volume Function) can be expressed as:

$$( {V}_{d} {e}^{- \frac{KV}{{V}_{d}}} ) + KV - {V}_{d} = ( \frac{2 {V}_{p}}{{2}^{16} - 2} {e}^{\frac{t}{2} ln ( {2}^{30 / i} )} - \frac{2 {V}_{p}}{{2}^{16} - 2} ) \times β$$

where:
KV is indicative of the relevant cumulative delivery volume;
V_{d} is the dilution chamber volume input;
i is the time input;
t is a relevant time that is indicative of a time at which the cumulative delivery volume function is being solved;
β is a volume parameter; and
Vₚ is the volume input.

In some embodiments, the volume parameter (β) is determined by calculating:

$$β = {V}_{p} - {V}_{d} ( 1 - {e}^{- \frac{{V}_{p}}{{V}_{d}}} )$$

In these embodiments, the cumulative delivery volume function may be referred to as a Sadleir Cumulative Volume Function.

In some embodiments, the volume input Vₚ is increased. The increased volume input Vₚ₂ is determined by calculating:

$${V}_{p 2} = {V}_{d} + {V}_{d} {W}_{0} ( - {e}^{- \frac{( ( {2}^{15} ) - 1 ) {V}_{0}}{32767 \times {V}_{d}} - 1} ) + \frac{{V}_{0} ( ( {2}^{15} ) - 1 )}{32767}$$

### Where:

Vₚ₂ is the increased volume input;
W₀ is a principle branch of a Lambert W function; and
V₀ is an intended delivery volume of the pharmaceutical preparation (that is, a volume of the pharmaceutical preparation that contains the amount of drug to be administered to the patient during the infusion)

In these embodiments, the cumulative delivery volume function may be referred to as a Sadleir Increased Volume Cumulative Function. The increased volume input Vₚ₂ is determined because some of the pharmaceutical preparation remains in the dilution chamber when using this method of delivery.

Solving for KV in the cumulative delivery volume function provides an indication of the cumulative volume of a fluid that is to be expelled from the medication delivery apparatus 10 between the initial time and the relevant time (t).

Therefore, in some embodiments, at 5606, the infusion computing device processor determines the first cumulative delivery volume (KV₁) by calculating:

$$( {V}_{d} {e}^{- \frac{{KV}_{1}}{{V}_{d}}} ) + {KV}_{1} - {V}_{d} = ( \frac{2 {V}_{p}}{{2}^{16} - 2} {e}^{\frac{{t}_{i}}{2} ln ( {2}^{30 / i} )} - \frac{2 {V}_{p}}{{2}^{16} - 2} ) \times β$$

where:
KV₁ is indicative of the first cumulative delivery volume; and
tᵢ is indicative of the initial infusion step time of the target infusion step.

The first cumulative delivery volume KV₁ provides an indication of the cumulative volume of the fluid that is to be expelled from the medication delivery apparatus 10 between the initial time and the initial infusion step time (tₜ).

In some embodiments, at 5608, the infusion computing device determines a second cumulative delivery volume (KV₂) for the target infusion step. In particular, the infusion computing device processor determines the second cumulative delivery volume (KV₂) for the target infusion step. The second cumulative delivery volume (KV₂) is indicative of a cumulative volume of a fluid that is to be expelled from the medication delivery apparatus 10 between the initial time and a subsequent infusion step time of the target infusion step. The cumulative volume of the fluid that is to be expelled from the medication delivery apparatus 10 between the initial time and the subsequent infusion step time may be referred to as a second cumulative volume. The fluid that is expelled from the medication delivery apparatus 10 is the fluid that is stored in the dilution chamber 32 at the time of the target infusion step. That is, the fluid is the diluted pharmaceutical preparation. As described herein, the initial time corresponds to a start of the infusion process. The subsequent infusion step time corresponds to an end of the target infusion step. The subsequent infusion step time may be indexed with respect to the initial time. For example, the subsequent infusion step time may be the number of milliseconds or seconds at which the target infusion step ends after the initial time.

The infusion computing device processor determines the second cumulative delivery volume (KV₂) using the variable cumulative delivery volume function.

In particular, the infusion computing device processor determines the second cumulative delivery volume (KV₂) by calculating:

$$( {V}_{d} {e}^{- \frac{{KV}_{2}}{{V}_{d}}} ) + {KV}_{2} - {V}_{d} = ( \frac{2 {V}_{p}}{{2}^{16} - 2} {e}^{\frac{{t}_{s}}{2} ln ( {2}^{30 / i} )} - \frac{2 {V}_{p}}{{2}^{16} - 2} ) \times β$$

where:
KV₂ is indicative of the second cumulative delivery volume; and
tₛ₂ is indicative of the subsequent infusion step time of the target infusion step.

When determining the second cumulative delivery volume KV₂, the infusion computing device processor uses the same volume parameter (β) as was used to determine the first cumulative delivery volume KV₁. That is, the volume parameter (β) is determined in the same way when determining the first cumulative delivery volume KV₁ and the second cumulative delivery volume KV₂.

The second cumulative delivery volume KV₂ provides an indication of the cumulative volume of the fluid that is to be expelled from the medication delivery apparatus 10 between the initial time and the subsequent infusion step time (tₛ).

At 5610, the infusion computing device determines an infusion volume for the target infusion step. In particular, the infusion computing device processor determines the infusion volume. The infusion volume is indicative of a volume of the fluid that is to be expelled from the medication delivery apparatus 10 during the target infusion step. The infusion computing device processor determines the infusion volume based at least in part on the first cumulative delivery volume (KV₁) and the second cumulative delivery volume (KV₂). In some embodiments, the infusion computing device determines the infusion volume in a case where the first cumulative delivery volume (KV₁) and the second cumulative delivery volume (KV₂) were determined using the constant cumulative delivery volume function. In some embodiments, the infusion computing device determines the infusion volume in a case where the first cumulative delivery volume (KV₁) and the second cumulative delivery volume (KV₂) were determined using the Varying Cumulative Volume Function.

In either case, the infusion computing device processor determines the infusion volume by determining a difference between the second cumulative delivery volume (KV₂) and the first cumulative delivery volume (KV₁). That is, the infusion computing device processor subtracts the first cumulative delivery volume (KV₁) from the second cumulative delivery volume (KV₂). The result of this subtraction is the infusion volume.

At 5612, the infusion computing device determines a target flow rate for the target infusion step. In particular, the infusion computing device processor determines the first target flow rate. The target flow rate is a flow rate at which it is aimed to deliver the target infusion step. The infusion computing device processor determines the target flow rate based at least in part on the first infusion volume.

The medication delivery system 1 is configured to deliver the target infusion step over a target infusion step duration. The target infusion step duration is a time period (e.g. 20 s). In some embodiments, determining the target flow rate comprises dividing the infusion volume by the target infusion step duration. That is, the infusion computing device processor may determine the target flow rate by dividing the infusion volume by an infusion step duration of the target infusion step.

It should be noted that in some embodiments, the infusion step durations of each of the number of infusion steps (h) may be the same. Alternatively, the infusion step durations of at least some of the infusion steps may be different. For example, the target infusion step duration may be different to an infusion step duration of another infusion step of the number of infusion steps (h).

In some embodiments, the target infusion step duration is less than an infusion step duration of another infusion step of the number of infusion steps (h). In such cases, the infusion step of the number of infusion steps (h) may be delivered closer to the initial time than the another infusion step of the number of infusion steps (h). Alternatively, the target infusion step may be delivered further away from the initial time than the another infusion step of the number of infusion steps (h).

The infusion computing device processor generates an infusion process based at least in part on the first infusion volume. The infusion computing device processor generates the infusion process based at least in part on the second infusion volume. The infusion process may be referred to as an infusion program. The infusion process defines the rate at which the first plunger is to be actuated during the infusion to deliver the active agent as intended. That is, the infusion process defines the volume of fluid that is to be expelled by the medication delivery apparatus 10 over predetermined units of time. The infusion computing device processor stores the infusion process in the infusion computing device memory. In particular, the infusion computing device processor stores the infusion process in the infusion computing device memory in the form of an infusion process file.

In some embodiments, the clinician saves the infusion process file on a removable storage medium such as a USB memory module. The clinician may transfer the infusion process file to the infusion device. The infusion device may then perform the infusion process using the infusion process file. In some embodiments, the infusion computing device processor transmits the infusion process file to the infusion device. For example, a communications network may be in the form of a network (e.g. the Internet or a Local Area Network). In these cases, the infusion computing device processor may transmit the infusion process file to the infusion device via the communications network.

At 5614, the infusion device performs the infusion process. In particular, at 5614, the infusion device actuates a plunger of the medication delivery apparatus 10. In particular, the infusion device actuates the plunger 21. The infusion device processor sends a control signal to the infusion device actuator to actuate the plunger 21. The infusion device may therefore be said to actuate the infusion device actuator to actuate the plunger 21, in some embodiments.

Where the infusion device actuator is in the form of a syringe driver (or another contact-based actuator), the infusion device may actuate the syringe driver (or the other actuator) to actuate the plunger 21. The infusion device may move the infusion device actuator such that it contacts the plunger 21. The infusion device may continue to move the infusion device actuator to cause movement of the plunger 21 within the container. In particular, the infusion device actuator may be actuated to move the plunger 21 towards the outlet 25. This movement of the plunger 21 can be considered actuation of the plunger 21.

As described herein, in some embodiments, the infusion device may be in the form of a vacuum infusion device. The vacuum infusion device may apply a vacuum pressure to the outlet 25, which can cause motion of the plunger 21. This motion of the plunger 21 is considered actuation of the plunger 21. Therefore, in some embodiments, the infusion device actuates the plunger 21 at least in part by way of applying the vacuum pressure to the outlet 25.

The infusion device actuates the plunger 21 such that the infusion volume of the fluid is expelled from the medication delivery apparatus 10 (i.e. from the dilution chamber 32) during the target infusion step. In other words, the infusion device actuates the plunger 21 to expel the infusion volume of the fluid from the medication delivery apparatus 10 during the target infusion. The infusion device actuates the plunger 21 such that the infusion volume of the fluid is expelled from the medication delivery apparatus 10 during the target infusion step at the target flow rate.

In some embodiments, the concentration of the active agent in the fluid that is to be expelled from the medication delivery apparatus 10 during the target infusion step is a first concentration. The concentration of the active agent in the fluid that is to be expelled from the medication delivery apparatus 10 during another infusion step of the number of infusion steps (h) is a second concentration. In some embodiments, the first concentration is at least one order of magnitude lower than the second concentration. That is, in some embodiments, a concentration of the active agent in the infusion volume of the fluid that is to be expelled from the medication delivery apparatus 10 during the target infusion step is at least one order of magnitude lower than a concentration of the active agent in another infusion volume of the fluid that is to be expelled from the medication delivery apparatus 10. In some embodiments, the first concentration is at least one order of magnitude greater than the second concentration. That is, in some embodiments, a concentration of the active agent in the infusion volume of the fluid that is to be expelled from the medication delivery apparatus 10 during the target infusion step is at least one order of magnitude greater than a concentration of the active agent in another infusion volume of the fluid that is to be expelled from the medication delivery apparatus 10. In some embodiments, the first concentration is equal to the second concentration. In some embodiments, the first concentration is a same order of magnitude as the second concentration. The another infusion volume may be a previous infusion volume (i.e. it may be delivered in a previous infusion step that is delivered prior to the target infusion step—e.g. earlier in the time window). The another infusion volume may be a subsequent infusion volume (i.e. it may be delivered in a subsequent infusion step that is delivered after the target infusion step—e.g. later in the time window).

### Constant Dose Infusion Cumulative Volume Function

In some embodiments, the infusion device processor is configured to perform the infusion as per the method 5600 for a first infusion portion, and is configured to perform the method according to a constant dose process for a second infusion portion. The infusion computing device processor is configured to determine a maximum dose time. The maximum dose time is a time within the time window at which the dose rate of the infusion process reaches a maximum dose threshold. Where the maximum dose time is within the first time window, rather than using the cumulative volume function described above, the infusion device processor is governed by a constant dose cumulative volume function. For the method 5500, the constant dose cumulative volume function may be referred to as a Sadleir Constant Dose Cumulative Volume Function. The Sadleir Constant Dose Cumulative Volume Function may be:

$${V}_{d} {e}^{- \frac{v}{{V}_{d}}} + KV - {V}_{d} = \frac{{V}_{p}}{{i}_{2}} t \times \frac{( {V}_{p} - {V}_{d} \times ( 1 - {e}^{- \frac{{V}_{p}}{{V}_{d}}} ) )}{{V}_{p}}$$

Referring again to FIG. 43, in some embodiments, a plunger lock 134 can be used to ensure the volume of the active agent chamber is fixed. In some embodiments, the Kelly function (e.g. the cumulative delivery volume function of the method 5500) may be performed with the plunger lock 134 locking the first plunger 92. This case may be equivalent to the Sadleir Increased Volume Cumulative Function case described herein, as it is equivalent to the Tansy function with Vₚ specified as above.

### Method 5700 for Delivering a Pharmaceutical Preparation to a Patient

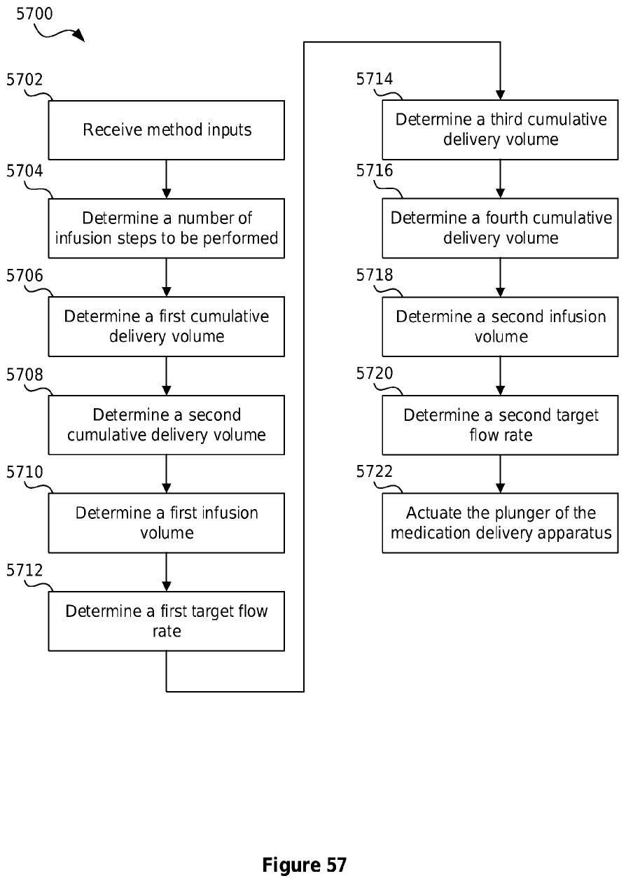

FIG. 57 is a process flow diagram of a method 5700 for delivering a pharmaceutical preparation to a patient, according to some embodiments. The method 5700 may be performed by the medication delivery system 1 and/or the medication delivery apparatus 10 described with reference to FIGS. 1 to 11e. The method 5700 may be performed by the medication delivery system 91 and/or the medication delivery apparatus 90 described with reference to FIG. 30 to 34a or 35 to 43. The method 5700 may be performed by the medication delivery system 91 and/or the medication delivery apparatus 136 described with respect to FIGS. 44 to 47A. The medication delivery system 91 comprises the medication delivery apparatus 136 and the infusion device 93, as is described herein. The pharmaceutical preparation is delivered to the patient by an infusion process that commences at an initial time. Although the method 5700 is described with reference to the medication delivery system 91 and the medication delivery apparatus 136 described with respect to FIGS. 44 to 47A, it will be understood that the description is also applicable to the medication delivery system 1 and/or the medication delivery apparatus 10 described with reference to FIGS. 1 to 11e and the medication delivery system 91 and/or medication delivery apparatus 90 described with reference to FIG. 30 to 34a or 35 to 43.

Some or all of the method 5700 may be performed by the infusion device processor (i.e. the processor of the infusion device 93). Some or all of the method 5700 may be performed by the infusion computing device 151. Some or all of the method 5700 may be performed by another computing device. The method 5700 may therefore be considered a computer-implemented method.

At 5702, the infusion computing device 151 receives one or more method inputs. Step S702 of the method 5700 may the same as, or similar to step S502 of the method 5500 and/or step S602 of the method 5600. In particular, the infusion computing device processor 256 receives the one or more method inputs. In some embodiments, the one or more method inputs comprises a plurality of method inputs. The method inputs may be received via the infusion computing device user interface 260 (e.g. via the display and/or the keyboard). In some embodiments, at least one of the method inputs is an input of a cumulative delivery volume function. In some embodiments, at least one of the method inputs is an input of a dose function. The clinician may input the one or more method inputs.

In some embodiments, the one or more method inputs comprises a concentration input (Cₚ). The concentration input (Cₚ) may be as described herein. In some embodiments, the one or more method inputs comprises a volume input (Vₚ). The volume input (Vₚ) may be as is described herein. In some embodiments, the one or more method inputs comprises a dilution chamber volume input (V_{d}). The dilution chamber volume input (V_{d}) may be as is described herein. In some embodiments, the one or more method inputs comprises a time input (i). The time input (i) may be as is described herein. The time input (i) is indicative of a time window over which the pharmaceutical preparation is to be delivered.

The method 5700 comprises performing a number of infusion steps (h) within the time window. A first number of infusion steps (h₁) are performed within the first time window. A second number of infusion steps (h₂) are performed within the second time window.

At 5704, the infusion computing device 151 determines the number of infusion steps (h) that are to be performed within the time window. Step S704 of the method 5700 may the same as, or similar to step S504 of the method 5500.

The infusion computing device processor 256 determines the number of infusion steps (h) that are to be performed within the time window. The infusion computing device 151 (e.g. the infusion computing device processor 256) determines the first number of infusion steps (h₁) that are to be performed within the first time window. The infusion computing device 151 determines the second number of infusion steps (h₂) that are to be performed within the second time window. The time window, first time window, second time window, number of infusion steps (h), first number of infusion steps (h₁) and/or second number of infusion steps (h₂) may be as described herein.

In some embodiments, determining the number of infusion steps (h) comprises receiving the number of infusion steps (h). The number of infusion steps (h) may be received (e.g. by the infusion computing device processor 256) as an infusion step input. Alternatively, the one or more method inputs may comprise the number of infusion steps (h).

In some embodiments, determining the number of infusion steps (h) comprises calculating a product of a number of infusion steps (per minute) that are to be performed within the time window (g) and the time input (i). That is, to determine the number of infusion steps (h), the infusion computing device processor 256 calculates:

$$h = g \times i$$

where h is the number of infusion steps that are to be performed within the time window, g is the number of infusion steps per minute that are to be performed during the infusion process and (i) is the time input. The time input (i) may therefore indicate a length of the infusion process in minutes. In these embodiments, the infusion computing device processor 256 may store the number of infusion steps (h) in the infusion computing device memory 258. The infusion computing device processor 256 may subsequently issue a read transaction to the infusion computing device memory 258 relating to the number of infusion steps (h), and may receive the number of infusion steps (h) in response. Thus, receiving the number of infusion steps (h) may comprise determining the number of infusion steps (h).

At 5706, the infusion computing device 151 determines a first cumulative delivery volume (KV₁) for an infusion step of the first number of infusion steps (h₁). In particular, the infusion computing device processor 256 determines the first cumulative delivery volume (KV₁) for the infusion step of the first number of infusion steps (h₁). The first cumulative delivery volume (KV₁) is indicative of a cumulative volume of a fluid that is to be expelled from the medication delivery apparatus 136 between an initial time and an initial infusion step time. The fluid that is expelled from the medication delivery apparatus 136 is the fluid that is stored in the dilution chamber 100 at the time of the infusion step of the first number of infusion steps (h₁). That is, the fluid is the diluted pharmaceutical preparation. As described herein, the initial time corresponds to a start of the infusion process. For example, the initial time may be 0. The initial infusion step time corresponds to a start of the infusion step of the first number of infusion steps (h₁). The initial infusion step time may be indexed with respect to the initial time. For example, the initial infusion step time may be the number of milliseconds or seconds that the infusion step of the first number of infusion steps (h₁) starts at after the initial time.

The infusion computing device processor 256 determines the first cumulative delivery volume (KV₁) using a cumulative delivery volume function. Step S706 may otherwise be substantially the same as, or similar to step S506 of the method 5500. The infusion computing device processor 256 may determine the first cumulative delivery volume (KV₁) using the cumulative delivery volume function described with reference to step S506 of the method 5500.

The cumulative delivery volume function used at step S706 can be:

$$K V = {V}_{d} + {V}_{d} {W}_{0} ( - {e}^{- \frac{( {( {2}^{\frac{30}{i}} )}^{\frac{t}{2}} - 1 ) {V}_{p}}{32767 \times {V}_{d}} - 1} ) + \frac{{V}_{p} ( {( {2}^{\frac{30}{i}} )}^{\frac{c}{2}} - 1 )}{32767}$$

where:
KV is indicative of the relevant first cumulative delivery volume;
V_{d} is the dilution chamber volume input;
W₀ is the principle branch of the Lambert W function;
i is the time input;
t is a relevant time that is indicative of a time at which the cumulative delivery volume function is being solved; and
Vₚ is the volume input.

Solving for KV in the cumulative delivery volume function provides an indication of the cumulative volume of a fluid that is to be expelled from the medication delivery apparatus 136 between the initial time and the relevant time (t).

Therefore, the infusion computing device processor 256 determines the first cumulative delivery volume (KV₁) using the above cumulative delivery volume function at tᵢ, where tᵢ is indicative of the initial infusion step time of the infusion step of the first number of infusion steps (h₁). This is as described with reference to step S506.

At 5708, the infusion computing device 151 determines a second cumulative delivery volume (KV₂) for the infusion step of the first number of infusion steps (h₁). In particular, the infusion computing device processor 256 determines the second cumulative delivery volume (KV₂) for the infusion step of the first number of infusion steps (h₁). The second cumulative delivery volume (KV₂) is indicative of a cumulative volume of a fluid that is to be expelled from the medication delivery apparatus 136 between the initial time and a subsequent infusion step time. Step S708 may be substantially the same as, or similar to step S508 of the method 5500.

The cumulative volume of the fluid that is to be expelled from the medication delivery apparatus 136 between the initial time and the subsequent infusion step time may be referred to as a second cumulative volume. The fluid that is expelled from the medication delivery apparatus 136 is the fluid that is stored in the dilution chamber 100 at the time of the infusion step of the first number of infusion steps (h₁). That is, the fluid is the diluted pharmaceutical preparation. As described herein, the initial time corresponds to a start of the infusion process. The subsequent infusion step time corresponds to an end of the infusion step of the first number of infusion steps (h₁). The subsequent infusion step time may be indexed with respect to the initial time. For example, the subsequent infusion step time may be the number of milliseconds or seconds at which the infusion step of the first number of infusion steps (h₁) ends after the initial time.

The infusion computing device processor 256 determines the second cumulative delivery volume (KV₂) using the cumulative delivery volume function as described with reference to step S508 of the method 5500.

At 5710, the infusion computing device 151 determines a first infusion volume for the infusion step of the first number of infusion steps (h₁). In particular, the infusion computing device processor 256 determines the first infusion volume. The first infusion volume is indicative of a volume of the fluid that is to be expelled from the medication delivery apparatus 136 during the infusion step of the first number of infusion steps (h₁). The infusion computing device processor 256 determines the first infusion volume based at least in part on the first cumulative delivery volume (KV₁) and the second cumulative delivery volume (KV₂). Step S710 may be substantially the same as, or similar to step S510 of the method 5500.

In some embodiments, the infusion computing device processor 256 determines the first infusion volume by determining a difference between the second cumulative delivery volume (KV₂) and the first cumulative delivery volume (KV₁). That is, the infusion computing device processor 256 subtracts the first cumulative delivery volume (KV₁) from the second cumulative delivery volume (KV₂). The result of this subtraction is the first infusion volume.

At 5712, the infusion computing device 151 determines a first target flow rate for the infusion step of the first number of infusion steps (h₁). In particular, the infusion computing device processor 256 determines the first target flow rate. The first target flow rate is a target flow rate of the infusion step of the first number of infusion steps (h₁). The infusion computing device processor 256 determines the first target flow rate based at least in part on the first infusion volume of the infusion step of the first number of infusion steps (h₁). Step S712 may be substantially the same as, or similar to step S512 of the method 5500.

The medication delivery system 91 is configured to deliver the infusion step of the first number of infusion steps (h₁) over a first infusion step duration. The first infusion step duration is a time period (e.g. 20s). In some embodiments, determining the first target flow rate comprises dividing the first infusion volume by the first infusion step duration.

It should be noted that in some embodiments, the infusion step durations of each of the infusion steps of the first number of infusion steps (h₁) may be the same. For example, this may be as described with reference to the method 5500.

Alternatively, the infusion step durations of at least some of the infusion steps of the first number of infusion steps (h₁) may be different. For example, this may be as described with reference to the method 5500.

At 5714, the infusion computing device 151 determines a third cumulative delivery volume (KV₃) for an infusion step of the second number of infusion steps (h₂). In particular, the infusion computing device processor 256 determines the third cumulative delivery volume (KV₃) for the infusion step of the second number of infusion steps (h₂). The third cumulative delivery volume (KV₃) is indicative of a cumulative volume of a fluid that is to be expelled from the medication delivery apparatus 136 between an initial time and a second initial infusion step time. The fluid that is expelled from the medication delivery apparatus 136 is the fluid that is stored in the dilution chamber 100 at the time of the infusion step of the second number of infusion steps (h₂). That is, the fluid is the diluted pharmaceutical preparation. As described herein, the initial time corresponds to a start of the infusion process. The second initial infusion step time corresponds to a start of the infusion step of the second number of infusion steps (h₂). The second initial infusion step time may be indexed with respect to the initial time. For example, the second initial infusion step time may be the number of milliseconds or seconds that the infusion step of the second number of infusion steps (h₂) starts at after the initial time.

The infusion computing device processor 256 determines the third cumulative delivery volume (KV₃) using a cumulative delivery volume function. The cumulative delivery volume function may be referred to as a Wood Cumulative Volume Function. The cumulative delivery volume function may be a third cumulative delivery volume function. The cumulative delivery volume function has one or more inputs. The one or more inputs of the cumulative delivery volume function comprise one or more of the second initial infusion step time of the infusion step of the second number of infusion steps (h₂), the time input (i), the dilution chamber volume input (V_{d}) and the volume input (Vₚ). Thus, infusion computing device processor 256 determines the third cumulative delivery volume (KV₃) based at least in part on one or more of the second initial infusion step time of the infusion step of the second number of infusion steps (h₂), the time input (i), the dilution chamber volume input (V_{d}) and the volume input (Vₚ).

The cumulative delivery volume function can be expressed as:

$$\frac{1}{2} ( - 2 {V}_{d} + 2 K V - \frac{1}{2 {V}_{d}} ( - {e}^{\frac{( V p - V d )}{{V}_{d}}} ) {( - {V}_{d} - {V}_{p} + K V )}^{2} ) = \frac{2 {V}_{p}}{{2}^{16} - 2} {e}^{\frac{t}{2} l n ( {2}^{30 / i} )} - \frac{2 {V}_{p}}{{2}^{16} - 2}$$

where:
KV is indicative of the volume of the fluid that has been delivered by the medication delivery apparatus at the relevant time t;
V_{d} is the dilution chamber volume input;
t is indicative of the relevant time; and
Vₚ is the volume input.

Solving for KV in the cumulative delivery volume function provides an indication of the cumulative volume of a fluid that is to be expelled from the medication delivery apparatus 136 between the initial time and the relevant time (t).

Therefore, the infusion computing device processor 256 determines the third cumulative delivery volume (KV₃) by calculating:

$$\frac{1}{2} ( - 2 {V}_{d} + 2 K {V}_{3} - \frac{1}{2 {V}_{d}} ( - {e}^{- \frac{( V p - V d )}{{V}_{d}}} ) {( - {V}_{d} - {V}_{p} + K {V}_{3} )}^{2} ) = \frac{2 {V}_{p}}{{2}^{16} - 2} {e}^{\frac{{t}_{i 2}}{2} l n ( {2}^{30 / i} )} - \frac{2 {V}_{p}}{{2}^{16} - 2}$$

where:
KV₃ is indicative of the third cumulative delivery volume;
V_{d} is the dilution chamber volume input;
tᵢ₂ is indicative of the second initial infusion step time of the infusion step of the second number of infusion steps (h₂); and
Vₚ is the volume input.

The third cumulative delivery volume KV₃ provides an indication of the cumulative volume of the fluid that is to be expelled from the medication delivery apparatus 136 between the initial time and the second initial infusion step time (tᵢ₂).

At 5716, the infusion computing device 151 determines a fourth cumulative delivery volume (KV₄) for the infusion step of the second number of infusion steps (h₂). In particular, the infusion computing device processor 256 determines the fourth cumulative delivery volume (KV₄) for the infusion step of the second number of infusion steps (h₂). The fourth cumulative delivery volume (KV₄) is indicative of a cumulative volume of a fluid that is to be expelled from the medication delivery apparatus 136 between the initial time and a second subsequent infusion step time. The cumulative volume of the fluid that is to be expelled from the medication delivery apparatus 136 between the initial time and the second subsequent infusion step time may be referred to as a fourth cumulative volume. The fluid that is expelled from the medication delivery apparatus 136 is the fluid that is stored in the dilution chamber 100 at the time of the infusion step of the second number of infusion steps (h₂). That is, the fluid is the diluted pharmaceutical preparation. As described herein, the initial time corresponds to a start of the infusion process. The second subsequent infusion step time corresponds to an end of the infusion step of the second number of infusion steps (h₂). The second subsequent infusion step time may be indexed with respect to the initial time. For example, the second subsequent infusion step time may be the number of milliseconds or seconds at which the infusion step of the second number of infusion steps (h₂) ends after the initial time.

The infusion computing device processor 256 determines the fourth cumulative delivery volume (KV₄) using the cumulative delivery volume function.

In particular, the infusion computing device processor 256 determines the fourth cumulative delivery volume (KV₄) by calculating:

$$\frac{1}{2} ( - 2 {V}_{d} + 2 K {V}_{3} - \frac{1}{2 {V}_{d}} ( - {e}^{- \frac{( V p - V d )}{{V}_{d}}} ) {( - {V}_{d} - {V}_{p} + K {V}_{3} )}^{2} ) = \frac{2 {V}_{p}}{{2}^{16} - 2} {e}^{\frac{{t}_{s 2}}{2} l n ( {2}^{30 / i} )} - \frac{2 {V}_{p}}{{2}^{16} - 2}$$

where:
KV₄ is indicative of the fourth cumulative delivery volume;
V_{d} is the dilution chamber volume input;
tₛ₂ is indicative of the second initial infusion step time of the infusion step of the second number of infusion steps (h₂); and
Vₚ is the volume input.

### Determining the Third and Fourth Cumulative Volumes Using the Wood Constant Infusion Function

In some embodiments, the infusion computing device processor 256 determines the third cumulative delivery volume (KV₃) using a Wood Constant Infusion Function. The Wood Constant Infusion Function may be expressed as:

$$\frac{1}{2} ( - 2 {V}_{d} + 2 K V - \frac{1}{2 {V}_{d}} ( - {e}^{- \frac{( V p - V d )}{{V}_{d}}} ) {( - {V}_{d} - {V}_{p} + K V )}^{2} ) = \frac{{V}_{p}}{i} t$$

### Where:

KV is indicative of the volume of the fluid that has been delivered by the medication delivery apparatus at the relevant time t;
V_{d} is the dilution chamber volume input;
i is the time input;
t is indicative of the relevant time; and
Vₚ is the volume input.

In these embodiments, the infusion computing device processor 256 determines the third cumulative volume (KV₃) by calculating:

$$\frac{1}{2} ( - 2 {V}_{d} + 2 K {V}_{3} - \frac{1}{2 {V}_{d}} ( - {e}^{- \frac{( V p - V d )}{{V}_{d}}} ) {( - {V}_{d} - {V}_{p} + K {V}_{3} )}^{2} ) = \frac{{V}_{p}}{i} {t}_{i 2}$$

The third cumulative delivery volume KV₃ provides an indication of the cumulative volume of the fluid that is to be expelled from the medication delivery apparatus 136 between the initial time and the second initial infusion step time (tᵢ₂). This is in accordance with a constant infusion regime driven by the Wood Function.

In some embodiments, the infusion computing device processor 256 determines the fourth cumulative delivery volume (KV₄) using the Wood Constant Infusion Function. In these embodiments, the infusion computing device processor 256 determines the fourth cumulative volume (KV₄) by calculating:

$$\frac{1}{2} ( - 2 {V}_{d} + 2 K {V}_{4} - \frac{1}{2 {V}_{d}} ( - {e}^{- \frac{( V p - V d )}{{V}_{d}}} ) {( - {V}_{d} - {V}_{p} + K {V}_{4} )}^{2} ) = \frac{{V}_{p}}{i} {t}_{s 2}$$

The fourth cumulative delivery volume KV₄ provides an indication of the cumulative volume of the fluid that is to be expelled from the medication delivery apparatus 136 between the initial time and the second subsequent infusion step time (tₛ₂).

At 5718, the infusion computing device 151 determines a second infusion volume for the infusion step of the second number of infusion steps (h₂). In particular, the infusion computing device processor 256 determines the second infusion volume. The second infusion volume is indicative of a volume of the fluid that is to be expelled from the medication delivery apparatus 136 during the infusion step of the second number of infusion steps (h₂). The infusion computing device processor 256 determines the second infusion volume based at least in part on the third cumulative delivery volume (KV₃) and the fourth cumulative delivery volume (KV₄). Step S718 may be substantially the same as, or similar to step S510 of the method 5500.

In some embodiments, the infusion computing device processor 256 determines the second infusion volume by determining a difference between the third cumulative delivery volume (KV₃) and the fourth cumulative delivery volume (KV₄). That is, the infusion computing device processor 256 subtracts the third cumulative delivery volume (KV₃) from the fourth cumulative delivery volume (KV₄). The result of this subtraction is the second infusion volume.

At 5720, the infusion computing device 151 determines a second target flow rate for the infusion step of the second number of infusion steps (h₂). In particular, the infusion computing device processor 256 determines the second target flow rate. The second target flow rate is a target flow rate of the infusion step of the second number of infusion steps (h₂). The infusion computing device processor 256 determines the second target flow rate based at least in part on the second infusion volume of the infusion step of the second number of infusion steps (h₂). Step S720 may be substantially the same as, or similar to step S512 of the method 5500.

The medication delivery system 91 is configured to deliver the infusion step of the second number of infusion steps (h₂) over a second infusion step duration. The second infusion step duration is a time period (e.g. 20s). In some embodiments, determining the second target flow rate comprises dividing the second infusion volume by the second infusion step duration.

It should be noted that in some embodiments, the infusion step durations of each of the infusion steps of the second number of infusion steps (h₂) may be the same. For example, this may be as described with reference to the method 5500. Alternatively, the infusion step durations of at least some of the infusion steps of the second number of infusion steps (h₂) may be different. For example, this may be as described with reference to the method 5500.

The infusion computing device processor 256 generates an infusion process based at least in part on the first infusion volume. The infusion computing device processor 256 generates the infusion process based at least in part on the second infusion volume. The infusion process defines the rate at which the first plunger is to be actuated during the infusion to deliver the active agent as intended. That is, the infusion process defines the volume of fluid that is to be expelled by the medication delivery apparatus 90 over predetermined units of time. The infusion computing device processor 256 stores the infusion process in the infusion computing device memory 258. In particular, the infusion computing device processor 256 stores the infusion process in the infusion computing device memory 258 in the form of an infusion process file.

In some embodiments, the clinician saves the infusion process file on a removable storage medium such as a USB memory module. The clinician may transfer the infusion process file to the infusion device 93. The infusion device 93 may then perform the infusion process using the infusion process file. In some embodiments, the infusion computing device processor 256 transmits the infusion process file to the infusion device 93. For example, a communications network may be in the form of a network (e.g. the Internet or a Local Area Network). In these cases, the infusion computing device processor 256 may transmit the infusion process file to the infusion device 93 via the communications network 264.

At 5722, the infusion device 93 actuates a plunger of the medication delivery apparatus 136. In particular, the infusion device 93 actuates the first plunger 92. The infusion device processor sends a control signal to the infusion device actuator to actuate the first plunger 92. The infusion device 93 may therefore be said to actuate the infusion device actuator to actuate the first plunger 92, in some embodiments. Step S722 may be the same as, or substantially similar to step S526 of the method 5500.

The infusion device 93 actuates the first plunger 92 such that the first infusion volume of the fluid is expelled from the medication delivery apparatus 136 during the infusion step of the first number of infusion steps (h₁). In other words, the infusion device 93 actuates the first plunger 92 to expel the first infusion volume of the fluid from the medication delivery apparatus 136 during the infusion step of the first number of infusion steps (h₁). The infusion device 93 actuates the first plunger 92 such that the first infusion volume of the fluid is expelled from the medication delivery apparatus 136 during the infusion step of the first number of infusion steps (h₁) at the first target flow rate.

The infusion device 93 actuates the first plunger 92 such that the second infusion volume of the fluid is expelled from the medication delivery apparatus 136 during the infusion step of the second number of infusion steps (h₂). In other words, the infusion device 93 actuates the first plunger 92 to expel the second infusion volume of the fluid from the medication delivery apparatus 136 during the infusion step of the second number of infusion steps (h₂). The infusion device 93 actuates the first plunger 92 such that the second infusion volume of the fluid is expelled from the medication delivery apparatus 136 during the infusion step of the second number of infusion steps (h₂) at the second target flow rate.

In some embodiments, the concentration of the active agent in the fluid that is to be expelled from the medication delivery apparatus 136 during the infusion step of the first number of infusion steps (h₁) is a first concentration. The concentration of the active agent in the fluid that is to be expelled from the medication delivery apparatus 136 during the infusion step of the second number of infusion steps (h₂) is a second concentration. In some embodiments, the first concentration is at least one order of magnitude lower than the second concentration. That is, in some embodiments, a concentration of the active agent in the infusion volume of the fluid that is to be expelled from the medication delivery apparatus 136 during the infusion step of the first number of infusion steps (h₁) is at least one order of magnitude lower than a concentration of the active agent in second infusion volume of the fluid that is to be expelled from the medication delivery apparatus 136 during the infusion step of the second number of infusion steps (h₂). In some embodiments, the first concentration is at least one order of magnitude greater than the second concentration. In some embodiments, the first concentration is at least one order of magnitude less than the second concentration. In some embodiments, the first concentration is equal to the second concentration.

In some embodiments, the infusion device processor is configured to perform the infusion as per the method 5700 for a first infusion portion, and is configured to perform the method according to a constant dose process for a second infusion portion. The infusion computing device processor 256 is configured to determine a maximum dose time. The maximum dose time is a time within the time window at which the dose rate of the infusion process reaches a maximum dose threshold. Where the maximum dose time is within the first time window, rather than using the cumulative volume function described above, the infusion device processor is governed by a constant dose cumulative volume function. For the method 5700, the constant dose cumulative volume function may be referred to as a Wood Constant Dose Cumulative Volume Function. The Wood Constant Dose Cumulative Volume Function may be:

$$\frac{1}{2} ( - 2 {V}_{d} + 2 v - \frac{1}{2 {V}_{d}} ( - {e}^{- \frac{( Vp - V d}{{V}_{d}}} ) {( - {V}_{d} - {V}_{p} + v )}^{2} ) = \frac{{V}_{p}}{i} t$$

### Derivation of the Constant Cumulative Volume Function

The sequential emptying embodiments of the present disclosure (methods 5500, 5600, 5700) may also be used to deliver a constant-dose infusion. This particularly useful when a maximum dosing rate is reached for a particular active agent when using the Kelly function (e.g. the cumulative delivery volume function of the method 5500), as the method can switch to using this function to deliver the remainder of the infusion at the maximum allowable dosing rate for the second time window. In these cases, during the subsequent phase when the active agent chamber 98 is empty and the dilution chamber 100 is emptying, the rate remains constant as the dilution chamber concentration does not change. The equation compensates for changes in the dilution chamber 100 drug concentration to give a constant dose per unit time (infusion rate will decrease over time). The relationship between cumulative volume (v) and time (t) is, for the Kelly function (i.e. the cumulative delivery function of the method 5500, which may be referred to as the Kelly Constant Dose Cumulative Volume Function):

$${V}_{d} {e}^{- \frac{v}{{V}_{d}}} + v - {V}_{d} = \frac{{V}_{p}}{i} t$$

Solved for cumulative volume:

$$v = \frac{1}{i} ( {V}_{d} i + {v}_{d} {iW}_{0} ( - {e}^{- ( {V}_{p} t / {V}_{d} i ) - 1} ) + {V}_{p} t )$$

Where W₀ is the principle branch (branch 0) of the Lambert W function, defined as the inverse function of we^{w}.

The derivative of volume with respect to time, or the infusion rate is:

$$infusion rate = \frac{{V}_{p}}{i + i {W}_{0} ( - {e}^{- ( {V}_{p} t / {( {V}_{d} i )}^{- 1}} )}$$

In some embodiments of the disclosure, the above cumulative volume function and/or the above infusion rate function are used to determine the second infusion volume and/or the second target flow rate of a method disclosed herein.

### Derivation of the Sadleir Cumulative Volume Function

The Sadleir Cumulative Volume Function (one of the cumulative volume functions disclosed herein) is used for a medication delivery apparatus 10, 90 that may be disposable. It is used in cases where the dilution chamber 32, 100 is not automatically emptied, and it is desired that the emptying of the active agent chamber occurs over a length of the infusion time. The exact solution is the same as the described Kelly function (i.e. the cumulative delivery volume function of the method 5500), with the modification that the entered value for 8 in the equation:

$$( {V}_{d} {e}^{- \frac{v}{{V}_{d}}} ) + v - {V}_{d} = ( \frac{2 {V}_{p}}{{2}^{16} - 2} {e}^{\frac{t}{2} l n ( {2}^{30 / t} )} - \frac{2 {V}_{p}}{{2}^{16} - 2} ) \times β$$

is modified to be equal to:

$$β = \frac{( {V}_{p} - {V}_{d} \times ( 1 - {e}^{- \frac{{V}_{p}}{{V}_{d}}} ) )}{{V}_{p}}$$

Rather than B=1, which is equivalent to the Kelly function.
So the Sadleir Cumulative Volume Function equals:

$$( {V}_{d} {e}^{- \frac{v}{{V}_{d}}} ) + v - {V}_{d} = ( \frac{2 {V}_{p}}{{2}^{16} - 2} {e}^{\frac{t}{2} l n ( {2}^{30 / i} )} - \frac{2 {V}_{p}}{{2}^{16} - 2} ) \times ( {V}_{p} - {V}_{d} \times ( 1 - {e}^{- \frac{{V}_{p}}{{V}_{d}}} ) )$$

Where v is the cumulative volume.

### Derivation of the Sadleir Increased Volume Cumulative Function

The Sadleir Increase Volume Cumulative Function (one of the cumulative volume functions disclosed herein) is used for a disposable medication delivery apparatus 10, 90 where the dilution chamber is discarded at the end of the infusion. It is desired that the emptying of the drug chamber occurs over the length of the set infusion time and results in the entire dose of the Tansy function being given.

The exact solution is the same as for the Sadleir Cumulative Volume Function (i.e. the cumulative delivery volume function of the method 5600) with the modification that the entered value for Vₚ in the equation is modified to be equal to:

$${V}_{p 2} = {V}_{d} + {V}_{d} {W}_{0} ( - {e}^{- \frac{( ( {2}^{15} ) - 1 ) {V}_{p}}{32767 \times {V}_{d}} - 1} ) + \frac{{V}_{p} ( ( {2}^{15} ) - 1 )}{32767}$$

So the Sadleir Increased Volume Cumulative Function equals:

$$( {V}_{d} {e}^{- \frac{v}{{V}_{d}}} ) + v - {V}_{d} = ( \frac{2 {V}_{p}}{{2}^{16} - 2} {e}^{\frac{t}{2} l n ( {2}^{\frac{30}{i}} )} - \frac{2 {V}_{p}}{{2}^{16} - 2} ) \times ( {V}_{p} - {V}_{d} \times ( 1 - {e}^{- \frac{{V}_{p}}{{V}_{d}}} ) )$$

Where v is the cumulative volume.

The Sadleir Increased Rate Function (one of the rate functions described herein) is equal to the derivative of the cumulative volume function with respect to time, which is

$$Rate = \frac{{V}_{p} ln ( {2}^{\frac{30}{i}} ) {( {2}^{\frac{30}{i}} )}^{\frac{t}{2}}}{65534 ( {W}_{0} ( - {e}^{- \frac{( {( {2}^{\frac{30}{i}} )}^{\frac{t}{2}} - 1 ) ( {V}_{p} + {V}_{d} ( 1 - {e}^{- \frac{{V}_{p}}{{V}_{d}}} ) )}{32767 {V}_{d}} - 1} ) + 1 )}$$

The Sadleir Constant Dose Cumulative Volume Function is:

$${V}_{d} {e}^{- \frac{v}{{V}_{d}}} + v - {V}_{d} = \frac{{V}_{p}}{{i}_{2}} t \times \frac{( {V}_{p} - {V}_{d} \times ( 1 - {e}^{- \frac{{V}_{p}}{{V}_{d}}} ) )}{{V}_{p}}$$

Method of Delivery where a Maximum Delivery Rate is Exceeded or a Maximum Tolerable Dose is Exceeded

The maximum rate of delivery of drug may be exceeded during the infusion process as a consequence of user settings. In order to ensure this does not happen, the medication delivery system can check that each infusion step does not exceed the maximum allowable dosing rate by estimating the dilution chamber drug concentration and the fluid infusion rate. The dilution chamber drug concentration as a function of cumulative drug volume infused (V) is given by the following equation:

$${C}_{d} = {C}_{p} * ( 1 - {e}^{- \frac{v}{{V}_{d}}} )$$

C_{d} is the concentration of drug in the dilution chamber
Cₚ is the original concentration of drug in the drug delivery flask or syringe or container
V_{d} is the volume of the dilution chamber
V is cumulative volume infused into the dilution chamber or patient

The integral of dilution chamber concentration with respect to volume infused (area under the curve) is equal to the dose delivered.

$${D}_{t} = {\int }_{0}^{V} {C}_{p} * ( 1 - {e}^{- \frac{v}{{V}_{d}}} ) dV$$

$${D}_{t} = ( {C}_{p} * ( {V}_{d} * {e}^{- \frac{v}{{V}_{d}}} + V - {V}_{d} )$$

Dₜ is the cumulative dose delivered from commencement to cumulative volume, V
C_{d} is the concentration of drug in the dilution chamber
Cₚ is the original concentration of drug in the drug delivery flask or syringe or container
V_{d} is the volume of the dilution chamber
V is cumulative volume infused into the dilution chamber or patient

### Cisplatin Dosing

For example, current dosing for a man is 40 mg/m2 over 1 hour in 1000 mL of diluent. This protocol (i.e. the medication delivery system) will deliver 72 mg of Cisplatin in 1000 mL over 60 minutes, which is a fluid injection rate of 16.7 ml/min, and a dose rate of 1.2 mg/min.

If this is delivered using the previously disclosed medication delivery apparatus 90, one can prepare the 72 mg of Cisplatin in 1000 mL of diluent in a flask, connected to the medication delivery apparatus 90 by a peristaltic fluid pump. The dilution chamber 100 can be set at 50 mL volume. The Sadleir Cumulative Volume Function and the Sadleir Constant Dose Function for the periods before and after the infusion duration are limited by the maximum dosing rate.

The duration of infusion can be set to 30 minutes. Using this arrangement, the dose rate increases exponentially over the duration of the infusion until the maximum dose rate is achieved. So:

$$Sadleir Dose rate = {C}_{p} \times {T}_{m} ( t )$$

$$where {T}_{m} ( t ) is the modified Tansy Rate Function$$

$$MaxDoseRate = \frac{Dose}{{V}_{p}} \times \frac{{V}_{p} ln {2}^{\frac{30}{i}}}{{2}^{16} - 2} {e}^{\frac{t}{2} l n ( {2}^{\frac{30}{i}} )} \times \frac{{V}_{p} - {V}_{d} ( 1 - {e}^{- \frac{{V}_{p}}{{V}_{d}}} )}{{V}_{p}}$$

$$t = \frac{2}{ln ( {2}^{\frac{30}{i}} )} ln ( \frac{MaxDoseRate}{{C}_{p}} \times \frac{{2}^{16} - 2}{{V}_{p} ln ( {2}^{\frac{30}{i}} )} \times \frac{{V}_{p}}{{V}_{p} - {V}_{d} ( 1 - {e}^{- \frac{{V}_{p}}{{V}_{d}}} )} )$$

$$t = \frac{2}{ln ( {2}^{\frac{30}{30}} )} ln ( \frac{1.2}{0.072} \times \frac{{2}^{16} - 2}{1000 \times ln ( {2}^{\frac{30}{30}} )} \times \frac{1000}{1000 - 50 ( 1 - {e}^{- \frac{1000}{50}} )} )$$

$$t = 21.39 minutes$$

The maximal allowable dose rate (1.2 mg/min) is reached at 21 minutes and 23 seconds.

The cumulative dose given to this point is given by the modified Tansy Dose Function:

$${ose ( t )}_{m} = {C}_{p} ( \frac{2 {V}_{p}}{{2}^{16} - 2} {e}^{\frac{t}{2} l n ( {2}^{\frac{30}{i}} )} - \frac{2 {V}_{p}}{{2}^{16} - 2} ) \times \frac{( {V}_{p} - {V}_{d} \times ( 1 - {e}^{- \frac{{V}_{p}}{{V}_{d}}} ) )}{{V}_{p}}$$

$${Dose ( t )}_{m} = 0.0 \; 72 ( \frac{2 \times 1000}{{2}^{16} - 2} {e}^{\frac{21.39}{2} l n ( {2}^{\frac{30}{30}} )} - \frac{2 \times 1000}{{2}^{16} - 2} ) \times \frac{( 1000 - 50 \times ( 1 - {e}^{- \frac{1000}{50}} ) )}{1000}$$

$${Dose ( t )}_{m} = 3.46 mg$$

The cumulative volume v for the Sadleir cumulative volume function at this point is calculated by:

$$( {V}_{d} {e}^{- \frac{v}{{V}_{d}}} ) + v - {V}_{d} = ( \frac{2 {V}_{p}}{{2}^{16} - 2} {e}^{\frac{t}{2} l n ( {2}^{\frac{30}{i}} )} - \frac{2 {V}_{p}}{{2}^{16} - 2} ) \times \frac{( {V}_{p} - {V}_{d} \times ( 1 - {e}^{- \frac{{V}_{p}}{{V}_{d}}} ) )}{{V}_{p}}$$

### Or:

$$( {V}_{d} {e}^{- \frac{v}{{V}_{d}}} ) + v - {V}_{d} = \frac{{Dose ( t )}_{m}}{{C}_{p}}$$

Which can be solved for v:

$$v = {V}_{d} + \frac{{Dose ( t )}_{m}}{{C}_{p}} + {V}_{d} {W}_{0} ( - {e}^{- \frac{{Dose ( t )}_{m}}{{V}_{d} {C}_{p}} - 1} )$$

$$v = 89.75 ml$$

When the cumulative volume administered has been 89.75 mL, the dilution chamber concentration is given by:

$${C}_{d c} = {C}_{p} ( 1 - {e}^{- \frac{v}{Vd}} )$$

$${C}_{d c} = 0.0 \; 72 ( 1 - {e}^{\frac{- 89.75}{50}} )$$

$${C}_{d c} = 0.0 \; 60 mg / ml$$

The subsequent infusion period is controlled by the Sadleir Constant Dose function. This gives a constant dose-rate (dose per unit time) for all subsequent periods of the infusion. This algorithm will deliver the remaining 68.54 mg at a rate of 1.2 mg/min and therefore over a period of 57.1 minutes. The equation for the Sadleir Constant Dose Cumulative function is:

$${V}_{d} {e}^{- \frac{v}{{V}_{d}}} + v - {V}_{d} = \frac{{V}_{p}}{{i}_{2}} t \times \frac{( {V}_{p} - {V}_{d} \times ( 1 - {e}^{- \frac{{V}_{p}}{{V}_{d}}} ) )}{{V}_{p}}$$

### Where:

v is the cumulative volume; and
i₂ is a duration of a Sadleir Constant Dose Function that would administer the total Tansy Modified Dose

$$( i . e . 72 mg \times \frac{( {V}_{p} - {V}_{d} \times ( 1 - {e}^{- \frac{{V}_{p}}{{V}_{d}}} ) )}{{V}_{p}} = 68.4 mg )$$

of cisplatin at a rate of 1.2 mg/min (i.e. 68.4/1.2=57 minutes).
   The starting point of the Sadleir Constant Dose Function will be the time when 89.75 mL has been delivered.

$$50 {e}^{- \frac{89.75}{50}} + 89.75 - 50 = \frac{1000}{57} t \times \frac{( 1000 - 50 \times ( 1 - {e}^{- \frac{1000}{50}} ) )}{1000}$$

$$t = 2.88 min$$

The infusion will proceed for (75-2.88)=54.12 minutes until the 1000 mL flask is empty. At this point, 95% of the 72 mg dose will have been administered over a total of (21.39+54.12)=75.51 minutes. The remaining 50 ml of pharmaceutical preparation remaining in the dilution chamber can be administered manually.

The duration of infusion can be set to 180 minutes using 360 constant infusion steps of 30 seconds. Using this arrangement, the dose rate increases exponentially over the duration of the infusion. The minimum infusion flow rate will be 0.1 mL/min (6 ml/hr). The maximum allowable infusion dose rate (1.2 mg/min) is exceeded at 158 minutes and 29 seconds, therefore the infusion will be limited to the infusion rate for the subsequent interval (starting at 158 minutes and 30 seconds). The cumulative volume delivered is 338.5 mL and the infusion rate 16.7 ml/min. The dilution chamber concentration is estimated at 0.0719 mg/mL and so the allowable infusion rate is 16.7 mL/min for all subsequent intervals. The remaining 661.5 mL infusion will complete in a further 40 minutes (total infusion duration approximately 198 minutes). After 1000 mL of the drug infusion has been infused, the dilution chamber can be collapsed to deliver the final 50 mL of solution, or an additional 50 ml of drug infusion can be delivered from the drug flask via the dilution chamber.

### Rocuronium Dosing

Rocuronium is a non-depolarising neuromuscular blocking agent and is chosen as an example of a drug that can only have a part of its therapeutic dose administered slowly (the rest having to be administered either quickly or slowly when anaesthetized). It is administered intravenously in a dose of 0.6 mg/kg (50 mg for an 80 kg patient). This is usually administered as a push after anaesthesia is induced.

Rocuronium may be administered to an awake patient up to a dose of approximately 0.03 mg/kg (2.4 mg in an 80 kg patient). This will cause minor, tolerable side-effects (blurred vision).

Test doses or desensitisation may be administered by diluting 50 mg of rocuronium in an infusion volume Vₚ of 50 mL, with a 10 mL dilution chamber, infused over 30 minutes, but pausing the infusion for induction of anaesthesia once 0.03 mg/kg has been administered. Then the remainder of the infusion can either be given as a push (if relaxation is required immediately at induction) or by continuing the remainder of the infusion.

Using the medication delivery system 91 (the Diocles device, method 5500) with this protocol, 2.4 mg is administered after 21 minutes and 14 seconds. The infusion rate at this point is 1.43 ml/min and 7.83 ml of solution has been infused.

### Method of Calculating Infusion Rate and Cumulative Volume Delivered Using the Medication Delivery System 91

As described, the dilution chamber drug concentration as a function of cumulative drug volume infused (V) is given by the following equation:

$${C}_{d} = {C}_{p} * ( 1 - {e}^{- \frac{v}{{V}_{d}}} )$$

C_{d} is the concentration of drug in the dilution chamber
Cₚ is the original concentration of drug in the drug delivery flask or syringe or container
V_{d} is the volume of the dilution chamber
V is cumulative volume infused into the dilution chamber or patient

The integral of dilution chamber concentration with respect to volume infused (area under the curve) is equal to the dose delivered:

$${D}_{t} = {\int }_{0}^{V} {C}_{p} ⋆ ( 1 - {e}^{- \frac{v}{{v}_{d}}} ) dV$$

$${D}_{t} = {C}_{p} ⋆ ( {V}_{d} ⋆ {e}^{- \frac{v}{{v}_{d}}} + V - {V}_{d} )$$

Dₜ is the cumulative dose delivered from commencement to cumulative volume, V
C_{d} is the concentration of drug in the dilution chamber
Cₚ is the original concentration of drug in the drug delivery flask or syringe or container
V_{d} is the volume of the dilution chamber
V is cumulative volume infused into the dilution chamber or patient

The target dose administered is that produced by the Tansy function:

$${Dose}_{cumulative} = {C}_{p} ⋆ {\int }_{0}^{t} {T}_{t} dt = {C}_{p} ⋆ ( \frac{2 {V}_{p}}{{2}^{16} - 2} {e}^{( \frac{1}{2} ) ln {2}^{( \frac{30}{i} )}} - \frac{2 {V}_{p}}{{2}^{16} - 2} )$$

Dose_{cumulative} is the cumulative dose administered at time t (minutes)
Tₜ is the Tansy rate function
Cₚ is the concentration of drug in the drug chamber syringe or flask or bag
Vₚ is the volume of the drug solution in the drug chamber syringe or flask or bag
i is the chosen duration of the total infusion in minutes

And the cumulative volume, V, can now be expressed in terms of elapsed infusion time, t:

$${D}_{t} = {C}_{p} * ( {V}_{d} * {e}^{- \frac{v}{{v}_{d}}} + V - {V}_{d} ) = {C}_{p} * ( \frac{2 {V}_{p}}{{2}^{16} - 2} {e}^{( \frac{1}{2} ) ln {2}^{( \frac{30}{i} )}} - \frac{2 {V}_{p}}{{2}^{16} - 2} )$$

which can be simplified to:

$$( {V}_{d} * {e}^{- \frac{v}{{v}_{d}}} + V - {V}_{d} ) = ( \frac{2 {V}_{p}}{{2}^{16} - 2} {e}^{( \frac{1}{2} ) ln {2}^{( \frac{30}{i} )}} - \frac{2 {V}_{p}}{{2}^{16} - 2} )$$

and expresses cumulative volume in terms of time, t
Dₜ is the cumulative dose administered at time t (minutes)
Cₚ is the concentration of drug in the drug chamber syringe or flask or bag
Vₚ is the volume of the drug solution in the drug chamber syringe or flask or bag
V_{d} is the volume of the drug dilution chamber
i is the chosen duration of the total infusion in minutes
V is cumulative volume, V, infused into the dilution chamber or patient
t is elapsed time in minutes

### Advantageous Uses of the Disclosed Methods

The methods disclosed herein (e.g. the methods 5500, 5600, 5700) provide exact solutions to the cumulative volume calculations used to control the medication delivery apparatuses disclosed herein, and therefore, the infusion. The methods 5500, 5600, 5700 can be altered to ensure that a maximum dose rate for a particular active agent is not exceeded. For example, the infusion computing device 151 may determine that a dose rate at which the active agent is to be delivered has reached, or has exceeded a dose rate threshold. The infusion computing device 151 may determine the dose rate using the dose functions disclosed herein. The method inputs may comprise the active agent. The method inputs may comprise the dose rate threshold. That is, the clinician may pre-set the dose rate threshold. The dose rate threshold may be stored in the memory of the infusion computing device 151. In some embodiments, the infusion computing device 151 may retrieve the dose rate threshold from a database.

### Example Vancomycin Infusion

Vancomycin can be used as an example of limiting the dose rate to a maximum. Vancomycin is an antibiotic which has a maximum administration rate of 10 mL/min. Given by a constant infusion, the duration of infusion would normally be 100 minutes. If 1000 mg is constituted in 50 mL with a 10 mL dilution chamber, and the double plunger syringe program can be set to deliver the infusion over 30 minutes.

The clinician can provide an input to the infusion computing device 151 that is indicative of the active agent. For example, the clinician can provide an input indicating that the active agent is vancomycin.

In some embodiments, the clinician looks up a maximum infusion rate for the active agent. This may be 10 mg/min for vancomycin. In some embodiments, the infusion computing device 151 retrieves the maximum infusion rate based on the input indicating the active agent. For example, the infusion computing device 151 may retrieve the maximum infusion rate from a database.

The clinician enters the desired infusion duration (i.e. provides the time input (i)). The time input (i) may be 30 minutes for vancomycin.

The clinician enters the volume input (Vₚ) and the dilution chamber volume input (V_{d}). For a vancomycin infusion, the volume input (Vₚ) may be 50 mL and the dilution chamber volume input (V_{d}) may be 10 mL.

The infusion computing device 151 determines a maximum dose time. The maximum dose time is indicative of a time at which the maximum infusion rate is reached during the infusion process. This time may be a dose temporal point. The infusion computing device 151 determines the maximum dose time using a dose function. The dose function may be as described herein. So the infusion computing device 151 determines when the 10 mg/min maximum infusion rate is equal to the dose function:

$$Dose rate = {C}_{p} \times T ( t )$$

$$MaxDoseRate = \frac{D o s e}{{V}_{p}} \times \frac{50 ln {2}^{30 / i}}{{2}^{16} - 2} {e}^{\frac{t}{2} ln ( {2}^{30 / i} )}$$

$$10 mg / min = \frac{1000}{50} \times \frac{50 ln 2}{{2}^{16} - 2} {e}^{\frac{t}{2} ln 2}$$

$$10 = 20 \times \frac{50 ln 2}{{2}^{16} - 2} {e}^{\frac{t}{2} ln 2}$$

$$0.5 \times \frac{{2}^{16} - 2}{50 ln 2} = {e}^{\frac{t}{2} ln 2}$$

$$ln ( 0.5 \times \frac{{2}^{16} - 2}{50 ln 2} ) = \frac{t}{2} ln 2$$

$$t = \frac{2}{ln 2} ln ( 0.5 \times \frac{{2}^{16} - 2}{50 ln 2} )$$

$$t = 19.77 minutes$$

Note, the general form of this equation is:

$$t = \frac{2}{ln ( {2}^{30 / i} )} ln ( \frac{MaxDos e R a t e}{{C}_{p}} \times \frac{{2}^{16} - 2}{{V}_{p} ln ( {2}^{30 / i} )} )$$

The infusion computing device 151 determines a relative position of the maximum dose time. That is, the infusion computing device 151 checks whether this is in the first time window or the second time window. If the maximum dose time is in the first time window, the infusion computing device 151 changes from using a first cumulative volume function to a second cumulative volume function. The first cumulative volume function may be the Kelly function described herein. The second cumulative volume function may be the constant cumulative volume function described herein. If the maximum dose time is within the second time window, the infusion is continued in accordance with the infusion program already being performed, as the concentration of the active agent in the dilution chamber is constant. In order to do this, the infusion computing device 151 determines the transitional time. The transitional time may be determined as described herein. In this case:

$$Transition time = \frac{ln ( {V}_{p} - {V}_{d} ( 1 - {e}^{- \frac{{V}_{p}}{{V}_{d}}} ) + \frac{2 {V}_{p}}{{2}^{16} - 2} ) - ln ( \frac{2 {V}_{p}}{{2}^{16} - 2} )}{\frac{1}{2} ln ( {2}^{30 / i} )} \\ Transition time = \frac{ln ( 50 - 10 ( 1 - {e}^{- \frac{50}{10}} ) + \frac{2 \times 50}{{2}^{16} - 2} ) - ln ( \frac{2 \times 50}{{2}^{16} - 2} )}{\frac{1}{2} ln ( 2 )} \\ Transition time = 29.36 minutes$$

Therefore, the maximum dose time occurs during the first time window.

The infusion computing device 151 may then use the Kelly Cumulative Volume Function to calculate the cumulative volume administered at the maximum dose time:

$$v = {V}_{d} + {V}_{d} {W}_{0} ( - {e}^{- \frac{( ( {( {2}^{30 / i} )}^{t / 2} ) - 1 ) {V}_{p}}{32767 {V}_{d}} - 1} ) + \frac{{V}_{p} ( {( {2}^{30 / i} )}^{t / 2} ) - 1}{32767} \\ v = 10 + 10 \times {W}_{0} ( - {e}^{- \frac{( ( {( 2 )}^{19.77 / 2} ) - 1 ) \times 50}{32767 \times 10} - 1} ) + \frac{50 \times ( {( 2 )}^{19.77 / 2} ) - 1}{32767} \\ V = 5.9 mL$$

The infusion computing device 151 uses the Constant Cumulative Volume Function to determine when the same volume would be delivered if the infusion device were using the Constant Cumulative Volume Function. Note that the Constant Cumulative Volume Function will deliver the maximum dose rate allowed, and t=100 minutes (the total dose divided by the maximum dose rate).

$${V}_{d} {e}^{- \frac{v}{{V}_{d}}} + v - {V}_{d} = \frac{{V}_{p}}{i} t \\ t = \frac{i}{{V}_{p}} ( {V}_{d} {e}^{- \frac{v}{{V}_{d}}} + v - {V}_{d} ) \\ t = \frac{30}{50} * ( 10 {e}^{- \frac{11.7}{10}} + 11.7 - 10 ) \\ t = 2.8 \; 82 min$$

The infusion computing device 151 will therefore use the Kelly Infusion Volume Function to determine the infusion process until the time reaches the maximum dose time. After the time passes the maximum dose time, the infusion computing device 151 will use the Constant Cumulative Volume Function to determine the infusion process (i.e. the infusion device will actuate the first plunger in accordance with the Constant Cumulative Volume Function until the active agent chamber is empty and the concentration of the active agent in the dilution chamber is constant).

So in this case, the Constant Cumulative Volume Function will be used after 19.77 minutes, using the following settings:

$${V}_{p} = 50$$

$${V}_{d} = 10$$

$$t = 2.88 minutes$$

$$i = 100 minutes$$

The infusion computing device 151 will use the Constant Cumulative Volume Function until the dilution chamber is emptied. This will occur at a cumulative volume of Vₚ mL. This will occur at the value of t when the infusion device has delivered Vₚ mL.

$$t = \frac{i}{{V}_{p}} ( {V}_{d} {e}^{- \frac{v}{{V}_{d}}} + v - {V}_{d} ) \\ t = \frac{100}{50} ( {V}_{d} {e}^{- \frac{{V}_{p}}{{V}_{d}}} + {V}_{p} - {V}_{d} ) \\ t = 80.13 min$$

This is 80.13-2.88 min=77.25 min after the second phase of the infusion method started.

The remainder of the infusion will occur at a constant (fluid) infusion rate (as the concentration in the dilution chamber will no longer change with time). This will be equal to the concentration of active agent in the dilution chamber divided by the maximum infusion rate. Therefore:

$$infusion rate = \frac{Max DoseRate}{{C}_{d}} \\ infusion rate = \frac{Max DoseRate}{{C}_{p} ( 1 - {e}^{- ( \frac{{V}_{p}}{{V}_{d}} )} )} \\ infusion rate = \frac{10}{20 \times ( 1 - {e}^{- \frac{50}{10}} )} \\ infusion rate = 0.5 mL / min$$

The dose given in the first 19.77 minutes is given by the Tansy Dose Function:

$$o s e = {C}_{p} \times ( \frac{2 {V}_{p}}{{2}^{16} - 2} {e}^{\frac{t}{2} ln ( {2}^{30 / i} )} - \frac{2 {V}_{p}}{{2}^{16} - 2} ) \\ Dose = 20 \times ( \frac{100}{65534} {e}^{\frac{19.77}{2} ln ( {2}^{30 / 30}} - \frac{100}{65534} ) \\ Dose = 28.8 mg$$

The remaining 971.2 mg is given at the maximum dosing rate of 10 mg/min over 91.1 minutes, giving a total infusion time of 116.9 minutes.

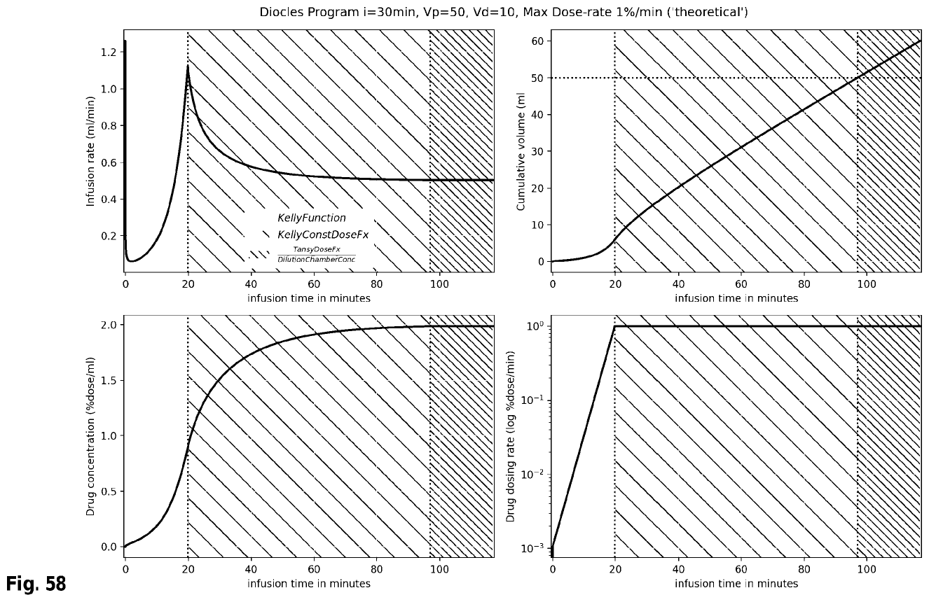

FIG. 58 is a illustrates a chart of an infusion rate over an infusion, a chart of a cumulative volume delivered over the infusion, a chart of a concentration of an active agent in a dilution chamber of a medication delivery apparatus over the infusion, and a drug administration rate for the above-described example infusion of Vancomycin, according to some embodiments. As the charts illustrate, a first portion of the infusion is controlled by the Kelly Cumulative Volume Function (no shading), a second portion is controlled by the Kelly Constant Dose Function (light shading) and a third portion is controlled by the dose function (dark shading).

### Example Timentin Infusion

An example infusion where timentin is the active agent is described. In some embodiments, the infusion involves delivery of a 4 g dose of ticarcillin with 500 mg of clavulanic acid. This may be dissolved in 50 ml of sterile water. In this case, Vₚ=50 mL, V_{d}=10 mL, i=30 mins. A maximum infusion rate may be 3.33% of the total dose per minute.

The example infusion may use the medication delivery apparatus 2 described with reference to FIGS. 30 to 34a. In this case, the method 5500 is used. The infusion computing device processor 256 uses the Tansy function to determine when the dose rate of 3.33%/min is reached.

$$t = \frac{2}{ln ( {2}^{30 / i} )} ln ( \frac{Max DoseRate}{{C}_{p}} \times \frac{{2}^{16} - 2}{{V}_{p} ln ( {2}^{30 / i} )} ) \\ t = \frac{2}{ln ( {2}^{30 / 30} )} ln ( \frac{3.33}{2} \times \frac{{2}^{16} - 2}{50 ln ( {2}^{30 / 30} )} ) \\ t = 2.8 \; 85 * ln ( 3151.5 \; 16 ) \\ t = 23.24 min$$

The infusion computing device processor 256 checks to see if the maximum dose rate is within the first time window or the second time window. To do so, the infusion computing device processor 256 determines the transitional time and compares this to the first time window and/or the second time window.

$$Transition time = \frac{ln ( {V}_{p} - {V}_{d} ( 1 - {e}^{- \frac{{V}_{p}}{{V}_{d}}} ) + \frac{2 {V}_{p}}{{2}^{16} - 2} ) - ln ( \frac{2 {V}_{p}}{{2}^{16} - 2} )}{\frac{1}{2} ln ( {2}^{30 / i} )} \\ Transition time = \frac{ln ( 50 - 10 ( 1 - {e}^{- \frac{50}{10}} ) + \frac{2 \times 50}{{2}^{16} - 2} ) - ln ( \frac{2 \times 50}{{2}^{16} - 2} )}{\frac{1}{2} ln ( 2 )} \\ Transition time = 29.36 minutes$$

As the transitional time is greater than the time at which the infusion delivers the 3.33%/min dose rate (i.e. the maximum dose rate), the maximum dose time occurs within the first time window.

The infusion computing device processor 256 therefore uses the Kelly Cumulative Volume Function to determine the cumulative volume administered to this point:

$$KV = {V}_{d} + {V}_{d} {W}_{0} ( - {e}^{- \frac{( ( {( {2}^{30 / i} )}^{t / 2} ) - 1 ) {V}_{p}}{32767 {V}_{d}} - 1} ) + \frac{{V}_{p} ( {( {2}^{30 / i} )}^{t / 2} ) - 1}{32767}$$

$$KV = 10 + 10 \times {W}_{0} ( - {e}^{\frac{( ( {( 2 )}^{23.24 / 2} ) - 1 ) \times 50}{32 \; 767 \times 10} - 1} ) + \frac{50 \times ( {( 2 )}^{23.24 / 2} ) - 1}{32767}$$

$$KV = 11.7 mL$$

The infusion computing device processor 256 uses the Kelly Constant Dose Cumulative Volume Function to determine when the same volume would be delivered if a constant flow algorithm was used. It should be noted that the constant algorithm will deliver the maximum dose rate allowed (in this case, 3.33%/min) and therefore the setting for i will be 30 minutes).

$${V}_{d} {e}^{- \frac{v}{{V}_{d}}} + v - {V}_{d} = \frac{{V}_{p}}{i} t$$

$$t = \frac{i}{{V}_{p}} ( {V}_{d} {e}^{- \frac{v}{{V}_{d}}} + v - {V}_{d} )$$

$$t = \frac{30}{50} * ( 10 {e}^{- \frac{11.7}{10}} + 11.7 - 10 )$$

$$t = 2.882 min$$

Thus, in this infusion, the Kelley Cumulative Delivery Volume Function is used for the first 23.24 minutes of the infusion, then, once the maximum infusion rate is achieved, the method will use the constant infusion algorithm until the active agent chamber is empty and the concentration of the active agent in the dilution chamber is a constant.

The constant infusion method will commence after 23.24 minutes, using the following settings: Vₚ=50, V_{d}=10, t=2.88, i=30.

The constant infusion method will continue until the volume of the dilution chamber is emptied. This will occur when Vₚ−11.7 mL have been delivered (38.3 mL). this will occur at the value of t when the constant infusion method has delivered Vₚ mL.

$$Δ t = \frac{i}{{V}_{p}} ( {V}_{d} {e}^{- \frac{v}{{V}_{d}}} + v - {V}_{d} ) - \frac{i}{{V}_{p}} ( {V}_{d} {e}^{- \frac{v}{{V}_{d}}} + v - {V}_{d} )$$

$$t = \frac{30}{50} ( {V}_{d} {e}^{- \frac{{V}_{p}}{{V}_{d}}} + {V}_{p} - {V}_{d} )$$

$$t = 24.04 min$$

This is 24.04−2.88 min=21.16 min after the constant infusion method started, at an elapsed overall infusion time of 44.4 minutes.

The remainder of the infusion will occur at a constant (fluid) infusion rate (as the concentration in the dilution chamber will no longer change with time). This will be equal to the concentration of active agent in the dilution chamber divided by the maximum infusion rate.

$$infusion rate = \frac{Max D o s e R a t e}{{C}_{d}}$$

$$infusion rate = \frac{Max D o s e R a t e}{{C}_{p} ( 1 - {e}^{- ( \frac{{V}_{p}}{{V}_{d}} )} )}$$

$$infusion rate = \frac{3.33}{2 \times ( 1 - {e}^{- \frac{50}{10}} )}$$

$$infusion rate = 1.678 mL / min$$

The dose given in the first 23.24 minutes is given by the Tansy Dose Function:

$$Dose = {C}_{p} \times ( \frac{2 {V}_{p}}{{2}^{16} - 2} {e}^{\frac{t}{2} ln ( {2}^{30 / i} )} - \frac{2 {V}_{p}}{{2}^{16} - 2} )$$

$$Dose = 2 \times ( \frac{100}{65534} {e}^{\frac{2324}{2} ln ( {2}^{30 / 30} )} - \frac{100}{65534} )$$

$$Dose = 9.6 \%$$

The remaining 90.4% is given at the maximum dose rate of 3.33%/min over 27.12 minutes, giving a total infusion time of 50.36 minutes. The infusion rate is 1.678 mL/min.

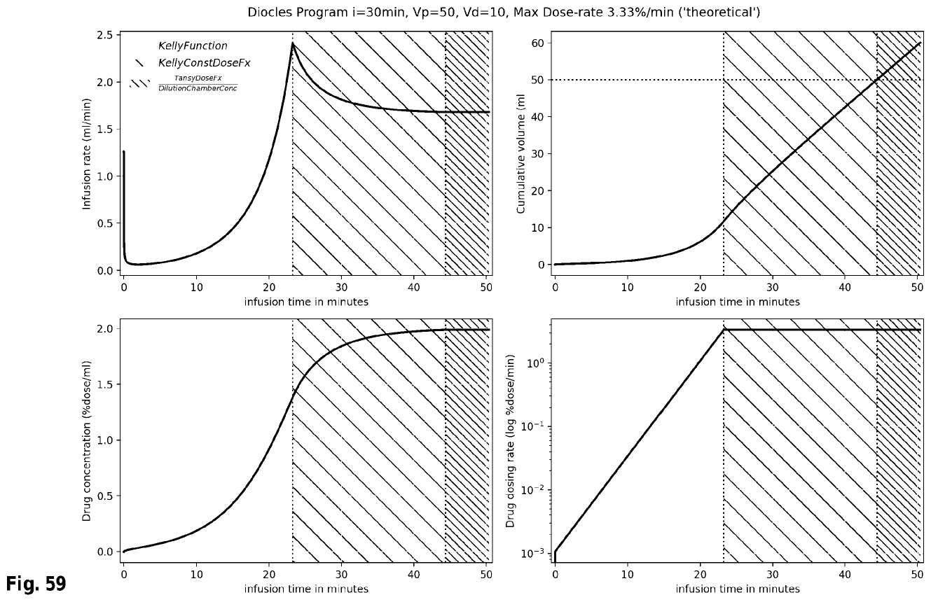

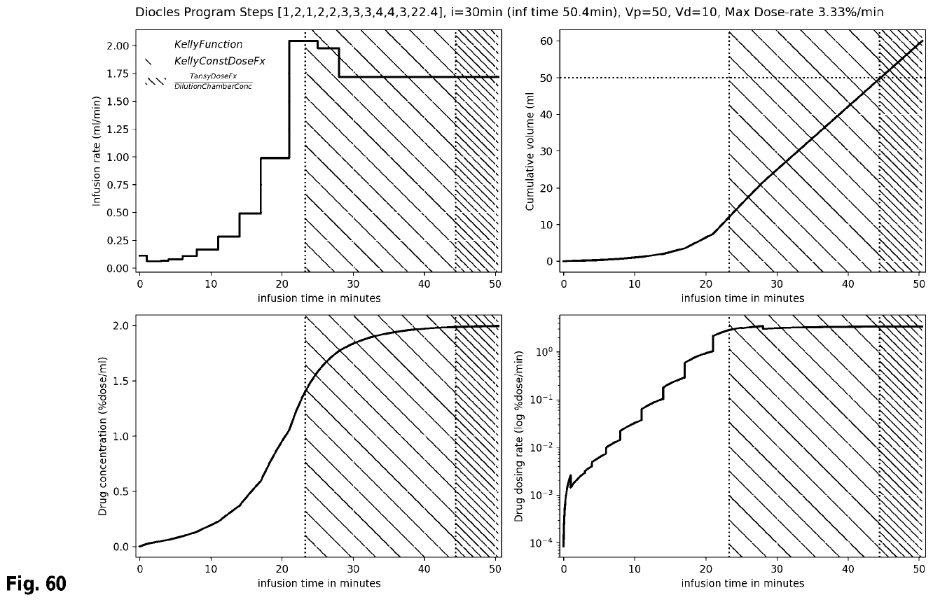

FIG. 59 illustrates a number of charts of the above example infusion. In particular, FIG. 59 illustrates an infusion rate over the infusion, a chart of a cumulative volume delivered over the infusion, a chart of a concentration of an active agent in a dilution chamber of a medication delivery apparatus over the infusion, and a drug administration rate for an example infusion, according to some embodiments. As the charts illustrate, a first portion of the infusion is controlled by the Kelly Cumulative Volume Function (no shading), a second portion is controlled by the Kelly Constant Dose Function (light shading) and a third portion is controlled by the dose function (dark shading).

This can be approximated by a program of constant-rate steps, for example, 12 steps. FIG. 60 illustrates a chart of an infusion rate over an infusion with 12 infusion steps, a chart of a cumulative volume delivered over the infusion, a chart of a concentration of an active agent in a dilution chamber of a medication delivery apparatus over the infusion, and a drug administration rate for an example infusion, according to some embodiments. The table below describes the duration of the infusion steps and the rates and volumes for the infusion.

| | Step | Step infusion | Step infusion |
|---|---|---|---|
| Step | duration (min) | rate (ml/min) | volume (ml) |
| 1 | 1 | 0.113 | 0.113 |
| 2 | 2 | 0.062 | 0.124 |
| 3 | 1 | 0.067 | 0.067 |
| 4 | 2 | 0.081 | 0.162 |
| 5 | 2 | 0.109 | 0.218 |
| 6 | 3 | 0.167 | 0.501 |
| 7 | 3 | 0.283 | 0.850 |
| 8 | 3 | 0.492 | 1.477 |
| 9 | 4 | 0.989 | 3.955 |
| 10 | 4 | 2.041 | 8.166 |
| 11 | 3 | 1.977 | 5.932 |
| 12 | 22.4 | 1.717 | 38.458 |

### Other Examples

As described herein, in some cases, a pharmaceutical preparation may be administered as a constant-rate infusion. That is, the rate that the cumulative dose increases as the infusion proceeds is constant. The present disclosure differs as the rate of the infusion is not constant. Similarly, the rate at which the cumulative dose of the active agent is delivered to the patient increases, rather than being constant. The rate the cumulative dose of the active agent increases as the infusion proceeds will increase as the infusion proceeds.

For example, if you deliver 100 mg over 30 minutes as a constant infusion you will deliver 0.1 mg after 0.03 minutes and 1 mg after 0.3 minutes, so the interval is 0.27/30 or 0.009 of the total infusion time. If you deliver 100 mg over 30 minutes as a constant infusion you will deliver 5 mg after 1.5 minutes and 50 mg after 15 minutes, so the interval is 13.5/30 or 0.45 of the total infusion time.

Using one or more of the methods disclosed herein, 0.1 mg can be delivered after 10.2 min and 1 mg after 16.7 minutes (an interval of 0.22 of the total infusion time). Therefore, a property of the disclosed methods is that orders of magnitude of cumulative dose are separated by a time interval that varies less than it does with a constant infusion. This would be a general property of any exponential function that controls cumulative dose. This is clinically important because any order of magnitude can be the difference between a mild and severe adverse reaction, so it is advantageous to separate all in time similarly.

Although the methods disclosed herein have been described as being performed by the infusion computing device 151, it will be understood that in some embodiments, one or more of the steps of one or more of the methods 5500, 5600, 5700 may be performed by another computing device or the infusion device 93. In these cases, the relevant determinations may be made by the other computing device and stored in memory of the computing device. Alternatively, the relevant determinations may be made by the infusion device processor 250 and stored in the infusion device memory 252. The relevant determinations may be stored as an infusion file. The infusion file may be provided (e.g. via a communications network) to the medication delivery system (e.g. the infusion device), to be performed, if done by another computing device. The infusion file may be in the form of an infusion program. The infusion program may be referred to herein as an infusion process. The infusion device may be configured to execute the infusion program to perform the methods disclosed herein.

Functions in which the dose rate doubles every b minutes, may be generally referred to as Tansy Functions. In some examples, a Tansy function may be expressed as an exponential dose rate function having an exponent of the form t/b, for example (t/b)*ln(2\hat{ }(30/i), where t represents the elapsed time, b is a constant and represents the time over which the dose rate doubles, and i is the duration of the infusion in minutes (“i” may also referred to as the “predetermined infusion time”). Dose profiles which have a dose rate defined by a Tansy functions may be particularly useful for controlling an infusion in order to detect an adverse reaction, perform or provocation test, or desensitization infusion.

An example of a Tansy function, in which the doubling time b is equal to 2 minutes, is given below.

$$infusion rate ( ml / min ) = \frac{{V}_{p} \times ln ( {2}^{30 / i} )}{{2}^{16} - 2} {e}^{\frac{t}{2} ln ( {2}^{30 / i} )} ,$$

where t=time elapsed, i=duration of the infusion and Vₚ=volume of the pharmaceutical preparation solution in a primary chamber.

$$dose rate ( mg / min ) = {C}_{p} \times \frac{{V}_{p} \times ln ( {2}^{30 / i} )}{{2}^{16} - 2} {e}^{\frac{t}{2} ln ( {2}^{30 / i} )} ,$$

where Cₚ is the concentration of the active ingredient of the pharmaceutical preparation solution in the primacy chamber

The Tansy cumulative volume function is the integral of the rate function and is:

$$cumulative volume ( ml ) = {\int }_{0}^{t} \frac{{V}_{p} \times ln ( {2}^{30 / i} )}{{2}^{16} - 2} {e}^{\frac{t}{2} ln ( {2}^{30 / i )}} dt$$

### Or:

$$cumulative volume ( ml ) = \frac{2 \times {V}_{p}}{{2}^{16} - 2} {e}^{\frac{t}{2} ln ( {2}^{30 / i} )} - \frac{2 \times {V}_{p}}{{2}^{16} - 2}$$

The Tansy cumulative dose function is thus:

$$cumulative dose ( mg ) = {C}_{p} \times ( \frac{2 \times {V}_{p}}{{2}^{16} - 2} {e}^{\frac{t}{2} ln ( {2}^{30 / i} )} - \frac{2 \times {V}_{p}}{{2}^{16} - 2} )$$

However, the above example of a Tansy function, is a special case of a more general series of equations (Tansy Functions) that are particularly suitable for controlling infusions in a manner suitable to detect an adverse reaction, or provocation test, or desensitization infusion.

The general form of the infusion rate Tansy function is:

$$infusion rate ( ml / min ) = \frac{a \times {V}_{p} \times \frac{ln ( {2}^{30 / i} )}{b}}{{2}^{16} - 2} {e}^{\frac{t}{b} ln ( {2}^{30 / i} ) ,}$$

where a and b are constants. The constant b expresses time taken for the rate to double. When i=30, b is equal to the number of minutes taken for the rate to double. That is, if i=30 and b=2, then it will take 2 minutes for the dose rate to double.

The general form of the dose rate Tansy function is:

$$dose rate ( mg / min ) = {C}_{p} \times \frac{a \times {V}_{p} \times \frac{ln ( {2}^{30 / i} )}{b}}{{2}^{16} - 2} {e}^{\frac{t}{b} ln ( {2}^{30 / i} )}$$

The general form of the cumulative dose function is:

$$cumulative dose ( mg ) = {C}_{p} \times ( \frac{a \times {V}_{p}}{{2}^{16} - 2} {e}^{\frac{t}{b} ln ( {2}^{30 / i} )} - \frac{a \times {V}_{p}}{{2}^{16} - 2} )$$

### Where:

$$a = \frac{{2}^{16} - 2}{{2}^{30 / b} - 1}$$

And the general form of the dose rate Tansy function can be simplified (as can the derived functions above be also in a similar way) to:

$$infusion rate ( mL / min ) = \frac{{V}_{p} \times ln ( {2}^{30 / i} )}{{2}^{30 / b} - 1} {e}^{\frac{t}{b} ln ( {2}^{30 / i} )}$$

The values of b can be chosen to alter the Order Magnitude Delay (OMD). The OMD refers to the period of time which it will take for the delivered cumulative dose to increase from the currently stated dose to 10 times the currently stated dose. For instance the OMD 0.01% is the time taken for the cumulative dose to increase from 0.01% of the therapeutic dose to 0.1% of the therapeutic dose (i.e. to increase by an order of magnitude). The OMD 0.1% is the time taken for the cumulative dose to increase from 0.1% of the therapeutic dose to 1% of the therapeutic dose. The OMD 0.3% is the time taken for the cumulative dose to increase from 0.3% of the therapeutic dose to 3% of the therapeutic dose etc. The OMD of a dose function may vary depending on the current cumulative dose, i.e. it may vary between earlier and later in the infusion.

The inventor has found that by changing the value of b, the OMD of the Tansy dose rate function can be changed. The inventor has further found that values of b in the range 1.5 to 4 provide useful dose profiles for the purposes of detecting an adverse reaction, performing a provocation test or desensitization. The inventor has found that, in general, higher values of b will result in longer OMD for higher cumulative doses (e.g. 1%) but at a cost of lower OMD for lower cumulative doses (e.g. 0.01%). That is, increasing b will decrease the time required to reach lower cumulative doses, but stretch out the time required to reach higher cumulative doses.

It will be appreciated that while the constant rate infusion function is not suitable for detecting adverse reactions at low dose rates or cumulative doses, the various Tansy functions with b between 1.5 and 4 are suitable for this purpose. It will further be appreciated that as varying b changes the characteristics of the function, certain values of b will be more suitable for certain patient populations or certain drugs, as for certain patient populations or drugs it will be better to have a larger OMD earlier in the infusion and for other patient populations or drugs it will be better to have a larger OMD later in the infusion.

For example, if a patient population has a distribution of drug provocation thresholds for a particular drug were 0.01% of the therapeutic dose is the minimum reactive threshold (minimum threshold at which an adverse reaction may occur), and 5 minutes is the latent period of the adverse reaction, b=2 may work well. This would ensure the OMD at 0.01% and greater is greater than 5 minutes.

For example, if a patient population has a distribution of drug provocation thresholds for a particular drug where 0.1% of the therapeutic dose is the minimum reactive threshold, and 5 minutes is the latent period of the adverse reaction, a dose profile approximating a Tansy function with b=3.5 may work well. This would ensure the OMD at 0.1% and greater is greater than 5 minutes.

For example, if a patient population has a distribution of drug provocation thresholds for a particular drug were 0.1% of the therapeutic dose is the minimum reactive threshold, and 7 minutes is the latent period of adverse reaction, a dose profile approximating a Tansy function in which b=3 may work well. This would ensure the OMD at 0.1% and greater is greater than 7 minutes.

For example, if a patient population has a distribution of drug provocation thresholds for a particular drug were 1% of the therapeutic dose is the minimum reactive threshold, and 10 minutes is the latent period of the drug reaction, a dose profile approximating a Tansy function in which b=4 may work well. This would ensure the OMD at 1% and greater is greater than 10 minutes.

The inventor envisages that Tansy functions with values of b between 2.25 and 4 or between 1.5 and 1.8 may be particularly useful and may in certain circumstances provide superior dose profiles for detecting adverse reactions to certain drugs compared to Tansy functions with a value of b of 2.

Various dose profiles, characterized in various different ways, have been described above. While delivering a drug to patient according to the above described dose profiles has various advantages for detecting adverse reactions, controlling a medication delivery apparatus to deliver such a dose profile is not a trivial matter. There are various practical difficulties in implementing such a system. One difficulty is translating the desired dose profile into a succession of flow rates which will deliver a desired dose profile.

Another difficulty is that infusion drivers may not be able to accurately deliver and control low infusion rates. For instance, a syringe driver, may not be able to accurately control a syringe at low infusion rates. However, as discussed above, the most important part of the dose profile is the early parts which have low dose rates.

Accordingly, in some examples, the medication delivery apparatus comprises an active agent chamber for receiving the pharmaceutical preparation and a dilution chamber for receiving pharmaceutical preparation ejected from the active agent chamber and diluting the pharmaceutical preparation with a diluent, the dilution chamber comprising an dilution chamber outlet for delivering the diluted pharmaceutical preparation to the patient. By diluting the pharmaceutical preparation in a dilution chamber, a relatively higher infusion rate may be used to deliver a relatively low dose rate. However, when this approach is used, the concentration of the pharmaceutical preparation may vary over the infusion process, which further complicates the calculation of appropriate flow rates to the patient as the varying concentration needs to be determined, or modelled, in advance and taken into account.

In some examples, the dilution chamber may have a volume of at least 10 ml or at least ⅕ of the volume of the activate agent chamber and the dilution chamber outlet may have a smaller area than a cross sectional area of the dilution chamber in the direction perpendicular the central axis of the dilution chamber.

The Tansy Functions discussed above are developed on the basis of an apparatus which delivers the pharmaceutical preparation directly from the active agent chamber to the patient. Functions have been developed which takes into account the complexity introduced by the dilution chamber and may be used to control an apparatus with a dilution chamber to deliver the same dose rate and dose profile as a Tansy Function. These functions are referred to generally as Sadleir Functions.

In some cases, the infusion device may be configured such that the dilution chamber has a fixed volume in a first portion of the predetermined infusion time and a variable volume in a second portion of the predetermined infusion time. This may example, enable the infusion driver to empty all of the drug (pharmaceutical preparation) out of the dilution chamber in the second time period after the drug chamber (active agent chamber) is empty, so that none of the drug is wasted. Functions generally referred to as Diocles Functions model this situation with first and second time periods and enable such an arrangement to deliver at a dose rate the same as a Tansy function in the first time period and a constant dose rate in the second time period.

The Tansy, Sadleir and Diocles functions discussed above are just examples and other cumulative volume functions and dose functions which deliver a desired dose profile may be used and applied to the techniques disclosed herein.

The processor of the infusion device may control the medication delivery apparatus to deliver the pharmaceutical preparation according to the predetermined profile by controlling an actuator of the infusion device. For example the actuator may controlled to drive a plunger, or a pump, of the medication delivery apparatus such that the pharmaceutical preparation is delivered according to the predetermined dose profile. For instance, the processor may divide the predetermined infusion time into a number of infusion steps and determine a target flow rate or a target output volume for each infusion step such that the predetermined dose profile is realized when the actuator is controlled according to the target flow rate or target output volume for each infusion step. The target flow rates or target output volumes for the infusion steps for a predetermined dose profile may be determined by referring to a lookup table stored in the memory. The lookup table may be populated according to the techniques described herein, for instance calculating the target flow rate or target output volume for each infusion step based on modelling of the predetermined dose profile. In other examples, target flow rate or target output volume for each infusion step may be calculated by the processor in real time

Modifications and variations as would be apparent to a skilled addressee are deemed to be within the scope of the present disclosure.

Further, it should be appreciated that the scope of the disclosure is not limited to the scope of the embodiments disclosed.

Throughout this specification, unless the context requires otherwise, the word “comprise” or variations such as “comprises” or “comprising”, will be understood to imply the inclusion of a stated integer or group of integers but not the exclusion of any other integer or group of integers.

1.-21. (canceled)

22. An infusion device for use with a medication delivery apparatus comprising an active agent chamber for receiving a pharmaceutical preparation, a dilution chamber for receiving a diluent and a dilution chamber opening through which diluted pharmaceutical preparation can be expelled for intravenous delivery to a patient; the infusion device comprising:
a processor and a memory storing instructions executable by the processor to cause the medication delivery apparatus to deliver the pharmaceutical preparation to the patient according to a dose profile, wherein the dose profile delivers a therapeutic dose of the pharmaceutical preparation to the patient over an infusion time, wherein the dose profile comprises a first stage and a second stage; and
wherein during the first stage, a concentration of the pharmaceutical preparation in the dilution chamber increases and a dose rate at which the pharmaceutical preparation is delivered to the patient increases until a maximum dose rate for the pharmaceutical preparation is reached;
wherein in the second stage, a concentration of the pharmaceutical preparation in the dilution chamber increases and a flow rate at which the diluted pharmaceutical preparation exits the dilution chamber decreases so that the maximum dose rate is not exceeded.

23. The infusion device of claim 22, wherein the dose rate in the second stage is constant.

24. The infusion device of claim 23, wherein the dose rate in the second stage is the maximum dose rate for the pharmaceutical preparation.

25. The infusion device of claim 22, wherein the dose profile further comprises a third stage in which the concentration of the pharmaceutical preparation in the dilution chamber is constant.

26. The infusion device of claim 25, wherein the dose rate in the third stage is constant and may, for example, be the maximum dose rate for the pharmaceutical preparation.

27. The infusion device of claim 22, wherein the dose profile is such that for at least a part of the first stage of the dose profile, a cumulative dose of pharmaceutical preparation delivered to the patient increases exponentially over time.

28. The infusion device of claim 22, wherein the first stage of the dose profile includes a first time period between the cumulative dose reaching 0.01% and 0.1% of the therapeutic dose and a second time period between the cumulative dose reaching 0.1% and 1% of the therapeutic dose; wherein the first period of time and the second period of time are selected from the group comprising: at least 6 minutes, at least 5 minutes, at least 4 minutes, at least 3 minutes between 2 minutes and 10 minutes, and at least the latent period of adverse reaction.

29. The infusion device of claim 22, wherein the medication delivery device comprises a container, a first plunger and a second plunger in the container arranged so that the active agent chamber is defined by a space between the first plunger and the second plunger and the dilution chamber is defined by a space between the second plunger and a distal end of the container; and wherein the first stage and second stage of the dose profile correspond to a first time window in which the first plunger is moved towards the second plunger so as to expel pharmaceutical preparation from the active agent chamber to the dilution chamber for mixing with diluent and output of diluted pharmaceutical preparation through the dilution chamber opening.

30. The infusion device of claim 25, wherein the third stage of the dose profile corresponds to a second time window in which the first plunger is in contact with the second plunger and the second plunger is moved towards the distal end of the container so as to reduce the volume of the dilution chamber and expel pharmaceutical preparation from the dilution chamber out through the dilution chamber opening.

31. The infusion device of claim 22, wherein the dilution chamber opening is to be attached to a conduit of predetermined volume through which diluted pharmaceutical preparation is delivered to the patient by intravenous infusion; and wherein:
the instructions stored in the memory and executable by the processor include priming instructions to prime the medication delivery device and the conduit of predetermined volume by controlling the medication delivery device to expel pharmaceutical preparation from the active agent chamber into the dilution chamber to mix with the diluent and flow out through the dilution chamber opening into the tubing of known volume so as to fill the conduit of predetermined volume with diluted pharmaceutical preparation in such a manner that the diluted pharmaceutical in the conduit of predetermined volume has a concentration profile in accordance with accordance with a desired dose profile for a first part of the intravenous infusion.

32. The infusion device of claim 31, wherein the concentration profile is such that a concentration of the diluted pharmaceutical preparation decreases along a length of the conduit of predetermined volume.

33.-35. (canceled)

36. The infusion device of claim 31, wherein an infusion rate used to prime the medication delivery apparatus and conduit of predetermined volume is higher than an initial infusion rate of the dose profile.

37. An infusion device for use with a medication delivery apparatus comprising a syringe including an active agent chamber for receiving a pharmaceutical preparation, a dilution chamber for receiving a diluent and a dilution chamber opening through which diluted pharmaceutical preparation can be expelled for intravenous delivery to a patient; the infusion device comprising:
a processor and a memory storing dose delivery instructions executable by the processor to cause the medication delivery apparatus to deliver the pharmaceutical preparation to the patient according to a dose profile which delivers a therapeutic dose of the pharmaceutical preparation to the patient over an infusion time in a manner that facilitates safe detection of an adverse reaction of the patient to the pharmaceutical preparation, or desensitization the patient to the pharmaceutical preparation;
and wherein the dose delivery instructions comprise instructions to cause the infusion device to move a plunger of the syringe towards the dilution chamber opening in a plurality of infusion steps which implement the dose profile, wherein a maximum infusion rate is reached after 50% of the infusion time has passed and for infusion steps taking place after a first 3% of the infusion time and prior to the maximum dose rate being reached, each infusion step has a higher dose rate than the previous infusion step.

38. The infusion device according to claim 37, wherein the infusion device is a pump, peristaltic pump, vacuum pump or a syringe driver.

39. An infusion device for controlling a medication delivery apparatus to deliver a pharmaceutical preparation to a patient, the infusion device comprising a processor and a memory storing instructions executable by the processor to:
determine, a number of infusion steps (h) that are to be performed within a time window, the time window comprising a first time window and a second time window, wherein a first number of infusion steps (h₁) are to be performed within the first time window and a second number of infusion steps (h₂) are to be performed within the second time window;
determine, for an infusion step of the first number of infusion steps (h₁), a first infusion volume, using a cumulative delivery volume function;
determine, for an infusion step of the second number of infusion steps (h₂), a second infusion volume, using a dose function;
control the medication delivery apparatus such that the first infusion volume of a fluid is expelled from the medication delivery apparatus during the infusion step of the first number of infusion steps (h₁); and
the second infusion volume of the fluid is expelled from the medication delivery apparatus during the infusion step of the second number of infusion steps (h₂).

40. The infusion device of claim 39, wherein a concentration of an active agent in the first infusion volume of the fluid that is to be expelled from the medication delivery apparatus is at least one order of magnitude lower than a concentration of the active agent in the second infusion volume of the fluid that is to be expelled from the medication delivery apparatus.

41. The infusion device of claim 39, wherein a rate of a cumulative dose of an active agent of the pharmaceutical preparation that is expelled from the medication delivery apparatus increases over the time window.

42. The infusion device of claim 39, wherein the cumulative delivery volume function is solved analytically to determine the first infusion volume.

43. The infusion device of claim 39, wherein the cumulative delivery volume function defines at least part of a dose profile for delivering a therapeutic dose of the pharmaceutical preparation to the patient, wherein the cumulative delivery volume function is such that the cumulative dose delivered to the patient increases exponentially, or increases at a rate that increases over time, over a time period between a first time at which 0.1% of the therapeutic dose has been delivered to the patient and a second time at which 10% of the therapeutic dose has been delivered to the patient.

44. The infusion device of claim 39, wherein the cumulative delivery volume function defines at least part of a dose profile for delivering a therapeutic dose of the pharmaceutical preparation to the patient, wherein the cumulative delivery volume function is such that there is a first time period between the cumulative dose reaching 0.01% and 0.1% of therapeutic dose and a second time period between the cumulative dose reaching 0.1% and 1% of the therapeutic dose; wherein the first period of time and the second period of time are selected from the group comprising: at least 6 minutes, at least 5 minutes, at least 4 minutes, at least 3 minutes between 2 minutes and 10 minutes, and at least the latent period of adverse reaction.
